(19) United States
(12) Patent Application Publication
(10) Pub. No.: US 2025/0213727 A1
(43) Pub. Date: Jul. 3, 2025

(54) DOMINANT-NEGATIVE TUMOR NECROSIS FACTOR ALPHA ADENO-ASSOCIATED VIRUS GENE THERAPY

(71) Applicant: Regenxbio Inc., Rockville, MD (US)

(72) Inventors: Danielle SLITER, Rockville, MD (US); Joseph BRUDER, Gaithersburg, MD (US); Devin MCDOUGALD, Boston, MA (US); Benjamin HEITHOFF, Rockville, MD (US)

(21) Appl. No.: 18/850,471

(22) PCT Filed: Mar. 24, 2023
(86) PCT No.: PCT/US2023/016312

§ 371 Date: Sep. 24, 2024

Related U.S. Application Data
(60) Provisional application No. 63/323,737, filed on Mar. 25, 2022.

(51) Int. Cl. A61K 48/00; A61P 29/00; C07K 14/525; C12N 15/86

(52) U.S. Cl. CPC A61K 48/0058; A61P 29/00; C07K 14/525; C12N 15/86; C12N 2750/14143; C12N 2750/14145

(57) ABSTRACT

The present disclosure provides AAV vector constructs that encode and deliver DN-TNFα polypeptides, compositions comprising the same and methods of their use in treating inflammatory conditions, such as neuroinflammatory conditions, systemic and peripheral inflammatory conditions, and ocular conditions. This disclosure also provides methods of making the AAV vector constructs that encode and deliver DN-TNFα polypeptides.

### CROSS-REFERENCE TO RELATED APPLICATIONS

This application claims the benefit of U.S. Provisional Patent Application No. 63/323,737, filed on Mar. 25, 2022, the contents of which are incorporated herein by reference in their entirety.

### TECHNICAL FIELD

This disclosure relates to dominant-negative Tumor Necrosis Factor alpha (DN-TNFα) therapy using viral vectors to treat various neuroinflammation, osteoarthritis, and ocular conditions, and related compositions and uses thereof. This disclosure also provides related polynucleotides and vectors encoding the DN-TNFα.

### BACKGROUND

Tumor necrosis factor (TNF) is a key regulator of inflammatory responses and has been implicated in many pathological conditions, including rheumatoid arthritis (RA) and cancer, which are associated with elevated serum levels of TNF (Steed P M, et al., Science. 2003 Sep. 26; 301 (5641): 1895-8; Dong Y et al., Antibodies 2015, 4 (4), 369-408). In addition, soluble TNFα has been implicated as a hallmark of acute and chronic neuroinflammation and is a key regulator of inflammatory responses in many neurodegenerative disorders, including Multiple Sclerosis (MS), Parkinson's disease (PD), Alzheimer's disease (AD), and Huntington's disease (HD) (Fatoba O. et al., Front. Immunol., 25 Feb. 2020; (11): Article 337). The several systemic and neurological conditions associated with elevated TNF are a significant burden for healthcare systems.

While anti-TNF agents have successfully been applied in several inflammatory diseases characterized by dysregulated TNF levels, such agents are associated with undesirable side effects and serious complications, (such as risk for opportunistic infections, reactivation of latent tuberculosis, and malignancy), paradoxical adverse events (such as worsening inflammatory processes, and demyelination in neurological diseases), prohibitive annual costs per patient, and a high number of patents that do not respond to therapy (Hsiao H Y et al., Human Molecular Genetics, 2014, Vol. 23, No. 16:4328-4344; Steeland S, et al., Int J Mol Sci. 2018 May 11; 19 (5): 1442; and Susanna F N and Pavesion C; J Ophthalmic Inflamm Infect. 2020; 10 (1): 11). Thus, there is a need for improved anti-TNF therapeutics which selectively target tissues to minimize undesirable systemic effects, and are safe and efficacious to use in inflammatory and neurological conditions.

Dominant negative TNFα is an anti-TNF biologic that forms heterotrimers with soluble TNF monomers and reduces the binding affinity of the resultant complex to TNF receptors (TNFRs), thereby diminishing proinflammatory signaling, without disturbing the signaling and function of transmembrane TNF (Hsiao H Y et al., Human Molecular Genetics, 2014, Vol. 23, No. 16:4328-4344; 2014, Steeland S, et al., Int J Mol Sci. 2018 May 11; 19 (5): 1442). Administered as a biologic molecule, DN-TNFα has shown efficacy in several models of Alzheimer's disease, Parkinson's disease, ischemia, spinal cord injury, multiple sclerosis, depression, and colitis, and may also be effective in indications where TNF inhibition is the standard of care like rheumatoid arthritis and non-infectious uveitis. However, as DN-TNFα is a recombinant protein, it requires continual, repetitive injections. There is a need to provide more effective DN-TNFα therapeutics via methods that sustain durability, selectively target tissues of interest, and improve delivery of the molecule in tissues (e.g., to improve blood-brain-barrier penetrance).

### SUMMARY

This disclosure relates to adeno-associated virus (AAV) constructs that encode and deliver DN-TNFα polypeptides and methods of their use. The DN-TNFα compositions described herein can be used to treat neuroinflammatory conditions (e.g., Alzheimer's disease), systemic and peripheral inflammation (e.g., rheumatoid arthritis), and ocular conditions (e.g., uveitis). This disclosure is based, at least in part, on the findings that novel adeno-associated viruses encoding a DN-TNFα transgene operably linked to a heterologous signal sequence and a promoter provide therapeutic expression levels in human cells, can be directly administered to the brain and can unexpectedly reduce the accumulation of amyloid beta in an Alzheimer's disease mouse model.

In one aspect, compositions are provided that include, comprise, or consist of a recombinant adeno-associated virus (rAAV) vector including: (a) an AAV capsid (b) an artificial genome comprising an expression cassette flanked by AAV Inverted Terminal Repeats (ITRs), wherein the expression cassette comprises a transgene encoding a dominant-negative tumor necrosis factor alpha (DN-TNFα) polypeptide, operably linked to a heterologous signal sequence, and one or more regulatory sequences that control expression of the transgene in human cells.

In some embodiments, the composition includes, comprises, or consists of a recombinant adeno-associated virus (rAAV) vector including: (a) an AAV capsid (b) an artificial genome comprising an expression cassette flanked by AAV Inverted Terminal Repeats (ITRs), wherein the expression cassette comprises a transgene encoding a dominant-negative tumor necrosis factor alpha (DN-TNFα) polypeptide, operably linked to a heterologous signal sequence, and one or more regulatory sequences that control expression of the transgene in human cells, with the proviso that the signal sequence is not the native TNF signal sequence or the signal sequence does not comprise a TACE recognition sequence.

In another aspect, this disclosure features pharmaceutical compositions for treating neuroinflammation in a human subject in need thereof, including or consisting of an adeno-associated virus (AAV) vector including or consisting of: (a) a viral capsid that is at least 95% (e.g., 96, 97, 98, 99, or 100%) identical to the amino acid sequence of AAV9 capsid (SEQ ID NO: 5); AAV.hDyn capsid (SEQ ID NO: 6); AAV.PHP.eB capsid (SEQ ID NO: 7); AAV.PHP.B capsid (SEQ ID NO: 8); AAV.PHP.S capsid (SEQ ID NO: 9); AAV.PHP.SH capsid (SEQ ID NO: 10); AAV8 capsid (SEQ ID NO: 11); AAV8.BBB capsid (SEQ ID NO: 12); AAV8.BBB.LD capsid (SEQ ID NO: 13); AAV9.BBB capsid (SEQ ID NO: 14); AAV9.BBB.LD capsid (SEQ ID NO: 15); AAVrh10 capsid (SEQ ID NO: 16); AAVrh.10.LD capsid (SEQ ID NO: 17); AAV9.496NNN/AAA498 capsid (SEQ ID NO: 18); VOY101 capsid (SEQ ID NO: 19); VOY201 capsid (SEQ ID NO: 20); VOY701 capsid (SEQ ID NO: 21); VOY801 capsid (SEQ ID NO: 22); VOY1101 capsid (SEQ ID NO: 23); or AAV.S454. Tfr3 (SEQ ID NO: 24); (b) an artificial genome comprising an expression cassette flanked by AAV Inverted Terminal Repeats (ITRs), wherein the expression cassette comprises a transgene encoding a dominant-negative tumor necrosis factor alpha (DN-TNFα) polypeptide, operably linked to a heterologous signal sequence, and one or more regulatory sequences that control expression of the transgene in human cells; wherein said AAV vector is formulated for administration to the subject.

In some embodiments, the one or more regulatory sequences are promoters selected from Table 7 or Table 8. In some embodiments, the one or more regulatory sequences are (a) a promoter selected from Table 7 or Table 8, and (b) a polyA selected from Rabbit β-globin polyA (SEQ ID NO: 109), and β-globin PolyA signal (SEQ ID NO: 110). In some embodiments, the expression cassette comprises the nucleotide sequence set forth in any one of SEQ ID NOs: 165-170.

In some embodiments, the neuroinflammation is associated with Alzheimer's Disease (AD), frontotemporal dementia (FD), tauopathies, progressive supranuclear palsy, chronic traumatic encephalopathy, Pick's Complex, primary age-related tauopathy, Huntington's Disease (HD), Juvenile Huntington's Disease, Parkinson's Disease (PD), synucleinopathies, Amyotrophic Lateral Sclerosis (ALS), migraines, cluster headaches, stroke, depression, post-traumatic stress disorder (PTSD), or traumatic brain injury (TBI).

In some embodiments, the transgene encoding the dominant-negative tumor necrosis factor alpha (DN-TNFα) polypeptide includes or consists of the codon optimized nucleotide sequence set forth in SEQ ID NO: 3. In some embodiments, the transgene encoding the dominant-negative tumor necrosis factor alpha (DN-TNFα) polypeptide includes or consists of the nucleotide sequence set forth in SEQ ID NO: 4.

In the above embodiments, the transgene encoding the DN-TNFα polypeptide is preceded by a nucleic acid sequence encoding a signal peptide. The signal peptide can be Mutant interleukin 2 (mIL2) signal peptide (SEQ ID NO: 115), VEGF-A signal peptide (SEQ ID NO: 118), Fibulin-1 signal peptide (SEQ ID NO: 119), vitronectin signal peptide (SEQ ID NO: 120), Complement Factor H signal peptide (SEQ ID NO: 121), opticin signal peptide (SEQ ID NO: 114), Albumin signal peptide (SEQ ID NO: 123), chymotrypsinogen signal peptide (SEQ ID NO: 124), Interleukin-2 signal peptide (SEQ ID NO: 125), and Trypsinogen-2 signal peptide (SEQ ID NO: 126).

In another aspect, the disclosure features pharmaceutical compositions for treating Alzheimer's Disease in a human subject in need thereof, including or consisting of an adeno-associated virus (AAV) vector comprising: (a) a viral capsid that is at least 95% identical to the amino acid sequence of AAV9 capsid (SEQ ID NO: 5); AAV.hDyn capsid (SEQ ID NO: 6); AAV.PHP.eB capsid (SEQ ID NO: 7); AAV.PHP.B capsid (SEQ ID NO: 8); AAV.PHP.S capsid (SEQ ID NO: 9); AAV.PHP.SH capsid (SEQ ID NO: 10); AAV8 capsid (SEQ ID NO: 11); AAV8.BBB capsid (SEQ ID NO: 12); AAV8.BBB.LD capsid (SEQ ID NO: 13); AAV9.BBB capsid (SEQ ID NO: 14); AAV9.BBB.LD capsid (SEQ ID NO: 15); AAVrh10 capsid (SEQ ID NO: 16); AAVrh.10.LD capsid (SEQ ID NO: 17); AAV9.496NNN/AAA498 capsid (SEQ ID NO: 18); VOY101 capsid (SEQ ID NO: 19); VOY201 capsid (SEQ ID NO: 20); VOY701 capsid (SEQ ID NO: 21); VOY801 capsid (SEQ ID NO: 22); VOY1101 capsid (SEQ ID NO: 23); or AAV.S454. Tfr3 (SEQ ID) NO: 24); and (b) an artificial genome comprising an expression cassette flanked by AAV Inverted Terminal Repeats (ITRs), wherein the expression cassette comprises a transgene encoding a dominant-negative tumor necrosis factor alpha (DN-TNFα) polypeptide, operably linked to a heterologous signal sequence, and one or more regulatory sequences that control expression of the transgene in human brain cells; wherein said AAV vector is formulated for administration to the subject.

In some embodiments, the regulatory sequence is one or more nucleic acid sequences selected from the group consisting of Chimeric Intron (SEQ ID NO: 107), VH4 Intron (SEQ ID NO: 108), Rabbit β-globin polyA (SEQ ID NO: 109), β-globin PolyA signal (SEQ ID NO: 110), and any of the promoters in Table 7 and Table 8.

In some embodiments, the ITRs are (a) a 5′ ITR selected from the group consisting of 5′ITR-A (SEQ ID NO: 111), and 5′-ITR-B (SEQ ID NO: 112); and (b) a 3′ ITR selected from 3′-ITR. AAV (SEQ ID NO: 113), and Alternative 3′-ITR (SEQ ID NO: 183). In some embodiments, the ITRs are selected from the ITR sequences described in Table 11 herein, or in Earley, L. et al. (Hum Gene Ther. February 2020; 31 (3-4): 151-162. doi: 10.1089/hum.2019. 274). 5′ITR sequences for ITR1, 2, 3, 4, and 7 are described in GenBank: ITR1: NC_002077.1, nucleotide sequence (nts) 1-143, ITR2: NC_001401.2, nts 1-145, ITR3: JB292182.1, nts 1-143, ITR4: NC_001829.1, nts 1-146, ITR7: NC_006260.1, nts 1-145. 3′ ITR sequences are the reverse complement of the corresponding 5′-ITR sequence.

In some embodiments, the DN-TNFα polypeptide is a variant sequence relative to the sequence encoding wild-type TNF-α polypeptide. In some embodiments, the DN-TNFα polypeptide has the amino acid substitution A145R and Y87H.

In another aspect, the disclosure features pharmaceutical compositions for delivering dominant-negative tumor necrosis factor alpha (DN-TNFα) to the brain to treat Alzheimer's Disease, prevent or inhibit the onset of AD, or reduce cognitive or functional decline in AD, in a human subject in need or at risk thereof, the compositions including or consisting of a recombinant AAV comprising or consisting of a transgene encoding DN-TNFα operably linked to a heterologous signal sequence, and one or more regulatory sequences that control expression of the transgene in brain cells, wherein the recombinant AAV is administered to the human subject at a dose of about 5×10¹² to about 2×10¹⁴ genome copies, e.g., 1×10¹³ to about 1×10¹⁴, or 5×10¹³ to about 1×10¹⁴, to the brain of the human subject.

In yet another aspect, the disclosure features methods of treating Alzheimer's Disease (AD), inhibiting the onset of AD, or reducing cognitive or functional decline in AD, in a human subject in need or at risk thereof, the methods including or consisting of administering a recombinant adeno-associated virus (AAV) vector comprising or consisting of a transgene encoding dominant-negative tumor necrosis factor alpha (DN-TNFα) to the brain of the subject, wherein the transgene encoding the DN-TNFα is operably linked to a heterologous signal sequence, and one or more regulatory sequences that control expression of the transgene in brain cells, wherein the recombinant AAV is administered to the human subject at a dose of about 5×10¹² to about 2×10¹⁴ genome copies to the brain of the human subject.

In some embodiments, the regulatory sequence in the AAV vector is one or more nucleic acid sequences selected from the group consisting of Chimeric Intron (SEQ ID NO: 107), VH4 Intron (SEQ ID NO: 108), Rabbit β-globin polyA (SEQ ID NO: 109), and β-globin PolyA signal (SEQ ID NO: 110), and any of the promoters in Table 7 and Table 8.

In some embodiments, the ITRs are (a) a 5′ ITR selected from the group consisting of S′ITR-A (SEQ ID NO: 111), and S′-ITR-B (SEQ ID NO: 112); and (b) a 3′ ITR selected from 3′-ITR AAV (SEQ ID NO: 113), and Alternative 3′-ITR. (SEQ ID NO: 183).

In the above embodiments, the transgene encoding the DN-TNFα polypeptide in the AAV vector is preceded by a nucleic acid sequence encoding a signal peptide. The signal peptide can be Mutant interleukin 2 (mIL2) signal peptide (SEQ ID NO: 115), VEGF-A signal peptide (SEQ ID NO: 118), Fibulin-1 signal peptide (SEQ ID NO: 119), Vitronectin signal peptide (SEQ ID NO: 120), Complement Factor H signal peptide (SEQ ID NO: 121), Opticin signal peptide (SEQ ID NO: 122), Albumin signal peptide (SEQ ID NO: 123), Chymotrypsinogen signal peptide (SEQ ID NO: 124), Interleukin-2 signal peptide (SEQ ID NO: 125), and Trypsinogen-2 signal peptide (SEQ ID NO: 126).

In some embodiments of the above methods, the administration of the recombinant AAV results in reduced levels of one or more of the following parameters: (a) AB accumulation; (b) amyloid plaques; (c) Tau accumulation; (d) neuroinflammation; (e) white matter free water (WMFW); and/or (f) one or more of CCL8, OLR1, IL2, CXCL9, TGFA, IL6, TNFSF12, CCL11, HGF, FLT3LG, IL17F, IL7, IL18, CCL13, TNFSF10, CXCL10, IFNG, IL10, 1L15, CCL3, CXCL8, MMP12, CSF2, VEGFA, IL17C, CCL2, IL17A, OSM, CSF1, CCL4, CXCL11, LTA, CCL7, and MMP1.

In some embodiments, the administration of the recombinant AAV results in enhanced cognitive function and/or increased microglial phagocytosis.

In some embodiments, the level(s) of one or more parameters in (a)-(f) is/are lower by at least 10%, as compared to corresponding reference level(s) in the subject or in a control.

In some embodiments, the subject's cognitive function is enhanced by at least about 10%, as measured on one or more tests selected from the group consisting of the Alzheimer's Disease Assessment Scale-cognitive subscale (ADAS-cog); clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB); the Sandoz Clinical Assessment-Geriatric (SCAG), the Buschke Selective Reminding Test; the Verbal Paired Associates subtest; the Logical Memory subtest: the Visual Reproduction subtest of the Wechsler Memory Scale-Revised (WMS-R); the explicit 3-alternative forced choice task; and the Benton Visual Retention Test.

In some embodiments, the subject is also treated with one or more agents selected from the group consisting of a cholinesterase inhibitor, an N-methyl-D-aspartate (NMDA) receptor antagonist, a hormone, a vitamin, an antipsychotic, a tricyclic antidepressant, a benzodiazepine, insulin, adeno-associated virus delivery of nerve growth factor (NGF), beta-blocker, human amyloid vaccine, beta or gamma secretase inhibitor, nicotinic or muscarinic agonist, and an antibody.

In some embodiments, a cognitive decline is assessed by determining the subject's score before and after administration of said AAV vector comprising the transgene encoding DN-TNFα using an Alzheimer's Disease Assessment Scale-Cognition (ADAS-Cog) test. In some embodiments, the reduction in cognitive decline as measured by ADAS-Cog is at least 10%, relative to a placebo.

In some embodiments, the subject has mild, moderate, or severe AD. In some embodiments, the treatment is prophylactic for completely or partially inhibiting or reducing AD or symptoms thereof in the subject. In some embodiments, the treatment is therapeutic for partially or completely curing AD or symptoms associated with AD in the subject. In some embodiments, the recombinant AAV is administered intravenously (IV), intraparenchymally, intracerebroventricularly (ICV), intracisternally (IC), or by lumbar intrathecal (IT) delivery. In some embodiments, the intraparenchymal administration is intrastriatal or intrahippocampal.

In another aspect, the disclosure features pharmaceutical compositions for delivering a dominant-negative tumor necrosis factor alpha (DN-TNFα) polypeptide to the brain of a human or animal subject in need thereof, including or consisting of administering an adeno-associated virus (AAV) vector including or consisting of: (a) an AAV viral capsid that transduces brain cells; and (b) an artificial genome comprising an expression cassette flanked by AAV Inverted Terminal Repeats (ITRs), wherein the expression cassette comprises a transgene encoding DN-TNFα, operably linked to a heterologous signal sequence, and a promoter that directs expression of the transgene in brain cells.

In some embodiments, the pharmaceutical composition for delivering a dominant-negative tumor necrosis factor alpha (DN-TNFα) polypeptide to the brain of a human or animal subject in need thereof, includes, comprises, or consists of administering an adeno-associated virus (AAV) vector including or consisting of: (a) an AAV viral capsid that transduces brain cells; and (b) an artificial genome comprising an expression cassette flanked by AAV Inverted Terminal Repeats (ITRs), wherein the expression cassette comprises a transgene encoding DN-TNFα, operably linked to a heterologous signal sequence, and a promoter, with the proviso that the signal sequence is not the native TNFα signal sequence or the signal sequence does not comprise a TACE recognition sequence.

In yet another aspect, the disclosure features methods of treating Alzheimer's Disease (AD), inhibiting the onset of AD, or reducing cognitive or functional decline in AD, in a human subject in need or at risk thereof, the method comprising delivering a dominant-negative tumor necrosis factor alpha (DN-TNFα) polypeptide to the brain of the human subject in need thereof, comprising administering an adeno-associated virus (AAV) vector comprising: (a) an AAV viral capsid that transduces brain cells; and (b) an artificial genome comprising an expression cassette flanked by AAV Inverted Terminal Repeats (ITRs), wherein the expression cassette comprises a transgene encoding a DN-TNFα, operably linked to a heterologous signal sequence, and a promoter that directs expression of the transgene in brain cells.

In some embodiments, the method of treating Alzheimer's Disease (AD), inhibiting the onset of AD, or reducing cognitive or functional decline in AD, in a human subject in need or at risk thereof, includes, comprises, or consists of delivering a DN-TNF polypeptide to the brain of the human subject in need thereof, comprising administering an adeno-associated virus (AAV) vector including, comprising, or consisting of: (a) an AAV viral capsid that transduces brain cells; and (b) an artificial genome comprising an expression cassette flanked by AAV Inverted Terminal Repeats (ITRs), wherein the expression cassette comprises a transgene encoding a DN-INFα, operably linked to a heterologous signal sequence, and a promoter, with the proviso that the signal sequence is not the native TNFα signal sequence or the signal sequence does not comprise a TACE recognition sequence.

In some embodiments, the administration of the AAV vector is intravenous, intracerebral, intraparenchymal, intracerebroventricular (ICV), intracisternal (IC), intraventricular, lumbar intrathecal (IT), or by a brain implant. In some embodiments, the intraparenchymal administration is intrastriatal or intrahippocampal.

In some embodiments, the promoter in the AAV vector is CAG (SEQ ID NO: 48); CB/CBA promoter (SEQ ID NO: 49); UbC promoter (SEQ ID NO: 50); mU1a (SEQ ID NO: 51); EF-1α (SEQ ID NO: 52); Human Synapsin Promoter 1 (hSyn-1; (SEQ ID NO: 53); Human Synapsin Promoter 2 (hSyn-2) (SEQ ID NO: 54); Human Synapsin Promoter 3 (hSyn-3) (SEQ ID NO: 55); Human Synapsin Promoter 4 (hSyn-4) (SEQ ID NO: 56); Human Synapsin Promoter 5 (hSyn-5) (SEQ ID NO: 57); Mecp2 promoter (SEQ ID NO: 58); hGFAP promoter (SEQ ID NO: 59); Rat NSE/RUS' promoter (SEQ ID NO: 60); NeuN (SEQ ID NO: 61); CaMKII_1 (SEQ ID NO: 62); C1ql2 1 (SEQ ID NO: 63); C1ql2 2 (SEQ ID NO: 64); DRD1 (SEQ ID NO: 65); DRD2 isoform 1 (SEQ ID NO: 66); DRD2 isoform 2 (SEQ ID NO: 67); POMC (SEQ ID NO: 68); PROX1 isoform 1 (SEQ ID NO: 69); PROX1 isoform 2 (SEQ ID NO: 70); MAP1B isoform 1 (SEQ ID NO: 71); MAP1B isoform 2 (SEQ ID NO: 72); MAP1B isoform 3 (SEQ ID NO: 73); or Ta-1/TUBA1A isoform 1 (SEQ ID NO: 74).

In another aspect, the disclosure features pharmaceutical compositions for treating Rheumatoid Arthritis in a human or animal subject in need thereof, including or consisting of an adeno-associated virus (AAV) vector including or consisting of: (a) a viral capsid that is at least 95% identical to the amino acid sequence of AAV8 capsid (SEQ ID NO: 11); AAV9 capsid (SEQ ID NO: 5); AAV.hu37 capsid (SEQ ID NO: 25); AAVrh74 version 1 capsid (SEQ ID NO: 26); AAVrh74 version 2 capsid (SEQ ID NO: 27); AAV.hu.31 capsid (SEQ ID NO: 28); or AAV.hu32 capsid (SEQ ID NO: 29); and (b) an artificial genome including or consisting of an expression cassette flanked by AAV Inverted Terminal Repeats (ITRs), wherein the expression cassette comprises a transgene encoding a dominant-negative tumor necrosis factor alpha (DN-TNFα) polypeptide, operably linked to a heterologous signal sequence, and one or more regulatory sequences that control expression of the transgene in human muscle, liver, and/or synovial cells; wherein said AAV vector is formulated for administration to the subject. In some embodiments, the transgene encoding the dominant-negative tumor necrosis factor alpha (DN-TNFα) polypeptide comprises the sequence set forth in SEQ ID NO: 3 or SEQ ID NO: 4.

In some embodiments, the regulatory sequence is one or more nucleic acid sequences selected from the group consisting of Chimerie Intron (SEQ ID NO: 107), VH4 Intron (SEQ ID NO: 108), Rabbit β-globin polyA (SEQ ID NO: 109), and β-globin PolyA signal (SEQ ID NO: 110), and any of the promoters in Table 7 and Table 9.

In some embodiments, the ITRs are (a) a 5′ ITR selected from 5′ITR-A (SEQ ID NO: 111), and 5′-ITR-B (SEQ ID NO: 112); and (b) a 3′ ITR selected from 3′-ITR AAV (SEQ ID NO: 113), and Alternative 3′-ITR (SEQ ID NO: 183).

In the above embodiments, the transgene encoding the DN-TNFα polypeptide is preceded by a nucleic acid sequence encoding a signal peptide. The signal peptide can be Mutant interleukin 2 (mIL2) signal peptide (SEQ ID NO: 115), VEGF-A signal peptide (SEQ ID NO: 118), Fibulin-1 signal peptide (SEQ ID NO: 119), vitronectin signal peptide (SEQ ID NO: 120), Complement Factor H signal peptide (SEQ ID NO: 121), opticin signal peptide (SEQ ID NO: 122), Albumin signal peptide (SEQ ID NO: 123), chymotrypsinogen signal peptide (SEQ ID NO: 124), Interleukin-2 signal peptide (SEQ ID NO: 125), and Trypsinogen-2 signal peptide (SEQ ID NO: 126).

In some embodiments, the DN-TNFα polypeptide is a variant sequence relative to the sequence encoding wild-type TNF-α polypeptide. In some embodiments, the DN-TNFα polypeptide has the amino acid substitution A145R and Y87H.

In yet another aspect, the disclosure features pharmaceutical compositions for delivering dominant-negative tumor necrosis factor alpha (DN-TNFα) to the human or animal subject to treat Rheumatoid Arthritis (RA), prevent or inhibit the onset of RA, or reduce inflammation in RA, in a human or animal subject in need or at risk thereof, the composition including a recombinant AAV including or consisting of a transgene encoding DN-TNFα operably linked to a heterologous signal sequence, and one or more regulatory sequences that control expression of the transgene in muscle, liver and/or synovial cells, wherein the recombinant AAV is administered to the human or animal subject at a dose of 1×10¹⁰ to 1×10¹⁶ genome copies.

In some embodiments, the pharmaceutical composition for delivering DN-TNF to the human or animal subject to treat RA, prevent or inhibit the onset of RA, or reduce inflammation in RA, in a human or animal subject in need or at risk thereof, includes or comprises the use of a recombinant AAV including or consisting of a transgene encoding DN-TNFα operably linked to a heterologous signal sequence, and one or more regulatory sequences, with the proviso that the signal sequence is not the native TNFα signal sequence or the signal sequence does not include or comprise a TACE recognition sequence.

In another aspect, the disclosure features methods of treating RA, preventing or inhibiting the onset of RA, or reducing inflammation in RA, in a human or animal subject in need or at risk thereof, the methods including or consisting of administering a recombinant adeno-associated virus (AAV) vector including or consisting of a transgene encoding dominant-negative tumor necrosis factor alpha (DN-TNFα) to the subject, wherein the transgene encoding the DN-TNFα is operably linked to a heterologous signal sequence, and one or more regulatory sequences that control expression of the transgene in muscle, liver, and/or synovial cells, wherein the recombinant AAV is administered to the human or animal subject at a dose of 1×10¹⁰ to 1×10¹⁶, e.g., 1×10¹¹ to 1×10¹⁵, 1×10¹² to 1×10¹⁴, or 5×10¹² to 5×10¹³, genome copies.

In some embodiments, the regulatory sequence in the AAV vector is one or more nucleic acid sequences selected from the group consisting of Chimeric Intron (SEQ ID NO: 107), VH4 Intron (SEQ ID NO: 108), Rabbit β-globin polyA (SEQ ID NO: 109), and β-globin PolyA signal (SEQ ID NO: 110), and any of the promoters in Table 7 and Table 9.

In some embodiments, the ITRs are derived from AAV2. In some embodiments, the ITRs are (a) a 5′ ITR selected from S′ITR-A (SEQ ID NO: 111), and 5′-ITR-B (SEQ ID NO: 112); and (b) a 3′ ITR selected from 3′-ITR AAV (SEQ ID NO: 113), and Alternative 3′-ITR (SEQ ID NO: 183).

In the above embodiments, the transgene encoding the DN-TNFα polypeptide is preceded by a nucleic acid sequence encoding a signal peptide. The signal peptide can be Mutant 118), Fibulin-1 signal peptide (SEQ ID NO: 119), vitronectin signal peptide (SEQ ID NO: 120), Complement Factor H signal peptide (SEQ ID NO: 121), opticin signal peptide (SEQ ID NO: 122), Albumin signal peptide (SEQ ID NO: 123), chymotrypsinogen signal peptide (SEQ ID NO: 124), Interleukin-2 signal peptide (SEQ ID NO: 125), and Trypsinogen-2 signal peptide (SEQ ID NO: 126).

In some embodiments, the administration of the recombinant AAV results in one or more of the following parameters: a reduction in the American College of Rheumatology (ACR) score, a reduction in the Disease Activity Score 28 (DAS28), a reduction in total joint score progression, a reduction in serum C-reactive protein (CRP) and a reduction in circulating soluble TNF receptors.

In some embodiments, the administration of the recombinant AAV results in an improvement in the Visual Analog Scale (VAS).

In some embodiments, the level(s) of one or more parameters is/are lower by at least 20%, as compared to corresponding reference level(s) in the subject or in a control.

In some embodiments, the subject is concurrently treated with one or more agents selected from the group consisting of A Disease Modifying Anti-Rheumatic Drug (DMARD) or a Nonsteroidal Anti-Inflammatory Drug (NSAID) and a steroid.

In some embodiments, the treatment is prophylactic for completely or partially inhibiting or reducing RA or symptoms thereof in the subject. In some embodiments, the treatment is therapeutic for partially or completely curing RA or symptoms associated with RA in the subject. In some embodiments, the recombinant AAV is administered by intravenous, intramuscular, intrasynovial, intra-articular, or peri-articular delivery.

In another aspect, the disclosure features a pharmaceutical composition for delivery of a dominant-negative tumor necrosis factor alpha (DN-TNFα) polypeptide in the muscle of a human subject in need thereof, comprising an adeno-associated virus (AAV) vector comprising: (a) an AAV viral capsid that infects muscle, liver and/or synovial cells; and (b) an artificial genome comprising an expression cassette flanked by AAV Inverted Terminal Repeats (ITRs), wherein the expression cassette comprises a transgene encoding a DN-TNFα, operably linked to a promoter that directs expression in muscle, liver and/or synovial cells.

In yet another aspect, the disclosure features methods of treating Rheumatoid arthritis (RA), preventing or inhibiting the onset of RA, or reducing inflammation in RA, in a human or animal subject in need or at risk thereof, the method comprising administering a dominant-negative tumor necrosis factor alpha (DN-TNFα) polypeptide in the muscle of the human or animal subject in need thereof, including or consisting of an adeno-associated virus (AAV) vector including or consisting of: (a) an AAV viral capsid that infects muscle, liver and/or synovial cells; and (b) an artificial genome comprising an expression cassette flanked by AAV Inverted Terminal Repeats (ITRs), wherein the expression cassette comprises a transgene encoding a DN-TNFα, operably linked to a promoter that directs expression in muscle, liver and/or synovial cells.

In some embodiments, the administration of the AAV vector is intravenous, intramuscular, intrasynovial, intra-articular, or peri-articular.

In some embodiments, the promoter in the AAV vector is CAG (SEQ ID NO: 48); CB/CBA promoter (SEQ ID NO: 49); UbC promoter (SEQ ID NO: 50); mU1a (SEQ ID NO: 51); EF-1α (SEQ ID NO: 52); LSPX1 (SEQ ID NO: 75); LSPX2 (SEQ ID NO: 76); LTP1 (SEQ ID NO: 77); LTP2 (SEQ ID NO: 78); LTP3 (SEQ ID NO: 79); LMTP6 (SEQ ID NO: 80); LMTP13 (SEQ ID NO: 81); LMTP14 (SEQ ID NO: 82); LMTP15 (SEQ ID NO: 83); LMTP18 (SEQ ID NO: 84); LMTP19 (SEQ ID NO: 85); LMTP20 (SEQ ID NO: 86); LBTP1 (SEQ ID NO: 87); LBTP2 (SEQ ID NO: 88); hAAT (SEQ ID NO: 89); ApoE.hAAT (SEQ ID NO: 90); TBG (SEQ ID NO: 91); CK8 (SEQ ID NO: 92); SPe5-12 (SEQ ID NO: 93); MCK7 (SEQ ID NO: 94); truncatedMCK (IMCK) (SEQ ID NO: 95); Mouse skeletal muscle alpha actin acta1 (SEQ ID NO: 96); Human muscle creatine kinase (MCK) (SEQ ID NO: 97); Human desmin (SEQ ID NO: 98); Human desmin 2 (SEQ ID NO: 99); Human skeletal muscle alpha actin acta1 (SEQ ID NO: 100); Mouse muscle creatine kinase (MCK) (SEQ ID NO: 101); Mouse desmin (SEQ ID NO: 102); or CXCL10 (SEQ ID NO: 103).

In another aspect, the disclosure features pharmaceutical compositions for treating uveitis in a human or animal subject in need thereof, including or consisting of an adeno-associated virus (AAV) vector including or consisting of: (a) a viral capsid that is at least 95% identical to the amino acid sequence of AAV8 capsid (SEQ ID NO: 11); AAV9 capsid (SEQ ID NO: 5); AAV2 capsid (SEQ ID NO: 30); AAV3B capsid (SEQ ID NO: 31); AAV2.7m8 capsid (SEQ ID NO: 32); AAV.rh.34 capsid (SEQ ID NO: 33); AAV.hu.31 capsid (SEQ ID NO: 28); AAV.rh.31 capsid (SEQ ID NO: 34); AAV.hu.12 capsid (SEQ ID NO: 35); AAV.hu.13 capsid (SEQ ID NO: 36); AAV.hu.21 capsid (SEQ ID NO: 37); AAV.hu.26 capsid (SEQ ID NO: 38); AAV.hu.53 capsid (SEQ ID NO: 39); AAV.hu.56 capsid (SEQ ID NO: 40); AAV.rh.24 capsid (SEQ ID NO: 41); AAV.hu.38 capsid (SEQ ID NO: 42); AAV.rh.72 capsid (SEQ ID NO: 43); AAV.cy.5 capsid (SEQ ID NO: 44); AAV.cy.6 capsid (SEQ ID NO: 45); AAV.rh.46 capsid (SEQ ID NO: 46); or AAV.rh.2 capsid (SEQ ID NO: 47); and (b) an artificial genome including or consisting of an expression cassette flanked by AAV Inverted Terminal Repeats (ITRs), wherein the expression cassette comprises a transgene encoding a dominant-negative tumor necrosis factor alpha (DN-TNFα) polypeptide, operably linked to a heterologous signal sequence, and one or more regulatory sequences that control expression of the transgene in human ocular cells, retinal pigment epithelial cells, and/or retinal cells; wherein said AAV vector is formulated for administration to the subject.

In some embodiments, the transgene encoding the dominant-negative tumor necrosis factor alpha (DN-TNFα) polypeptide comprises the sequence set forth in SEQ ID NO: 3 or SEQ ID NO: 4.

In some embodiments, the regulatory sequence is one or more nucleic acid sequences selected from the group consisting of Chimeric Intron (SEQ ID NO: 107), VH4 Intron (SEQ ID NO: 108), Rabbit β-globin polyA (SEQ ID NO: 109), and β-globin PolyA signal (SEQ ID NO: 110), and any of the promoters in Table 7 and Table 10.

In some embodiments, the ITRs are (a) a 5′ ITR selected from 5′ITR-A (SEQ ID NO: 111), and 5′-ITR-B (SEQ ID NO: 112); and (b) a 3′ ITR selected from 3′-ITR AAV (SEQ ID NO: 113), and Alternative 3′-ITR (SEQ ID NO: 183).

In the above embodiments, the transgene encoding the DN-TNFα polypeptide is preceded by a nucleic acid sequence encoding a signal peptide. The signal peptide can be Mutant interleukin 2 (mIL2) signal peptide (SEQ ID NO: 115), VEGF-A signal peptide (SEQ ID NO: 118), Fibulin-1 signal peptide (SEQ ID NO: 119), Vitronectin signal peptide (SEQ ID NO: 120), Complement Factor H signal peptide (SEQ ID NO: 121), Opticin signal peptide (SEQ ID NO: 122), Albumin signal peptide (SEQ ID NO: 123), Chymotrypsinogen signal peptide (SEQ ID NO: 124), Interleukin-2 signal peptide (SEQ ID NO: 125), and Trypsinogen-2 signal peptide (SEQ ID NO: 126).

In some embodiments, the DN-TNFα polypeptide is a variant sequence relative to the sequence encoding wild-type TNF-α polypeptide. In some embodiments, the DN-TNFα polypeptide has the amino acid substitution A145R and Y87H.

In yet another aspect, the disclosure features pharmaceutical compositions for delivering dominant-negative tumor necrosis factor alpha (DN-TNFα) to human or animal ocular cells, retinal pigment epithelial cells, and/or retinal cells to treat uveitis, prevent or inhibit the onset of uveitis, or reduce inflammation in uveitis, in a human or animal subject in need or at risk thereof, the composition including or consisting of a recombinant AAV including or consisting of a transgene encoding DN-TNFα operably linked to a heterologous signal sequence, and one or more regulatory sequences that control expression of the transgene in human ocular cells, retinal pigment epithelial cells, and/or retinal cells, wherein the recombinant AAV is administered to the human subject at a dose of about 2×10¹⁰ GC to about 6×10¹⁰ GC per eye, e.g., 3×10¹⁰ GC to about 5×10¹⁰ GC per eye.

In another aspect, the disclosure features methods of treating uveitis, preventing or inhibiting the onset of uveitis, or reducing inflammation in uveitis, in a human or animal subject in need or at risk thereof, the methods including or consisting of administering a recombinant adeno-associated virus (AAV) vector including or consisting a transgene encoding dominant-negative tumor necrosis factor alpha (DN-TNFα) to the human or animal ocular cells, retinal pigment epithelial cells, and/or retinal cells of the subject, wherein the transgene encoding the DN-TNFα is operably linked to a heterologous signal sequence, and one or more regulatory sequences that control expression of the transgene in human or animal ocular cells, retinal pigment epithelial cells, and/or retinal cells, wherein the recombinant AAV is administered to the human or animal subject at a dose of about 2×10¹⁰ GC to about 6×10¹⁰ GC per eye.

In some embodiments, the regulatory sequence in the AAV vector is one or more nucleic acid sequences selected from the group consisting of Chimeric Intron (SEQ ID NO: 107), VH4 Intron (SEQ ID NO: 108), Rabbit β-globin polyA (SEQ ID NO: 109), and β-globin PolyA signal (SEQ ID NO: 110), and any of the promoters in Table 7 and Table 10.

In some embodiments, the ITRs are (a) a 5′ ITR selected from 5′ITR-A (SEQ ID NO: 111), and 5′-ITR-B (SEQ ID NO: 112); and (b) a 3′ ITR selected from 3′-ITR. AAV (SEQ ID NO: 113), and Alternative 3′-ITR (SEQ ID NO: 183).

In the above embodiments, the transgene encoding the DN-TNFα polypeptide is preceded by a nucleic acid sequence encoding a signal peptide. The signal peptide can be Mutant 118), Fibulin-1 signal peptide (SEQ ID NO: 119), Vitronectin signal peptide (SEQ ID NO: 120), Complement Factor H signal peptide (SEQ ID NO: 121), Opticin signal peptide (SEQ ID NO: 122), Albumin signal peptide (SEQ ID NO: 123), Chymotrypsinogen signal peptide (SEQ ID NO: 124), Interleukin-2 signal peptide (SEQ ID NO: 125), and Trypsinogen-2 signal peptide (SEQ ID NO: 126).

In general, the signal sequence is not a native tumor necrosis factor alpha (TNFα) signal sequence. In some embodiments, the signal sequence does not comprise a TNF-alpha-converting enzyme (TACE) recognition sequence.

In some embodiments, the administration of the recombinant AAV results in one or more of the following parameters: reduction of visual haze, decrease of inflammatory lesions, decrease in tissue destruction, decrease in biomarkers of autoimmunity and/or inflammation, decrease in vasculitis, decrease in cellular infiltration, or decrease in edema.

In some embodiments, the administration of the recombinant AAV results in improvement in clinical symptoms of uveitis and/or improvement of vision. In some embodiments, the level(s) of one or more parameters is/are lower by at least 20%, as compared to corresponding reference level(s) in the subject or in a control.

In some embodiments, the vision is enhanced by at least about 20%, as measured on one or more tests selected from the group consisting of Applanation Tonometry, Corneal Topography, Fluorescein Angiogram, Dilated Pupillary Exam, Refraction, Slit-Lamp Exam, Non-Contact Tonometry, Retinal Tomography, Ultrasound, Visual Acuity Testing, and Visual Field Test.

In some embodiments, the subject is concurrently treated with one or more agents selected from the group consisting of an anti-inflammatory agent, an anti-fungal agent, and an immunosuppressive agent, a corticosteroid, an A3 adenosine receptor selective agonist), corticotropin zinc hydroxide, cyclopentolate, cyclosporine, cyclosporine A, dexchlorpheniramine, LFG-316 (anti-C5), homatropine, hyoscyamine sulfate, phenylephrine, an anti-IL-6R monoclonal antibody), an anti-IL-17A monoclonal antibody, an mTOR inhibitor, an IL-1 beta antagonist, an anti-TNF monoclonal antibody, a muscarinic receptor antagonist, methotrexate, azathioprine, acyclovir, gentamycin, neomycin, polymyxin B, rolitetracycline, sulfacetamide, valacyclovir, chloramphenicol, mycophenolate, fluocinolone, neomycin, polymyxin B, prednisolone and sulfacetamide.

In some embodiments, the uveitis is anterior uveitis, intermediate uveitis and/or posterior uveitis. In some embodiments, the treatment is prophylactic for completely or partially preventing uveitis or symptoms thereof in the subject. In some embodiments, the treatment is therapeutic for partially or completely curing uveitis or symptoms associated with uveitis in the subject. In some embodiments, the recombinant AAV is administered by subretinal, intravitreal, suprachoroidal, or intracameral delivery.

In another aspect, the disclosure features pharmaceutical compositions for delivery of a dominant-negative tumor necrosis factor alpha (DN-TNFα) polypeptide into human or animal ocular cells, retinal pigment epithelial cells, and/or retinal cells of a human or animal subject in need thereof, including or consisting of an adeno-associated virus (AAV) vector comprising: (a) an AAV viral capsid that infects human or animal ocular cells, retinal pigment epithelial cells, and/or retinal cells; and (b) an artificial genome comprising an expression cassette flanked by AAV Inverted Terminal Repeats (ITRs), wherein the expression cassette includes or consists of a transgene encoding a DN-TNFα, operably linked to a promoter that directs expression in human or animal ocular cells, retinal pigment epithelial cells, and/or retinal cells.

In yet another aspect, the disclosure features methods of treating uveitis, inhibiting or preventing the onset of uveitis, or reducing inflammation in uveitis, in a human or animal subject in need or at risk thereof, the methods including or consisting of administering a dominant-negative tumor necrosis factor alpha (DN-TNFα) polypeptide in the eye of the human or animal subject in need thereof, including or consisting of an adeno-associated virus (AAV) vector comprising: (a) an AAV viral capsid that infects human or animal ocular cells, retinal pigment epithelial cells, and/or retinal cells; and (b) an artificial genome including or consisting of an expression cassette flanked by AAV Inverted Terminal Repeats (ITRs), wherein the expression cassette comprises a transgene encoding a DN-TNFα, operably linked to a promoter that directs expression in human or animal ocular cells, retinal pigment epithelial cells, and/or retinal cells.

In some embodiments, the administration of the AAV vector is subretinal, intravitreal, suprachoroidal, or intracameral. In some embodiments, the promoter in the AAV vector is CAG (SEQ ID NO: 48); CB/CBA promoter (SEQ ID NO: 49); UbC promoter (SEQ ID NO: 50); mU1a promoter (SEQ ID NO: 51); EF-1α promoter (SEQ ID NO: 52); RPE65 promoter (SEQ ID NO: 104); red cone opsin promoter (SEQ ID NO: 105); or BST1 promoter (SEQ ID NO: 106).

In some embodiments of any of the pharmaceutical compositions and methods disclosed herein, the heterologous signal sequence controls the secretion of the DN-TNFα polypeptide, e.g., to the extracellular space, such as through the Endoplasmic Reticulum.

In other embodiments, a method of inducing pro-inflammatory and/or pro-survival TNF receptor signaling in a human tissue is provided, the method comprising: delivering dominant-negative tumor necrosis factor alpha (DN-TNFα) polypeptide, operably linked to a heterologous signal sequence, and one or more regulatory sequences that control expression of the DN-TNFα in human cells by administration of a recombinant adeno-associated virus (rAAV) vector comprising:
(a) an rAAV capsid and
(b) an artificial genome comprising an expression cassette flanked by AAV Inverted Terminal Repeats (ITRs), wherein the expression cassette comprises a transgene encoding the dominant-negative tumor necrosis factor alpha (DN-TNFα) polypeptide operably linked to the heterologous signal sequence and the one or more regulatory sequences.

In more embodiments, a method of treating Duchenne muscular dystrophy (DMD), or reducing inflammation associated with DMD, in a human subject in need or at risk thereof, is provided, the method comprising delivering a dominant-negative tumor necrosis factor alpha (DN-TNFα) polypeptide in a muscle of the human subject in need thereof, comprising administering an adeno-associated virus (AAV) vector comprising:
(a) an AAV viral capsid that infects human muscle cells; and
(b) an artificial genome comprising an expression cassette flanked by AAV Inverted Terminal Repeats (ITRs), wherein the expression cassette comprises a transgene encoding a DN-TNFα, operably linked to a heterologous signal sequence and a promoter that directs expression in human muscle. In another embodiment, the method further comprises administering to the human subject an agent capable of restoring a functional fragment of dystrophin, such as AAV-microdystrophin or an exon-skipping therapy.

The novel compositions and methods of this disclosure have several advantages. First, the compositions facilitate targeted delivery of nucleic acid compositions encoding a recombinant polypeptide (DN-TNFα) result in expression of DN-TNFα in tissues that are difficult to reach, such as the brain, due to the increased penetrance of the disclosed AAV compositions across the blood-brain barrier. Second, because the polypeptide therapeutic is expressed from a viral vector (i.e., an AAV vector), it facilitates direct administration of the disclosed nucleic acid compositions into the brain, peripheral tissue (such as muscle, liver, synovial tissue), and tissues in the eye. Third, once delivered, the compositions allow for continual production of the DN-TNFα at the target site (e.g., specific areas of the brain) over a prolonged period of time. Thus, the compositions and methods of the disclosure require fewer injections, thereby eliminating the need for continual repetitive injections, and result in greater therapeutic efficacy, fewer delivery-related side-effects, and lesser generation of anti-AAV antibodies. To date, there is no effective viral vector delivered DN-TNFα treatment in the clinic to treat neuroinflammatory conditions (such as Alzheimer's disease), systemic conditions (such as rheumatoid arthritis), or ocular conditions (such as uveitis).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic illustration of the DN-TNFα construct containing nucleic acid that is inserted into the DNTNF.001 cis plasmid. The DNTNF.001 AAV vector plasmid contains the CAG promoter. This is followed by a codon-optimized and CpG-depleted nucleic acid sequence encoding the Dominant-negative tumor necrosis factor alpha (DN-TNFα) construct and a poly A tail. ITR: inverted terminal repeats.

FIG. 2 is a schematic illustration of the DN-TNFα construct containing nucleic acid that is inserted into the DNTNF.002 cis plasmid. The DNTNF.002 AAV vector plasmid contains the human Synapsin promoter (hSyn), followed by the human immunoglobin heavy chain variable region (Vh4) intron (NCBI Accession No.: AB019438 Region: 5902 to 5983), the optimized DN-TNFα construct and a polyA tail. ITR: inverted terminal repeats. ITRm: mutant ITR. δD: the D sequence and the terminal resolution site (trs) were deleted in the mutant ITR to enable production of a self-complementary AAV (scAAV).

FIG. 3 is a bar graph that shows expression of human DN-TNFα (pg/ml) as measured by ELISA, 18 weeks following direct injection of various AAV vectors into the brains of 5XFAD transgenic mice. Viral vector doses of 2 μl 1e10 vg were intraparenchymally injected bilaterally into the subiculum of 6 month old mice. Mice with ID nos. 1995-1997 received AAV9.CAG.GFP (control). Mice with ID nos. 1998-2000 received AAV9.CAG.DN-TNFα (AAV9.DNTNF.001). The numbers 1-6 above each bar correspond to the tissues indicated in the figure legend.

FIGS. 4A-4D are a series of representative microscope images that show amyloid beta expression in brain slices of mice immunostained with anti-amyloid precursor protein. Amyloid beta levels were quantified using ImageJ.

FIG. 4E is a bar graph that shows quantification of amyloid beta positive aggregates in the subiculum in 6 month old 5XFAD mice.

FIG. 5 is a bar graph that shows expression of AAV-delivered human DN-TNFα (pg/ml) as measured by ELISA in various tissues and organs, 4 weeks following unilateral intracerebroventricular (ICV) injection of AAV9.CAG.DN-TNF (AAV9.DNTNF001) into the right hemispheres of the brains of 6 month old 5XFAD transgenic mice. Viral vector doses of 5 μl 1e10 or 1e11 vg were used. The numbers 1-7 above each bar correspond to the tissues indicated in the figure legend.

FIG. 6 is a Clustal Multiple Sequence Alignment of AAV capsids 1-9. Amino acid substitutions (shown in bold in the bottom rows) can be made to AAV9 and AAV8 capsids by “recruiting” amino acid residues from the corresponding position of other aligned AAV capsids. The various hypervariable regions (HVR) s HVR1-HVR12 are indicated by boxes around the HVRs in the sequences. The amino acid sequences of the AAV capsids are assigned SEQ ID NOs as follows: AAV1 is SEQ ID NO: 177; AAV2 is SEQ ID NO: 30; AAV3-3 (also known as AAV3A) is SEQ ID NO: 178; AAV4-4 (also known as AAV4) is SEQ ID NO: 179; AAV5 is SEQ ID NO: 180; AAV6 is SEQ ID NO: 181; AAV7 is SEQ ID NO: 182; AAV8 is SEQ ID NO: 11; AAV9 is SEQ ID NO: 5; AAVrh10 is SEQ ID NO: 16; hu31 is SEQ ID NO: 28; and hu32 is SEQ ID NO: 29.

FIGS. 7A-7D are a series of representative microscope images that show the puncta in the subiculum area following anti-AB42 staining.

FIG. 8 is a bar graph that illustrates the overall percentage of the subiculum area having puncta (AB42 plaques 4.65% of the total subiculum area) in 6 month old 5XFAD mice treated with control (injection of AAV-GFP control vector at 2 months) compared to the number of plaques in the subiculum area (1.54% of subiculum area) four months following a single administration of AAV-CAG.DN-TNFα vector to the hippocampus.

### DETAILED DESCRIPTION

The present disclosure provides compositions and methods for the delivery of DN-TNFα to a human subject diagnosed with a disease or condition indicated for treatment with therapeutic DN-TNFα. This disclosure is based, at least in part, on the surprising findings that direct injection of an AAV9-delivered human DN-TNFα transgene in the brain of transgenic mice overexpressing human amyloid beta (ABeta) precursor protein results in long-term expression of human DN-TNFα protein and subsequent reduced accumulation of ABeta aggregates.

This disclosure also provides polypeptides, polynucleotides, vectors, and compositions comprising the DN-TNFα, methods of making the compositions, and methods of delivering and using the compositions.

This disclosure is based on the findings that AAV-DN-TNFα can be successfully delivered to various tissues, such as brain tissue, muscle tissue, and eye tissue, providing therapeutic expression of the DN-TNFα transgene that is operably linked to a heterologous secretory signal sequence. Furthermore, long-term expression of DN-TNFα can be maintained in the specific tissues without the need for repeated administration of the AAV vector. Lastly, administration of AAV-DN-TNFα to the specific tissues reduces inflammation and is associated with improvements in clinical symptoms of various diseases (e.g., improvements in cognitive function in Alzheimer's Disease, improved clinical symptoms of uveitis and/or improvement of vision, improvements in disease scores and/or joint inflammation in Rheumatoid Arthritis), improvements in pathological manifestations of the disease (e.g., reduced accumulation of amyloid beta plaques in Alzheimer's Disease), and/or reductions in inflammatory cytokines in inflammatory diseases.

### Definitions

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “vector” refers to a macromolecule or association of genetic elements that comprises or associates with a polynucleotide and which can be used to mediate delivery of the polynucleotide to a cell. Examples of vectors include a plasmid, phage, transposon, cosmid, chromosome, virus, virion, etc. In the context of this disclosure, the term includes viral vectors, cloning vehicles, and expression vehicles. The vector generally comprises genetic elements, e.g., regulatory elements, operatively linked to a gene to facilitate expression of the gene in a target cell.

The term “AAV” is an abbreviation for adeno-associated virus, and may be used to refer to the virus itself or a derivative thereof. The term covers all serotypes, subtypes, and both naturally occurring and recombinant forms, except where required otherwise. The abbreviation “rAAV” refers to recombinant adeno-associated virus, also referred to as a recombinant AAV vector (or “rAAV vector”).

An “rAAV vector” refers to an AAV vector comprising a polynucleotide sequence not of AAV origin (i.e., a polynucleotide heterologous to AAV), typically a sequence of interest for the genetic transformation of a cell. In some examples, the heterologous polynucleotide is flanked by at least one, and generally by two, AAV inverted terminal repeat sequences (“ITRs”).

The term “AAV” or “AAV particle” or “rAAV vector particle” refers to a viral particle composed of at least one “viral capsid” protein and an encapsidated polynucleotide rAAV vector.

The term “expression cassette” as used herein refers to the combination of regulatory elements and a gene or genes to which they are operably linked for expression.

The term “operably linked” refers to the juxtaposition of genetic elements, e.g., a promoter, an enhancer, a polyadenylation sequence, etc., wherein the elements are in a relationship permitting them to operate in the expected manner. For instance, a regulatory element, which can comprise promoter and/or enhancer sequences, is operatively linked to a transgene coding region if the regulatory element helps initiate transcription of the transgene coding sequence. There may be intervening residues between the regulatory element and the coding region so long as the functional relationship is maintained.

The term “artificial genome” refers to a cis expression cassette flanked by AAV ITRs, wherein the cis expression cassette comprises a DN-TNFα transgene operably linked to expression control elements that will control expression of the DN-TNFα in human cells.

The term “TNFα” refers to a human cytokine that exists as a 17 kD soluble form (sTNFα) and a 26 kD membrane-associated form (mTNF), the biologically active form of sTNFα is composed of three noncovalently bound sTNFα molecules (sTNFα trimer) which engages with a cell-surface TNF receptor 1 (TNFR1) complex and a cell-surface TNF receptor 2 (TNFR2) complex, further inducing downstream cellular mechanisms. The structure of human TNFα is described further in, for example, Pennica, D., et al. (1984) Nature 312:724-719; Davis, J M. et al. (1987) Biochemistry 26:1322-1326; and Jones, E Y., et al. (1989) Nature 338:225-228. In the context of the present disclosure, TNFα may also be referred to as TNF or TNFα.

The term “DN-TNFα” refers to a dominant negative mutant of TNFα that differs from the corresponding wild type TNFα protein by at least one amino acid. In some embodiments, the DN-TNFα protein has the amino acid sequence of SEQ ID NO: 2. Other DN-INFa proteins that can be used are disclosed in detail in U.S. Pat. No. 7,446,174, which is incorporated herein in its entirety by reference. TNFα and DN-TNFα proteins of this disclosure can be monomers, dimers, or trimers. In the context of the present disclosure, DN-TNFα may also be referred to as DN-TNF or DN-TNFα.

The term “pharmaceutically acceptable” refers to a substance approved or approvable by a regulatory agency or listed in the U.S. Pharmacopeia, European Pharmacopeia, or other generally recognized pharmacopeia for use in animals, including humans.

The terms “pharmaceutically acceptable” “excipient”, “carrier”, or “adjuvant” each refer to an excipient, carrier, or adjuvant that can be administered to a subject, together with at least one vector of the disclosure, and that is generally understood to be safe, non-toxic, and has no effect on the pharmacological activity of the therapeutic agent. In general, those of skill in the art and the U.S. FDA consider a pharmaceutically acceptable excipient, carrier, or adjuvant to be an inactive ingredient of a formulation.

The term “pharmaceutical composition” refers to a preparation that is prepared in such form that permits the biological activity of DN-TNFα. A pharmaceutical formulation or composition generally comprises additional components, such as pharmaceutically acceptable excipients, carriers, adjuvants, buffers, etc.

The term “inflammation” refers to a localized or systemic inflammatory response in an effected cell, tissue or system, as the case may be. This inflammation may be mediated by the production of cytokines, chemokines, reactive oxygen species, and/or secondary messengers, any of which may be considered markers of inflammation, in general. In some embodiments, a measurement of biological markers may be performed to determine the level of inflammation in an effected cell, tissue or system, thus determining the effectiveness of a therapy to reduce inflammation.

The term “neuroinflammation” refers to the inflammatory response within the central nervous system, i.e., brain or spinal cord. In some embodiments, the compositions of this disclosure can reduce the level of one or more neuroinflammatory markers in the brain and/or spinal cord, including, but not limited to Monocyte Chemoattractant Protein-1 (MCP1), Regulated upon Activation, Normal T Cell Expressed and Presumably Secreted (RANTES), Interleukin 6 (IL-6), Interleukin 8 (IL-8), Interleukin-1 beta (IL-1B), C-Reactive Protein (CRP), TNF, chemokines (CCL2, CCL5, CXCL1), secondary messengers (NO and prostaglandins), and reactive oxygen species, Cluster of Differentiation 45 (CD45), Glial Fibrillary Acidic Protein (GFAP) and/or reduce the microglial density and/or reduce microglial activation. See, e.g., DiSabato D J et al., J. Neurochem; 2016; 139(2): 136-153); Alto L T et al., PLOS One May 2014; 9(5): e96544. In some embodiments, the compositions of this disclosure can reduce pathology associated with neuroinflammation, e.g., amyloid beta plaques in AD, tau accumulation in PD, and degeneration of GABAergic medium spiny neurons (MSN) in HD.

The term “systemic and peripheral inflammation” refers to the activation of the innate or adaptive immune system and release of proinflammatory cytokines against various pathological stimuli outside of the CNS. During systemic inflammation, the innate immune system is chronically activated. In some embodiments, the compositions of this disclosure can reduce the level of one or more inflammatory markers in the periphery, including, but not limited to C-reactive protein (CRP), erythrocyte sedimentation rate (ESR), and plasma viscosity (PV), neutrophil-to-lymphocyte ratio (NLR), derived-NLR (d-NLR), platelet-to-lymphocyte ratio (PLR), systemic immune-inflammation index (SII=NLR×platelets), D-dimer, serum ferritin, serum lactate dehydrogenase (LDH), complete blood count (CBC), and various cytokines, including, but not limited to IL-2, IL-4, IL-6, IL-9, IL-10, IL-13, TNF, and Interferon-γ (IFN-γ). See Watson J, et al. Br J Gen Pract. 2019; 69(684):e462-e469; and Luo Y, Zheng S G. Front Immunol. 2016 Dec. 19; 7:604.

The term “ocular inflammation” refers to the activation of the innate or adaptive immune system and release of proinflammatory cytokines against various pathological stimuli in the eye, including in the retina, cornea, and sclera. Ocular or eye inflammation can cause acute anterior uveitis (iritis), sterile conjunctivitis, keratitis, or episcleritis. In some embodiments, the compositions of this disclosure can reduce the level of one or more inflammatory markers in the eye (e.g., measured in tears, aqueous humor, vitreous humor), including, but not limited to IL-6, TNF-α, IL-1, IL-2, IL-4, IL-5, IL-10, interferon γ-induced protein 10 kDa (IP-10), Granulocyte colony stimulating factor (G-CSF), and IFNγ, as well as reduce vascular endothelial growth factor (VEGF) in the eye (Zahir-Jouzdani, F., et al., Pathophysiology 2017, 24 (3): 123-131; Balamurugan, S. et. al., Indian Journal of Ophthalmology: 2020, 68 (9): 1750-1763; Bonacini M., et al., Front. Immunol., 10 Mar. 2020, Article 358; Takase, H. et al, Invest Ophthalmol & Vis Sci, April 2006, Vol. 47, No. 4).

The term “treatment” or “treating” refers to an improvement, alleviation, or amelioration of at least one symptom of a disclosed condition. The treatment can inhibit deterioration or worsening of a symptom of the condition, or may cause the condition to develop more slowly and/or to a lesser degree (e.g., lesser inflammation or fewer symptoms) in a subject than it would have absent the treatment. For example, a treatment with the compositions of this disclosure will be said to have “treated” the condition if it is given during the condition, e.g., during an early diagnosis of a neuroinflammatory condition (e.g., early AD), and results in the subject's experiencing fewer and/or milder symptoms of the condition than otherwise expected. A treatment with the compositions of this disclosure will be said to have “treated” the condition if the treatment results in a reduction in the pathology of the condition (e.g., amyloid beta aggregates in AD, tau accumulation in PD, etc.).

The term “treatment” or “treating” in the context of a systemic inflammatory condition such as Rheumatoid Arthritis refers to therapeutic treatment, as well as prophylactic treatment, for the treatment of rheumatoid arthritis. For example, the term treatment may include administration of a compositions of this disclosure prior to or following the onset of rheumatoid arthritis thereby inhibiting, reducing, or removing signs of the disease or disorder. In another example, the compositions of this disclosure can be administered after clinical manifestation of rheumatoid arthritis to combat the symptoms and/or complications and disorders associated with rheumatoid arthritis. In some embodiment, treatment of rheumatoid arthritis in a subject comprises inhibiting the progression of structural damage. In one embodiment, treatment of rheumatoid arthritis comprises improving physical function in patients with moderately to severely active disease.

The terms “effective amount” or “therapeutically effective amount” refer to an amount of a composition described herein that is sufficient to affect the intended application or indication, including but not limited to, treatment of inflammation, as defined herein. The therapeutically effective amount may vary depending upon the intended treatment application (in a cell or in vivo), or the subject and inflammatory condition being treated, e.g., the weight and age of the subject, the severity of the disease condition, the manner of administration and the like, which can readily be determined by one of ordinary skill in the art after reading the present disclosure. The terms also apply to a dose that will induce a particular response in a target cell. The specific dose will vary depending on, for example, the particular composition chosen, the dosing regimen to be followed, whether it is administered in combination with other compounds, timing of administration, the tissue to which it is administered, and the physical delivery system in which it is carried.

As used herein, references to “about” or “approximately” a value or parameter include (and describe) embodiments that are directed to that value or parameter. For example, a description referring to “about X” includes description of “X.” “About X” means+/−10% of X. So, “about 10” means a value between 9 and 11.

### TNF and DN-TNFα

TNF is a multifunctional cytokine that can complex with, e.g., specifically bind to, either of two TNF receptors, TNFR1 (p55) and TNFR2 (p75), to activate signaling cascades controlling apoptosis, inflammation, cell proliferation, and the immune response (Steed 2003). The 26-kD type II transmembrane TNF precursor protein, expressed on many cell types, is proteolytically converted into a soluble 52-kD homotrimer. Recombinant Dominant-Negative TNF (DN-TNF) variants can form heterotrimers with TNF molecules or homotrimers with other DN-TNF molecules, which modify or disrupt the interaction with TNFR1 while maintaining prosurvival and homeostatic TNFR2 signaling. The DN-TNFα-encoding transgenes of the present disclosure comprise the nucleic acid sequence of SEQ ID NO: 3 or SEQ ID NO: 4. Additional DN-TNFα amino acid sequences and nucleic acids encoding DN-TNFα are described in U.S. Pat. No. 7,446,174, or US Patent Application Publication No. US 2015/0239951, which are incorporated herein by reference in their entireties. In certain embodiments, the DN-TNFα transgene of the disclosure encodes for a protein harboring two amino acid mutations-A145R and Y87H, which allow DN-TNFα to form heterotrimeric structures with native TNFα that interact with TNF Receptor 1 (TNFR1), but disrupt agonist interactions with TNFR1, and thus diminish proinflammatory signaling. In some embodiments, the nucleic acid sequence of the DN-TNFα transgene is codon-optimized, as described herein. In some embodiments, the nucleic acid sequence of the DN-TNFα transgene is depleted of CpG motifs, as described herein. In certain embodiments, the DN-TNFα transgene comprises the nucleotide sequence set forth in SEQ ID NO: 3. In certain embodiments, the DN-TNFα transgene comprises the nucleotide sequence set forth in SEQ ID NO: 168. SEQ ID NO: 3 is codon optimized and CpG-depleted. In some embodiments, the DN-TNFα transgene of the present disclosure encodes for the amino acid sequence set forth in SEQ ID NO: 2.

Wild-type human TNFα amino acid sequence; Uniprot ID No. P01375
(SEQ ID NO: 1)
VRSSSRTPSDKPVAHVVANPQAEGQLQWLNRRANALLANGVELRDNQLVVPSEGLYLI
YSQVLFKGQGCPSTHVLLTHTISRIAVSYQTKVNLLSAIKSPCQRETPEGAEAKPWYEPIY
LGGVFQLEKGDRLSAEINRPDYLDFAESGQVYFGIIAL
DN-TNFα amino acid sequence
(SEQ ID NO: 2)
VRSSSRTPSDKPVAHVVANPQAEGQLQWLNRRANALLANGVELRDNQLVVPSEGLYLI
YSQVLFKGQGCPSTHVLLTHTISRIAVSHQTKVNLLSAIKSPCQRETPEGAEAKPWYEPIY
LGGVFQLEKGDRLSAEINRPDYLDFRESGQVYFGIIAL
Nucleotide sequence #1 encoding DN-TNFα
(SEQ ID NO: 3)
GTGAGATCCTCCTCAAGAACCCCTTCTGATAAACCTGTGGCTCATGTGGTGGCTAAC
CCTCAGGCAGAAGGACAGCTGCAGTGGCTGAATAGGAGGGCCAATGCACTGCTGGC
TAATGGAGTGGAACTGAGGGACAACCAGCTGGTGGTCCCTTCAGAAGGACTGTACC
TGATCTATAGCCAGGTGCTGTTCAAGGGCCAGGGATGTCCCAGTACTCATGTGCTGC
TGACTCACACCATCTCCAGAATTGCTGTCTCCCATCAGACAAAGGTGAACCTGCTGT
CAGCCATCAAAAGTCCCTGCCAGAGAGAGACACCAGAAGGAGCTGAGGCCAAGCC
ATGGTATGAGCCAATCTACCTGGGGGGAGTGTTTCAGCTGGAGAAAGGAGACAGGC
TGAGTGCAGAAATCAACAGACCAGACTATCTGGACTTTAGAGAATCAGGGCAGGTG
TATTTTGGCATTATTGCACTGTGA*
Nucleotide sequence #2 encoding DN-TNFα
(SEQ ID NO: 4)
GTACGCTCCTCCTCCCGCACTCCGTCCGACAAACCGGTAGCTCACGTAGTAGCTAAC
CCGCAGGCTGAAGGTCAGCTGCAGTGGCTGAACCGCCGCGCTAACGCTCTGCTGGC
TAACGGTGTAGAACTGCGCGACAACCAGCTGGTAGTACCGTCCGAAGGTCTGTACCT
GATCTACTCCCAGGTACTGTTCAAAGGTCAGGGTTGTCCGTCCACTCACGTACTGCT
GACTCACACTATCTCCCGCATCGCTGTATCCTACCAGACTAAAGTAAACCTGCTGTC
CGCTATCAAATCCCCGTGTCAGCGCGAAACTCCGGAAGGTGCTGAAGCTAAACCGT
GGTACGAACCGATCTACCTGGGTGGTGTATTCCAGCTGGAAAAAGGTGACCGCCTGT
CCGCTGAAATCAACCGCCCGGACTACCTGGACTTCGCTGAATCCGGTCAGGTATACT
TCGGTATCATCGCTCTGTGA*

*The underlined sequence is a representative stop codon. That sequence can be any known stop codon-TGA, TAG, or TAA

As used herein, variant TNFα or TNFα proteins include TNFα monomers, dimers, or trimers. Included within the definition of “variant TNFα” are competitive inhibitor TNFα variants. While certain variants are described herein, one of skill in the art will understand that other variants can be made while retaining the function of inhibiting soluble, but not transmembrane, TNFα.

The present disclosure provides methods of treating neuroinflammation, systemic inflammation, and/or ocular inflammation characterized by elevated TNFα.

### Viral Vectors and Constructs

rAAV Particles

This disclosure provides non-replicating recombinant AAV (“rAAV”) particles encoding DN-TNFα. The viral vectors described herein can be delivered to a target cell using any suitable method for delivery to a target cell. In some embodiments, the vector is a targeted vector, e.g., a vector targeted to CNS cells, muscle cells, liver cells, ocular cells, synovial cells, retinal cells, or retinal pigment epithelial cells.

The methods disclosed herein can be used in the production of rAAV particles comprising a capsid protein from an AAV capsid serotype described herein. In some embodiments, the rAAV particles comprise a capsid protein from an AAV capsid serotype selected from AAV1, AAV2, rAAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise a capsid protein that is a derivative, modification, or pseudotype of AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 capsid protein.

In some embodiments, the rAAV particles comprise a capsid protein from an AAV capsid serotype selected from AAV8 and AAV9. In some embodiments, the rAAV particles have an AAV capsid serotype of AAV8. In some embodiments, the rAAV particles have an AAV capsid serotype of AAV9.

In some embodiments, the rAAV particles comprise a capsid protein from an AAV capsid serotype selected from the group consisting of AAV.rh8, AAV.rh10, AAV.rh20, AAV.rh39, AAV.Rh74, AAV.RHM4-1, AAV.hu37, AAV.hu32, AAV.PHB, and AAV.7m8. In some embodiments, the rAAV particles comprise a capsid protein with high sequence homology to AAV8 or AAV9 such as, AAV.rh10, AAV.rh20, AAV.rh39, AAV.Rh74, AAV.RHM4-1, and AAV.hu37.

In some embodiments, the rAAV particles comprise a capsid protein that is a derivative, modification, or pseudotype of AAV8 or AAV9 capsid protein. In some embodiments, the rAAV particles comprise a capsid protein that has an AAV8 or AAV9 capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e., up to 100% identical, to the VP1, VP2, and/or VP3 sequence of AAV8 or AAV9 capsid protein.

In some embodiments, the rAAV particles comprise a capsid protein that has at least 80% or more identity, e.g., 85%, 86%, 87%, 88%, 89%, 90%, 91%, 92%, 93%, 94%, 95%, 96%, 97%, 98%, 99%, 99.5%, etc., i.e., up to 100% identity, to the VP1, VP2 and/or VP3 sequence of AAV.rh8, AAV.rh10, AAV.rh20, AAV.rh39, AAV.Rh74, AAV.RHM4-1, AAV.hu37, AAV.hu32, AAV.PHB, or AAV.7m8 capsid protein. In some embodiments, the rAAV particles comprise a capsid protein that has at least 80% or more identity, e.g., 85%, 86%, 87%, 88%, 89%, 90%, 91%, 92%, 93%, 94%, 95%, 96%, 97%, 98%, 99%, 99.5%, etc., i.e. up to 100% identity, to the VP1, VP2 and/or VP3 sequence of an AAV capsid protein with high sequence homology to AAV8 or AAV9 such as, AAV.rh10, AAV.rh20, AAV.rh39, AAV.Rh74, AAV.RHM4-1, and AAV.hu37.

In additional embodiments, the rAAV particles comprise a mosaic capsid. In additional embodiments, the rAAV particles comprise a pseudotyped rAAV particle. In additional embodiments, the rAAV particles comprise a capsid containing a capsid protein chimera of two or more AAV capsid serotypes.

The methods disclosed herein are suitable for use in the production of any isolated recombinant AAV particles. As such, the rAAV can be of any serotype, modification, or derivative known in the art, or any combination thereof (e.g., a population of rAAV particles that comprises two or more serotypes, e.g., comprising two or more of rAAV2, rAAV8, and rAAV9 particles) known in the art. In some embodiments, the rAAV particles are rAAV1, rAAV2, rAAV3, rAAV4, TAAV5, rAAV6, TAAV7, TAAV8, TAAV9, rAAV10, rAAV11, rAAV12, rAAV13, rAAV14, AAV15, rAAV16, rAAV.rh8, rAAV.rh10, rAAV.rh20, rAAV.rh39, rAAV.Rh74, rAAV.RHM4-1, rAAV.hu37, rAAV.hu32, rAAV.Anc80, rAAV.Anc80L65, rAAV.7m8, rAAV.PHP.B, rAAV.PHP.eB, rAAV2.5, rAAV2tYF, rAAV3B, rAAV.LK03, rAAV.HSC1, rAAV.HSC2, rAAV.HSC3, rAAV.HSC4, rAAV.HSC5, rAAV.HSC6, TAAV.HSC7, rAAV.HSC8, rAAV.HSC9, rAAV.HSC10, rAAV.HSC11, rAAV.HSC12, rrAAV.HSC13, AAV.HSC14, rAAV.HSC15, or rAAV.HSC16 or other rAAV particles, or combinations of two or more thereof.

In some embodiments, rAAV particles have a capsid protein from an AAV serotype selected from AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or a derivative, modification, or pseudotype thereof. In some embodiments, rAAV particles comprise a capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e., up to 100% identical, to e.g., VP1, VP2 and/or VP3 sequence of an AAV capsid serotype selected from AAV1, AAV2, AAV3, AAV4, AAV5, AAV6, AAV7, AAV8, AAV9, AAV10, AAV11, AAV12, AAV13, AAV14, AAVIS, AAV16, AAV.rh8, AAV.rh10, AAV.rh20, AAV.rh39, AAV.Rh74, AAV.RHM4-1, AAV.hu37, AAV.hu32, AAV.Anc80, rAAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise a capsid protein from an AAV capsid serotype selected from AAV1, AAV1, AAV2, rAAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof. In some embodiments, rAAV particles comprise a capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise the capsid of Anc80 or Anc80L65, as described in Zinn E et al., 2015, Cell Rep. 12 (6): 1056-1068, which is incorporated by reference in its entirety. In certain embodiments, the rAAV particles comprise the capsid with one of the following amino acid insertions: LGETTRP (SEQ ID NO: 172) or LALGETTRP (SEQ ID NO: 173), as described in U.S. Pat. Nos. 9,193,956; 9,458,517; and 9,587,282 and US Patent Application Publication No. US 2016/0376323, each of which is incorporated herein by reference in its entirety. In some embodiments, rAAV particles comprise the capsid of AAV.7m8, as described in U.S. Pat. Nos. 9,193,956; 9,458,517; and 9,587,282 and US Patent Application Publication No. US 2016/0376323, each of which is incorporated herein by reference in its entirety. In some embodiments, rAAV particles comprise any AAV capsid disclosed in U.S. Pat. No. 9,585,971, such as AAV.PHP.B. In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis A, et al., Gene Ther. 2016 December; 23 (12): 857-862. Epub 2016 Sep. 22; Georgiadis, A., et al., Gene Ther 25, 450 (2018), each of which is incorporated by reference in its entirety.

In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo F et al., 2017, Sci. Transl. Med. 29 (9): 418, which is incorporated by reference in its entirety. In some embodiments, rAAV particles comprise any AAV capsid disclosed in U.S. Pat. Nos. 8,628,966; 8,927,514; 9,923,120, and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles comprise any AAV capsid disclosed in WO2019222329A1 and WO2020077165A1, such as VOY101, VOY201, VOY701, VOY801, or VOY1101, each of which is incorporated herein by reference in its entirety.

In some embodiments, rAAV particles comprise an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US Patent Application Publication Nos. US 2015/0374803; US 2015/0126588; US 2017/0067908; US 2013/0224836; US 2016/0215024; and US 2017/0051257; and International Patent Application Nos. PCT/US2015/034799 and PCT/EP2015/053335.

In some embodiments, rAAV particles have a capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US Patent Application Publication Nos. US 2015/0374803; US 2015/0126588; US 2017/0067908; US 2013/0224836; US 2016/0215024; and US 2017/0051257; and International Patent Application Nos. PCT/US2015/034799; PCT/EP2015/053335.

In some embodiments, rAAV particles have a capsid protein disclosed in Intl. Appl. Publ. No. WO 2003/052051 (see, e.g., SEQ ID NO: 2 of '051), WO 2005/033321 (see, e.g., SEQ ID NOs: 123 and 88 of '321), WO 03/042397 (see, e.g., SEQ ID NOs: 2, 81, 85, and 97 of '397), WO 2006/068888 (see, e.g., SEQ ID NOs: 1 and 3-6 of '888), WO 2006/110689, (see, e.g., SEQ ID NOs: 5-38 of '689) WO2009/104964 (see, e.g., SEQ ID NOs: 1-5, 7, 9, 20, 22, 24 and 31 of '964), WO 2010/127097 (see, e.g., SEQ ID NOs: 5-38 of '097), and WO 2015/191508 (see, e.g., SEQ ID NOs: 80-294 of '508), and U.S. Patent Application Publication No. US 2015/0023924 (see, e.g., SEQ ID NOs: 1, 5-10 of '924), the contents of each of which is herein incorporated by reference in its entirety. In some embodiments, rAAV particles have a capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e., up to 100% identical, to the VP1, VP2 and/or VP3 sequence of an AAV capsid disclosed in Intl. Appl. Publ. No. WO 2003/052051 (see, e.g., SEQ ID NO: 2 of '051), WO 2005/033321 (see, e.g., SEQ ID NOs: 123 and 88 of '321), WO 03/042397 (see, e.g., SEQ ID NOs: 2, 81, 85, and 97 of 397), WO 2006/068888 (see, e.g., SEQ ID NOs: 1 and 3-6 of '888), WO 2006/110689 (see, e.g., SEQ ID NOs: 5-38 of '689) WO2009/104964 (see, e.g., SEQ ID NOs: 1-5, 7, 9, 20, 22, 24 and 31 of '964), WO 2010/127097 (see, e.g., SEQ ID NOs: 5-38 of '097), and WO 2015/191508 (see, e.g., SEQ ID NOs: 80-294 of '508), and U.S. Appl. Publ. No. 20150023924 (see, e.g., SEQ ID NOs: 1, 5-10 of '924).

Nucleic acid sequences of AAV based viral vectors and methods of making recombinant AAV and AAV capsids are taught, for example, in U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; International Patent Application Nos. PCT/US2015/034799; PCT/EP2015/053335; WO 2003/052051, WO 2005/033321, WO 03/042397, WO 2006/068888, WO 2006/110689, WO2009/104964, WO 2010/127097, and WO 2015/191508, and U.S. Appl. Publ. No. 20150023924.

The provided methods are suitable for use in the production of recombinant AAV encoding DN-TNFα (e.g., SEQ ID NO:2) described earlier. In some embodiments, the rAAV genome comprises a vector comprising the following components: (1) AAV inverted terminal repeats that flank an expression cassette; (2) regulatory control elements, such as a) promoter/enhancers, b) a poly A signal, and c) optionally an intron; and (3) nucleic acid sequences coding for a transgene. These components are described in greater detail elsewhere in the disclosure.

In other embodiments for expressing a DN-TNFα transgene, the rAAV genome comprises a vector comprising the following components: (1) AAV inverted terminal repeats that flank an expression cassette; (2) regulatory control elements, such as a) promoter/enhancers, b) a poly A signal, and c) optionally an intron; and (3) nucleic acid sequences coding for DN-TNFα.

In some embodiments, the rAAV particles are rAAV viral vectors encoding DN-TNFα. In other embodiments, the rAAV particles are rAAV8-based viral vectors encoding an DN-TNFα. In other embodiments, the rAAV particles are rAAV9-based viral vectors encoding an DN-TNFα. In some embodiments, the rAAV particles are rAAV10-based viral vectors encoding an DN-TNFα.

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan D. et al., J. Virol., 75:7662-7671 (2001); Halbert C L et al., J. Virol., 74:1524-1532 (2000); Zolotukhin S. et al., Methods 28:158-167 (2002); and Auricchio A et al., Hum. Molec. Genet. 10:3075-3081, (2001).

In additional embodiments, rAAV particles comprise a capsid containing a capsid protein chimeric of two or more AAV capsid serotypes. In some embodiments, the capsid protein is a chimeric of 2 or more AAV capsid proteins from AAV serotypes selected from AAV1, AAV1, AAV2, AAV3, AAV4, AAV5, AAV6, AAV7, AAV8, AAV9, AAV10, AAV11, AAV12, AAV13, AAV14, AAV15 and AAV-16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B. AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In certain embodiments, a single-stranded AAV (ssAAV) can be used. In certain embodiments, a self-complementary vector, e.g., scAAV, can be used (see, e.g., Wu J. et al., 2007, Human Gene Therapy, 18 (2): 171-82; McCarty D M et al., 2001, Gene Ther. 2001 August; 8 (16): 1248-54; and U.S. Pat. Nos. 6,596,535; 7,125,717; and 7,456,683, each of which is incorporated herein by reference in its entirety).

In some embodiments, the rAAV particles comprise a capsid protein from an AAV capsid serotype selected from AAV-8 or AAV-9. In some embodiments, the rAAV particles have an AAV capsid serotype of AAV-8. In some embodiments, the rAAV particles have an AAV capsid serotype of AAV-9.

In some embodiments, the rAAV particles comprise a capsid protein that is a derivative, modification, or pseudotype of AAV8 or AAV9 capsid protein. In some embodiments, the rAAV particles comprise a capsid protein that has an AAV8 capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV8 capsid protein.

In some embodiments, the rAAV particles comprise a capsid protein that is a derivative, modification, or pseudotype of AAV9 capsid protein. In some embodiments, the rAAV particles comprise a capsid protein that has an AAV9 capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV9 capsid protein.

In additional embodiments, the rAAV particles comprise a mosaic capsid. Mosaic AAV particles are composed of a mixture of viral capsid proteins from different serotypes of AAV. In some embodiments, the rAAV particles comprise a mosaic capsid containing capsid proteins of a serotype selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise a mosaic capsid containing capsid proteins of a serotype selected from AAV-1, AAV-2, AAV-5, AAV-6, AAV-7, AAV-8, AAV-9, AAV-10, AAVrh.8, AAVrh.10, AAV.hu37, AAV.hu32, AAVrh.20, and AAVrh.74.

In additional embodiments, the rAAV particles comprise a pseudotyped rAAV particle. In some embodiments, the pseudotyped rAAV particle comprises (a) a nucleic acid vector comprising AAV ITRs and (b) a capsid comprised of capsid proteins derived from AAVx (e.g., AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16). In additional embodiments, the rAAV particles comprise a pseudotyped rAAV particle comprised of a capsid protein of an AAV serotype selected from AAV1, AAV2, AAV5, AAV6, AAV7, AAV8, AAV9, AAV10, AAVrh.8, and AAVrh.10, AAV.hu37, AAV.hu32, AAVrh.20, and AAVrh.74. In additional embodiments, the rAAV particles comprise a pseudotyped rAAV particle containing AAV-8 capsid protein. In additional embodiments, the rAAV particles comprise a pseudotyped rAAV particle is comprised of AAV9 capsid protein. In some embodiments, the pseudotyped rAAV8 or rAAV9 particles are rAAV2/8 or rAAV2/9 pseudotyped particles. Methods for producing and using pseudotyped rAAV particles are known in the art (see, e.g., Duan D. et al., J. Virol., 75:7662-7671 (2001); Halbert C L et al., J. Virol., 74:1524-1532 (2000); Zolotukhin S. et al., Methods 28:158-167 (2002); and Auricchio A. et al., Hum. Molec. Genet. 10:3075-3081, (2001).

In additional embodiments, the rAAV particles comprise a capsid containing a capsid protein chimeric of two or more AAV capsid serotypes. In some embodiments, the rAAV particles comprise an AAV capsid protein chimeric of AAV-8 capsid protein and one or more AAV capsid proteins from an AAV serotype selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise an AAV capsid protein chimeric of AAV8 capsid protein and one or more AAV capsid proteins from an AAV serotype selected from AAV1, AAV2, AAV5, AAV6, AAV7, AAV9, AAV10, rAAVrh10, AAVrh.8, AAVrh.10, AAV.hu37, AAV.hu32, AAVrh.20, and AAVrh.74. In some embodiments, the rAAV particles comprise an AAV capsid protein chimeric of AAV9 capsid protein the capsid protein of one or more AAV capsid serotypes selected from AAV1, AAV2, AAV3, AAV4, AAV5, AAV6, AAV7, AAV8, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise an AAV capsid protein chimeric of AAV9 capsid protein the capsid protein of one or more AAV capsid serotypes selected from AAV1, AAV2, AAV3, AAV4, AAV5, AA6, AAV7, AAV8, AAV9, AAVrh.8, AAVrh.10, AAV.hu37, AAV.hu32, AAVrh.20, and AAVrh.74.

The rAAV particles comprising the DN-TNFα transgene have one or more of the components disclosed below.

### Viral Vectors

Viruses of the Parvoviridae family are small non-enveloped icosahedral capsid viruses characterized by a single stranded DNA genome. Parvoviridae family viruses consist of two subfamilies: Parvovirinae, which infect vertebrates, and Densovirinae, which infect invertebrates. Due to its relatively simple structure, easily manipulated using standard molecular biology techniques, this virus family is useful as a biological tool. The genome of the virus may be modified to contain a minimum of components for the assembly of a functional recombinant virus, or viral particle, which is loaded with or engineered to express or deliver a desired payload (e.g., the DN-TNFα), which may be delivered to a target tissue.

The parvoviruses and other members of tire Parvoviridae family are generally described in Kenneth 1, Berns, ‘Parvoviridae: The Viruses and Their Replication,” Chapter 69 in FIELDS VIROLOGY (3d Ed. 1996), the contents of which are incorporated by reference in their entirety.

The parvoviral vectors that are useful in the compositions and methods of this disclosure include adeno-associated viruses (AAV, e.g., AAV8, AAV9, AAVrh10, etc). In certain embodiments, the viral vectors provided herein are recombinant viral vectors. In certain embodiments, the viral vectors provided herein are altered such that they are replication-deficient in humans. In certain embodiments, the viral vectors are hybrid vectors, e.g., an AAV vector placed into a “helpless” adenoviral vector. In certain embodiments, provided herein are viral vectors comprising a viral capsid from a first virus and viral envelope proteins from a second virus. In specific embodiments, the second virus is vesicular stomatitus virus (VSV). In more specific embodiments, the envelope protein is VSV-G protein.

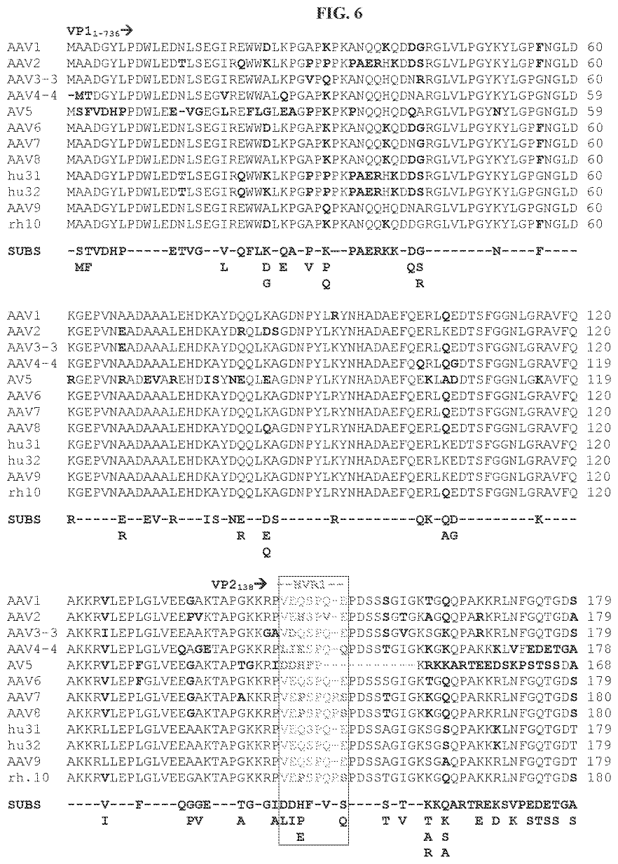

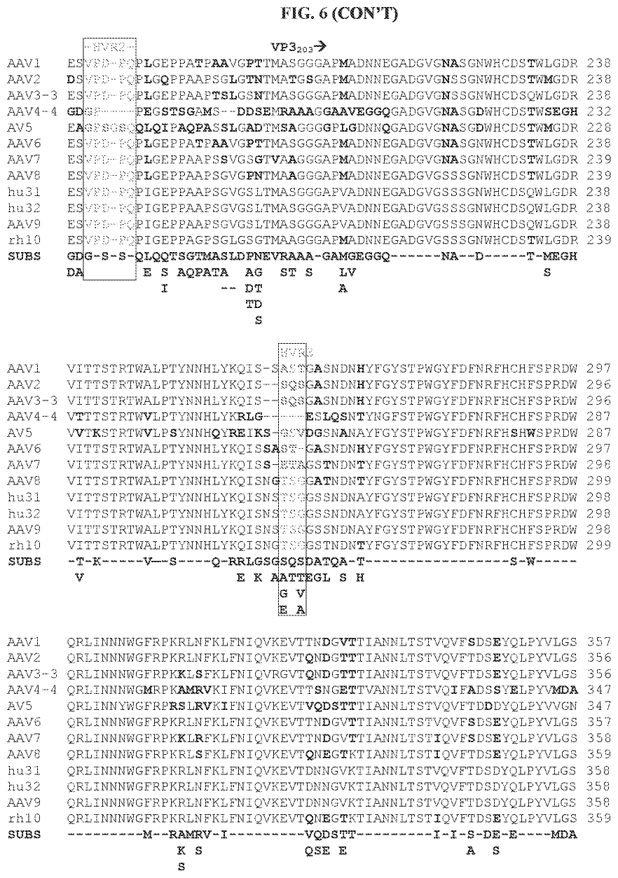

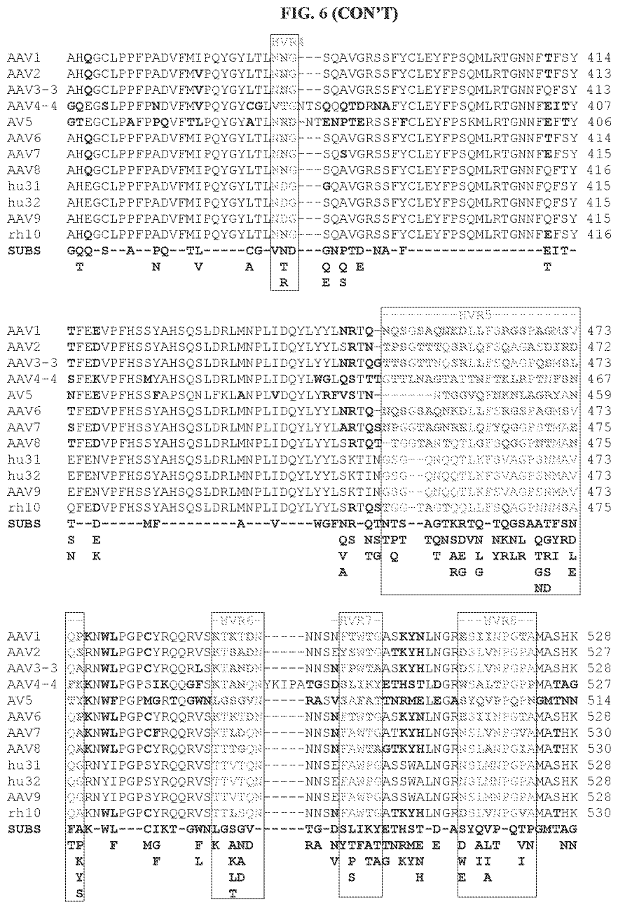

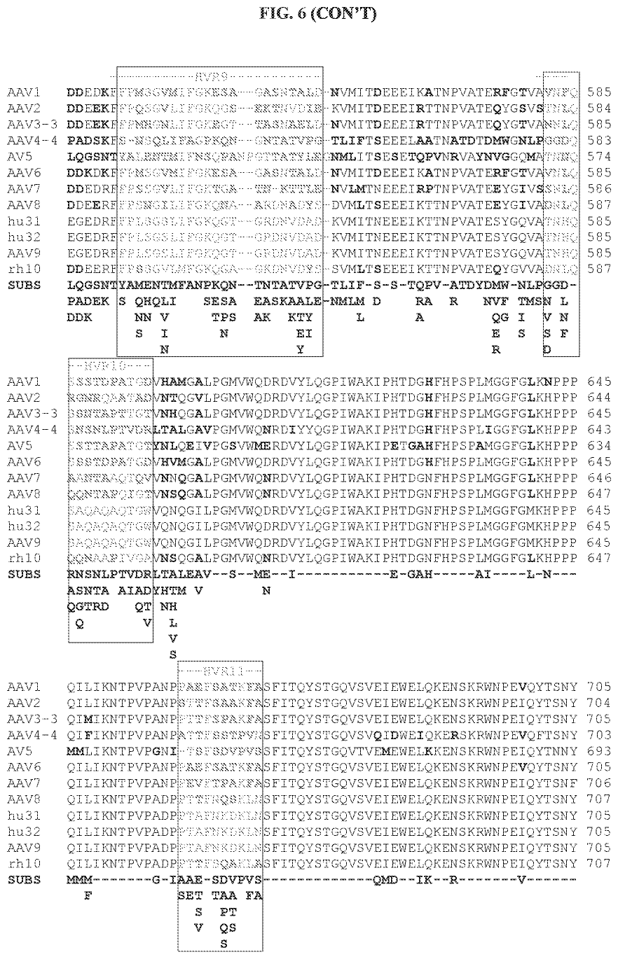

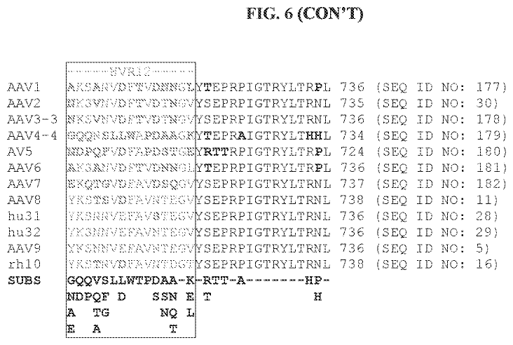

In certain embodiments, the viral vectors provided herein are AAV based viral vectors. In certain embodiments, the AAV-based vectors provided herein do not encode the AAV rep gene (required for replication) and/or the AAV cap gene (required for synthesis of the capsid proteins) (the rep and cap proteins may be provided by the packaging cells in trans). Multiple AAV serotypes have been identified. In certain embodiments, AAV-based vectors provided herein comprise components from one or more serotypes of AAV. In certain embodiments, AAV based vectors provided herein comprise capsid components from one or more of AAV8 (SEQ ID NO: 11); AAVrh10 (SEQ ID NO: 16); AAV9 (SEQ ID NO: 5); AAV.PHP.eB (SEQ ID NO: 7); AAV.hDyn (SEQ ID NO: 6); AAV.PHP.B (SEQ ID NO: 8); AAV.PHP.S (SEQ ID NO: 9); AAV.PHP.SH (SEQ ID NO: 10); AAV8.BBB (SEQ ID NO: 12); AAV8.BBB.LD (SEQ ID NO: 13); AAV9.BBB (SEQ ID NO: 14); AAV9.BBB.LD (SEQ ID NO: 15); AAVrh.10.LD (SEQ ID NO: 17); AAV9.496NNN/AAA498 (SEQ ID NO: 18); VOY101 (SEQ ID NO: 19); VOY201 (SEQ ID NO: 20); VOY701 (SEQ ID NO: 21); VOY801 (SEQ ID NO: 22); VOY1101 (SEQ ID NO: 23); AAV.S454.Tfr3 (SEQ ID NO: 24); AAV.hu37 (SEQ ID NO: 25); AAVrh74 version 1 (SEQ ID NO: 26); AAVrh74 version 2 (SEQ ID NO: 27); AAV.hu.31 (SEQ ID NO: 28); AAV.hu32 (SEQ ID NO: 29); AAV2 (SEQ ID NO: 30); AAV3B (SEQ ID NO: 31); AAV2.7m8 (SEQ ID NO: 32); AAV.rh.34 (SEQ ID NO: 33); AAV.rh.31 (SEQ ID NO: 34); AAV.hu.12 (SEQ ID NO: 35); AAV.hu.13 (SEQ ID NO: 36); AAV.hu.21 (SEQ ID NO: 37); AAV.hu.26 (SEQ ID NO: 38); AAV.hu.53 (SEQ ID NO: 39); AAV.hu.56 (SEQ ID NO: 40); AAV.rh.24 (SEQ ID NO: 41); AAV.hu.38 (SEQ ID NO: 42); AAV.rh.72 (SEQ ID NO: 43); AAV.cy.5 (SEQ ID NO: 44); AAV.cy.6 (SEQ ID NO: 45); AAV.rh.46 (SEQ ID NO: 46); or AAV.rh.2 (SEQ ID NO: 47). In certain embodiments, AAV based vectors provided herein comprise components from one or more of AAV8, AAV9, or AAVrh10 serotypes. Provided are viral vectors in which the capsid protein is a variant of the AAV8 capsid protein (SEQ ID NO: 11), AAV9 capsid protein (SEQ ID NO: 5), or AAVrh10 capsid protein (SEQ ID NO: 16), and the capsid protein is e.g., at least 95%, 96%, 97%, 98%, 99% or 99.9% identical to the amino acid sequence of any of the AAV8 capsid protein (SEQ ID NO: 11), the AAV9 capsid protein (SEQ ID NO: 5), or the AAVrh10 capsid protein (SEQ ID NO: 6), while retaining a biological function of the native capsid. In certain embodiments, the encoded AAV capsid has the sequence of SEQ ID NO: 11, 5, or 16 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining a biological function of the AAV8, AAV9, or AAVrh10 capsid. FIG. 21 in WO2020/219868, incorporated by reference here in its entirety (FIG. 6 in this disclosure), provides a comparative alignment of the amino acid sequences of the capsid proteins of different AAV serotypes with potential amino acids that may be substituted at certain positions in the aligned sequences based upon the comparison in the row labeled SUBS. Accordingly, in specific embodiments, the AAV vector comprises an AAV8, AAV9 or AAVrh10 capsid variant that has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions that are not present at that position in the native AAV capsid sequence as identified in the SUBS row of FIG. 6. Amino acid sequence for AAV8, AAV9, and AAVrh10 capsids are provided in FIG. 6.

Variants of viral vectors retaining a biological function of the native capsid have a variant capsid that maintains the ability to form full capsids, thus packaging a genome under conditions that favor viral vector formation. Variant viral vectors are generally capable of transducing cells and may or may not display the same tropism as a native capsid, or may display enhanced tropism for one of more tissues or cell types.

AAV8-based, AAV9-based, and AAVrh10-based viral vectors, and other viral vectors and variants thereof, are used in certain of the methods described herein. Nucleotide sequences of AAV based viral vectors and methods of making recombinant AAV and AAV capsids are taught, for example, in U.S. Pat. Nos. 7,282,199; 7,790,449; 8,318,480; 8,962,332; 7,906,111; 8,524,446; 8,999,678; 8,628,966; 8,927,514; 8,734,809; 9,284,357; 9,409,953; 9,169,299; 9,193,956; 9,458,517; and 9,587,282; and International Patent Application Nos. PCT/US02/33629; PCT/US02/33630; PCT/US2004/028817; PCT/US2015/034799; PCT/EP2015/053335, each of which is incorporated herein by reference in its entirety. In one aspect, provided herein are AAV (e.g., AAV8, AAV9, AAVrh10, or other AAV serotype)-based viral vectors encoding a transgene (e.g., a DN-TNF). The amino acid sequences of various AAV capsids, including AAV8, AAV9 and AAVrh10 are provided in FIG. 6.

The terms “sequence identity”, “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, e.g. a capsid sequence or a transgene sequence, or a fragment or portion thereof, for example over a nucleotide sequence encoding a VP1, or a VP2, or a VP3 capsid protein. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment or portion thereof, such as for VP proteins.

Alignments are performed using any of a variety of publicly or commercially available Multiple Sequence Alignment Programs, such as Clustal W, accessible through web servers on the internet. There are a number of algorithms known in the art which can be used to measure nucleotide sequence identity, including those contained in the programs described herein. Similar programs are available for amino acid sequences, e.g., the Clustal X program (Soding, J. (2005) Bioinformatics 21, 951-960; Thompson J D et al., (1994) Nucleic Acids Res. 22 (22): 4673-4680; Larkin, M A et al. (2007) Bioinformatics, 23, 2947-2948). Generally, any of these programs can be used at default settings, although one of skill in the art can alter these settings as needed. Alternatively, one of skill in the art can utilize another algorithm or computer program which provides at least the level of identity or alignment as that provided by the referenced algorithms and programs (Higgins D G et al., (2005) PNAS USA; 102 (30): 10411-10412; Raghava and Barton (2006) BMC Bioinformatics, 7:415).

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. In some instances, the homology is over a full-length sequence, or an open reading frame thereof, e.g., a cap sequence, a rep sequence, a transgene, a promoter, or another suitable fragment which is at least 15 nucleotides in length. Examples of suitable fragments are described herein. The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. In some instances, the homology is over a full-length sequence, or a protein thereof, e.g., a cap protein, a rep protein, a therapeutic protein, or a fragment or portion thereof which is at least 8 amino acids, or at least 15 amino acids in length. Examples of suitable fragments are described herein.

In some embodiments, AAV-based vectors comprise components from one or more serotypes of AAV. In some embodiments, AAV based vectors provided herein comprise capsid components from one or more of AAV8, AAVrh10, AAV9, AAV.PHP.eB, AAV.hDyn, AAV.PHP.B, AAV.PHP.S, AAV.PHP.SH, AAV8.BBB, AAV8.BBB.LD, AAV9.BBB, AAV9.BBB.LD, AAVrh.10.LD, AAV9.496NNN/AAA498, VOY101, VOY201, VOY701, VOY801, VOY1101, AAV.hu37, AAVrh74 version 1, AAVrh74 version 2, AAV.hu.31, AAV.hu32, AAV2, AAV3B, AAV2.7m8, AAV.rh.34, AAV.hu.31, AAV.rh.31, AAV.hu.12, AAV.hu.13, AAV.hu.21, AAV.hu.26, AAV.hu.53, AAV.hu.56, AAV.rh.24, AAV.hu.38, AAV.rh.72, AAV.cy.5, AAV.cy.6, AAV.rh.46, or AAV.rh.2, or other rAAV particles, or combinations of two or more thereof. In some embodiments, AAV based vectors provided herein comprise components from one or more of AAV8, AAVrh10, AAV9, AAV.PHP.eB, AAV.hDyn, AAV.PHP.B, AAV.PHP.S, AAV.PHP.SH, AAV8.BBB, AAV8.BBB.LD, AAV9.BBB, AAV9.BBB.LD, AAVrh.10.LD, AAV9.496NNN/AAA498, VOY101, VOY201, VOY701, VOY801, VOY1101, AAV.hu37, AAVrh74 version 1, AAVrh74 version 2, AAV.hu.31, AAV.hu32, AAV2, AAV3B, AAV2.7m8, AAV.rh.34, AAV.hu.31, AAV.rh.31, AAV.hu.12, AAV.hu.13, AAV.hu.21, AAV.hu.26, AAV.hu.53, AAV.hu.56, AAV.rh.24, AAV.hu.38, AAV.rh.72, AAV.cy.5, AAV.cy.6, AAV.rh.46, or AAV.rh.2, or other rAAV particles, or combinations of two or more thereof serotypes. In some embodiments, rAAV particles comprise a capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV8, AAVrh10, AAV9, AAV.PHP.eB, AAV.hDyn, AAV.PHP.B, AAV.PHP.S, AAV.PHP.SH, AAV8.BBB, AAV8.BBB.LD, AAV9.BBB, AAV9.BBB.LD, AAVrh.10.LD, AAV9.496NNN/AAA498, VOY101, VOY201, VOY701, VOY801, VOY1101, AAV.hu37, AAVrh74 version 1, AAVrh74 version 2, AAV.hu.31, AAV.hu32, AAV2, AAV3B, AAV2.7m8, AAV.rh.34, AAV.hu.31, AAV.rh.31, AAV.hu.12, AAV.hu.13, AAV.hu.21, AAV.hu.26, AAV.hu.53, AAV.hu.56, AAV.rh.24, AAV.hu.38, AAV.rh.72, AAV.cy.5, AAV.cy.6, AAV.rh.46, or AAV.rh.2, or a derivative, modification, or pseudotype thereof.

In particular embodiments, the recombinant AAV for use in compositions and methods herein is Anc80 or Anc80L65 (see, e.g., Zinn E et al., 2015, Cell Rep. 12 (6): 1056-1068, which is incorporated by reference in its entirety). In particular embodiments, the recombinant AAV for use in compositions and methods herein is AAV.7m8 (including variants thereof) (see, e.g., U.S. Pat. Nos. 9,193,956; 9,458,517; 9,587,282; US 2016/0376323, and WO 2018/075798, each of which is incorporated herein by reference in its entirety). In particular embodiments, the AAV for use in compositions and methods herein is any AAV disclosed in U.S. Pat. No. 9,585,971, such as AAV.PHP.B. In particular embodiments, the AAV for use in compositions and methods herein is an AAV2/Rec2 or AAV2/Rec3 vector, which has hybrid capsid sequences derived from AAV8 and serotypes cy5, rh20 or rh39 (see, e.g., Issa P C, et al., PLOS One. 2013 Apr. 9; 8(4):e60361., which is incorporated by reference herein for these vectors). In particular embodiments, the AAV for use in compositions and methods herein is an AAV disclosed in any of the following, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; 9,587,282; US 2015/0374803; US 2015/0126588; US 2017/0067908; US 2013/0224836; US 2016/0215024; US 2017/0051257; PCT/US2015/034799; PCT/EP2015/053335, PCT/US2019/032387, and PCT/US2019/055756. In some embodiments, rAAV particles have a capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e., up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799, PCT/EP2015/053335.

In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis A, et al., Gene Ther. 2016 December; 23 (12): 857-862 and Georgiadis, A., et al., Gene Ther 25, 450 (2018), each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo F. et al., 2017, Sci. Transl. Med. 29 (9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles have a capsid protein disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of 888 publication), WO 2006/110689, (see, e.g., SEQ ID NOs: 5-38 of '689 publication) WO2009/104964 (see, e.g., SEQ ID NOs: 1-5, 7, 9, 20, 22, 24 and 31 of '964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication), the contents of each of which is herein incorporated by reference in its entirety. In some embodiments, rAAV particles have a capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689 (see, e.g., SEQ ID NOs: 5-38 of '689 publication) WO2009/104964 (see, e.g., SEQ ID NOs: 1-5, 7, 9, 20, 22, 24 and 31 of 964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication).

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan D. et al., J. Virol., 75:7662-7671 (2001); Halbert C L. et al., J. Virol., 74:1524-1532 (2000); Zolotukhin S. et al., Methods 28:158-167 (2002); and Auricchio A. et al., Hum. Molec. Genet. 10:3075-3081, (2001).

AAV8-based, AAV9-based, and AAVrh10-based viral vectors are used in certain of the methods described herein. Nucleotide sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV (e.g., AAV8, AAV9 or AAVrh10)-based viral vectors encoding the DN-TNFα transgene.

Representative amino acid sequences of AAV capsids that can be used to target CNS tissues are provided in Table 1 below. Modified AAV9 capsids include: AAV9; AAV.PHP.eB; AAV.hDyn (AAV9 with TLAAPFK (SEQ ID NO: 174) between 588-589 with no other amino acid modifications to the capsid sequence); AAV.PHP.S; and AAV.PHP.SH. AAV.PHP.B (GenBank entry: ALU85156.1-Deverman B E et al., 2016 Nature Biotech 34 (2): 204-9)) is a capsid having a TLAVPFK (SEQ ID NO: 175) insertion in AAV9 capsid between amino acid residues 588-589, with no other amino acid modifications to the capsid sequence. AAV.PHP.eB (Chan et al 2017, Nat Neurosci 20 (8): 1172-1179) is a capsid having a TLAVPFK (SEQ ID NO: 175) insertion in AAV9 capsid, with having two amino acid modifications of the capsid sequence upstream of the PHP.B insertion. Likewise, peptide insertions were made to AAV9 to generate AAV.PHP.S and AAV.PHP.SH (Chan et al 2017, Nat Neurosci 20 (8): 1172-1179). Additional AAV9 modified vectors are found in WO2020206189A1, incorporated by reference herein in its entirety.

TABLE 1
Representative Capsids for CNS Tropism

| Capsid | Amino Acid Sequence |
|---|---|
| AAV9 | MAADGYLPDWLEDNLSEGIREWWALKPGAPQPKANQQHQDNARGLVL |
| (SEQ ID NO: 5) | PGYKYLGPGNGLDKGEPVNAADAAALEHDKAYDQQLKAGDNPYLKYNHA |
| | DAEFQERLKEDTSFGGNLGRAVFQAKKRLLEPLGLVEEAAKTAPGKKRPVE |
| | QSPQEPDSSAGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQRLI |
| | NNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSKTINGSGQN |
| | QQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKV |
| | MITNEEEIKTTNPVATESYGQVATNHQSAQAQAQTGWVQNQGILPGMVWQ |
| | DRDVYLQGPIWAKIPHTDGNFHPSPLMGGFGMKHPPPQILIKNTPVPADPPT |
| | AFNKDKLNSFITQYSTGQVSVEIEWELQKENSKRWNPEIQYTSNYYKSNNVE |
| | FAVNTEGVYSEPRPIGTRYLTRNL |
| AAV.hDyn | MAADGYLPDWLEDNLSEGIREWWALKPGAPQPKANQQHQDNARGLVL |
| (SEQ ID NO: 6) | PGYKYLGPGNGLDKGEPVNAADAAALEHDKAYDQQLKAGDNPYLKYNHA |
| | DAEFQERLKEDTSFGGNLGRAVFQAKKRLLEPLGLVEEAAKTAPGKKRPVE |
| | QSPQEPDSSAGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQRLI |
| | NNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSRTINGSGQN |
| | QQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKV |
| | MITNEEEIKTTNPVATESYGQVATNHQSAQTLAAPFKAQAQTGWVQNQGIL |
| | PGMVWQDRDVYLQGPIWAKIPHTDGNFHPSPLMGGFGMKHPPPQILIKNTP |
| | VPADPPTAFNKDKLNSFITQYSTGQVSVEIEWELQKENSKRWNPEIQYTSNY |
| | YKSNNVEFAVNTEGVYSEPRPIGTRYLTRNL |
| AAV.PHP.eB | MAADGYLPDWLEDNLSEGIREWWALKPGAPQPKANQQHQDNARGLVL |
| (SEQ ID NO: 7) | PGYKYLGPGNGLDKGEPVNAADAAALEHDKAYDQQLKAGDNPYLKYNHA |
| | DAEFQERLKEDTSFGGNLGRAVFQAKKRLLEPLGLVEEAAKTAPGKKRPVE |
| | QSPQEPDSSAGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQRLI |
| | NNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSRTINGSGQN |
| | QQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKV |
| | MITNEEEIKTTNPVATESYGQVATNHQSDGTLAVPFKAQAQTGWVQNQGIL |
| | PGMVWQDRDVYLQGPIWAKIPHTDGNFHPSPLMGGFGMKHPPPQILIKNTP |
| | VPADPPTAFNKDKLNSFITQYSTGQVSVEIEWELQKENSKRWNPEIQYTSNY |
| | YKSNNVEFAVNTEGVYSEPRPIGTRYLTRNL |
| AAV.PHP.B | MAADGYLPDWLEDNLSEGIREWWALKPGAPQPKANQQHQDNARGLVL |
| (SEQ ID NO: 8) | PGYKYLGPGNGLDKGEPVNAADAAALEHDKAYDQQLKAGDNPYLKYNHA |
| | DAEFQERLKEDTSFGGNLGRAVFQAKKRLLEPLGLVEEAAKTAPGKKRPVE |
| | QSPQEPDSSAGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQRLI |
| | NNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSRTINGSGQN |
| | QQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKV |
| | MITNEEEIKTTNPVATESYGQVATNHQSAQTLAVPFKAQAQTGWVQNQGIL |
| | PGMVWQDRDVYLQGPIWAKIPHTDGNFHPSPLMGGFGMKHPPPQILIKNTP |
| | VPADPPTAFNKDKLNSFITQYSTGQVSVEIEWELQKENSKRWNPEIQYTSNY |
| | YKSNNVEFAVNTEGVYSEPRPIGTRYLTRNL |
| AAV.PHP.S | MAADGYLPDWLEDNLSEGIREWWALKPGAPQPKANQQHQDNARGLVL |
| (SEQ ID NO: 9) | PGYKYLGPGNGLDKGEPVNAADAAALEHDKAYDQQLKAGDNPYLKYNHA |
| | DAEFQERLKEDTSFGGNLGRAVFQAKKRLLEPLGLVEEAAKTAPGKKRPVE |
| | QSPQEPDSSAGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQRLI |
| | NNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSRTINGSGQN |
| | QQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKV |
| | MITNEEEIKTTNPVATESYGQVATNHQSAQQAVRTSLAQAQTGWVQNQGIL |
| | PGMVWQDRDVYLQGPIWAKIPHTDGNFHPSPLMGGFGMKHPPPQILIKNTP |
| | VPADPPTAFNKDKLNSFITQYSTGQVSVEIEWELQKENSKRWNPEIQYTSNY |
| | YKSNNVEFAVNTEGVYSEPRPIGTRYLTRNL |
| AAV.PHP.SH | MAADGYLPDWLEDNLSEGIREWWALKPGAPQPKANQQHQDNARGLVL |
| (SEQ ID NO: 10) | PGYKYLGPGNGLDKGEPVNAADAAALEHDKAYDQQLKAGDNPYLKYNHA |
| | DAEFQERLKEDTSFGGNLGRAVFQAKKRLLEPLGLVEEAAKTAPGKKRPVE |
| | QSPQEPDSSAGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQRLI |
| | NNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSRTINGSGQN |
| | QQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKV |
| | MITNEEEIKTTNPVATESYGQVATNHQSAQQAVRTSHAQAQTGWVQNQGIL |
| | PGMVWQDRDVYLQGPIWAKIPHTDGNFHPSPLMGGFGMKHPPPQILIKNTP |
| | VPADPPTAFNKDKLNSFITQYSTGQVSVEIEWELQKENSKRWNPEIQYTSNY |
| | YKSNNVEFAVNTEGVYSEPRPIGTRYLTRNL |
| AAV8 | MAADGYLPDWLEDNLSEGIREWWALKPGAPKPKANQQKQDDGRGLVL |
| (SEQ ID NO: 11) | PGYKYLGPFNGLDKGEPVNAADAAALEHDKAYDQQLQAGDNPYLRYNHA |
| | DAEFQERLQEDTSFGGNLGRAVFQAKKRVLEPLGLVEEGAKTAPGKKRPVE |
| | PSPQRSPDSSTGIGKKGQQPARKRLNFGQTGDSESVPDPQPLGEPPAAPSGVG |
| | PNTMAAGGGAPMADNNEGADGVGSSSGNWHCDSTWLGDRVITTSTRTWA |
| | LPTYNNHLYKQISNGTSGGATNDNTYFGYSTPWGYFDFNRFHCHFSPRDWQ |
| | RLINNNWGFRPKRLSFKLFNIQVKEVTQNEGTKTIANNLTSTIQVFTDSEYQL |
| | PYVLGSAHQGCLPPFPADVFMIPQYGYLTLNNGSQAVGRSSFYCLEYFPSQM |
| | LRTGNNFQFTYTFEDVPFHSSYAHSQSLDRLMNPLIDQYLYYLSRTQTTGGT |
| | ANTQTLGFSQGGPNTMANQAKNWLPGPCYRQQRVSTTTGQNNNSNFAWT |
| | AGTKYHLNGRNSLANPGIAMATHKDDEERFFPSNGILIFGKQNAARDNADY |
| | SDVMLTSEEEIKTTNPVATEEYGIVADNLQQQNTAPQIGTVNSQGALPGMV |
| | WQNRDVYLQGPIWAKIPHTDGNFHPSPLMGGFGLKHPPPQILIKNTPVPADP |
| | PTTFNQSKLNSFITQYSTGQVSVEIEWELQKENSKRWNPEIQYTSNYYKSTSV |
| | DFAVNTEGVYSEPRPIGTRYLTRNL |
| AAV8.BBB | MAADGYLPDWLEDNLSEGIREWWALKPGAPKPKANQQKQDDGRGLVL |
| (AAV8 A269S) | PGYKYLGPFNGLDKGEPVNAADAAALEHDKAYDQQLQAGDNPYLRYNHA |
| (SEQ ID NO: 12) | DAEFQERLQEDTSFGGNLGRAVFQAKKRVLEPLGLVEEGAKTAPGKKRPVE |
| | PSPQRSPDSSTGIGKKGQQPARKRLNFGQTGDSESVPDPQPLGEPPAAPSGVG |
| | PNTMAAGGGAPMADNNEGADGVGSSSGNWHCDSTWLGDRVITTSTRTWA |
| | LPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQL |
| | PYVLGSAHQGCLPPFPADVFMIPQYGYLTLNNGSQAVGRSSFYCLEYFPSQM |
| | LRTGNNFQFTYTFEDVPFHSSYAHSQSLDRLMNPLIDQYLYYLSRTQTTGGT |
| | ANTQTLGFSQGGPNTMANQAKNWLPGPCYRQQRVSTTTGQNNNSNFAWT |
| | AGTKYHLNGRNSLANPGIAMATHKDDEERFFPSNGILIFGKQNAARDNADY |
| | SDVMLTSEEEIKTTNPVATEEYGIVADNLQQQNTAPQIGTVNSQGALPGMV |
| | WQNRDVYLQGPIWAKIPHTDGNFHPSPLMGGFGLKHPPPQILIKNTPVPADP |
| | PTTFNQSKLNSFITQYSTGQVSVEIEWELQKENSKRWNPEIQYTSNYYKSTSV |
| | DFAVNTEGVYSEPRPIGTRYLTRNL |
| AAV8.BBB.LD | MAADGYLPDWLEDNLSEGIREWWALKPGAPKPKANQQKQDDGRGLVL |
| (AAV8 A269S, | PGYKYLGPFNGLDKGEPVNAADAAALEHDKAYDQQLQAGDNPYLRYNHA |
| 498_NNN/ | DAEFQERLQEDTSFGGNLGRAVFQAKKRVLEPLGLVEEGAKTAPGKKRPVE |
| AAA_500) | PSPQRSPDSSTGIGKKGQQPARKRLNFGQTGDSESVPDPQPLGEPPAAPSGVG |
| (SEQ ID NO: 13) | PNTMAAGGGAPMADNNEGADGVGSSSGNWHCDSTWLGDRVITTSTRTWA |
| | LPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQL |
| | PYVLGSAHQGCLPPFPADVFMIPQYGYLTLNNGSQAVGRSSFYCLEYFPSQM |
| | LRTGNNFQFTYTFEDVPFHSSYAHSQSLDRLMNPLIDQYLYYLSRTQTTGGT |
| | ANTQTLGFSQGGPNTMANQAKNWLPGPCYRQQRVSTTTGQAAASNFAWT |
| | AGTKYHLNGRNSLANPGIAMATHKDDEERFFPSNGILIFGKQNAARDNADY |
| | SDVMLTSEEEIKTTNPVATEEYGIVADNLQQQNTAPQIGTVNSQGALPGMV |
| | WQNRDVYLQGPIWAKIPHTDGNFHPSPLMGGFGLKHPPPQILIKNTPVPADP |
| | PTTFNQSKLNSFITQYSTGQVSVEIEWELQKENSKRWNPEIQYTSNYYKSTSV |
| | DFAVNTEGVYSEPRPIGTRYLTRNL |
| AAV9.BBB | MAADGYLPDWLEDNLSEGIREWWALKPGAPQPKANQQHQDNARGLVL |
| (AAV9 S263G/ | PGYKYLGPGNGLDKGEPVNAADAAALEHDKAYDQQLKAGDNPYLKYNHA |
| S269T/A273T) | DAEFQERLKEDTSFGGNLGRAVFQAKKRLLEPLGLVEEAAKTAPGKKRPVE |
| (SEQ ID NO: 14) | QSPQEPDSSAGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNGTSGGSTNDNTYFGYSTPWGYFDFNRFHCHFSPRDWQRL |
| | INNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSKTINGSGQN |
| | QQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKV |
| | MITNEEEIKTTNPVATESYGQVATNHQSAQAQAQTGWVQNQGILPGMVWQ |
| | DRDVYLQGPIWAKIPHTDGNFHPSPLMGGFGMKHPPPQILIKNTPVPADPPT |
| | AFNKDKLNSFITQYSTGQVSVEIEWELQKENSKRWNPEIQYTSNYYKSNNVE |
| | FAVNTEGVYSEPRPIGTRYLTRNL |
| AAV9.BBB.LD | MAADGYLPDWLEDNLSEGIREWWALKPGAPQPKANQQHQDNARGLVL |
| (AAV9 S263G/ | PGYKYLGPGNGLDKGEPVNAADAAALEHDKAYDQQLKAGDNPYLKYNHA |
| S269T/A273T, | DAEFQERLKEDTSFGGNLGRAVFQAKKRLLEPLGLVEEAAKTAPGKKRPVE |
| 496_NNN/ | QSPQEPDSSAGIGKSGAQPAKKRLNFGQTGDTESVPDPQPIGEPPAAPSGVGS |
| AAA_498) | LTMASGGGAPVADNNEGADGVGSSSGNWHCDSQWLGDRVITTSTRTWALP |
| (SEQ ID NO: 15) | TYNNHLYKQISNGTSGGSTNDNTYFGYSTPWGYFDFNRFHCHFSPRDWQRL |
| | INNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSKTINGSGQN |
| | QQTLKFSVAGPSNMAVQGRNYIPGPSYRQQRVSTTVTQAAASEFAWPGASS |
| | WALNGRNSLMNPGPAMASHKEGEDRFFPLSGSLIFGKQGTGRDNVDADKV |
| | MITNEEEIKTTNPVATESYGQVATNHQSAQAQAQTGWVQNQGILPGMVWQ |
| | DRDVYLQGPIWAKIPHTDGNFHPSPLMGGFGMKHPPPQILIKNTPVPADPPT |
| | AFNKDKLNSFITQYSTGQVSVEIEWELQKENSKRWNPEIQYTSNYYKSNNVE |
| | FAVNTEGVYSEPRPIGTRYLTRNL |
| AAVrh10 | MAADGYLPDWLEDNLSEGIREWWDLKPGAPKPKANQQKQDDGRGLVL |
| (SEQ ID NO: 16) | PGYKYLGPFNGLDKGEPVNAADAAALEHDKAYDQQLKAGDNPYLRYNHA |
| | DAEFQERLQEDTSFGGNLGRAVFQAKKRVLEPLGLVEEGAKTAPGKKRPVE |
| | PSPQRSPDSSTGIGKKGQQPAKKRLNFGQTGDSESVPDPQPIGEPPAGPSGLG |
| | SGTMAAGGGAPMADNNEGADGVGSSSGNWHCDSTWLGDRVITTSTRTWA |
| | LPTYNNHLYKQISNGTSGGSTNDNTYFGYSTPWGYFDFNRFHCHFSPRDWQ |
| | RLINNNWGFRPKRLNFKLFNIQVKEVTQNEGTKTIANNLTSTIQVFTDSEYQL |
| | PYVLGSAHQGCLPPFPADVFMIPQYGYLTLNNGSQAVGRSSFYCLEYFPSQM |
| | LRTGNNFEFSYQFEDVPFHSSYAHSQSLDRLMNPLIDQYLYYLSRTQSTGGT |
| | AGTQQLLFSQAGPNNMSAQAKNWLPGPCYRQQRVSTTLSQNNNSNFAWTG |
| | ATKYHLNGRDSLVNPGVAMATHKDDEERFFPSSGVLMFGKQGAGKDNVD |
| | YSSVMLTSEEEIKTTNPVATEQYGVVADNLQQQNAAPIVGAVNSQGALPGM |
| | VWQNRDVYLQGPIWAKIPHTDGNFHPSPLMGGFGLKHPPPQILIKNTPVPAD |
| | PPTTFSQAKLASFITQYSTGQVSVEIEWELQKENSKRWNPEIQYTSNYYKSTN |
| | VDFAVNTDGTYSEPRPIGTRYLTRNL |
| AAVrh.10.LD | MAADGYLPDWLEDNLSEGIREWWDLKPGAPKPKANQQKQDDGRGLVL |
| (rh10 498_NNN/ | PGYKYLGPFNGLDKGEPVNAADAAALEHDKAYDQQLKAGDNPYLRYNHA |
| AAA_500) | DAEFQERLQEDTSFGGNLGRAVFQAKKRVLEPLGLVEEGAKTAPGKKRPVE |
| (SEQ ID NO: 17) | PSPQRSPDSSTGIGKKGQQPAKKRLNFGQTGDSESVPDPQPIGEPPAGPSGLG |
| | SGTMAAGGGAPMADNNEGADGVGSSSGNWHCDSTWLGDRVITTSTRTWA |
| | LPTYNNHLYKQISNGTSGGSTNDNTYFGYSTPWGYFDFNRFHCHFSPRDWQ |
| | RLINNNWGFRPKRLNFKLFNIQVKEVTQNEGTKTIANNLTSTIQVFTDSEYQL |
| | PYVLGSAHQGCLPPFPADVFMIPQYGYLTLNNGSQAVGRSSFYCLEYFPSQM |
| | LRTGNNFEFSYQFEDVPFHSSYAHSQSLDRLMNPLIDQYLYYLSRTQSTGGT |
| | AGTQQLLFSQAGPNNMSAQAKNWLPGPCYRQQRVSTTLSQAAASNFAWTG |
| | ATKYHLNGRDSLVNPGVAMATHKDDEERFFPSSGVLMFGKQGAGKDNVD |
| | YSSVMLTSEEEIKTTNPVATEQYGVVADNLQQQNAAPIVGAVNSQGALPGM |
| | VWQNRDVYLQGPIWAKIPHTDGNFHPSPLMGGFGLKHPPPQILIKNTPVPAD |
| | PPTTFSQAKLASFITQYSTGQVSVEIEWELQKENSKRWNPEIQYTSNYYKSTN |
| | VDFAVNTDGTYSEPRPIGTRYLTRNL |
| AAV9.496NNN/AA | MAADGYLPDWLEDNLSEGIREWWALKPGAPQPKANQQHQDNARGLVL |
| A498 | PGYKYLGPGNGLDKGEPVNAADAAALEHDKAYDQQLKAGDNPYLKYNHA |
| (SEQ ID NO: 18) | DAEFQERLKEDTSFGGNLGRAVFQAKKRLLEPLGLVEEAAKTAPGKKRPVE |
| | QSPQEPDSSAGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQRLI |
| | NNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSKTINGSGQN |
| | QQTLKFSVAGPSNMAVQGRNYIPGPSYRQQRVSTTVTQAAASEFAWPGASS |
| | WALNGRNSLMNPGPAMASHKEGEDRFFPLSGSLIFGKQGTGRDNVDADKV |
| | MITNEEEIKTTNPVATESYGQVATNHQSAQAQAQTGWVQNQGILPGMVWQ |
| | DRDVYLQGPIWAKIPHTDGNFHPSPLMGGFGMKHPPPQILIKNTPVPADPPT |
| | AFNKDKLNSFITQYSTGQVSVEIEWELQKENSKRWNPEIQYTSNYYKSNNVE |
| | FAVNTEGVYSEPRPIGTRYLTRNL |
| VOY101 | MAADGYLPDWLEDNLSEGIREWWALKPGAPQPKANQQHQDNARGLVL |
| (SEQ ID NO: 19) | PGYKYLGPGNGLDKGEPVNAADAAALEHDKAYDQQLKAGDNPYLKYNHA |
| | DAEFQERLKEDTSFGGNLGRAVFQAKKRLLEPLGLVEEAAKTAPGKKRPVE |
| | QSPQEPDSSAGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQRLI |
| | NNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSKTINGSGQN |
| | QQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKV |
| | MITNEEEIKTTNPVATESYGQVATNHQSDGTLAVPFKAQAQTGWVQNQGIL |
| | PGMVWQDRDVYLQGPIWAKIPHTDGNFHPSPLMGGFGMKHPPPQILIKNTP |
| | VPADPPTAFNKDKLNSFITQYSTGQVSVEIEWELQKENSKRWNPEIQYTSNY |
| | YKSNNVEFAVNTEGVYSEPRPIGTRYLTRNL |
| VOY201 | MAADGYLPDWLEDNLSEGIREWWALKPGAPQPKANQQHQDNARGLVL |
| (SEQ ID NO: 20) | PGYKYLGPGNGLDKGEPVNAADAAALEHDKAYDQQLKAGDNPYLKYNHA |
| | DAEFQERLKEDTSFGGNLGRAVFQAKKRLLEPLGLVEEAAKTAPGKKRPVE |
| | QSPQEPDSSAGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQRLI |
| | NNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSKTINGSGQN |
| | QQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKV |
| | MITNEEEIKTTNPVATESYGQVATNHQSAQTLAVPFKAQAQTGWVQNQGIL |
| | PGMVWQDRDVYLQGPIWAKIPHTDGNFHPSPLMGGFGMKHPPPQILIKNTP |
| | VPADPPTAFNKDKLNSFITQYSTGQVSVEIEWELQKENSKRWNPEIQYTSNY |
| | YKSNNVEFAVNTEGVYSEPRPIGTRYLTRNL |
| VOY701 | MAADGYLPDWLEDNLSEGIREWWALKPGAPQPKANQQHQDNARGLVL |
| (SEQ ID NO: 21) | PGYKYLGPGNGLDKGEPVNAADAAALEHDKAYDQQLKAGDNPYLKYNHA |
| | DAEFQERLKEDTSFGGNLGRAVFQAKKRLLEPLGLVEEAAKTAPGKKRPVE |
| | QSPQEPDSSAGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQRLI |
| | NNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSRTINGSGQN |
| | QQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKV |
| | MITNEEEIKTTNPVATESYGQVATNHQSESTLAVPFKAQAQTGWVQNQGILP |
| | GMVWQDRDVYLQGPIWAKIPHTDGNFHPSPLMGGFGMKHPPPQILIKNTPV |
| | PADPPTAFNKDKLNSFITQYSTGQVSVEIEWELQKENSKRWNPEIQYTSNYY |
| | KSNNVEFAVNTEGVYSEPRPIGTRYLTRNL |
| VOY801 | MAADGYLPDWLEDNLSEGIREWWALKPGAPQPKANQQHQDNARGLVL |
| (SEQ ID NO: 22) | PGYKYLGPGNGLDKGEPVNAADAAALEHDKAYDQQLKAGDNPYLKYNHA |
| | DAEFQERLKEDTSFGGNLGRAVFQAKKRLLEPLGLVEEAAKTAPGKKRPVE |
| | QSPQEPDSSAGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQRLI |
| | NNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSRTINGSGQN |
| | QQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKV |
| | MITNEEEIKTTNPVATESYGQVATNHQSGGTLAVPFKAQAQTGWVQNQGIL |
| | PGMVWQDRDVYLQGPIWAKIPHTDGNFHPSPLMGGFGMKHPPPQILIKNTP |
| | VPADPPTAFNKDKLNSFITQYSTGQVSVEIEWELQKENSKRWNPEIQYTSNY |
| | YKSNNVEFAVNTEGVYSEPRPIGTRYLTRNL |
| VOY1101 | MAADGYLPDWLEDNLSEGIREWWALKPGAPQPKANQQHQDNARGLVL |
| (SEQ ID NO: 23) | PGYKYLGPGNGLDKGEPVNAADAAALEHDKAYDQQLKAGDNPYLKYNHA |
| | DAEFQERLKEDTSFGGNLGRAVFQAKKRLLEPLGLVEEAAKTAPGKKRPVE |
| | QSPQEPDSSAGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQRLI |
| | NNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSRTINGSGQN |
| | QQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKV |
| | MITNEEEIKTTNPVATESYGQVATNHQSDGTLATPFKAQAQTGWVQNQGIL |
| | PGMVWQDRDVYLQGPIWAKIPHTDGNFHPSPLMGGFGMKHPPPQILIKNTP |
| | VPADPPTAFNKDKLNSFITQYSTGQVSVEIEWELQKENSKRWNPEIQYTSNY |
| | YKSNNVEFAVNTEGVYSEPRPIGTRYLTRNL |
| AAV.S454.Tfr3 | MAADGYLPDWLEDNLSEGIREWWALKPGAPQPKANQQHQDNARGLVL |
| (SEQ ID NO: 24) | PGYKYLGPGNGLDKGEPVNAADAAALEHDKAYDQQLKAGDNPYLKYNHA |
| | DAEFQERLKEDTSFGGNLGRAVFQAKKRLLEPLGLVEEAAKTAPGKKRPVE |
| | QSPQEPDSSAGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQRLI |
| | NNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSKTINGSRTIG |
| | PSVGQNQQTLKFSVAGPSNMAVQGRNYIPGPSYRQQRVSTTVTQNNNSEFA |
| | WPGASSWALNGRNSLMNPGPAMASHKEGEDRFFPLSGSLIFGKQGTGRDNV |
| | DADKVMITNEEEIKTTNPVATESYGQVATNHQSAQAQAQTGWVQNQGILPG |
| | MVWQDRDVYLQGPIWAKIPHTDGNFHPSPLMGGFGMKHPPPQILIKNTPVP |
| | ADPPTAFNKDKLNSFITQYSTGQVSVEIEWELQKENSKRWNPEIQYTSNYYK |
| | SNNVEFAVNTEGVYSEPRPIGTRYLTRNL |
| AAV1 | MAADGYLPDWLEDNLSEGIREWWDLKPGAPKPKANQQKQDDGRGLVLPG |
| (SEQ ID NO: 177) | YKYLGPFNGLDKGEPVNAADAAALEHDKAYDQQLKAGDNPYLRYNHADA |
| | EFQERLQEDTSFGGNLGRAVFQAKKRVLEPLGLVEEGAKTAPGKKRPVEQS |
| | PQEPDSSSGIGKTGQQPAKKRLNFGQTGDSESVPDPQPLGEPPATPAAVGPTT |
| | MASGGGAPMADNNEGADGVGNASGNWHCDSTWLGDRVITTSTRTWALPT |
| | YNNHLYKQISSASTGASNDNHYFGYSTPWGYFDFNRFHCHFSPRDWQRLIN |
| | NNWGFRPKRLNFKLFNIQVKEVTTNDGVTTIANNLTSTVQVFSDSEYQLPYV |
| | LGSAHQGCLPPFPADVFMIPQYGYLTLNNGSQAVGRSSFYCLEYFPSQMLRT |
| | GNNFTFSYTFEEVPFHSSYAHSQSLDRLMNPLIDQYLYYLNRTQNQSGSAQN |
| | KDLLFSRGSPAGMSVQPKNWLPGPCYRQQRVSKTKTDNNNSNFTWTGASK |
| | YNLNGRESIINPGTAMASHKDDEDKFFPMSGVMIFGKESAGASNTALDNVMI |
| | TDEEEIKATNPVATERFGTVAVNFQSSSTDPATGDVHAMGALPGMVWQDR |
| | DVYLQGPIWAKIPHTDGHFHPSPLMGGFGLKNPPPQILIKNTPVPANPPAEFS |
| | ATKFASFITQYSTGQVSVEIEWELQKENSKRWNPEVQYTSNYAKSANVDFT |
| | VDNNGLYTEPRPIGTRYLTRPL |
| AAV7 | MAADGYLPDWLEDNLSEGIREWWDLKPGAPKPKANQQKQDNGRGLVLPG |
| (SEQ ID NO: 182) | YKYLGPFNGLDKGEPVNAADAAALEHDKAYDQQLKAGDNPYLRYNHADA |
| | EFQERLQEDTSFGGNLGRAVFQAKKRVLEPLGLVEEGAKTAPAKKRPVEPSP |
| | QRSPDSSTGIGKKGQQPARKRLNFGQTGDSESVPDPQPLGEPPAAPSSVGSGT |
| | VAAGGGAPMADNNEGADGVGNASGNWHCDSTWLGDRVITTSTRTWALPT |
| | YNNHLYKQISSETAGSTNDNTYFGYSTPWGYFDFNRFHCHFSPRDWQRLIN |
| | NNWGFRPKKLRFKLFNIQVKEVTTNDGVTTIANNLTSTIQVFSDSEYQLPYV |
| | LGSAHQGCLPPFPADVFMIPQYGYLTLNNGSQSVGRSSFYCLEYFPSQMLRT |
| | GNNFEFSYSFEDVPFHSSYAHSQSLDRLMNPLIDQYLYYLARTQSNPGGTAG |
| | NRELQFYQGGPSTMAEQAKNWLPGPCFRQQRVSKTLDQNNNSNFAWTGAT |
| | KYHLNGRNSLVNPGVAMATHKDDEDRFFPSSGVLIFGKTGATNKTTLENVL |
| | MTNEEEIRPTNPVATEEYGIVSSNLQAANTAAQTQVVNNQGALPGMVWQN |
| | RDVYLQGPIWAKIPHTDGNFHPSPLMGGFGLKHPPPQILIKNTPVPANPPEVF |
| | TPAKFASFITQYSTGQVSVEIEWELQKENSKRWNPEIQYTSNFEKQTGVDFA |
| | VDSQGVYSEPRPIGTRYLTRNL |

Representative amino acid sequences of AAV capsids that can be used to target systemic tissues, for example, muscle, liver, synovial fluid, or lung, are provided in Table 2 below.

TABLE 2
Representative Capsids for Muscle/Liver/Synovial Tropism

| Capsid | Amino Acid Sequence |
|---|---|
| AAV8 | MAADGYLPDWLEDNLSEGIREWWALKPGAPKPKANQQKQDDGRGLVL |
| (SEQ ID NO: 11) | PGYKYLGPFNGLDKGEPVNAADAAALEHDKAYDQQLQAGDNPYLRYNHA |
| | DAEFQERLQEDTSFGGNLGRAVFQAKKRVLEPLGLVEEGAKTAPGKKRPVE |
| | PSPQRSPDSSTGIGKKGQQPARKRLNFGQTGDSESVPDPQPLGEPPAAPSGVG |
| | PNTMAAGGGAPMADNNEGADGVGSSSGNWHCDSTWLGDRVITTSTRTWA |
| | LPTYNNHLYKQISNGTSGGATNDNTYFGYSTPWGYFDFNRFHCHFSPRDWQ |
| | RLINNNWGFRPKRLSFKLFNIQVKEVTQNEGTKTIANNLTSTIQVFTDSEYQL |
| | PYVLGSAHQGCLPPFPADVFMIPQYGYLTLNNGSQAVGRSSFYCLEYFPSQM |
| | LRTGNNFQFTYTFEDVPFHSSYAHSQSLDRLMNPLIDQYLYYLSRTQTTGGT |
| | ANTQTLGFSQGGPNTMANQAKNWLPGPCYRQQRVSTTTGQNNNSNFAWT |
| | AGTKYHLNGRNSLANPGIAMATHKDDEERFFPSNGILIFGKQNAARDNADY |
| | SDVMLTSEEEIKTTNPVATEEYGIVADNLQQQNTAPQIGTVNSQGALPGMV |
| | WQNRDVYLQGPIWAKIPHTDGNFHPSPLMGGFGLKHPPPQILIKNTPVPADP |
| | PTTFNQSKLNSFITQYSTGQVSVEIEWELQKENSKRWNPEIQYTSNYYKSTSV |
| | DFAVNTEGVYSEPRPIGTRYLTRNL |
| AAV9 | MAADGYLPDWLEDNLSEGIREWWALKPGAPQPKANQQHQDNARGLVL |
| (SEQ ID NO: 5) | PGYKYLGPGNGLDKGEPVNAADAAALEHDKAYDQQLKAGDNPYLKYNHA |
| | DAEFQERLKEDTSFGGNLGRAVFQAKKRLLEPLGLVEEAAKTAPGKKRPVE |
| | QSPQEPDSSAGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQRLI |
| | NNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSKTINGSGQN |
| | QQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKV |
| | MITNEEEIKTTNPVATESYGQVATNHQSAQAQAQTGWVQNQGILPGMVWQ |
| | DRDVYLQGPIWAKIPHTDGNFHPSPLMGGFGMKHPPPQILIKNTPVPADPPT |
| | AFNKDKLNSFITQYSTGQVSVEIEWELQKENSKRWNPEIQYTSNYYKSNNVE |
| | FAVNTEGVYSEPRPIGTRYLTRNL |
| AAV.hu37 | MAADGYLPDWLEDNLSEGIREWWDLKPGAPKPKANQQKQDDGRGLVL |
| (SEQ ID NO: 25) | PGYKYLGPFNGLDKGEPVNAADAAALEHDKAYDQQLKAGDNPYLRYNHA |
| | DAEFQERLQEDTSFGGNLGRAVFQAKKRVLEPLGLVEEAAKTAPGKKRPVE |
| | PSPQRSPDSSTGIGKKGQQPAKKRLNFGQTGDSESVPDPQPIGEPPAGPSGLG |
| | SGTMAAGGGAPMADNNEGADGVGSSSGNWHCDSTWLGDRVITTSTRTWA |
| | LPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQL |
| | PYVLGSAHQGCLPPFPADVFMIPQYGYLTLNNGSQAVGRSSFYCLEYFPSQM |
| | LRTGNNFEFSYTFEDVPFHSSYAHSQSLDRLMNPLIDQYLYYLSRTQSTGGT |
| | QGTQQLLFSQAGPANMSAQAKNWLPGPCYRQQRVSTTLSQNNNSNFAWTG |
| | ATKYHLNGRDSLVNPGVAMATHKDDEERFFPSSGVLMFGKQGAGRDNVDY |
| | SSVMLTSEEEIKTTNPVATEQYGVVADNLQQTNTGPIVGNVNSQGALPGMV |
| | WQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTN |
| | VDFAVNTEGTYSEPRPIGTRYLTRNL |
| AAVrh74 version 1 | MAADGYLPDWLEDNLSEGIREWWDLKPGAPKPKANQQKQDNGRGLVL |
| (SEQ ID NO: 26) | PGYKYLGPFNGLDKGEPVNAADAAALEHDKAYDQQLQAGDNPYLRYNHA |
| | DAEFQERLQEDTSFGGNLGRAVFQAKKRVLEPLGLVESPVKTAPGKKRPVE |
| | PSPQRSPDSSTGIGKKGQQPAKKRLNFGQTGDSESVPDPQPIGEPPAGPSGLG |
| | SGTMAAGGGAPMADNNEGADGVGSSSGNWHCDSTWLGDRVITTSTRTWA |
| | LPTYNNHLYKQISNGTSGGSTNDNTYFGYSTPWGYFDFNRFHCHFSPRDWQ |
| | RLINNNWGFRPKRLNFKLFNIQVKEVTQNEGTKTIANNLTSTIQVFTDSEYQL |
| | PYVLGSAHQGCLPPFPADVFMIPQYGYLTLNNGSQAVGRSSFYCLEYFPSQM |
| | LRTGNNFEFSYNFEDVPFHSSYAHSQSLDRLMNPLIDQYLYYLSRTQSTGGT |
| | AGTQQLLFSQAGPNNMSAQAKNWLPGPCYRQQRVSTTLSQNNNSNFAWTG |
| | ATKYHLNGRDSLVNPGVAMATHKDDEERFFPSSGVLMFGKQGAGKDNVD |
| | YSSVMLTSEEEIKTTNPVATEQYGVVADNLQQQNAAPIVGAVNSQGALPGM |
| | VWQNRDVYLQGPIWAKIPHTDGNFHPSPLMGGFGLKHPPPQILIKNTPVPAD |
| | PPTTFNQAKLASFITQYSTGQVSVEIEWELQKENSKRWNPEIQYTSNYYKST |
| | NVDFAVNTEGTYSEPRPIGTRYLTRNL |
| AAVrh74 version 2 | MAADGYLPDWLEDNLSEGIREWWDLKPGAPKPKANQQKQDNGRGLVL |
| (SEQ ID NO: 27) | PGYKYLGPFNGLDKGEPVNAADAAALEHDKAYDQQLQAGDNPYLRYNHA |
| | DAEFQERLQEDTSFGGNLGRAVFQAKKRVLEPLGLVESPVKTAPGKKRPVE |
| | PSPQRSPDSSTGIGKKGQQPAKKRLNFGQTGDSESVPDPQPIGEPPAAPSGVG |
| | PNTMAAGGGAPMADNNEGADGVGSSSGNWHCDSTWLGDRVITTSTRTWA |
| | LPTYNNHLYKQISNGTSGGSTNDNTYFGYSTPWGYFDFNRFHCHFSPRDWQ |
| | RLINNNWGFRPKRLNFKLFNIQVKEVTQNEGTKTIANNLTSTIQVFTDSEYQL |
| | PYVLGSAHQGCLPPFPADVFMIPQYGYLTLNNGSQAVGRSSFYCLEYFPSQM |
| | LRTGNNFEFSYNFEDVPFHSSYAHSQSLDRLMNPLIDQYLYYLSRTQSTGGT |
| | AGTQQLLFSQAGPNNMSAQAKNWLPGPCYRQQRVSTTLSQNNNSNFAWTG |
| | ATKYHLNGRDSLVNPGVAMATHKDDEERFFPSSGVLMFGKQGAGKDNVD |
| | YSSVMLTSEEEIKTTNPVATEQYGVVADNLQQQNAAPIVGAVNSQGALPGM |
| | VWQNRDVYLQGPIWAKIPHTDGNFHPSPLMGGFGLKHPPPQILIKNTPVPAD |
| | PPTTFNQAKLASFITQYSTGQVSVEIEWELQKENSKRWNPEIQYTSNYYKST |
| | NVDFAVNTEGTYSEPRPIGTRYLTRNL |
| AAV.hu.31 | MAADGYLPDWLEDTLSEGIRQWWKLKPGPPPPKPAERHKDDSRGLVLP |
| (SEQ ID NO: 28) | GYKYLGPGNGLDKGEPVNAADAAALEHDKAYDQQLKAGDNPYLKYNHAD |
| | AEFQERLKEDTSFGGNLGRAVFQAKKRLLEPLGLVEEAAKTAPGKKRPVEQ |
| | SPQEPDSSAGIGKSGSQPAKKKLNFGQTGDTESVPDPQPIGEPPAAPSGVGSL |
| | TMASGGGAPVADNNEGADGVGSSSGNWHCDSQWLGDRVITTSTRTWALPT |
| | YNNHLYKQISNSTSGGSSNDNAYFGYSTPWGYFDFNRFHCHFSPRDWQRLI |
| | NNNWGFRPKRLNFKLFNIQVKEVTDNNGVKTIANNLTSTVQVFTDSDYQLP |
| | YVLGSAHEGCLPPFPADVFMIPQYGYLTLNDGGQAVGRSSFYCLEYFPSQML |
| | RTGNNFQFSYEFENVPFHSSYAHSQSLDRLMNPLIDQYLYYLSKTINGSGQN |
| | QQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKV |
| | MITNEEEIKTTNPVATESYGQVATNHQSAQAQAQTGWVQNQGILPGMVWQ |
| | DRDVYLQGPIWAKIPHTDGNFHPSPLMGGFGMKHPPPQILIKNTPVPADPPT |
| | AFNKDKLNSFITQYSTGQVSVEIEWELQKENSKRWNPEIQYTSNYYKSNNVE |
| | FAVSTEGVYSEPRPIGTRYLTRNL |
| AAV.hu32 | MAADGYLPDWLEDTLSEGIRQWWKLKPGPPPPKPAERHKDDSRGLVLP |
| (SEQ ID NO: 29) | GYKYLGPGNGLDKGEPVNAADAAALEHDKAYDQQLKAGDNPYLKYNHAD |
| | AEFQERLKEDTSFGGNLGRAVFQAKKRLLEPLGLVEEAAKTAPGKKRPVEQ |
| | SPQEPDSSAGIGKSGSQPAKKKLNFGQTGDTESVPDPGQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQRLI |
| | NNNWGFRPKRLNFKLFNIQVKEVTDNNGVKTIANNLTSTVQVFTDSDYQLP |
| | YVLGSAHEGCLPPFPADVFMIPQYGYLTLNDGSQAVGRSSFYCLEYFPSQML |
| | RTGNNFQFSYEFENVPFHSSYAHSQSLDRLMNPLIDQYLYYLSKTINGSGQN |
| | QQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKV |
| | MITNEEEIKTTNPVATESYGQVATNHQSAQAQAQTGWVQNQGILPGMVWQ |
| | DRDVYLQGPIWAKIPHTDGNFHPSPLMGGFGMKHPPPQILIKNTPVPADPPT |
| | AFNKDKLNSFITQYSTGQVSVEIEWELQKENSKRWNPEIQYTSNYYKSNNVE |
| | FAVNTEGVYSEPRPIGTRYLTRNL |
| AAV3A | MAADGYLPDWLEDNLSEGIREWWALKPGVPQPKANQQHQDNRRGLVLPG |
| (SEQ ID NO: 178) | YKYLGPGNGLDKGEPVNEADAAALEHDKAYDQQLKAGDNPYLKYNHADA |
| | EFQERLQEDTSFGGNLGRAVFQAKKRILEPLGLVEEAAKTAPGKKGAVDQS |
| | PQEPDSSSGVGKSGKQPARKRLNFGQTGDSESVPDPQPLGEPPAAPTSLGSNT |
| | MASGGGAPMADNNEGADGVGNSSGNWHCDSQWLGDRVITTSTRTWALPT |
| | YNNHLYKQISSQSGASNDNHYFGYSTPWGYFDFNRFHCHFSPRDWQRLINN |
| | NWGFRPKKLSFKLFNIQVRGVTQNDGTTTIANNLTSTVQVFTDSEYQLPYVL |
| | GSAHQGCLPPFPADVFMVPQYGYLTLNNGSQAVGRSSFYCLEYFPSQMLRT |
| | GNNFQFSYTFEDVPFHSSYAHSQSLDRLMNPLIDQYLYYLNRTQGTTSGTTN |
| | QSRLLFSQAGPQSMSLQARNWLPGPCYRQQRLSKTANDNNNSNFPWTAAS |
| | KYHLNGRDSLVNPGPAMASHKDDEEKFFPMHGNLIFGKEGTTASNAELDNV |
| | MITDEEEIRTTNPVATEQYGTVANNLQSSNTAPTTGTVNHQGALPGMVWQD |
| | RDVYLQGPIWAKIPHTDGHFHPSPLMGGFGLKHPPPQIMIKNTPVPANPPTTF |
| | SPAKFASFITQYSTGQVSVEIEWELQKENSKRWNPEIQYTSNYNKSVNVDFT |
| | VDTNGVYSEPRPIGTRYLTRNL |
| AAV4 | MTDGYLPDWLEDNLSEGVREWWALQPGAPKPKANQQHQDNARGLVLPGY |
| (SEQ ID NO: 179) | KYLGPGNGLDKGEPVNAADAAALEHDKAYDQQLKAGDNPYLKYNHADAE |
| | FQQRLQGDTSFGGNLGRAVFQAKKRVLEPLGLVEQAGETAPGKKRPLIESPQ |
| | QPDSSTGIGKKGKQPAKKKLVFEDETGAGDGPPEGSTSGAMSDDSEMRAAA |
| | GGAAVEGGQGADGVGNASGDWHCDSTWSEGHVTTTSTRTWVLPTYNNHL |
| | YKRLGESLQSNTYNGFSTPWGYFDFNRFHCHFSPRDWQRLINNNWGMRPK |
| | AMRVKIFNIQVKEVTTSNGETTVANNLTSTVQIFADSSYELPYVMDAGQEGS |
| | LPPFPNDVFMVPQYGYCGLVTGNTSQQQTDRNAFYCLEYFPSQMLRTGNNF |
| | EITYSFEKVPFHSMYAHSQSLDRLMNPLIDQYLWGLQSTTTGTTLNAGTATT |
| | NFTKLRPTNFSNFKKNWLPGPSIKQQGFSKTANQNYKIPATGSDSLIKYETHS |
| | TLDGRWSALTPGPPMATAGPADSKFSNSQLIFAGPKQNGNTATVPGTLIFTSE |
| | EELAATNATDTDMWGNLPGGDQSNSNLPTVDRLTALGAVPGMVWQNRDI |
| | YYQGPIWAKIPHTDGHFHPSPLIGGFGLKHPPPQIFIKNTPVPANPATTFSSTP |
| | VNSFITQYSTGQVSVQIDWEIQKERSKRWNPEVQFTSNYGQQNSLLWAPDA |
| | AGKYTEPRAIGTRYLTHHL |
| AAV5 | MSFVDHPPDWLEEVGEGLREFLGLEAGPPKPKPNQQHQDQARGLVLPGYN |
| (SEQ ID NO: 180) | YLGPGNGLDRGEPVNRADEVAREHDISYNEQLEAGDNPYLKYNHADAEFQ |
| | EKLADDTSFGGNLGKAVFQAKKRVLEPFGLVEEGAKTAPTGKRIDDHFPKR |
| | KKARTEEDSKPSTSSDAEAGPSGSQQLQIPAQPASSLGADTMSAGGGGPLGD |
| | NNQGADGVGNASGDWHCDSTWMGDRVVTKSTRTWVLPSYNNHQYREIKS |
| | GSVDGSNANAYFGYSTPWGYFDFNRFHSHWSPRDWQRLINNYWGFRPRSL |
| | RVKIFNIQVKEVTVQDSTTTIANNLTSTVQVFTDDDYQLPYVVGNGTEGCLP |
| | AFPPQVFTLPQYGYATLNRDNTENPTERSSFFCLEYFPSKMLRTGNNFEFTYN |
| | FEEVPFHSSFAPSQNLFKLANPLVDQYLYRFVSTNNTGGVQFNKNLAGRYA |
| | NTYKNWFPGPMGRTQGWNLGSGVNRASVSAFATTNRMELEGASYQVPPQP |
| | NGMTNNLQGSNTYALENTMIFNSQPANPGTTATYLEGNMLITSESETQPVNR |
| | VAYNVGGQMATNNQSSTTAPATGTYNLQEIVPGSVWMERDVYLQGPIWAK |
| | IPETGAHFHPSPAMGGFGLKHPPPMMLIKNTPVPGNITSFSDVPVSSFITQYST |
| | GQVTVEMEWELKKENSKRWNPEIQYTNNYNDPQFVDFAPDSTGEYRTTRPI |
| | GTRYLTRPL |
| AAV6 | MAADGYLPDWLEDNLSEGIREWWDLKPGAPKPKANQQKQDDGRGLVLPG |
| (SEQ ID NO: 181) | YKYLGPFNGLDKGEPVNAADAAALEHDKAYDQQLKAGDNPYLRYNHADA |
| | EFQERLQEDTSFGGNLGRAVFQAKKRVLEPFGLVEEGAKTAPGKKRPVEQS |
| | PQEPDSSSGIGKTGQQPAKKRLNFGQTGDSESVPDPQPLGEPPATPAAVGPTT |
| | MASGGGAPMADNNEGADGVGNASGNWHCDSTWLGDRVITTSTRTWALPT |
| | YNNHLYKQISSASTGASNDNHYFGYSTPWGYFDFNRFHCHFSPRDWQRLIN |
| | NNWGFRPKRLNFKLFNIQVKEVTTNDGVTTIANNLTSTVQVFSDSEYQLPYV |
| | LGSAHQGCLPPFPADVFMIPQYGYLTLNNGSQAVGRSSFYCLEYFPSQMLRT |
| | GNNFTFSYTFEDVPFHSSYAHSQSLDRLMNPLIDQYLYYLNRTQNQSGSAQN |
| | KDLLFSRGSPAGMSVQPKNWLPGPCYRQQRVSKTKTDNNNSNFTWTGASK |
| | YNLNGRESIINPGTAMASHKDDKDKFFPMSGVMIFGKESAGASNTALDNVM |
| | ITDEEEIKATNPVATERFGTVAVNLQSSSTDPATGDVHVMGALPGMVWQDR |
| | DVYLQGPIWAKIPHTDGHFHPSPLMGGFGLKHPPPQILIKNTPVPANPPAEFS |
| | ATKFASFITQYSTGQVSVEIEWELQKENSKRWNPEVQYTSNYAKSANVDFT |
| | VDNNGLYTEPRPIGTRYLTRPL |
| AAV7 | MAADGYLPDWLEDNLSEGIREWWDLKPGAPKPKANQQKQDNGRGLVLPG |
| (SEQ ID NO: 182) | YKYLGPFNGLDKGEPVNAADAAALEHDKAYDQQLKAGDNPYLRYNHADA |
| | EFQERLQEDTSFGGNLGRAVFQAKKRVLEPLGLVEEGAKTAPAKKRPVEPSP |
| | QRSPDSSTGIGKKGQQPARKRLNFGQTGDSESVPDPQPLGEPPAAPSSVGSGT |
| | VAAGGGAPMADNNEGADGVGNASGNWHCDSTWLGDRVITTSTRTWALPT |
| | YNNHLYKQISSETAGSTNDNTYFGYSTPWGYFDFNRFHCHFSPRDWQRLIN |
| | NNWGFRPKKLRFKLFNIQVKEVTTNDGVTTIANNLTSTIQVFSDSEYQLPYV |
| | LGSAHQGCLPPFPADVFMIPQYGYLTLNNGSQSVGRSSFYCLEYFPSQMLRT |
| | GNNFEFSYSFEDVPFHSSYAHSQSLDRLMNPLIDQYLYYLARTQSNPGGTAG |
| | NRELQFYQGGPSTMAEQAKNWLPGPCFRQQRVSKTLDQNNNSNFAWTGAT |
| | KYHLNGRNSLVNPGVAMATHKDDEDRFFPSSGVLIFGKTGATNKTTLENVL |
| | MTNEEEIRPTNPVATEEYGIVSSNLQAANTAAQTQVVNNQGALPGMVWQN |
| | RDVYLQGPIWAKIPHTDGNFHPSPLMGGFGLKHPPPQILIKNTPVPANPPEVF |
| | TPAKFASFITQYSTGQVSVEIEWELQKENSKRWNPEIQYTSNFEKQTGVDFA |
| | VDSQGVYSEPRPIGTRYLTRNL |

Representative amino acid sequences of AAV capsids that can be used to target ocular tissues are provided in Table 3 below.

TABLE 3
Representative Capsids for Ocular Tropism

| Capsid | Amino Acid Sequence |
|---|---|
| AAV8 | MAADGYLPDWLEDNLSEGIREWWALKPGAPKPKANQQKQDDGRGLVLPGYKYLGP |
| (SEQ ID NO: 11) | FNGLDKGEPVNAADAAALEHDKAYDQQLQAGDNPYLRYNHADAEFQERLQEDTSFG |
| | GNLGRAVFQAKKRVLEPLGLVEEGAKTAPGKKRPVEPSPQRSPDSSTGIGKKGQQPAR |
| | KRLNFGQTGDSESVPDPQPLGEPPAAPSGVGPNTMAAGGGAPMADNNEGADGVG |
| | SSSGNWHCDSTWLGDRVITTSTRTWALPTYNNHLYKQISNGTSGGATNDNTYFGYST |
| | PWGYFDFNRFHCHFSPRDWQRLINNNWGFRPKRLSFKLFNIQVKEVTQNEGTKTIAN |
| | NLTSTIQVFTDSEYQLPYVLGSAHQGCLPPFPADVFMIPQYGYLTLNNGSQAVGRSSFY |
| | CLEYFPSQMLRTGNNFQFTYTFEDVPFHSSYAHSQSLDRLMNPLIDQYLYYLSRTQTTG |
| | GTANTQTLGFSQGGPNTMANQAKNWLPGPCYRQQRVSTTTGQNNNSNFAWTAG |
| | TKYHLNGRNSLANPGIAMATHKDDEERFFPSNGILIFGKQNAARDNADYSDVMLTSE |
| | EEIKTTNPVATEEYGIVADNLQQONTAPQIGTVNSQGALPGMVWQNRDVYLQGPIW |
| | AKIPHTDGNFHPSPLMGGFGLKHPPPQILIKNTPVPADPPTTFNQSKLNSFITQYSTGQ |
| | VSVEIEWELQKENSKRWNPEIQYTSNYYKSTSVDFAVNTEGVYSEPRPIGTRYLTRNL |
| AAV9 | MAADGYLPDWLEDNLSEGIREWWALKPGAPQPKANQQHQDNARGLVLPGYKYLG |
| (SEQ ID NO: 5) | PGNGLDKGEPVNAADAAALEHDKAYDQQLKAGDNPYLKYNHADAEFQERLKEDTSF |
| | GGNLGRAVFQAKKRLLEPLGLVEEAAKTAPGKKRPVEQSPQEPDSSAGIGKSGAQPAK |
| | KRLNFGQTGDTESVPDPQPIGEPPAAPSGVGSLTMASGGGAPVADNNEGADGVGSS |
| | SGNWHCDSQWLGDRVITTSTRTWALPTYNNHLYKQISNSTSGGSSNDNAYFGYSTP |
| | WGYFDFNRFHCHFSPRDWQRLINNNWGFRPKRLNFKLFNIQVKEVTDNNGVKTIAN |
| | NLTSTVQVFTDSDYQLPYVLGSAHEGCLPPFPADVFMIPQYGYLTLNDGSQAVGRSSF |
| | YCLEYFPSQMLRTGNNFQFSYEFENVPFHSSYAHSQSLDRLMNPLIDQYLYYLSKTING |
| | SGQNQQTLKFSVAGPSNMAVOGRNYIPGPSYRQQRVSTTVTQNNNSEFAWPGASS |
| | WALNGRNSLMNPGPAMASHKEGEDRFFPLSGSLIFGKQGTGRDNVDADKVMITNE |
| | EEIKTTNPVATESYGQVATNHQSAQAQAQTGWVQNQGILPGMVWQDRDVYLQGP |
| | IWAKIPHTDGNFHPSPLMGGFGMKHPPPQILIKNTPVPADPPTAFNKDKLNSFITQYS |
| | TGQVSVEIEWELQKENSKRWNPEIQYTSNYYKSNNVEFAVNTEGVYSEPRPIGTRYLT |
| | RNL |
| AAV2 | MAADGYLPDWLEDTLSEGIRQWWKLKPGPPPPKPAERHKDDSRGLVLPGYKYLGPF |
| (SEQ ID NO: 30) | NGLDKGEPVNEADAAALEHDKAYDRQLDSGDNPYLKYNHADAEFQERLKEDTSFGG |
| | NLGRAVFQAKKRVLEPLGLVEEPVKTAPGKKRPVEHSPVEPDSSSGTGKAGQQPARKR |
| | LNFGQTGDADSVPDPQPLGQPPAAPSGLGTNTMATGSGAPMADNNEGADGVGNS |
| | SGNWHCDSTWMGDRVITTSTRTWALPTYNNHLYKQISSQSGASNDNHYFGYSTPW |
| | GYFDFNRFHCHFSPRDWORLINNNWGFRPKRLNFKLFNIQVKEVTONDGTTTIANNL |
| | TSTVQVFTDSEYQLPYVLGSAHQGCLPPFPADVFMVPQYGYLTLNNGSQAVGRSSFY |
| | CLEYFPSQMLRTGNNFTFSYTFEDVPFHSSYAHSQSLDRLMNPLIDQYLYYLSRTNTPS |
| | GTTTQSRLQFSQAGASDIRDQSRNWLPGPCYRQQRVSKTSADNNNSEYSWTGATKY |
| | HLNGRDSLVNPGPAMASHKDDEEKFFPQSGVLIFGKQGSEKTNVDIEKVMITDEEEIR |
| | TTNPVATEQYGSVSTNLORGNRQAATADVNTQGVLPGMVWQDRDVYLQGPIWAKI |
| | PHTDGHFHPSPLMGGFGLKHPPPQILIKNTPVPANPSTTFSAAKFASFITQYSTGQVSV |
| | EIEWELQKENSKRWNPEIQYTSNYNKSVNVDFTVDTNGVYSEPRPIGTRYLTR |
| AAV3B | MAADGYLPDWLEDNLSEGIREWWALKPGVPQPKANQQHQDNRRGLVLPGYKYLGP |
| (SEQ ID NO: 31) | GNGLDKGEPVNEADAAÅLEHDKAYDQQLKAGDNPYLKYNHADAEFQERLQEDTSFG |
| | GNLGRAVFQAKKRILEPLGLVEEAAKTAPGKKRPVDQSPQEPDSSSGVGKSGKQPARK |
| | RLNFGQTGDSESVPDPQPLGEPPAAPTSLGSNTMASGGGAPMADNNEGADGVGNS |
| | SGNWHCDSQWLGDRVITTSTRTWALPTYNNHLYKQISSQSGASNDNHYFGYSTPWG |
| | YFDFNRFHCHFSPRDWORLINNNWGFRPKKLSFKLFNIQVKEVTQNDGTTTIANNLTS |
| | TVQVFTDSEYQLPYVLGSAHQGCLPPFPADVFMVPQYGYLTLNNGSQAVGRSSFYCL |
| | EYFPSQMLRTGNNFQFSYTFEDVPFHSSYAHSQSLDRLMNPLIDQYLYYLNRTQGTTS |
| | GTTNQSRLLFSQAGPQSMSLQARNWLPGPCYRQQRLSKTANDNNNSNFPWTAASK |
| | YHLNGRDSLVNPGPAMASHKDDEEKFFPMHGNLIFGKEGTTASNAELDNVMITDEEE |
| | IRTTNPVATEQYGTVANNLQSSNTAPTTRTVNDQGALPGMVWQDRDVYLQGPIWA |
| | KIPHTDGHFHPSPLMGGFGLKHPPPQIMIKNTPVPANPPTTFSPAKFASFITQYSTGQV |
| | SVEIEWELQKENSKRWNPEIQYTSNYNKSVNVDFTVDTNGVYSEPRPIGTRYLTRNL |
| AAV2.7m8 | MAADGYLPDWLEDTLSEGIRQWWKLKPGPPPPKPAERHKDDSRGLVLPGYKYLGPF |
| (SEQ ID NO: 32) | NGLDKGEPVNEADAAALEHDKAYDRQLDSGDNPYLKYNHADAEFQERLKEDTSFGG |
| | NLGRAVFQAKKRVLEPLGLVEEPVKTAPGKKRPVEHSPVEPDSSSGTGKAGQQPARKR |
| | LNFGQTGDADSVPDPQPLGQPPAAPSGLGTNTMATGSGAPMADNNEGADGVGNS |
| | SGNWHCDSTWMGDRVITTSTRTWALPTYNNHLYKQISSQSGASNDNHYFGYSTPW |
| | GYFDFNRFHCHFSPRDWQRLINNNWGFRPKRLNFKLFNIQVKEVTQNDGTTTIANNL |
| | TSTVQVFTDSEYQLPYVLGSAHQGCLPPFPADVFMVPQYGYLTLNNGSQAVGRSSFY |
| | CLEYFPSQMLRTGNNFTFSYTFEDVPFHSSYAHSQSLDRLMNPLIDQYLYYLSRTNTPS |
| | GTTTQSRLQFSQAGASDIRDQSRNWLPGPCYRQQRVSKTSADNNNSEYSWTGATKY |
| | HLNGRDSLVNPGPAMASHKDDEEKFFPQSGVLIFGKQGSEKTNVDIEKVMITDEEEIR |
| | TTNPVATEQYGSVSTNLQRGNLALGETTRPARQAATADVNTQGVLPGMVWQDRDV |
| | YLQGPIWAKIPHTDGHFHPSPLMGGFGLKHPPPQILIKNTPVPANPSTTFSAAKFASFIT |
| | QYSTGQVSVEIEWELQKENSKRWNPEIQYTSNYNKSVNVDFTVDTNGVYSEPRPIGTR |
| | YLTR |
| AAV.rh.34 | MAADGYLPDWLEDNLSEGIREWWDLKPGAPKPKANQQKQDDGRGLVLPGYEYLGP |
| (SEQ ID NO: 33) | FNGLDKGEPVNAADAAALEHDKAYDQQLKAGDNPYLRYNHADAEFQERLQEDTSFG |
| | GNLGRAVFQAKKRVLEPLGLVEEGAKTAPGKKRPLESPQEPDSSSGIGKKGKQPAKKR |
| | LNFEEDTGAGDGPPEGSDTSAMSSDIEMRAAPGGNAVDAGQGSDGVGNASGDWH |
| | CDSTWSEGKVTTTSTRTWVLPTYNNHLYLRLGTTSNSNTYNGFSTPWGYFDFNRFHC |
| | HFSPRDWORLINNNWGLRPKAMRVKIFNIQVKEVTTSNGETTVANNLTSTVQIFADS |
| | SYELPYVMDAGQEGSLPPFPNDVFMVPQYGYCGIVTGENQNQTDRNAFYCLEYFPS |
| | QMLRTGNNFETAYNFEKVPFHSMYAHSQSLDGLMNPLLDQYLWHLQSTTSGETLNQ |
| | GNAATTFGKIRSGDFAFYRKNWLPGPCVKQQRFSKTASQNYKIPASGGNALLKYDTHY |
| | TLNNRWSNIAPGPPMATAGPSDGDFSNAQLIFPGPSVTGNTTTSANNLLFTSEEEIAA |
| | TNPRDTDMFGQIADNNONATTAPITGNVTAMGVLPGMVWQNRDIYYQGPIWAKIP |
| | HADGHFHPSPLIGGFGLKHPPPQIFIKNTPVPAYPATTFTAARVDSFITQYSTGQVAVQI |
| | EWEIEKERSKRWNPEVQFTSNCGNQSSMLWAPDTTGKYTEPRVIGSRYLTNHL |
| AAV.rh.31 | KAYDQQLKAGDNPYLRYNHADAEFQERLQEDTSFGGNLGRAVFQAKKRVLEPLGLVE |
| (SEQ ID NO: 34) | TPAKTAPGKKRPVDSPDSTSGIGKKGQQPAKKRLNFGQTGDSESVPDPQPIGEPPAGP |
| | SGLGSGTMAAGGGAPMADNNEGADGVGNASGNWHCDSTWLGDRVITTSTRTWA |
| | LPTYNNHLYKQISSQSAGSTNDNVYFGYSTPWGYFDFNRFHCHFSPRDWORLINNN |
| | WGFRPKKLNFKLFNIQVKEVTTNDGVTTIANNLTSTVQVFSDSEYQLPYVLGSAHQGC |
| | LPPFPADVFMIPQYGYLTLNNGSQSVGRSSFYCLEYFPSQMLRTGNNFTFSYTFEDVPF |
| | HSSYAHSQSLDRLMNPLIDQYLYYLARTQSNAGGTAGNRELQFYQGGPTTMAEQAK |
| | NWLPGPCFRQQRVSKTLDQNNNSNFAWTGATKYHLNRNSLVNPGVAMATHKDDE |
| | ERFFPSSGVLIFGKTGAANKTTLENVLMTNEEEIRPTNPVATEEYGIVSSNLQAASTAA |
| | QTQVVNNQGALPGMVWQNRDVYLQGPIWAKIPHTDGNFHPSPLMGGFGLKHPPP |
| | QILIKNTPVPANPPEVFTPAKFASFITQYSTGQVSVEIEWELQKENSKRWNPEIQYTSNF |
| | DKQTGVDFAVDSQGVYSEP |
| AAV. hu.12 | MAADGYLPDWLEDTLSEGIRQWWKLKPGPPPPKPAERHQDDSRGLVLPGYKYLGPF |
| (SEQ ID NO: 35) | NGLDKGEPVNEADAAALEHDKAYDRQLDSGDNPYLKYNHADAEFQERLKEDTSFGG |
| | NLGRAVFQAKKRVLEPLGLVEEPVKTAPGKKRPVEHSPVEPDSSSGTGKAGHQPARKR |
| | LNFGQTGDADSVPDPQPLGQPPAAPTSLGSTTMATGSGAPMADNNEGADGVGNSS |
| | GNWHCDSQWLGDRVITTSTRTWALPTYNNHLYKQISSQSGASNDNHYFGYSTPWG |
| | YFDFNRFHCHFSPRDWQRLINNNWGFRPKRLNFKLFNIQVKEVTQNDGTTTIANNLT |
| | STVQVFTDSEYQLPYVLGSAHQGCLPPFPADVFMVPQYGYLTLNNGSQAVGRPSFYC |
| | LEYFPSQMLRTGNNFTFSYTFEDVPFHSSYAHSQSLDRLMNPLIDQYLYYLNRTQSNSG |
| | TLQQSRLLFSQAGPTSMSLOAKNWLPGPCYRQQRLSKQANDNNNSNFPWTAATKY |
| | HLNGRDSLVNPGPAMASHKDDEEKFFPMHGTLIFGKQGTNANDADLEHVMITDEEE |
| | IRTTNPVATEQYGNVSNNLONSNTGPTTENVNHQGALPGMVWQDRDVYLQGPIW |
| | AKIPHTDGHFHPSPLMGGFGLKHPPPQIMIKNTPVPANPPTNFSSAKFASFITQYSTG |
| | QVSVEIEWELQKENSKRWNPEIQYTSNYNKSVNVDFTVDTNGVYSEPRPIGTRYLTRN |
| | L |
| AAV.hu.13 | MAADGYLPDWLEDTLSEGIRQWWKLKPGPPPPKPAERHKDDSRGLVLPGYKYLGPF |
| (SEQ ID NO: 36) | NGLDKGEPVNEADAAALEHDKAYDROLDSGDNPYLKYNHADAEFQERLKEDTSFGG |
| | NLGRAVFQAKKRVLEPLGLVEEPVKTAPGKKRPVEHSPAEPDSSSGTGKAGQQPARK |
| | RLNFGQTGDADSVPDPQPLGQPPAAPSGLGTNTMASGSGAPMADNNEGADGVGN |
| | SSGNWHCDSTWMGDRVITTSTRTWALPTYNNHLYKQISSQSGASNDNHYFGYSTP |
| | WGYFDFNRFHCHFSPRDWQRLINNNWGFRPKRLNFKLFNIQVKEVTQNDGTTTIAN |
| | NLTSTVQVFTDSEYQLPYVLGSAHQGCLPPFPADVFMVPQYGYLTLNNGSQAVGRSS |
| | FYCLEYFPSOMLRTGNNFTFSYTFEDVPFHSSYAHSQSLDRLMNPLIDQYLYYLSRTNT |
| | PSGTTTQSRLQFSQAGASDIRDQSRNWLPGPCYRQQRVSKTSADNNNSEYSWTGAT |
| | KYHLNGRDSLVNPGPAMASHKDDEEKFFPQSGVLIFGKQGSEKTNVDIEKVMITDEEE |
| | IRTTNPVATEQYGSVSTNLQGGNTQAATADVNTQGVLPGMVWQDRDVYLQGPIW |
| | AKIPHTDGHFHPSPLMGGFGLKHPPPQILIKNTPVPANPSTTFSAAKFASFITQYSTGQ |
| | VSVEIEWELQKENSKRWNPEIQYTSNYNKSVNVDFTVDTNGVYSEPRPIGTRYLTRNL |
| AAV.hu.21 | MAADGYLPDWLEDTLSEGIRQWWKLKPGPPPPKPAERHKDDSRGLVLPGYKYLGPF |
| (SEQ ID NO: 37) | NGLDKGEPVNEADAAALEHDKAYDROLDSGDNPYLKYNHADAEFQERLKEDTSFGG |
| | NLGRÅVFQAKKRILEPLGLVEEPVKTAPGKKRPVEHSPAEPDSSSGTGKAGQQPARKR |
| | LNFGQTGDADSVPDPRPLGQPPAAPSGLGTNTMASGSGAPMADNNEGADGVGNS |
| | SGNWHCDSTWMGDRVITTSTRTWALPTYNNHLYKQISSQSGASNDNHYFGYSTPW |
| | GYFDFNRFHCHFSPRDWORLINNNWGFRPKRLSFKLFNIQVKEVTQNDGTTTIANNL |
| | TSTVQVFTDSEYQLPYVLGSAHQGCLPPFPADVFMVPQYGYLTLNNGSQAVGRSSFY |
| | CLEYFPSQMLRTGNNFTFSYTFEDVPFHSSYAHSQSLDRLMNPLIDQYLYYLSRTNTPS |
| | GTTTMSRLQFSQAGASDIRDQSRNWLPGPCYRQQRVSKTAADNNNSDYSWTGATK |
| | YHLNGRDSLVNPGPAMASHKDDEEKYFPQSGVLIFGKQDSGKTNVDIEKVMITDEEEI |
| | RTTNPVATEQYGSVSTNLQSGNTQAATSDVNTQGVLPGMVWQDRDVYLQGPIWA |
| | KIPHTDGHFHPSPLMGGFGLKHPPPQILIKNTPVPANPSTTFSAAKFASFITQYSTGQVS |
| | VEIEWELQKENSKRWNPEIQYTSNYNKSVNVDFTVDTNGVYSEPRPIGTRYLTRNL |
| AAV.hu.26 | MAADGYLPDWLEDTLSEGIRQWWKLKPGPPPPKPAERHKDDSRGLVLPGYKYLGPF |
| (SEQ ID NO: 38) | NGLDKGEPVNEADAAALEHDKAYDRQLDSGDNPYLKYNHADAEFQERLKEDTSFGG |
| | NLGRAVFQAKKRILEPLGLVEEPVKTAPGKKRPVEHSPAEPDSSSGTGKAGQQPARKR |
| | LNFGQTGDADSVPDPQPLGQPPAAPSGLGTNTMASGSGAPMADNNEGADGVGNS |
| | SGNWHCDSTWMGDRVITTSTRTWALPTYNNHLYKQISSQSGASNDNHYFGYSTPW |
| | GYFDFNRFHCHFSPRDWORLINNNWGFRPKRLSFKLFNIQVKEVTQNDGTTTIANNL |
| | TSTVQVFTDSEYQLPYVLGSAHQGCLPPFPADVFMVPQYGYLTLNNGSQAVGRSSFY |
| | CLEYFPSQMLRTGNNFTFSYTFEDVPFHSSYAHSQSLDRLMNPLIDQYLYYLSRTNTPS |
| | GTTTMSRLQFSQAGASDIRDQSRNWLPGPCYRQQRVSKTAADNNNSDYSWTGATK |
| | YHLNGRDSLVNPGPAMASHKDDEEKYFPQSGVLIFGKQDSGKTNVDIEKVMITDEEEI |
| | RTTNPVATEQYGSVSTNLQSGNTQAATSDVNTQGVLPGMVWQDRDVYLQGPIWA |
| | KIPHTDGHFHPSPLMGGFGLKHPPPQILIKNTPVPANPSTTFSAAKFASFITQYSTGQVS |
| | VEIEWELQKENSKRWNPEIQYTSNYNKSVNVDFTVDTNGVYSEPRPIGTRYLTRNL |
| AAV.hu.53 | MAADGYLPDWLEDTLSEGIRQWWKLKPGPPPPKPAERHKDDSRGLVLPGYKYLGPF |
| (SEQ ID NO: 39) | NGLDKGEPVNEADAAALEHDKAYDROLDSGDNPYLKYNHADAEFQERLKEDTSFGG |
| | NLGRAVFQAKKRVLEPLGLVEEPVKTAPGKKRPVEHSPAEPDSSSGTGKAGQQPARK |
| | RLNFGQTGDADSVPDPQPLRQPPAAPTSLGSTTMATGSGAPMADNNEGADGVGNS |
| | SGNWHCDSQWLGDRVITTSTRTWALPTYNNHLYKQISSQSGASNDNHYFGYSTPWG |
| | YFDFNRFHCHFSPRDWORLINNNWGFRPKRLNFKLFNIQVKEVTQNDGTTTIANNLT |
| | STVQVFTDSEYQLPYVLGSAHQGCLPPFPADVFMVPQYGYLTLNNGSQAVGRSSFYC |
| | LEYFPSQMLRTGNNFQFSYTFEDVPFHSSYAHSQSLDRLMNPLIDQYLYYLNRTQTAS |
| | GTQQSRLLFSQAGPTSMSLQAKNWLPGPCYRQQRLSKQANDNNNSNFPWTGATKY |
| | YLNGRDSLVNPGPAMASHKDDEEKFFPMHGTLIFGKEGTNATNAELENVMITDEEEI |
| | RTTNPVATEQYGYVSNNLONSNTAASTETVNHQGALPGMVWQDRDVYLQGPIWA |
| | KIPHTDGHFHPSPLMGGFGLKHPPPQIMIKNTPVPANPPTNFSSAKFASFITQYSTGQ |
| | VSVEIEWELQKENSKRWNPEIQYTSNYNKSVNVDFTVDTNGVYSEPRPIGTRYLTRNL |
| AAV.hu.56 | MAADGYLPDWLEDTLSEGIRQWWKLKPGPPPPKPAERHKDDSRGLVLPGYKYLGPF |
| (SEQ ID NO: 40) | NGLDKGEPVNEADAAALEHDKAYDRQLDSGDNPYLKYNHADAEFQERLKEDTSFGG |
| | NLGRAVFQAKKRVLEPLGLVEEPVKTAPGKKRPVEHSPVEPDSSSGTGKAGNQPARKR |
| | LNFGQTGDADSVPDPQPLGQPPASPSGLGTNTMATGSGAPMADNNEGADGVGNS |
| | SGNWHCDSTWMGDRVVTTSTRTWALPTYNNHLYKQISSQSGASNDNHYFGYSTPW |
| | GYFDFNRFHCHFSPRDWQRLINNNWGFRPKRLNFKLFNIQVKEVTQNDGTTTIANNL |
| | TSTVQVFTDLEYQLPYVLGSAHQGCLPPFPADVFMVPQYGYLTLNNGSQAVGRSSFY |
| | CLEYFPSQMLRTGNNFTFSYTFEDVPFHSSYAHSQSLDRLMNPLIDQYLYYLSRTNTPS |
| | GTTTQSRLQFSQAGASDIRDQSRNWLPGPCYRQQRVSKTAADNNNSEYSWTGATKY |
| | HLNGRDSLVNPGPAMASHKDDEEKFFPQSGVLIFGKQGSEKTNVDIEKVMITDEEEIR |
| | TTNPVATEQYGSVSTNLQSGNTQAATSDVNTQGVLPGMVWQDRDVYLQGPIWAKI |
| | PHTDGHFHPSPLMGGFGLKHPPPQILIKNTPVPANPSTTFSAAKFASFITQYSTGQVSV |
| | EIEWELQKENSKRWNPEIQYTSNYNKSVNVDFTVDTNGVYSEPRPIGTRYLTRNL |
| AAV.rh.24 | MAADGYLPDWLEDNLSEGIREWWDLKPGAPKPKANQQKQDDGRGLVLPGYKYLGP |
| (SEQ ID NO: 41) | FNGLDKGEPVNAADAAALEHDKAYDQQLKAGDNPYLRYNHADAEFQERLQEDTSFG |
| | GNLGRAVFQAKKRVLEPLGLVEEGAKTAPGKKRPIESPDSSTGIGKKGQQPAKKKLNF |
| | GQTGDSESVPDPQPIGEPPAGPSGLGSGTMAAGGGAPMADNNEGADGVGSSSGN |
| | WHCDSTWLGDRVITTSTRTWALPTYNNHLYKQISNGTSGGSTNDNTYFGYSTPWGY |
| | FDFNRFHCHFSPRDWQRLINNNWGFRPKRLNFKLFNIQVKEVTQNEGTKTIANNLTS |
| | TIQVFTDSEYQLPYVLGSAHQGCLPPFPADVFMIPQYGYLTLNNGSQAVGRSSFYCLEY |
| | FPSQMLRTGNNFEFSYQFEDVPFHSSYAHSQSLDRLMNPLIDQYLYYLSRTQSTGGTA |
| | GTQQLLFSQAGPNNMSAQAKNWLPGPCYRQQRVSTTVSQNNNSNFAWTGATKYH |
| | LNGRDSLVNPGVAMATHKGDEERFFPSSGVLMFGKQGAGKDNVDYSSVMLTSEEEI |
| | KTTNPVATEQYGVVADNLQQQNAAPIVGAVNSQGALPGMVWQNRDVYLQGPIWA |
| | KIPHTDGNFHPSPLMGGFGLKHPPPQILIKNTPVPADPPTTFSQAKLASFITQYSTGQV |
| | SVEIEWELQKENSKRWNPEIQYTSNYYKSTNVDFAVNTEGTYSEPRPIGTRYLTRSL |
| AAV.hu.38 | MAADGYLPDWLEDNLSEGIREWWDLKPGAPKPKANQQKQDDGRGLVLPGYKYLGP |
| (SEQ ID NO: 42) | FNGLDKGEPVNAADAAALEHDKAYDQQLKAGDNPYLRYNHADAEFQERLQEDTSFG |
| | GNLGRAVFQAKKRVLEPLGLVEEAAKTAPGKKRPVEPSPQRSPDSSTGIGKKGQQPAK |
| | KRLNFGQTGDSESVPDPQPIGEPPAGPSGLGSGTMAAGGGAPMADNNEGADGVGS |
| | SSGNWHCDSTWLGDRVITTSTRTWALPTYNNHLYKQISNGTSGGSTNDNTYFGYSTP |
| | WGYFDFNRFHCHFSPRDWQRLINNNWGFRPKRLSFKLFNIQVKEVTQNEGTKTIAN |
| | NLTSTIQVFTDSEYQLPYVLGSAHQGCLPPFPADVFMIPQYGYLTLNNGSQAVGRSSFY |
| | CLEYFPSQMLRTGNNFEFSYTFEDVPFHSSYAHSQSLDRLMNPLIDQYLYYLSRTQSTG |
| | GTQGTQQLLFSQAGPANMSAQAKNWLPGPCYRQQRVSTTLSONNNSNFAWTGAT |
| | KYHLNGRDSLVNPGVAMATHKDDEERFFPSSGVLMFGKQGAGRDNVDYSSVMLTSE |
| | EEIKTTNPVATEQYGVVADNLQQTNTGPIVGNVNSQGALPGMVWQNRDVYLQGPI |
| | WAKIPHTDGNCHPSPLMGGFGLKHPPPQILIKNTPVPÅDPPTTFSQAKLASFITQYSTG |
| | QVSVEIEWELQKENSKRWNPEIQYTSNYYKSTNVDFAVNTEGTYSEPRPIGTRYLTRNL |
| AAV.rh.72 | MAADGYLPDWLEDNLSEGIREWWDLKPGAPKPKANQQKQDDGRGLVLPGYKYLGP |
| (SEQ ID NO: 43) | FNGLDKGEPVNAADAAALEHDKAYDQQLKAGDNPYLRYNHADAEFQERLQEDTSFG |
| | GNLGRAVFQAKKRVLEPLGLVEEAAKTAPGKKRPVEPSPQRSPDSSTGIGKKGQQPAK |
| | KRLNFGQTGDSESVPDPQPIGEPPAGPSGLGSGTMAAGGGAPMADNNEGADGVGS |
| | SSGNWHCDSTWLGDRVITTSTRTWALPTYNNHLYKQISNGTSGGSTNDNTYFGYSTP |
| | WGYFDFNRFHCHFSPRDWQRLINNNWGFRPKRLSFKLFNIQVKEVTQNEGTKTIAN |
| | NLTSTIQVFTDSEYQLPYVLGSAHQGCLPPFPADVFMIPQYGYLTLNNGSQAVGRSSFY |
| | CLEYFPSQMLRTGNNFEFSYTFEDVPFHSSYAHSQSLDRLMNPLIDQYLYYLSRTQSTG |
| | GTQGTQQLLFSQAGPANMSAQAKNWLPGPCYRQQRVSTTLSONNNSNFAWTGAT |
| | KYHLNGRDSLVNPGVAMATHKDDEERFFPSSGVLMFGKQGAGRDNVDYSSVMLTSE |
| | EEIKTTNPVATEQYGVVADNLQQTNTGPIVGNVNSQGALPGMVWQNRDVYLQGPI |
| | WAKIPHTDGNFHPSPLMGGFGLKHPPPQILIKNTPVPADPPTTFSQAKLASFITQYSTG |
| | QVSVEIEWELQKENSKRWNPEIQYTSNYYKSTNVDFAVNTEGTYSEPRPIGTRYLTRNL |
| AAV.cy.5 | MAADGYLPDWLEGNLSEGIREWWDLKPGAPKPKANQQKQDDGRGLVLPGYRYLGP |
| (SEQ ID NO: 44) | FNGLDKGEPVNEADÅAALEHDKAYDKQLEQGDNPYLKYNHADAEFQERLQEDTSFG |
| | GNLGRAVFQAKKRVLEPLGLVEEGAKTAPGKKRPIESPDSSTGIGKNGQPPAKKKLNF |
| | GQTGDSESVPDPQPLGEPPAAPSGLGSGTMAAGGGAPMADNNEGADGVGNASGN |
| | WHCDSTWLGDRVITTSTRTWALPTYNNHLYKQISSQSGATNDNHFFGYSTPWGYFD |
| | FNRFHCHFSPRDWQRLINNNWGFRPRKLRFKLFNIQVKEVTTNDGVTTIANNLTSTIQ |
| | VFSDSEYQLPYVLGSAHQGCLPPFPADVFMIPQYGYLTLNNGSQSVGRSSFYCLEYFPS |
| | QMLRTGDNFEFSYTFEEVPFHSSYAHSQSLDRLMINPLIDQYLYYLARTQSTTGSTRELQ |
| | FHQAGPNTMAEQSKNWLPGPCYRQQRLSKNIDSNNNSNFAWTGATKYHLNGRNSL |
| | TNPGVAMATNKDDEDQFFPINGVLVFGKTGAANKTTLENVLMTSEEEIKTTNPVATE |
| | EYGVVSSNLQSSTAGPQTQTVNSQGALPGMVWQNRDVYLQGPIWAKIPHTDGNFH |
| | PSPLMGGFGLKHPPPQILIKNTPVPANPPEVFTPAKFASFITQYSTGQVSVEIEWELQK |
| | ENSKRWNPEIQYTSNYAKSNNVEFAVNNEGVYTEPRPIGTRYLTRNL |
| AAV.cy.6 | MAADGYLPDWLEDNLSEGIREWWDLKPGAPKPKANQQKQDDGRGLVLPGYKYLGP |
| (SEQ ID NO: 45) | FNGLDKGEPVNEADAAALEHDKAYDKQLEQGDNPYLKYNHADAEFQERLQEDTSFG |
| | GNLGRAVFQAKKRVLEPLGLVEEGAKTAPGKKRPIESPDSSTGIGKKGQQPAKKKLNF |
| | GQTGDSESVPDPQPLGEPPAAPSGLGSGTMAAGGGAPMADNNEGADGVGNASGN |
| | WHCDSTWLGDRVITTSTRTWALPTYNNHLYKQISSQSGATNDNHFFGYSTPWGYFD |
| | FNRFHCHFSPRDWQRLINNNWGFRPRKLRFKLFNIQVKEVTTNDGVTTIANNLTSTIQ |
| | VFSDSEYQLPYVLGSAHQGCLPPFPADVFMIPQYGYLTLNNGSQSMGRSSFYCLEYFP |
| | SQMLRTGNNFEFSYTFEEVPFHSSYAHSQSLDRLMINPLIDQYLYYLARTQSTTGSTREL |
| | QFHOAGPNTMAEQSKNWLPGPCYRQQRLSKNIDSNNNSNFAWTGATKYHLNGRN |
| | SLTNPGVAMATNKDDEGOFFPINGVLVFGKTGAANKTTLENVLMTSEEEIKTTNPVAT |
| | EEYGVVSSNLQSSTAGPQTQTVNSQGALPGMVWQNRDVYLQGPIWAKIPHTDGNF |
| | HPSPLMGGFGLKHPPPQILIKNTPVPANPPGVFTPALFASFITQYSTGQVSVEIEWELQ |
| | KENSKRWNPEIQYTSNYAKSNNVEFAVNNEGVYTEPRPIGTRYLTRNL |
| AAV.rh.46 | MAADGYLPDWLEDNLSEGIREWWDLKPGAPKPKANQQKQDDGRGLVLPGYKYLGP |
| (SEQ ID NO: 46) | FNGLDKGEPVNAADAAALEHDKAYDQQLKAGDNPYLRYNHADAEFQERLQEDTSFG |
| | GNLGRAVFQAKKRVLEPLGLVEEGAKTAPGKKRPVEPSPQRSPDSSTGIGKKGQQPAR |
| | KRLNFGQTGDSESVPDPQPIGEPPAAPSSVGSGTMAAGGGAPMADNNEGADGVGS |
| | SSGNWHCDSTWLGDRVITTSTRTWALPTYNNHLYKQISNGTSGGSTNDNTYFGYSTP |
| | WGYFDFNRFHCHFSPRDWQRLINNNWGFRPKRLSFKLFNIQVKEVTQNEGTKTIAN |
| | NLTSTIQVFTDSEYQLPYVLGSAHQGCLPPFPADVFMIPQYGYLTLNNGSQAVGRSSFY |
| | CLEYFPSQMLRTGNNFSFSYTFEDVPFHSSYAHSQSLDRLMNPLIDQYLYYLSRTQSTG |
| | GTAGTQQLLFSQAGPSNMSAQARNWLPGPCYRQQRVSTTLSQNNNSNFAWTGAT |
| | KYHLNGRDSLVNPGVAMATNKDDEDRFFPSSGILMFGKQGAGKDNVDYSNVMLTSE |
| | EEIKATNPVATEQYGVVADNLQQQNTAPIVGAVNSQGALPGMVWQNRDVYLQGPI |
| | WAKIPHTDGNFHPSPLMGGFGLKHPPPQILIKNTPVPADPPTAFNQAKLNSFITQYST |
| | GQVSVEIEWELQKENSKRWNPEIQYTSNYYKSTNVDFAVNTEGVYSEPRPIGTRYLTR |
| | NL |
| AAV.rh.2 | MAADGYLPDWLEDNLSEGIREWWDLKPGAPKPKANQQKQDDGRGLVLPGYKYLGP |
| (SEQ ID NO: 47) | FNGLDKGEPVNAADAAALEHDKAYDQQLKAGDNPYLRYNHADAEFQERLQEDTSFG |
| | GNLGRAVFQAKKRVLEPLGLVEEGAKTAPGKKRPVEPSPQRSPDSSTGIGKKGHQPAR |
| | KRLNFGQTGDSESVPDPQPIGEPPAGPSGLGSGTMAAGGGAPMADNNEGADGVGS |
| | SSGNWHCDSTWLGDRVITTSTRTWALPTYNNHLYKQISNGTSGGSTNDNTYFGYSTP |
| | WGYFDFNRFHCHFSPRDWORLINNNWGFRPKRLNFKLFNIQVKEVTQNEGTKTIAN |
| | NLTSTIQVFTDSEYQLPYVPGSAHQGCLPPFPADVFMIPQYGYLTLNNGSQAVGRSSF |
| | YCLEYFPSQMLRTGNNFEFSYTFEDVPFHSSYAHSQSLDRLMNPLIDQYLYYLSRTQST |
| | GGTQGTQQLLFSQAGPANMSAQAKNWLPGPCYRQQRVSTTLSQNNNSNFAWTGA |
| | TKYHLNGRDSLVNPGVAMATHKDDEERFFPSSGVLMFGKQGAGKDNVDYSSVMLTS |
| | EEEIKTTNPVATEQYGVVADNLQQTNGAPIVGTVNSQGALPGMVWQNRDVYLQGPI |
| | WAKIPHTDGNFHPSPLMGGFGLKHPPPQILVKNTPVPADPPTTFSQAKLASFITQYST |
| | GQVSVEIEWELQKENSKRWNPEIQYTSNYYKSTNVDFAVNTEGTYSEPRPIGTRYLTR |
| | NL |
| AAV1 | MAADGYLPDWLEDNLSEGIREWWDLKPGAPKPKANQQKQDDGRGLVLPGYKYLGPFNGLD |
| (SEQ ID NO: 177) | KGEPVNAADAAALEHDKAYDQQLKAGDNPYLRYNHADAEFQERLQEDTSFGGNLGRAVFQA |
| | KKRVLEPLGLVEEGAKTAPGKKRPVEQSPQEPDSSSGIGKTGQQPAKKRLNFGQTGDSESVPD |
| | PQPLGEPPATPAAVGPTTMASGGGAPMADNNEGADGVGNASGNWHCDSTWLGDRVITTS |
| | TRTWALPTYNNHLYKQISSASTGASNDNHYFGYSTPWGYFDFNRFHCHFSPRDWQRLINNN |
| | WGFRPKRLNFKLFNIQVKEVTTNDGVTTIANNLTSTVQVFSDSEYQLPYVLGSAHQGCLPPFPA |
| | DVFMIPQYGYLTLNNGSQAVGRSSFYCLEYFPSQMLRTGNNFTFSYTFEEVPFHSSYAHSQSLD |
| | RLMNPLIDQYLYYLNRTQNQSGSAQNKDLLFSRGSPAGMSVQPKNWLPGPCYRQQRVSKTK |
| | TDNNNSNFTWTGASKYNLNGRESIINPGTAMASHKDDEDKFFPMSGVMIFGKESAGASNTA |
| | LDNVMITDEEEIKATNPVATERFGTVAVNFQSSSTDPATGDVHAMGALPGMVWQDRDVYL |
| | QGPIWAKIPHTDGHFHPSPLMGGFGLKNPPPQILIKNTPVPANPPAEFSATKFASFITQYSTGQ |
| | VSVEIEWELQKENSKRWNPEVQYTSNYAKSANVDFTVDNNGLYTEPRPIGTRYLTRPL |

In certain embodiments, a single-stranded AAV (ssAAV) may be used as described here. In certain embodiments, a self-complementary vector, e.g., scAAV, may be used (see, e.g., Wu J et al., 2007, Human Gene Therapy, 18 (2): 171-82; McCarty D M et al., 2001, Gene Ther. 2001 August; 8 (16): 1248-54; and U.S. Pat. Nos. 6,596,535; 7,125,717; and 7,456,683, each of which is incorporated herein by reference in its entirety).

### Expression Cassettes

The present disclosure provides gene expression cassettes and rAAVs comprising gene expression cassettes in which expression of the transgene (DN-TNFα) is controlled by engineered nucleic acid regulatory elements that have more than one regulatory element (e.g. a promoter and/or enhancer, and other transcriptional elements), including regulatory elements that are arranged in tandem (two or three copies) that promote tissue-specific expression (e.g., brain-specific expression, liver-specific expression, muscle-specific expression, both liver-specific expression and muscle-specific expression, ocular-specific expression, etc.). Regulatory elements (or regulatory control elements) are cis-acting non-coding DNA regions that regulate the transcription of a gene, and include, but are not limited to, promoters, enhancers, silencers, polyA and introns. These individual elements are described elsewhere in this disclosure.

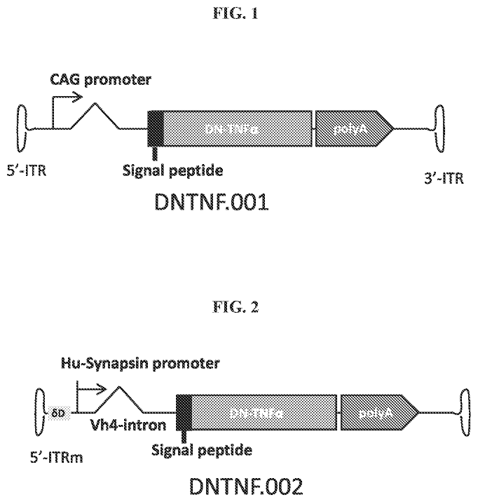

The expression cassette comprises an assembly of elements between two AAV inverted terminal repeat (ITR) sequences. In one example, as shown in FIG. 1, the expression cassette between a 5′ ITR sequence and a 3′-ITR sequence, comprises the following components from end to end: a promoter (e.g., CAG promoter), a signal peptide, the DN-TNFα transgene, and a polyA tail. In another example, as shown in FIG. 2, the expression cassette between a 5′ITR sequence and a 3′-ITR sequence comprises a promoter (e.g., hu-synapsin promoter), an intron (e.g., VH4 intron), a signal peptide, the DN-TNFα transgene, and a polyA tail.

The expression cassette is cloned into a plasmid (e.g. a cis plasmid) by methods known in the art. This process employs the use of a plasmid (e.g., a pSub201 plasmid or any plasmid known in the art) which contains an ori (e.g., section of the plasmid where replication begins) and optionally an antibiotic-resistance marker for the production and purification of a cell bank comprising the plasmid. Thereafter, the plasmid is utilized in making the AAV vector comprising the transgene (DN-TNFα) by a bioprocess including the cis plasmid, a trans plasmid (rep/cap genes) and helper plasmid and cells in which to form the vector. It is the artificial genome from the cis plasmid that becomes encapsidated in the capsid to become the AAV vector or product. Such AAV product is capable of transduction of host/target cells for therapeutic purposes, where the nucleus of the host cells directs transcription/translation of the transgene. Without being bound by theory, it is believed that the artificial genome is likely within an episome in the nucleus which is transcribed and translated.

Methods of producing rAAV particles comprising the expression cassette are described elsewhere in this disclosure. Briefly, the production of rAAV particles requires four elements: an rAAV vector plasmid containing the transgene flanked by AAV ITRs (often referred to as a cis plasmid), a plasmid that supplies the AAV viral proteins necessary for replicating and packaging the rAAV sequences (AAV rep/cap plasmid, or trans plasmid), a plasmid supplying the adenoviral helper genes (helper plasmid), and tissue culture cells. After co-transfection of the plasmids into the tissue culture cells, rAAV is produced and the cells are harvested. The rAAV is then purified by CsCl density gradient centrifugation, or other suitable purification technique, and dialyzed prior to storage, use in tissue culture cells, or use in hosts. Methods of cloning a transgene expression cassette, producing sufficient amounts of plasmids, and manufacturing rAAV vectors are known in the art. See e.g., Gray S J, et al., Curr Protoe Neurosci. 2011; Chapter 4: Unit4.17-4.17.

Thus, the expression cassette comprising the DN-TNFα transgene described herein can be used to produce AAV compositions of this disclosure, which compositions are then used in the methods of treating subjects upon administration, as also described herein.

The ITR to ITR nucleotide sequence elements of a first representative transgene cassette is disclosed in Table 4 below:

TABLE 4
Representative Transgene Cassette Elements

| Element | SEQ ID NO: |
|---|---|
| 5′ ITR-A | SEQ ID NO: 111 |
| CAG promoter | SEQ ID NO: 48 |
| Kozak sequence | SEQ ID NO: 176 |
| Mutant IL-2 leader | SEQ ID NO: 115 |
| peptide | |
| DN-TNFa coding | SEQ ID NO: 3 |
| sequence | |
| Rabbit B-globin polyA | SEQ ID NO: 109 |
| 3′ ITR AAV | SEQ ID NO: 113 |

The ITR to ITR nucleotide sequence elements of a second representative transgene cassette is disclosed in Table 5 below:

TABLE 5
Representative Transgene Cassette Elements

| Element | Amino Acid Sequence |
|---|---|
| 5′ mutated ITR (ITRm) (5′ ITR-B) | SEQ ID NO: 112 |
| human Synapsin promoter (hSyn) | SEQ ID NO: 53 |
| 5′-SS | CAG |
| VH4 intron | SEQ ID NO: 108 |
| 3′-SS | GTT |
| DN-TNFa coding sequence | SEQ ID NO: 3 |
| Rabbit B-globin polyA | SEQ ID NO: 109 |
| BGH polyA | SEQ ID NO: 114 |
| 3′ ITR | SEQ ID NO: 183 |

In some embodiments, the DN-TNFα transgene cassette has the S′-ITR to 3′-ITR nucleotide sequence set forth in SEQ ID NO: 165. In some embodiments, the DN-TNFα transgene cassette has the CAG promoter and transgene nucleotide sequence set forth in SEQ ID NO: 166. In some embodiments, the DN-TNFα transgene cassette has the CAG promoter, transgene and rabbit globin poly A nucleotide sequence set forth in SEQ ID NO: 167. In some embodiments, the DN-TNFα transgene cassette has the 5′-ITR to 3′-ITR nucleotide sequence set forth in SEQ ID NO: 168. In some embodiments, the DN-TNFα transgene cassette has the hSyn promoter and transgene nucleotide sequence set forth in SEQ ID NO: 169. In some embodiments, the DN-TNFα transgene cassette has the hSyn promoter, transgene and rabbit globin polyA nucleotide sequence set forth in SEQ ID NO: 170.

Representative Expression Cassettes of DN-TNFα are disclosed in Table 6 below:

TABLE 6
Representative Expression Cassettes of DN-TNF

| Description | |
|---|---|
| (SEQ ID NO) | Sequence |
| DNTNF.001 | CTGCGCGCTCGCTCGCTCACTGAGGCCGCCCGGGCAAAGCCCG |
| Full | GGCGTCGGGCGACCTTTGGTCGCCCGGCCTCAGTGAGCGAGCGAGC |
| transgene | GCGCAGAGAGGGAGTGGCCAACTCCATCACTAGGGGTTCCTTGTAG |
| cassette: 5′-ITR | TTAATGATTAACCCGCCATGCTACTTATCTACCAGGGTAATGGGGA |
| to 3′-ITR | TCCTCTAGAACTATAGCTAGTCGACATTGATTATTGACTAGTTATTA |
| nucleotide | ATAGTAATCAATTACGGGGTCATTAGTTCATAGCCCATATATGGAG |
| sequence | TTCCGCGTTACATAACTTACGGTAAATGGCCCGCCTGGCTGACCGC |
| (SEQ ID | CCAACGACCCCCGCCCATTGACGTCAATAATGACGTATGTTCCCAT |
| NO: 165) | AGTAACGCCAATAGGGACTTTCCATTGACGTCAATGGGTGGAGTAT |
| | TTACGGTAAACTGCCCACTTGGCAGTACATCAAGTGTATCATATGC |
| | CAAGTACGCCCCCTATTGACGTCAATGACGGTAAATGGCCCGCCTG |
| | GCATTATGCCCAGTACATGACCTTATGGGACTTTCCTACTTGGCAGT |
| | ACATCTACGTATTAGTCATCGCTATTACCATGGTCGAGGTGAGCCC |
| | CACGTTCTGCTTCACTCTCCCCATCTCCCCCCCCTCCCCACCCCCAA |
| | TTTTGTATTTATTTATTTTTTAATTATTTTGTGCAGCGATGGGGGCGG |
| | GGGGGGGGGGGGGGCGCGCGCCAGGCGGGGGGGGCGGGGCGAG |
| | GGGCGGGGCGGGGCGAGGCGGAGAGGTGCGGCGGCAGCCAATCAG |
| | AGCGGCGCGCTCCGAAAGTTTCCTTTTATGGCGAGGCGGCGGCGGC |
| | GGCGGCCCTATAAAAAGCGAAGCGCGCGGCGGGCGGGAGTCGCTG |
| | CGCGCTGCCTTCGCCCCGTGCCCCGCTCCGCCGCCGCCTCGCGCCGC |
| | CCGCCCCGGCTCTGACTGACCGCGTTACTCCCACAGGTGAGCGGGC |
| | GGGACGGCCCTTCTCCTCCGGGCTGTAATTAGCGCTTGGTTTAATGA |
| | CGGCTTGTTTCTTTTCTGTGGCTGCGTGAAAGCCTTGAGGGGCTCCG |
| | GGAGGGCCCTTTGTGCGGGGGGAGCGGCTCGGGGGGTGCGTGCGT |
| | GTGTGTGTGCGTGGGGAGCGCCGCGTGCGGCTCCGCGCTGCCCGGC |
| | GGCTGTGAGCGCTGCGGGCGCGGCGCGGGGCTTTGTGCGCTCCGCA |
| | GTGTGCGCGAGGGGAGCGCGGCCGGGGGCGGTGCCCCGCGGTGCG |
| | GGGGGGGCTGCGAGGGGAACAAAGGCTGCGTGCGGGGTGTGTGCG |
| | TGGGGGGGTGAGCAGGGGGTGTGGGCGCGTCGGTCGGGCTGCAAC |
| | CCCCCCTGCACCCCCCTCCCCGAGTTGCTGAGCACGGCCCGGCTTC |
| | GGGTGCGGGGCTCCGTACGGGGCGTGGCGCGGGGCTCGCCGTGCC |
| | GGGCGGGGGGGGCGGCAGGTGGGGGTGCCGGGGGGGGCGGGGCC |
| | GCCTCGGGCCGGGGAGGGCTCGGGGGAGGGGCGCGGCGGCCCCCG |
| | GAGCGCCGGCGGCTGTCGAGGCGCGGCGAGCCGCAGCCATTGCCTT |
| | TTATGGTAATCGTGCGAGAGGGCGCAGGGACTTCCTTTGTCCCAAA |
| | TCTGTGCGGAGCCGAAATCTGGGAGGCGCCGCCGCACCCCCTCTAG |
| | CGGGCGCGGGGCGAAGCGGTGCGGCGCCGGCAGGAAGGAAATGGG |
| | CGGGGAGGGCCTTCGTGCGTCGCCGCGCCGCCGTCCCCTTCTCCCTC |
| | TCCAGCCTCGGGGCTGTCCGCGGGGGGACGGCTGCCTTCGGGGGGG |
| | ACGGGGCAGGGCGGGGTTCGGCTTCTGGCGTGTGACCGGCGGCTCT |
| | AGAGCCTCTGCTAACCATGTTCATGCCTTCTTCTTTTTCCTACAGCT |
| | CCTGGGCAACGTGCTGGTTATTGTGCTGTCTCATCATTTTGGCAAAG |
| | AATTCGCCACCATGTATAGAATGCAGCTGCTGCTGCTGATTGCTCTG |
| | TCCCTGGCTCTGGTGACCAACTCTGTGAGATCCTCCTCAAGAACCCC |
| | TTCTGATAAACCTGTGGCTCATGTGGTGGCTAACCCTCAGGCAGAA |
| | GGACAGCTGCAGTGGCTGAATAGGAGGGCCAATGCACTGCTGGCT |
| | AATGGAGTGGAACTGAGGGACAACCAGCTGGTGGTCCCTTCAGAA |
| | GGACTGTACCTGATCTATAGCCAGGTGCTGTTCAAGGGCCAGGGAT |
| | GTCCCAGTACTCATGTGCTGCTGACTCACACCATCTCCAGAATTGCT |
| | GTCTCCCATCAGACAAAGGTGAACCTGCTGTCAGCCATCAAAAGTC |
| | CCTGCCAGAGAGAGACACCAGAAGGAGCTGAGGCCAAGCCATGGT |
| | ATGAGCCAATCTACCTGGGGGGAGTGTTTCAGCTGGAGAAAGGAG |
| | ACAGGCTGAGTGCAGAAATCAACAGACCAGACTATCTGGACTTTAG |
| | AGAATCAGGGCAGGTGTATTTTGGCATTATTGCACTGTGATAACTC |
| | GAGGACGGGGTGAACTACGCCTGAGGATCCGATCTTTTTCCCTCTG |
| | CCAAAAATTATGGGGACATCATGAAGCCCCTTGAGCATCTGACTTC |
| | TGGCTAATAAAGGAAATTTATTTTCATTGCAATAGTGTGTTGGAATT |
| | TTTTGTGTCTCTCACTCGGAAGCAATTCGTTGATCTGAATTTCGACC |
| | ACCCATAATACCCATTACCCTGGTAGATAAGTAGCATGGGGGTTA |
| | ATCATTAACTACAAGGAACCCCTAGTGATGGAGTTGGCCACTCCCT |
| | CTCTGCGCGCTCGCTCGCTCACTGAGGCCGGGCGACCAAAGGTCGC |
| | CCGACGCCCGGGCTTTGCCCGGGCGGCCTCAGTGAGCGAGCGAGCG |
| | CGCAG |
| DNTNF.001 | GACATTGATTATTGACTAGTTATTAATAGTAATCAATTACGGGG |
| CAG | TCATTAGTTCATAGCCCATATATGGAGTTCCGCGTTACATAACTTAC |
| promoter + | GGTAAATGGCCCGCCTGGCTGACCGCCCAACGACCCCCGCCCATTG |
| transgene | ACGTCAATAATGACGTATGTTCCCATAGTAACGCCAATAGGGACTT |
| nucleotide | TCCATTGACGTCAATGGGTGGAGTATTTACGGTAAACTGCCCACTT |
| sequence | GGCAGTACATCAAGTGTATCATATGCCAAGTACGCCCCCTATTGAC |
| (SEQ ID | GTCAATGACGGTAAATGGCCCGCCTGGCATTATGCCCAGTACATGA |
| NO: 166) | CCTTATGGGACTTTCCTACTTGGCAGTACATCTACGTATTAGTCATC |
| | GCTATTACCATGGTCGAGGTGAGCCCCACGTTCTGCTTCACTCTCCC |
| | CATCTCCCCCCCCTCCCCACCCCCAATTTTGTATTTATTTATTTTTTA |
| | ATTATTTTGTGCAGCGATGGGGGCGGGGGGGGGGGGGGGGCGCGC |
| | GCCAGGCGGGGCGGGGGGGGGCGAGGGGCGGGGCGGGGCGAGGC |
| | GGAGAGGTGCGGCGGCAGCCAATCAGAGCGGCGCGCTCCGAAAGT |
| | TTCCTTTTATGGCGAGGCGGCGGCGGCGGCGGCCCTATAAAAAGCG |
| | AAGCGCGCGGCGGGCGGGAGTCGCTGCGCGCTGCCTTCGCCCCGTG |
| | CCCCGCTCCGCCGCCGCCTCGCGCCGCCCGCCCCGGCTCTGACTGA |
| | CCGCGTTACTCCCACAGGTGAGCGGGCGGGACGGCCCTTCTCCTCC |
| | GGGCTGTAATTAGCGCTTGGTTTAATGACGGCTTGTTTCTTTTCTGT |
| | GGCTGCGTGAAAGCCTTGAGGGGCTCCGGGAGGGCCCTTTGTGCGG |
| | GGGGAGCGGCTCGGGGGGTGCGTGCGTGTGTGTGTGCGTGGGGAG |
| | CGCCGCGTGCGGCTCCGCGCTGCCCGGCGGCTGTGAGCGCTGCGGG |
| | CGCGGCGCGGGGCTTTGTGCGCTCCGCAGTGTGCGCGAGGGGAGCG |
| | CGGCCGGGGGCGGTGCCCCGCGGTGCGGGGGGGGCTGCGAGGGGA |
| | ACAAAGGCTGCGTGCGGGGTGTGTGCGTGGGGGGGTGAGCAGGGG |
| | GTGTGGGCGCGTCGGTCGGGCTGCAACCCCCCCTGCACCCCCCTCC |
| | CCGAGTTGCTGAGCACGGCCCGGCTTCGGGTGCGGGGCTCCGTACG |
| | GGGCGTGGCGCGGGGCTCGCCGTGCCGGGGGGGGGTGGCGGCAG |
| | GTGGGGGTGCCGGGGGGGGGGGGCCGCCTCGGGCCGGGGAGGGC |
| | TCGGGGGAGGGGCGCGGCGGCCCCCGGAGCGCCGGCGGCTGTCGA |
| | GGCGCGGCGAGCCGCAGCCATTGCCTTTTATGGTAATCGTGCGAGA |
| | GGGCGCAGGGACTTCCTTTGTCCCAAATCTGTGCGGAGCCGAAATC |
| | TGGGAGGCGCCGCCGCACCCCCTCTAGCGGGCGCGGGGCGAAGCG |
| | GTGCGGCGCCGGCAGGAAGGAAATGGGGGGGGAGGGCCTTCGTGC |
| | GTCGCCGCGCCGCCGTCCCCTTCTCCCTCTCCAGCCTCGGGGCTGTC |
| | CGCGGGGGGACGGCTGCCTTCGGGGGGGACGGGGCAGGGGGGGGT |
| | TCGGCTTCTGGCGTGTGACCGGCGGCTCTAGAGCCTCTGCTAACCA |
| | TGTTCATGCCTTCTTCTTTTTCCTACAGCTCCTGGGCAACGTGCTGG |
| | TTATTGTGCTGTCTCATCATTTTGGCAAAGAATTCGCCACCATGTAT |
| | AGAATGCAGCTGCTGCTGCTGATTGCTCTGTCCCTGGCTCTGGTGAC |
| | CAACTCTGTGAGATCCTCCTCAAGAACCCCTTCTGATAAACCTGTG |
| | GCTCATGTGGTGGCTAACCCTCAGGCAGAAGGACAGCTGCAGTGGC |
| | TGAATAGGAGGGCCAATGCACTGCTGGCTAATGGAGTGGAACTGA |
| | GGGACAACCAGCTGGTGGTCCCTTCAGAAGGACTGTACCTGATCTA |
| | TAGCCAGGTGCTGTTCAAGGGCCAGGGATGTCCCAGTACTCATGTG |
| | CTGCTGACTCACACCATCTCCAGAATTGCTGTCTCCCATCAGACAA |
| | AGGTGAACCTGCTGTCAGCCATCAAAAGTCCCTGCCAGAGAGAGAC |
| | ACCAGAAGGAGCTGAGGCCAAGCCATGGTATGAGCCAATCTACCT |
| | GGGGGGAGTGTTTCAGCTGGAGAAAGGAGACAGGCTGAGTGCAGA |
| | AATCAACAGACCAGACTATCTGGACTTTAGAGAATCAGGGCAGGTG |
| | TATTTTGGCATTATTGCACTGTGA |
| DNTNF.001 | GACATTGATTATTGACTAGTTATTAATAGTAATCAATTACGGGG |
| CAG | TCATTAGTTCATAGCCCATATATGGAGTTCCGCGTTACATAACTTAC |
| promoter, | GGTAAATGGCCCGCCTGGCTGACCGCCCAACGACCCCCGCCCATTG |
| transgene and | ACGTCAATAATGACGTATGTTCCCATAGTAACGCCAATAGGGACTT |
| rabbit globin | TCCATTGACGTCAATGGGTGGAGTATTTACGGTAAACTGCCCACTT |
| poly A nucleotide | GGCAGTACATCAAGTGTATCATATGCCAAGTACGCCCCCTATTGAC |
| sequence | GTCAATGACGGTAAATGGCCCGCCTGGCATTATGCCCAGTACATGA |
| (SEQ ID | CCTTATGGGACTTTCCTACTTGGCAGTACATCTACGTATTAGTCATC |
| NO: 167) | GCTATTACCATGGTCGAGGTGAGCCCCACGTTCTGCTTCACTCTCCC |
| | CATCTCCCCCCCCTCCCCACCCCCAATTTTGTATTTATTTATTTTTTA |
| | ATTATTTTGTGCAGCGATGGGGGCGGGGGGGGGGGGGGGGCGCGC |
| | GCCAGGCGGGGCGGGGCGGGGCGAGGGGGGGGGGGGGCGAGGC |
| | GGAGAGGTGCGGCGGCAGCCAATCAGAGCGGCGCGCTCCGAAAGT |
| | TTCCTTTTATGGCGAGGCGGCGGCGGCGGCGGCCCTATAAAAAGCG |
| | AAGCGCGCGGCGGGCGGGAGTCGCTGCGCGCTGCCTTCGCCCCGTG |
| | CCCCGCTCCGCCGCCGCCTCGCGCCGCCCGCCCCGGCTCTGACTGA |
| | CCGCGTTACTCCCACAGGTGAGCGGGCGGGACGGCCCTTCTCCTCC |
| | GGGCTGTAATTAGCGCTTGGTTTAATGACGGCTTGTTTCTTTTCTGT |
| | GGCTGCGTGAAAGCCTTGAGGGGCTCCGGGAGGGCCCTTTGTGCGG |
| | GGGGAGCGGCTCGGGGGGTGCGTGCGTGTGTGTGTGCGTGGGGAG |
| | CGCCGCGTGCGGCTCCGCGCTGCCCGGCGGCTGTGAGCGCTGCGGG |
| | CGCGGCGCGGGGCTTTGTGCGCTCCGCAGTGTGCGCGAGGGGAGCG |
| | CGGCCGGGGGCGGTGCCCCGCGGTGCGGGGGGGGCTGCGAGGGGA |
| | ACAAAGGCTGCGTGCGGGGTGTGTGCGTGGGGGGGTGAGCAGGGG |
| | GTGTGGGCGCGTCGGTCGGGCTGCAACCCCCCCTGCACCCCCCTCC |
| | CCGAGTTGCTGAGCACGGCCCGGCTTCGGGTGCGGGGCTCCGTACG |
| | GGGCGTGGCGCGGGGCTCGCCGTGCCGGGCGGGGGGGGCGGCAG |
| | GTGGGGGTGCCGGGCGGGGGGGGGCCGCCTCGGGCCGGGGAGGGC |
| | TCGGGGGAGGGGCGCGGCGGCCCCCGGAGCGCCGGCGGCTGTCGA |
| | GGCGCGGCGAGCCGCAGCCATTGCCTTTTATGGTAATCGTGCGAGA |
| | GGGCGCAGGGACTTCCTTTGTCCCAAATCTGTGCGGAGCCGAAATC |
| | TGGGAGGCGCCGCCGCACCCCCTCTAGCGGGCGCGGGGCGAAGCG |
| | GTGCGGCGCCGGCAGGAAGGAAATGGGCGGGGAGGGCCTTCGTGC |
| | GTCGCCGCGCCGCCGTCCCCTTCTCCCTCTCCAGCCTCGGGGCTGTC |
| | CGCGGGGGGACGGCTGCCTTCGGGGGGGACGGGGCAGGGGGGGGT |
| | TCGGCTTCTGGCGTGTGACCGGCGGCTCTAGAGCCTCTGCTAACCA |
| | TGTTCATGCCTTCTTCTTTTTCCTACAGCTCCTGGGCAACGTGCTGG |
| | TTATTGTGCTGTCTCATCATTTTGGCAAAGAATTCGCCACCATGTAT |
| | AGAATGCAGCTGCTGCTGCTGATTGCTCTGTCCCTGGCTCTGGTGAC |
| | CAACTCTGTGAGATCCTCCTCAAGAACCCCTTCTGATAAACCTGTG |
| | GCTCATGTGGTGGCTAACCCTCAGGCAGAAGGACAGCTGCAGTGGC |
| | TGAATAGGAGGGCCAATGCACTGCTGGCTAATGGAGTGGAACTGA |
| | GGGACAACCAGCTGGTGGTCCCTTCAGAAGGACTGTACCTGATCTA |
| | TAGCCAGGTGCTGTTCAAGGGCCAGGGATGTCCCAGTACTCATGTG |
| | CTGCTGACTCACACCATCTCCAGAATTGCTGTCTCCCATCAGACAA |
| | AGGTGAACCTGCTGTCAGCCATCAAAAGTCCCTGCCAGAGAGAGAC |
| | ACCAGAAGGAGCTGAGGCCAAGCCATGGTATGAGCCAATCTACCT |
| | GGGGGGAGTGTTTCAGCTGGAGAAAGGAGACAGGCTGAGTGCAGA |
| | AATCAACAGACCAGACTATCTGGACTTTAGAGAATCAGGGCAGGTG |
| | TATTTTGGCATTATTGCACTGTGATAACTCGAGGACGGGGTGAACT |
| | ACGCCTGAGGATCCGATCTTTTTCCCTCTGCCAAAAATTATGGGGA |
| | CATCATGAAGCCCCTTGAGCATCTGACTTCTGGCTAATAAAGGAAA |
| | TTTATTTTCATTGCAATAGTGTGTTGGAATTTTTTGTGTCTCTCACTC |
| | G |
| DNTNF.002 | CTGCGCGCTCGCTCGCTCACTGAGGCCGCCCGGGCAAAGCCCG |
| Full | GGCGTCGGGCGACCTTTGGTCGCCCGGCCTCAGTGAGCGAGCGAGC |
| transgene | GCGCAGAGAGGGAGTGGAATTCACGCGTGGTACCAGTGCAAGTGG |
| cassette: 5′- | GTTTTAGGACCAGGATGAGGCGGGGTGGGGGTGCCTACCTGACGAC |
| mITR to 3′-ITR | CGACCCCGACCCACTGGACAAGCACCCAACCCCCATTCCCCAAATT |
| nucleotide | GCGCATCCCCTATCAGAGAGGGGGAGGGGAAACAGGATGCGGCGA |
| sequence | GGCGCGTGCGCACTGCCAGCTTCAGCACCGCGGACAGTGCCTTCGC |
| (SEQ ID | CCCCGCCTGGCGGCGCGCGCCACCGCCGCCTCAGCACTGAAGGCGC |
| NO: 168) | GCTGACGTCACTCGCCGGTCCCCCGCAAACTCCCCTTCCCGGCCAC |
| | CTTGGTCGCGTCCGCGCCGCCGCCGGCCCAGCCGGACCGCACCACG |
| | CGAGGCGCGAGATAGGGGGGCACGGGCGCGACCATCTGCGCTGCG |
| | GCGCCGGCGACTCAGCGCTGCCTCAGTCTGCGGTGGGCAGCGGAGG |
| | AGTCGTGTCGTGCCTGAGAGCGCAGTCTAGACAGGTGAGTATCTCA |
| | GGGATCCAGACATGGGGATATGGGAGGTGCCTCTGATCCCAGGGCT |
| | CACTGTGGGTCTCTCTGTTCACAGGTTTCTAGAACTAGTAGTAATTC |
| | GCCACCATGTATAGAATGCAGCTGCTGCTGCTGATTGCTCTGTCCCT |
| | GGCTCTGGTGACCAACTCTGTGAGATCCTCCTCAAGAACCCCTTCTG |
| | ATAAACCTGTGGCTCATGTGGTGGCTAACCCTCAGGCAGAAGGACA |
| | GCTGCAGTGGCTGAATAGGAGGGCCAATGCACTGCTGGCTAATGGA |
| | GTGGAACTGAGGGACAACCAGCTGGTGGTCCCTTCAGAAGGACTGT |
| | ACCTGATCTATAGCCAGGTGCTGTTCAAGGGCCAGGGATGTCCCAG |
| | TACTCATGTGCTGCTGACTCACACCATCTCCAGAATTGCTGTCTCCC |
| | ATCAGACAAAGGTGAACCTGCTGTCAGCCATCAAAAGTCCCTGCCA |
| | GAGAGAGACACCAGAAGGAGCTGAGGCCAAGCCATGGTATGAGCC |
| | AATCTACCTGGGGGGAGTGTTTCAGCTGGAGAAAGGAGACAGGCT |
| | GAGTGCAGAAATCAACAGACCAGACTATCTGGACTTTAGAGAATCA |
| | GGGCAGGTGTATTTTGGCATTATTGCACTGTGATAACTCGAGAGCG |
| | GCCATCAAGCTTGATCTTTTTCCCTCTGCCAAAAATTATGGGGACAT |
| | CATGAAGCCCCTTGAGCATCTGACTTCTGGCTAATAAAGGAAATTT |
| | ATTTTCATTGCAATAGTGTGTTGGAATTTTTTGTGTCTCTCACTCGG |
| | CTAGCGAAGCAATTCTAGCAGGCATGCTGGGGAGAGATCGATCTGA |
| | GGAACCCCTAGTGATGGAGTTGGCCACTCCCTCTCTGCGCGCTCGC |
| | TCGCTCACTGAGGCCGCCCGGGCAAAGCCCGGGCGTCGGGCGACCT |
| | TTGGTCGCCCGGCCTCAGTGAGCGAGCGAGCGCGCAGAGAGGGAG |
| | TGGCCAA |
| DNTNF.002 | AGTGCAAGTGGGTTTTAGGACCAGGATGAGGCGGGGGGGGGT |
| hSyn | GCCTACCTGACGACCGACCCCGACCCACTGGACAAGCACCCAACCC |
| promoter + | CCATTCCCCAAATTGCGCATCCCCTATCAGAGAGGGGGAGGGGAAA |
| transgene | CAGGATGCGGCGAGGCGCGTGCGCACTGCCAGCTTCAGCACCGCG |
| nucleotide | GACAGTGCCTTCGCCCCCGCCTGGCGGCGCGCGCCACCGCCGCCTC |
| sequence | AGCACTGAAGGCGCGCTGACGTCACTCGCCGGTCCCCCGCAAACTC |
| (SEQ ID | CCCTTCCCGGCCACCTTGGTCGCGTCCGCGCCGCCGCCGGCCCAGC |
| NO: 169) | CGGACCGCACCACGCGAGGCGCGAGATAGGGGGGCACGGGCGCGA |
| | CCATCTGCGCTGCGGCGCCGGCGACTCAGCGCTGCCTCAGTCTGCG |
| | GTGGGCAGCGGAGGAGTCGTGTCGTGCCTGAGAGCGCAGtctagaCAG |
| | GTGAGTATCTCAGGGATCCAGACATGGGGATATGGGAGGTGCCTCT |
| | GATCCCAGGGCTCACTGTGGGTCTCTCTGTTCACAGGTTTCTAGAAC |
| | TAGTAGTAATTCGCCACCATGTATAGAATGCAGCTGCTGCTGCTGA |
| | TTGCTCTGTCCCTGGCTCTGGTGACCAACTCTGTGAGATCCTCCTCA |
| | AGAACCCCTTCTGATAAACCTGTGGCTCATGTGGTGGCTAACCCTC |
| | AGGCAGAAGGACAGCTGCAGTGGCTGAATAGGAGGGCCAATGCAC |
| | TGCTGGCTAATGGAGTGGAACTGAGGGACAACCAGCTGGTGGTCCC |
| | TTCAGAAGGACTGTACCTGATCTATAGCCAGGTGCTGTTCAAGGGC |
| | CAGGGATGTCCCAGTACTCATGTGCTGCTGACTCACACCATCTCCA |
| | GAATTGCTGTCTCCCATCAGACAAAGGTGAACCTGCTGTCAGCCAT |
| | CAAAAGTCCCTGCCAGAGAGAGACACCAGAAGGAGCTGAGGCCAA |
| | GCCATGGTATGAGCCAATCTACCTGGGGGGAGTGTTTCAGCTGGAG |
| | AAAGGAGACAGGCTGAGTGCAGAAATCAACAGACCAGACTATCTG |
| | GACTTTAGAGAATCAGGGCAGGTGTATTTTGGCATTATTGCACTGT |
| | GA |
| DNTNF.002 | AGTGCAAGTGGGTTTTAGGACCAGGATGAGGCGGGGTGGGGGT |
| hSyn | GCCTACCTGACGACCGACCCCGACCCACTGGACAAGCACCCAACCC |
| promoter, | CCATTCCCCAAATTGCGCATCCCCTATCAGAGAGGGGGAGGGGAAA |
| transgene and | CAGGATGCGGCGAGGCGCGTGCGCACTGCCAGCTTCAGCACCGCG |
| rabbit globin | GACAGTGCCTTCGCCCCCGCCTGGCGGCGCGCGCCACCGCCGCCTC |
| polyA nucleotide | AGCACTGAAGGCGCGCTGACGTCACTCGCCGGTCCCCCGCAAACTC |
| sequence | CCCTTCCCGGCCACCTTGGTCGCGTCCGCGCCGCCGCCGGCCCAGC |
| (SEQ ID | CGGACCGCACCACGCGAGGCGCGAGATAGGGGGGCACGGGCGCGA |
| NO: 170) | CCATCTGCGCTGCGGCGCCGGCGACTCAGCGCTGCCTCAGTCTGCG |
| | GTGGGCAGCGGAGGAGTCGTGTCGTGCCTGAGAGCGCAGTCTAGA |
| | CAGGTGAGTATCTCAGGGATCCAGACATGGGGATATGGGAGGTGC |
| | CTCTGATCCCAGGGCTCACTGTGGGTCTCTCTGTTCACAGGTTTCTA |
| | GAACTAGTAGTAATTCGCCACCATGTATAGAATGCAGCTGCTGCTG |
| | CTGATTGCTCTGTCCCTGGCTCTGGTGACCAACTCTGTGAGATCCTC |
| | CTCAAGAACCCCTTCTGATAAACCTGTGGCTCATGTGGTGGCTAAC |
| | CCTCAGGCAGAAGGACAGCTGCAGTGGCTGAATAGGAGGGCCAAT |
| | GCACTGCTGGCTAATGGAGTGGAACTGAGGGACAACCAGCTGGTG |
| | GTCCCTTCAGAAGGACTGTACCTGATCTATAGCCAGGTGCTGTTCA |
| | AGGGCCAGGGATGTCCCAGTACTCATGTGCTGCTGACTCACACCAT |
| | CTCCAGAATTGCTGTCTCCCATCAGACAAAGGTGAACCTGCTGTCA |
| | GCCATCAAAAGTCCCTGCCAGAGAGAGACACCAGAAGGAGCTGAG |
| | GCCAAGCCATGGTATGAGCCAATCTACCTGGGGGGAGTGTTTCAGC |
| | TGGAGAAAGGAGACAGGCTGAGTGCAGAAATCAACAGACCAGACT |
| | ATCTGGACTTTAGAGAATCAGGGCAGGTGTATTTTGGCATTATTGC |
| | ACTGTGATAACTCGAGAGCGGCCATCAAGCTTGATCTTTTTCCCTCT |
| | GCCAAAAATTATGGGGACATCATGAAGCCCCTTGAGCATCTGACTT |
| | CTGGCTAATAAAGGAAATTTATTTTCATTGCAATAGTGTGTTGGAAT |
| | TTTTTGTGTCTCTCACTCG |

Generally, the genome or “minigene” to be delivered by the AAV vector is packaged by an AAV capsid. This Cis expression cassette includes AAV inverted terminal repeats (ITRs) flanking the 5′ and 3′ ends of the genome, and the cis expression cassette comprises a DN-TNFα transgene operably linked to expression control elements that will control expression of the DN-TNFα in human cells, such as promoters and the like. The expression cassette may be constructed into a plasmid, for example a Cis plasmid, in order to effectuate the production of the AAV vector when combined with other AAV elements such as viral helper genes, rep genes, a cap gene. One or more plasmids, containing all the genes and elements required for production and packaging of an AAV vector, are transfected into eukaryotic cells for the purpose of production of the gene therapy vector product.

Thus, provided are recombinant nucleic acid molecules comprising an expression cassette comprising a transgene sequence encoding DN-TNFα operably linked to one or more expression control elements, and AAV ITR sequences flanking the 5′ and 3′ ends of the transgene. In some embodiments, the transgene sequence encoding DN-TNFα is selected from SEQ ID NO: 3 or SEQ ID NO: 4. Also provided are recombinant nucleic acid molecules encoding a AAV capsid protein, and optionally further comprising a rep gene. In some embodiments, the recombinant nucleic acid molecule encodes an AAV capsid protein selected from Tables 1-3. In some embodiments, the recombinant nucleic acid molecule is a plasmid.

Plasmid DNA may be amplified for the production of a batch of plasmids used for AAV vector production. As such, plasmid DNA containing one or more of the recombinant nucleic acid molecules described herein are transformed into bacterial cells for amplification, then purification of such plasmid DNA for further use in production of AAV gene therapy vectors in eukaryotic host cells.

Also provided are cultured host cells containing a recombinant nucleic acid molecule comprising a transgene encoding DN-TNFα, such as SEQ ID NOs: 3 or 4, including but not limited to the AAV capsids included in Tables 1-3 and FIG. 6, wherein the recombinant nucleic acid molecule further comprises a heterologous non-AAV sequence, and wherein the recombinant nucleic acid molecule is a plasmid. In certain embodiments, the plasmid further comprises an origin of replication. In some instances, cultured host cells refers to the cells harboring the transformed plasmid.

Provided are cultured host cells containing a recombinant nucleic acid molecule encoding an AAV capsid protein, including but not limited to the AAV capsids included in Tables 1-3 and FIG. 6, wherein the recombinant nucleic acid molecule further comprises a heterologous non-AAV sequence, and wherein the recombinant nucleic acid molecule is a plasmid. In certain embodiments, the plasmid further comprises a origin of replication. In certain embodiments, the plasmid further comprises a rep gene.

### Promoters and Modifiers of Gene Expression

In certain embodiments, the vectors provided herein comprise components that are part of the expression cassette and modulate gene delivery or gene expression (e.g., “expression control elements”). In certain embodiments, the vectors provided herein comprise components that modulate gene expression. In certain embodiments, the vectors provided herein comprise components that influence binding or targeting to cells. In certain embodiments, the vectors provided herein comprise components that influence the localization of the polynucleotide (e.g., the transgene) within the cell after uptake. In certain embodiments, the vectors provided herein comprise components that can be used as detectable or selectable markers, e.g., to detect or select for cells that have taken up the polynucleotide. In some embodiments, the expression control element is a regulatory element within the expression cassette, such as a promoter.

In certain embodiments, the viral vectors provided herein comprise one or more promoters that control expression of the transgene. In certain embodiments, the promoter is a constitutive promoter. In certain embodiments, the promoter is a CB7 (also referred to as a CAG promoter) (see Dinculescu A. et al., 2005, Hum Gene Ther 16:649-663, incorporated by reference herein in its entirety). In some embodiments, the CAG or CB7 promoter (SEQ ID NO: 48 or 49) includes other expression control elements that enhance expression of the transgene driven by the vector. In certain embodiments, the other expression control elements include chicken β-actin intron and/or rabbit β-globin poly A signal. In some embodiments, the promoter comprises a TATA box. In other embodiments, the promoter comprises one or more elements. In certain embodiments, the one or more promoter elements may be inverted or moved relative to one another. In some embodiments, the elements of the promoter are positioned to function cooperatively. In other embodiments, the elements of the promoter are positioned to function independently. In certain embodiments, the viral vectors provided herein comprise one or more promoters selected from the group consisting of the human CMV immediate early gene promoter, the SV40 early promoter, the Rous sarcoma virus (RS) long terminal repeat, and rat insulin promoter. In some embodiments, the vectors provided herein comprise one or more long terminal repeat (LTR) promoters selected from the group consisting of AAV, MLV, MMTV, SV40, RSV, HIV-1, and HIV-2 LTRs. In other embodiments, the vectors provided herein comprise one or more tissue specific promoters (e.g., a retinal pigment epithelial cell-specific promoter, a CNS-specific promoter, a liver-specific promoter or a muscle-specific promoter). In certain embodiments, the viral vectors provided herein comprise a RPE65 promoter, an opsin promoter (a retinal cell/CNS specific promoter) (Nicoletti, A. et al., (1998) Invest Ophthalmol Vis Sci. 39 (3): 637-44; Li Q, et al. (2008) Vision Res 48 (3): 332-8) or a BST-1 promoter. In some embodiments, the viral vectors provided herein comprises a liver cell specific promoter, such as, a TBG (Thyroxine-binding Globulin) promoter (SEQ ID NO: 91), a SERPINA1 (hAAT) promoter (SEQ ID NO:89), an ApoE.hAAT promoter (SEQ ID NO: 90). In some embodiments the viral vector comprises an APOA2 promoter or a MIR122 promoter (Corella D, et al., Int J Obes (Lond). 2011; 35(5):666-675; Li Z Y, et al. (2011) J. Hepatol. 55, 602-611; Paluschinski, et al., 2018, Zeitschrift für Gastroenterologie 56(01):E2-E89. In some embodiments, the viral vector provided herein comprises a muscle specific promoter, such as a human desmin promoter (Jonuschies J et al., Curr Gene Ther. 2014; 14(4):276-88), a CK8 promoter (SEQ ID NO: 92; Himeda et al., 2011, Chapter 1,Muscle Gene Therapy: Methods and Protocols, Methods in Molecular Biology, Dongsheng Duan (ed.), 709:3-19), or a Pitx3 promoter (Coulon V et al., 2007, The Journal of Biological Chemistry. 282:33192-200). In other embodiments, the viral vector comprises a VMD2 promoter. In certain embodiments, the viral vector herein comprises synthetic and tandem promoters, e.g. the promoters listed in Tables 4-7 below, depending on the tissue being targeted.

Representative promoters that are useful for the expression of the disclosed DN-TNFα proteins in mammalian cells (transduced with viral vector) include ubiquitous promoters such as, e.g., a phosphoglycerate kinase (PKG) promoter, CAG (composite of the (CMV) cytomegalovirus enhancer the chicken beta actin promoter (CBA) and the rabbit beta globin intron), the SV40 early promoter, murine mammary tumor virus LTR promoter; adenovirus major late promoter (Ad MLP); a herpes simplex virus (HSV) promoter, a CMV promoter such as the CMV immediate early promoter region (CMV-IE), rous sarcoma virus (RSV) promoter, and U6 promoter. For the purpose of driving cell-type specific expression of DN-TNFα sequences disclosed herein, cell-type specific promoters may be used.

Promoters for the universal tissue expression include, but are not limited to, CAG promoter, CB/CBA promoter, UbC promoter, mU1a promoter, EF-1α promoter, the sequences of which are provided in Table 7. The CAG promoter is a composite of Cytomegalovirus (CMV) early enhancer fused to chicken beta-actin promoter, the first exon and the first intron of chicken beta-actin gene, and the splice acceptor of the rabbit beta-globin gene. Promoters for CNS-specific expression, include, but are not limited to, Human Synapsin Promoter 1 (hSyn-1), Human Synapsin Promoter 2 (hSyn-2), Human Synapsin Promoter 3 (hSyn-3), Human Synapsin Promoter 4 (hSyn-4), Human Synapsin Promoter 5 (hSyn-5), Mecp2 promoter, Human Glial fibrillary acidic protein (hGFAP) promoter, Rat Neuron-specific enolase (NSE)/RU5′ promoter, neuronal nuclei (NeuN) promoter, Calcium/calmodulin-dependent protein kinases (CaMK) II_1 promoter, Complement C1q Like 2 (C1ql2 1) promoter, C1ql2 2 promoter, Dopamine Receptor D1 (DRD1) promoter, DRD2 (isoform 1) promoter, DRD2 (isoform 2) promoter, Pro-opiomelanocortin (POMC) promoter, Prospero Homeobox 1 (PROX1) (isoform 1) promoter, PROX1 (isoform 2) promoter, Microtubule Associated Protein 1B (MAP1B) (isoform 1) promoter, MAP1B (isoform 2) promoter, MAP1B (isoform 3) promoter, Tubulin Alpha 1a (Tα-1/TUBA1A) (isoform 1) promoter, the sequences of which are provided in Table 8. Promoters for liver-specific expression, include, but are not limited to, liver-specific promoter 1 (LSPX1) promoter, LSPX2 promoter, lipid transfer protein (LTP1) promoter, LTP2 promoter, LTP3 promoter, Apolipoprotein E enhancer; hAAT, human alpha-1 anti-trypsin promoter (ApoE.hAAT) promoter, and thyroxine binding globulin (TBG) promoter, the sequences of which are provided in Table 9. Promoters for muscle-specific expression include, but are not limited to, cytokeratin-8 (CK8) promoter, muscle-synthetic promoter (SPc5-12) promoter, muscle creatine kinase7 (MCK7) promoter, truncatedMCK (IMCK) promoter, Mouse skeletal muscle alpha actin acta1 promoter, Human muscle creatine kinase (MCK) promoter, Human desmin promoter, Human desmin 2 promoter, Human skeletal muscle alpha actin acta1 promoter, Mouse MCK promoter, and Mouse desmin promoter, the sequences of which are provided in Table 9. Promoters for synovial tissue-specific expression include, but are not limited to, CXCL10 promoter, the sequence of which are provided in Table 9. Promoters for liver-muscle-specific tandem expression include, but are not limited to, Liver-muscle tandem promoter (LMTP) 6 promoter, LMTP13 promoter, LMTP14 promoter, LMTP15 promoter, LMTP18 promoter, LMTP19 promoter, LMTP20 promoter, the sequence of which are provided in Table 9. Promoters for liver-bone-specific tandem expression, include, but are not limited to, Liver-bone tandem promoter (LBTP) 1 promoter, LBTP2 promoter, the sequence of which are provided in Table 9. Promoters for ocular-specific expression include, but are not limited to, Retinoid Isomerohydrolase (RPE65) promoter, Red cone opsin promoter, and Bone Marrow Stromal Cell Antigen 1 (BST1) promoter, the sequences of which are provided in Table 10.

Various promoter sequences are commercially available from various sources, e.g., Stratagene (San Diego, CA) or InvivoGen (San Diego, CA), or may be engineered using standard molecular biology techniques. Representative promoter sequences suitable for use in expression vectors (e.g., plasmid or viral vector, such as, e.g., an AAV or a lentiviral vector) are provided in Tables 1-4. Inducible promoters have been described, and provide regulatable transgene expression, including in the brain, utilizing, e.g. doxycycline-inducible viral vectors (Chtarto A et al., Methods & Clinical Development (2016) 5, 16027). Table 7 provides examples and nucleotide sequences of universal (ubiquitous) promoters for use herein.

TABLE 7
Representative Universal Promoters

| Promoter | Sequence |
|---|---|
| CAG | GACATTGATTATTGACTAGTTATTAATAGTAATCAATTACGGGGTCA |
| promoter | TTAGTTCATAGCCCATATATGGAGTTCCGCGTTACATAACTTACGGTAAA |
| (SEQ ID | TGGCCCGCCTGGCTGACCGCCCAACGACCCCCGCCCATTGACGTCAATA |
| NO: 48) | ATGACGTATGTTCCCATAGTAACGCCAATAGGGACTTTCCATTGACGTC |
| | AATGGGTGGAGTATTTACGGTAAACTGCCCACTTGGCAGTACATCAAGT |
| | GTATCATATGCCAAGTACGCCCCCTATTGACGTCAATGACGGTAAATGG |
| | CCCGCCTGGCATTATGCCCAGTACATGACCTTATGGGACTTTCCTACTTG |
| | GCAGTACATCTACGTATTAGTCATCGCTATTACCATGGTCGAGGTGAGC |
| | CCCACGTTCTGCTTCACTCTCCCCATCTCCCCCCCCTCCCCACCCCCAATT |
| | TTGTATTTATTTATTTTTTAATTATTTTGTGCAGCGATGGGGGGGGGGGG |
| | GGGGGGGGGGGGCGCGCCAGGCGGGGCGGGGCGGGGCGAGGGGGGGG |
| | GCGGGGCGAGGCGGAGAGGTGCGGCGGCAGCCAATCAGAGCGGCGCGC |
| | TCCGAAAGTTTCCTTTTATGGCGAGGCGGCGGCGGCGGCGGCCCTATAA |
| | AAAGCGAAGCGCGCGGCGGGCGGGAGTCGCTGCGCGCTGCCTTCGCCC |
| | CGTGCCCCGCTCCGCCGCCGCCTCGCGCCGCCCGCCCCGGCTCTGACTG |
| | ACCGCGTTACTCCCACAGGTGAGCGGGCGGGACGGCCCTTCTCCTCCGG |
| | GCTGTAATTAGCGCTTGGTTTAATGACGGCTTGTTTCTTTTCTGTGGCTG |
| | CGTGAAAGCCTTGAGGGGCTCCGGGAGGGCCCTTTGTGCGGGGGGAGC |
| | GGCTCGGGGGGTGCGTGCGTGTGTGTGTGCGTGGGGAGCGCCGCGTGCG |
| | GCTCCGCGCTGCCCGGCGGCTGTGAGCGCTGCGGGCGCGGCGCGGGGCT |
| | TTGTGCGCTCCGCAGTGTGCGCGAGGGGAGCGCGGCCGGGGGGGGTGC |
| | CCCGCGGTGCGGGGGGGGCTGCGAGGGGAACAAAGGCTGCGTGCGGGG |
| | TGTGTGCGTGGGGGGGTGAGCAGGGGGTGTGGGCGCGTCGGTCGGGCT |
| | GCAACCCCCCCTGCACCCCCCTCCCCGAGTTGCTGAGCACGGCCCGGCT |
| | TCGGGTGCGGGGCTCCGTACGGGGCGTGGCGCGGGGCTCGCCGTGCCGG |
| | GCGGGGGGTGGCGGCAGGTGGGGGTGCCGGGGGGGGCGGGGCCGCCTC |
| | GGGCCGGGGAGGGCTCGGGGGAGGGGCGCGGCGGCCCCCGGAGCGCCG |
| | GCGGCTGTCGAGGCGCGGCGAGCCGCAGCCATTGCCTTTTATGGTAATC |
| | GTGCGAGAGGGCGCAGGGACTTCCTTTGTCCCAAATCTGTGCGGAGCCG |
| | AAATCTGGGAGGCGCCGCCGCACCCCCTCTAGCGGGCGCGGGGCGAAG |
| | CGGTGCGGCGCCGGCAGGAAGGAAATGGGGGGGGAGGGCCTTCGTGCG |
| | TCGCCGCGCCGCCGTCCCCTTCTCCCTCTCCAGCCTCGGGGCTGTCCGCG |
| | GGGGGACGGCTGCCTTCGGGGGGGACGGGGCAGGGGGGGGTTCGGCTT |
| | CTGGCGTGTGACCGGCGGCTCTAGAGCCTCTGCTAACCATGTTCATGCCT |
| | TCTTCTTTTTCCTACAGCTCCTGGGCAACGTGCTGGTTATTGTGCTGTCTC |
| | ATCATTTTGGCAAAG |
| CB/CBA | CGCGTGGTACCTCTGGTCGTTACATAACTTACGGTAAATGGCCCGCC |
| promoter | TGGCTGACCGCCCAACGACCCCGCCCATTGACGTCAATAATGACGTATG |
| (SEQ ID | TTCCCATAGTAACGCCAATAGGGACTTTCCATTGACGTCAATGGGTGGA |
| NO: 49) | GTATTTACGGTAAACTGCCCACTTGGCAGTACATCAAGTGTATCATATG |
| | CCAAGTACGCCCCCTATTGACGTCAATGACGGTAAATGGCCCGCCTGGC |
| | ATTATGCCCAGTACATGACCTTATGGGACTTTCCTACTTGGCAGTACATC |
| | TACTCGAGGCCACGTTCTGCTTCACTCTCCCCATCTCCCCCCCCTCCCCA |
| | CCCCCAATTTTGTATTTATTTATTTTTTAATTATTTTGTGCAGCGATGGGG |
| | GCGGGGGGGGGGGGGGGGGGGGCGCGCGCCAGGCGGGGCGGGGGGGG |
| | GCGAGGGGCGGGGCGGGGCGAGGCGGAGAGGTGCGGCGGCAGCCAAT |
| | CAGAGCGGCGCGCTCCGAAAGTTTCCTTTTATGGCGAGGCGGCGGCGGC |
| | GGCGGCCCTATAAAAAGCGAAGCGCGCGGCGGGCGGGAGCGGGATCAG |
| | CCACCGCGG |
| UbC | TATATAAGGACGCGCCGGGTGTGGCACAGCTAGTTCCGTCGCAGCC |
| promoter | GGGATTTGGGTCGCAGTTCTTGTTTGTGGATCGCTGTGATCGTCACTTGG |
| (SEQ ID | TGAGTAGCGGGCTGCTGGGCTGGCCGGGGCTTTCGT |
| NO: 50) | |
| mU1a | ATGGAGGCGGTACTATGTAGATGAGAATTCAGGAGCAAACTGGGAA |
| promoter | AAGCAACTGCTTCCAAATATTTGTGATTTTTACAGTGTAGTTTTGGAAAA |
| (SEQ ID | ACTCTTAGCCTACCAATTCTTCTAAGTGTTTTAAAATGTGGGAGCCAGTA |
| NO: 51) | CACATGAAGTTATAGAGTGTTTTAATGAGGCTTAAATATTTACCGTAACT |
| | ATGAAATGCTACGCATATCATGCTGTTCAGGCTCCGTGGCCACGCAACT |
| | CATACT |
| EF-1a | GGGCAGAGCGCACATCGCCCACAGTCCCCGAGAAGTTGGGGGGAGG |
| promoter | GGTCGGCAATTGAACGGGTGCCTAGAGAAGGTGGCGCGGGGTAAACTG |
| (SEQ ID | GGAAAGTGATGTCGTGTACTGGCTCCGCCTTTTTCCCGAGGGTGGGGGA |
| NO: 52) | GAACCGTATATAAGTGCAGTAGTCGCCGTGAACGTTCTTTTTCGCAACG |
| | GGTTTGCCGCCAGAACACAG |

In some embodiments, neuron and/or astrocyte-specific expression of the DN-TNFα can be conferred using neuronal and/or astrocyte-specific promoters, such as, e.g., a human synapsin 1 (hSyn) promoter (SEQ ID NO: 53), methyl CpG-binding protein 2 (Mecp2, SEQ ID NO: 58), hGFAP promoter (SEQ ID NO: 59), NSE/RU5′ (SEQ ID NO: 60), hexaribonucleotide binding protein-3 (NeuN) promoter (SEQ ID NO: 61), Ca2+/calmodulin-dependent protein kinase II (CamKII) promoter (SEQ ID NO: 62, Wang, L., Bai, J., & Hu, Y. (2007). Molecular Biology Reports, 35 (1), 37-44)), tubulin alpha I (Tα-1) promoter (SEQ ID NO: 74), promoter of Dopamine-1 receptor (DRD1, SEQ ID NO: 65) and Dopamine-2 receptor (DRD2, SEQ ID NOs: 66-67), microtubule-associated protein 1B (MAP1B, SEQ ID NO: 71), complement component 1 q subcomponent-like 2 (C1ql2) promoter (SEQ ID NO: 63), pro-opiomelanocortin (POMC) promoter (SEQ ID NO: 68), and prospero homeobox protein 1 (PROX1) promoter (SEQ ID NO: 69). Promoters suitable for driving polynucleotide expression specifically in astrocytes include Glial fibrillary acidic protein (GFAP) (Griffin J M, et al., Gene Therapy (2019) 26:198-210), or variants thereof, e.g. GfaABCID promoter, and ALDH1L1 (Koh W. et al., Exp Neurobiol. 2017 December; 26 (6): 350-361). Promoters suitable for driving polynucleotide expression specifically in dentate gyrus cells of the hippocampus include the C1ql2, POMC, and PROX1 promoters. Synthetic promoters, hybrid promoters, and the like may also be used in conjunction with the methods and compositions disclosed herein. In addition, sequences derived from non-viral genes, such as the murine metallothionein gene, will also find use herein.

Table 8 provides examples and nucleotide sequences for CNS-specific promoters for use herein.

TABLE 8
Representative CNS-Specific Promoters

| Promoter | Sequence |
|---|---|
| Human Synapsin | AGTGCAAGTGGGTTTTAGGACCAGGATGAGGCGGGGTGGGG |
| promoter 1 | GTGCCTACCTGACGACCGACCCCGACCCACTGGACAAGCACCCA |
| (hSyn-1) | ACCCCCATTCCCCAAATTGCGCATCCCCTATCAGAGAGGGGGAG |
| (SEQ ID NO: 53) | GGGAAACAGGATGCGGCGAGGCGCGTGCGCACTGCCAGCTTCA |
| | GCACCGCGGACAGTGCCTTCGCCCCCGCCTGGCGGCGCGCGCCA |
| | CCGCCGCCTCAGCACTGAAGGCGCGCTGACGTCACTCGCCGGTC |
| | CCCCGCAAACTCCCCTTCCCGGCCACCTTGGTCGCGTCCGCGCCG |
| | CCGCCGGCCCAGCCGGACCGCACCACGCGAGGCGCGAGATAGG |
| | GGGGCACGGGCGCGACCATCTGCGCTGCGGCGCCGGCGACTCAG |
| | CGCTGCCTCAGTCTGCGGTGGGCAGCGGAGGAGTCGTGTCGTGC |
| | CTGAGAGCGCAG |
| Human Synapsin | CTGCAGAGGGCCCTGCGTATGAGTGCAAGTGGGTTTTAGGAC |
| promoter 2 | CAGGATGAGGCGGGGTGGGGGTGCCTACCTGACGACCGACCCCG |
| (hSyn-2) | ACCCACTGGACAAGCACCCAACCCCCATTCCCCAAATTGCGCAT |
| (SEQ ID NO: 54) | CCCCTATCAGAGAGGGGGAGGGGAAACAGGATGCGGCGAGGCG |
| | CGTGCGCACTGCCAGCTTCAGCACCGCGGACAGTGCCTTCGCCC |
| | CCGCCTGGCGGCGCGCGCCACCGCCGCCTCAGCACTGAAGGCGC |
| | GCTGACGTCACTCGCCGGTCCCCCGCAAACTCCCCTTCCCGGCCA |
| | CCTTGGTCGCGTCCGCGCCGCCGCCGGCCCAGCCGGACCGCACC |
| | ACGCGAGGCGCGAGATAGGGGGGCACGGGCGCGACCATCTGCG |
| | CTGCGGCGCCGGCGACTCAGCGCTGCCTCAGTCTGC |
| Human Synapsin | CTGCAGAGGGCCCTGCGTATGAGTGCAAGTGGGTTTTAGGAC |
| promoter 3 | CAGGATGAGGCGGGGTGGGGGTGCCTACCTGACGACCGACCCCG |
| (hSyn-3) | ACCCACTGGACAAGCACCCAACCCCCATTCCCCAAATTGCGCAT |
| (SEQ ID NO: 55) | CCCCTATCAGAGAGGGGGAGGGGAAACAGGATGCGGCGAGGCG |
| | CGTGCGCACTGCCAGCTTCAGCACCGCGGACAGTGCCTTCGCCC |
| | CCGCCTGGCGGCGCGCGCCACCGCCGCCTCAGCACTGAAGGCGC |
| | GCTGACGTCACTCGCCGGTCCCCCGCAAACTCCCCTTCCCGGCCA |
| | CCTTGGTCGCGTCCGCGCCGCCGCCGGCCCAGCCGGACCGCACC |
| | ACGCGAGGCGCGAGATAGGGGGGCACGGGCGCGACCATCTGCG |
| | CTGCGGCGCCGGCGACTCAGCGCTGCCTCAGTCTGCGGTGGGCA |
| | GCGGAGGAGTCGTGTCGTGCCTGAGAGCGCAGGGCGCGCC |
| Human Synapsin | CTGCAGAGGGCCCTGCGTATGAGTGCAAGTGGGTTTTAGGAC |
| promoter 4 | CAGGATGAGGCGGGGGGGGGTGCCTACCTGACGACCGACCCCG |
| (hSyn-4) | ACCCACTGGACAAGCACCCAACCCCCATTCCCCAAATTGCGCAT |
| (SEQ ID NO: 56) | CCCCTATCAGAGAGGGGGAGGGGAAACAGGATGCGGCGAGGCG |
| | CGTGCGCACTGCCAGCTTCAGCACCGCGGACAGTGCCTTCGCCC |
| | CCGCCTGGCGGCGCGCGCCACCGCCGCCTCAGCACTGAAGGCGC |
| | GCTGACGTCACTCGCCGGTCCCCCGCAAACTCCCCTTCCCGGCCA |
| | CCTTGGTCGCGTCCGCGCCGCCGCCGGCCCAGCCGGACCGCACC |
| | ACGCGAGGCGCGAGATAGGGGGGCACGGGCGCGACCATCTGCG |
| | CTGCGGCGCCGGCGACTCAGCGCTGCCTCAGTCTGCCAATTGCA |
| | GCGGAGGAGTCGTGTCGTGCCTGAGAGCGCAG |
| Human Synapsin | CCTGCAGGGCCCACTAGTATCTGCAGAGGGCCCTGCGTATGA |
| promoter 5 | GTGCAAGTGGGTTTTAGGACCAGGATGAGGCGGGGTGGGGGTGC |
| (hSyn-5) | CTACCTGACGACCGACCCCGACCCACTGGACAAGCACCCAACCC |
| (SEQ ID NO: 57) | CCATTCCCCAAATTGCGCATCCCCTATCAGAGAGGGGGAGGGGA |
| | AACAGGATGCGGCGAGGCGCGTGCGCACTGCCAGCTTCAGCACC |
| | GCGGACAGTGCCTTCGCCCCCGCCTGGCGGCGCGCGCCACCGCC |
| | GCCTCAGCACTGAAGGCGCGCTGACGTCACTCGCCGGTCCCCCG |
| | CAAACTCCCCTTCCCGGCCACCTTGGTCGCGTCCGCGCCGCCGCC |
| | GGCCCAGCCGGACCGCACCACGCGAGGCGCGAGATAGGGGGGC |
| | ACGGGCGCGACCATCTGCGCTGCGGCGCCGGCGACTCAGCGCTG |
| | CCTCAGTCTGCGGTGGGCAGCGGAGGAGTCGTGTCGTGCCTGAG |
| | AGCGCAGCTGTGCTCCTGGGCACCGCGCAGTCCGCCCCCGCGGC |
| | TCCTGGCCAGACCACCCCTAGGACCCCCTGCCCCAAGTCGCAGC |
| | C |
| Mecp2 promoter | AGCTGAATGGGGTCCGCCTCTTTTCCCTGCCTAAACAGACAG |
| (SEQ ID NO: 58) | GAACTCCTGCCAATTGAGGGCGTCACCGCTAAGGCTCCGCCCCA |
| | GCCTGGGCTCCACAACCAATGAAGGGTAATCTCGACAAAGAGCA |
| | AGGGGGGGGCGCGGGCGCGCAGGTGCAGCAGCACACAGGCTG |
| | GTCGGGAGGGCGGGGCGCGACGTCTGCCGTGCGGGGTCCCGGCA |
| | TCGGTTGCGCGC |
| hGFAP promoter | GTCTGCAAGCAGACCTGGCAGCATTGGGCTGGCCGCCCCCCA |
| (SEQ ID NO: 59) | GGGCCTCCTCTTCATGCCCAGTGAATGACTCACCTTGGCACAGAC |
| | ACAATGTTCGGGGTGGGCACAGTGCCTGCTTCCCGCCGCACCCC |
| | AGCCCCCCTCAAATGCCTTCCGAGAAGCCCATTGAGTAGGGGGC |
| | TTGCATTGCACCCCAGCCTGACAGCCTGGCATCTTGGGATAAAA |
| | GCAGCACAGCCCCCTAGGGGCTGCCCTTGCTGTGTGGCGCCACC |
| | GGCGGTGGAGAACAAGGCTCTATTCAGCCTGTGCCCAGGAAAGG |
| | GGATCAGGGGATGCCCAGGCATGGACAGTGGGTGGCAGGGGGG |
| | GAGAGGAGGGCTGTCTGCTTCCCAGAAGTCCAAGGACACAAATG |
| | GGTGAGGGGACTGGGCAGGGTTCTGACCCTGTGGGACCAGAGTG |
| | GAGGGCGTAGATGGACCTGAAGTCTCCAGGGACAACAGGGCCC |
| | AGGTCTCAGGCTCCTAGTTGGGCCCAGTGGCTCCAGCGTTTCCAA |
| | ACCCATCCATCCCCAGAGGTTCTTCCCATCTCTCCAGGCTGATGT |
| | GTGGGAACTCGAGGAAATAAATCTCCAGTGGGAGACGGAGGGG |
| | TGGCCAGGGAAACGGGGCGCTGCAGGAATAAAGACGAGCCAGC |
| | ACAGCCAGCTCATGCGTAACGGCTTTGTGGAGCTGTCAAGGCCT |
| | GGTCTCTGGGAGAGAGGCACAGGGAGGCCAGACAAGGAAGGGG |
| | TGACCTGGAGGGACAGATCCAGGGGCTAAAGTCCTGATAAGGCA |
| | AGAGAGTGCCGGCCCCCTCTTGCCCTATCAGGACCTCCACTGCC |
| | ACATAGAGGCCATGATTGACCCTTAGACAAAGGGCTGGTGTCCA |
| | ATCCCAGCCCCCAGCCCCAGAACTCCAGGGAATGAATGGGCAGA |
| | GAGCAGGAATGTGGGACATCTGTGTTCAAGGGAAGGACTCCAGG |
| | AGTCTGCTGGGAATGAGGCCTAGTAGGAAATGAGGTGGCCCTTG |
| | AGGGTACAGAACAGGTTCATTCTTCGCCAAATTCCCAGCACCTT |
| | GCAGGCACTTACAGCTGAGTGAGATAATGCCTGGGTTATGAAAT |
| | CAAAAAGTTGGAAAGCAGGTCAGAGGTCATCTGGTACAGCCCTT |
| | CCTTCCCTTTTTTTTTTTTTTTTTTTTTTGTGAGACAAGGTCTCTCT |
| | CTGTTGCCCAGGCTGGAGTGGCGCAAACACAGCTCACTGCAGCC |
| | TCAACCTACTGGGCTCAAGCAATCCTCCAGCCTCAGCCTOCCAA |
| | AGTGCTGGGATTACAAGCATGAGCCACCCCACTCAGCCCTTTCCT |
| | TCCTTTTTAATTGATGCATAATAATTGTAAGTATTCATCATGGTC |
| | CAACCAACCCTTTCTTGACCCACCTTCCTAGAGAGAGGGTCCTCT |
| | TGATTCAGCGGTCAGGGCCCCAGACCCATGGTCTGGCTCCAGGT |
| | ACCACCTGCCTCATGCAGGAGTTGGCGTGCCCAGGAAGCTCTGC |
| | CTCTGGGCACAGTGACCTCAGTGGGGTGAGGGGAGCTCTCCCCA |
| | TAGCTGGGCTGCGGCCCAACCCCACCCCCTCAGGCTATGCCAGG |
| | GGGTGTTGCCAGGGGCACCCGGGCATCGCCAGTCTAGCCCACTC |
| | CTTCATAAAGCCCTCGCATCCCAGGAGCGAGCAGAGCCAGAGCA |
| | T |
| Rat NSE/RU5′ | AGCTCTGAGCTCCTCCTCTGCTCGCCCAATCCTTCCAACCCCC |
| promoter | TATGGTGGTATGGCTGACACAGAAAATGTCTGCTCCTGTATGGG |
| (SEQ ID NO: 60) | ACATTTGCCCCTCTTCTCCAAATATAAGACAGGATGAGGCCTAG |
| | CTTTTGCTGCTCCAAAGTTTTAAAAGAACACATTGCACGGCATTT |
| | AGGGACTCTAAAGGGTGGAGGAGGAATGAGGGAATTGCATCAT |
| | GCCAAGGCTGGTCCTCATCCATCACTGCTTCCAGGGCCCAGAGT |
| | GGCTTCCAGGAAGTATTCTTACAAAGGAAGCCCGATCTGTAGCT |
| | AACACTCAGAGCCCATTTTCCTGCGTTAACCCCTCCCGACCTCAT |
| | ATACAGGAGTAACATGATCAGTGACCTGGGGGAGCTGGCCAAAC |
| | TGCGGGACCTGCCCAAGCTGAGGGCCTTGGTGCTGCTGGACAAC |
| | CCCTGTGCCGATGAGACTGACTACCGCCAGGAGGCCCTGGTGCA |
| | GATGGCACACCTAGAGCGCCTAGACAAAGAGTACTATGAGGAC |
| | GAGGACCGGGCAGAAGCTGAGGAGATCCGACAGAGGCTGAAGG |
| | AGGAACAGGAGCAAGAACTCGACCCGGACCAAGACATGGAACC |
| | GTACCTCCCGCCAACTTAGTGGCTCCTCTAGCCTGCAGGGACAGT |
| | AAAGGTGATGGCAGGAAGGCAGCCCCCGGAGGTCAAAGGCTGG |
| | GCACGCGGGAGGAGAGGCCAGAGTCAGAGGCTGCGGGTATCTC |
| | AGATATGAAGGAAAGATGAGAGAGGCTCAGGAAGAGGTAAGAA |
| | AAGACACAAGAGACCAGAGAAGGGAGAAGAATTAGAGAGGGA |
| | GGCAGAGGACCGCTGTCTCTACAGACATAGCTGGTAGAGACTGG |
| | GAGGAAGGGATGAACCCTGAGCGCATGAAGGGAAGGAGGTGGC |
| | TGGTGGTATATGGAGGATGTAGCTGGGCCAGGGAAAAGATCCTG |
| | CACTAAAAATCTGAAGCTAAAAATAACAGGACACGGGGTGGAG |
| | AGGCGAAAGGAGGGCAGAGTGAGGCAGAGAGACTGAGAGGCCT |
| | GGGGATGTGGGCATTCCGGTAGGGCACACAGTTCACTTGTCTTCT |
| | CTTTTTCCAGGAGGCCAAAGATGCTGACGTCAAGAACTCATAAT |
| | ACCCCAGTGGGGACCACCGCATTCATAGCCCTGTTACAAGAAGT |
| | GGGAGATGTTCCTTTTTGTCCCAGACTGGAAATCCGTTACATCCC |
| | GAGGCTCAGGTTCTGTGGTGGTCATCTCTGTGTGGCTTGTTCTGT |
| | GGGCCTACCTAAAGTCCTAAGCACAGCTCTCAAGCAGATCCGAG |
| | GCGACTAAGATGCTAGTAGGGGTTGTCTGGAGAGAAGAGCCGA |
| | GGAGGTGGGCTGTGATGGATCAGTTCAGCTTTCAAATAAAAAGG |
| | CGTTTTTATATTCTGTGTCGAGTTCGTGAACCCCTGTGGTGGGCT |
| | TCTCCATCTGTCTGGGTTAGTACCTGCCACTATACTGGAATAAGG |
| | GGACGCCTGCTTCCCTCGAGTTGGCTGGACAAGGTTATGAGCAT |
| | CCGTGTACTTATGGGGTTGCCAGCTTGGTCCTGGATCGCCCGGGC |
| | CCTTCCCCCACCCGTTCGGTTCCCCACCACCACCCGCGCTCGTAC |
| | GTGCGTCTCCGCCTGCAGCTCTTGACTCATCGGGGCCCCCGGGTC |
| | ACATGCGCTCGCTCGGCTCTATAGGCGCCGCCCCCTGCCCACCCC |
| | CCGCCCGCGCTGGGAGCCGCAGCCGCCGCCACTCCTGCTCTCTCT |
| | GCGCCGAAGCTTCGAGGGGCTCGCATCTCTCCTTCACGCGCCCG |
| | CCGCCCTACCTGAGGCCGCCATCCACGCCGGTTGAGTCGCGTTCT |
| | GCCGCCTCCCGCCTGTGGTGCCTCCTGAACTGCGTCCGCCGTCTA |
| | GGTAAGTTTAAAGCTCAGGTCGAGACCGGGCCTTTGTCCGGCGC |
| | TCCCTTGGAGCCTACCTAGACTCAGCCGGCTCTCCACGCTTTGCC |
| | TGACCCTGCTTGCTCAACTCTACGTCTTTGTTTCGTTTTCTGTTCT |
| | GCGCCGTTACAGATC |
| NeuN promoter | GAGGAGGAGGAGAGAGACCGGGAGGGCGCCCGGGAGGCAG |
| (NG_053112.1) | GGCGCGCGCACACTCCGAGG |
| (SEQ ID NO: 61) | |
| CaMKII_1 promoter | CATTATGGCCTTAGGTCACTTCATCTCCATGGGGTTCTTCTTC |
| (AJ222796.1) | TGATTTTCTAGAAAATGAGATGGGGGTGCAGAGAGCTTCCTCAG |
| (SEQ ID NO: 62) | TGACCTGCCCAGGGTCACATCAGAAATGTCAGAGCTAGAACTTG |
| | AACTCAGATTACTAATCTTAAATTCCATGCCTTGGGGGCATGCAA |
| | GTACGATATACAGAAGGAGTGAACTCATTAGGGCAGATGACCAA |
| | TGAGTTTAGGAAAGAAGAGTCCAGGGCAGGGTACATCTACACCA |
| | CCCGCCCAGCCCTGGGTGAGTCCAGCCACGTTCACCTCATTATAG |
| | TTGCCTCTCTCCAGTCCTACCTTGACGGGAAGCACAAGCAGAAA |
| | CTGGGACAGGAGCCCCAGGAGACCAAATCTTCATGGTCCCTCTG |
| | GGAGGATGGGTGGGGAGAGCTGTGGCAGAGGCCTCAGGAGGGG |
| | CCCTGCTGCTCAGTGGTGACAGATAGGGGTGAGAAAGCAGACAG |
| | AGTCATTCCGTCAGCATTCTGGGTCTGTTTGGTACTTCTTCTCAC |
| | GCTAAGGTGGCGGTGTGATATGCACAATGGCTAAAAAGCAGGG |
| | AGAGCTGGAAAGAAACAAGGACAGAGACAGAGGCCAAGTCAAC |
| | CAGACCAATTCCCAGAGGAAGCAAAGAAACCATTACAGAGACT |
| | ACAAGGGGGAAGGGAAGGAGAGATGAATTAGCTTCCCCTGTAA |
| | ACCTTAGAACCCAGCTGTTGCCAGGGCAACGGGGCAATACCTGT |
| | CTCTTCAGAGGAGATGAAGTTGCCAGGGTAACTACATCCTGTCTT |
| | TCTCAAGGACCATCCCAGAATGTGGCACCCACTAGCCGTTACCA |
| | TAGCAACTGCCTCTTTGCCCCACTTAATCCCATCCCGTCTGTTAA |
| | AAGGGCCCTATAGTTGGAGGTGGGGGAGGTAGGAAGAGCGATG |
| | ATCACTTGTGGACTAAGTTTGTTCGCATCCCCTTCTCCAACCCCC |
| | TCAGTACATCACCCTGGGGGAACAGGGTCCACTTGCTCCTGGGC |
| | CCACACAGTCCTGCAGTATTGTGTATATAAGGCCAGGGCAAAGA |
| | GGAGCAGGTTTTAAAGTGAAAGGCAGGCAGGTGTTGGGGAGGC |
| | AGTTACCGGGGCAACGGGAACAGGGCGTTTCGGAGGTGGTTGCC |
| | ATGGGGACCTGGATGCTGACGAAGGCTCGCGAGGCTGTGAGCAG |
| | CCACAGTGCCCTGCTCAGAAGCCCCAAGCTCGTCAGTCAAGCCG |
| | GTTCTCCGTTTGCACTCAGGAGCACGGGCAGGCGAGTGGCCCCT |
| | AGTTCTGGGGGCAGC |
| C1ql2 1 promoter | CCTCCGCCGCTCAGCCCCGGACTCCTTACGTCAGGGTAGCGG |
| (NM_182528) | GGTCCCCCCTCCGCGCGG |
| (SEQ ID NO: 63) | |
| C1ql2 2 promoter | CGATCCTATCACGAGACTAGCCTCGAGAAGCTTGATATCAGC |
| (AC01667.5 | ACCCACATAGCAGCTCACAAATGTCTGAAACTCCAATTCTTGGG |
| AC084310) | AATCTGACACGATCACACATGCAGGCAAAATACCAATGTACATG |
| (SEQ ID NO: 64) | AATTAAAAAAAAAAAAAACAACCTTTAAAAGAAACAAGGGTTC |
| | AGTACCACTACTGACATCTTGTTTCCCCAGAGGCCTTACTTTAAT |
| | TATTTATTGTTTCCACTTAGTTGCTCAATTAATTAATTTAGAGGTT |
| | TTTTTCTTCCTTTCTTTTTCTTTTTTCTTTCTCTCTTTTTTTTCTTCT |
| | TAAGACAGGGTTTCTCTGTGTAGCTCAGGCTATCCTGGAACTCAC |
| | TCTGTAGACCAGGCTGGCCTTGTACTCAAAGATCTGCCTGCCTCT |
| | GCCTCCCCAGTGCTGGGATTAAAGACATGCACCATCACTGCCCT |
| | GCTTTCCTCTTTTTATTTTGAAAATTGTTCATCAACAGTTACTAAA |
| | CGTGTTCGAATTCCAAGAGCTGACTAGACATATAAGACCATTCA |
| | GCCTTCTGAATAAGATGTAGGTGTGCCCCTCCTCTTACTCCTCTA |
| | TTTGGAAGTTGGTTACTTTCTGTATGTAGTATGCGAATCCCCCTC |
| | TGCCACCCCGCTTTCTGTTTTAAAACAGAAAAGGCTGCAACATA |
| | CAGTGTGTGCTTCTGTTCTTGAACTGGAAGCTTAGGCTGTCCTGG |
| | ACTTGGGTTGAGACCTGGGCTCATCCAGATAGGAAATGGATTTG |
| | GTGACCCCGCCAGGACTTCGCAGGCACCACATCGTGGTCGTGTG |
| | TGGGTGCTGTATGCACCCACTGATTGCGCGCGTGGGTTCCAGAG |
| | CTTGGTGGTCTGCGAGAGGAGAGTGGGCAAGAGTGGGTGTGTCT |
| | GTGGAGCCCCAGCTAGGGGCTGCTGCCCGCTGCTCCCACTTGTG |
| | GCTCCTGGGCGCCGCCAGCAGGCACATCTCCGGAGGACGCCGCG |
| | GGATGGGAGCTGATGACAGGAGAGCGCCGTCTCCCGAGTGATGG |
| | CAGCGCACGCTGCTGCCTCGCCGCCTCCGCCGCTCAGTCCTGATC |
| | TTACGTTAGGGTAGCTGGGTACCCCCTCCGCCCGGGAACCAGCT |
| | AGTAGAGGGAGAACAGAGCAGAGCGTGCGGCAGAGCCGATCCC |
| | GCGTCCCGCCGAACCCTGCCAAGCCCCGCCAATCCCAGCAGAGC |
| | AGGAACCAGCGCAGCTGAGCCAACACCGGACGCCGCACTGAGA |
| | CCCAGCATTCCCCAGCCGCCACTACCCGGTCCCCGCCGGGGTGC |
| | CGGGCTCGTCCTGTGAGCCCCTCGTCATGCGTGTCGGGCTCTTCG |
| | ACTCTCCAGATCAGTTCCAGAGCGCT |
| DRD1 promoter | GGGACGCGCGGGCGGGGTGGGCTGTGCCCCGCGGGAACCCC |
| (H. sapiens) | GCCGGCCTGTGCGCTTGCTG |
| NM_000794 | |
| (SEQ ID NO: 65) | |
| DRD2 promoter | CTCCCTCCCGCGCTCCCCGCGCTCGGGCGCCGCAGAGCTGTC |
| (isoform 1; | CAGCTTCAGTGCCGAACC |
| H. sapiens) | |
| NM_016574 | |
| (SEQ ID NO: 66) | |
| DRD2 promoter | GTACTGGTGTACAAGGACAAGGTGACTTTTTTTCTTTTCCCAG |
| (isoform 2; | ATTGAAAGGGCCAAAGA |
| H. sapiens) | |
| NM_016574 | |
| (SEQ ID NO: 67) | |
| POMC promoter | CCAGGAGAGCTCGGCAAGTATATAAGGACAGAGGAGCGCGG |
| (H. sapiens) | GACCAAGCGGCGGCGAAGG |
| NM_001319204 | |
| (SEQ ID NO: 68) | |
| PROX1 promoter | TTCCTTCAGCTGTGTCTTAAAGTAAATCTTGTTGTGGAGCGG |
| (isoform 1; | AGCCCTCAGCTGAGGGAG |
| H. sapiens) | |
| NM_002763 | |
| (SEQ ID NO: 69) | |
| PROX1 promoter | GTAAGTATCTTCTTCTTCCCCTCGTGAGTCCCTCCCCTTTTCC |
| (isoform 2) | AGAATCACTTGCACTGT |
| NM_002763 | |
| (SEQ ID NO: 70) | |
| MAP1B promoter | GGGGCGGAGCGGAGACAGTACCTTCGGAGATAATCCTTTCTC |
| (isoform 1; | CTGCCGCAGTGGAGAGGA |
| H. sapiens) | |
| NM_005909 | |
| (SEQ ID NO: 71) | |
| MAP1B promoter | CCCTGCCTAGTCTCCATATAAAAGCGGCGCCGCCTCCCCGCC |
| (isoform 2; | CTCTCTCACTCCCCGCTC |
| H. sapiens) | |
| NM_005909 | |
| (SEQ ID NO: 72) | |
| MAPI1B promoter | GGGCGGCCCAGCCCCAGGTTACGTCGTCCCCAGAAAGAATCT |
| (isoform 3; | GGCCAACAGTCTGGCCGT |
| H. sapiens) | |
| NM_005909 | |
| (SEQ ID NO: 73) | |
| Ta-1/TUBA1A | GGCAGGTTCTCTTACATCGACCGCTTAAGAGTCGCGCTGTAA |
| promoter | GAAGCAACACCTCCTCCTCGCCTCCGCCATCCACCCGGCAGCCG |
| (isoform 1; | CGAAGCAGCAACC |
| H. sapiens) | |
| NM_006009 | |
| (SEQ ID NO: 74) | |

Table 9 provides examples and nucleotide sequences for liver and muscle-specific promoters for use herein.

TABLE 9
Representative Liver and/or Muscle-specific Promoters

| Promoter | Sequence |
|---|---|
| LSPX1 promoter | AGGTTAATTTTTAAAAAGCAGTCAAAAGTCCAAGTGGCCCTTG |
| (synthetic, | GCAGCATTTACTCTCTCTGTTTGCTCTGGTTAATAATCTCAGG |
| liver-specific) | AGCACAAACATTCCAGATCCAGGTTAATTTTTAAAAAGCAGTC |
| (SEQ ID NO: 75) | AAAAGTCCAAGTGGCCCTTGGCAGCATTTACTCTCTCTGTTTG |
| | CTCTGGTTAATAATCTCAGGAGCACAAACATTCCAGATCCGGC |
| | GCGCCAGGGCTGGAAGCTACCTTTGTCTAGAAGGCTCAGAGGC |
| | ACACAGGAGTTTCTGGGCTCACCCTGCCCCCTTCCAACCCCTC |
| | AGTTCCCATCCTCCAGCAGCTGTTTGTGTGCTGCCTCTGAAGT |
| | CCACACTGAACAAACTTCAGCCTACTCATGTCCCTAAAATGGG |
| | CAAACATTGCAAGCAGCAAACAGCAAACACACAGCCCTCCCTG |
| | CCTGCTGACCTTGGAGCTGGGGCAGAGGTCAGAGACCTCTCTG |
| | GGCCCATGCCACCTCCAACATCCACTCGACCCCTTGGAATTTC |
| | GGTGGAGAGGAGCAGAGGTTGTCCTGGCGTGGTTTAGGTAGTG |
| | TGAGAGGGGTACCCGGGGATCTTGCTACCAGTGGAACAGCCAC |
| | TAAGGATTCTGCAGTGAGAGCAGAGGGCCAGCTAAGTGGTACT |
| | CTCCCAGAGACTGTCTGACTCACGCCACCCCCTCCACCTTGGA |
| | CACAGGACGCTGTGGTTTCTGAGCCAGGTACAATGACTCCTTT |
| | CGGTAAGTGCAGTGGAAGCTGTACACTGCCCAGGCAAAGCGTC |
| | CGGGCAGCGTAGGCGGGCGACTCAGATCCCAGCCAGTGGACTT |
| | AGCCCCTGTTTGCTCCTCCGATAACTGGGGTGACCTTGGTTAA |
| | TATTCACCAGCAGCCTCCCCCGTTGCCCCTCTGGATCCACTGC |
| | TTAAATACGGACGAGGACAGGGCCCTGTCTCCTCAGCTTCAGG |
| | CACCACCACTGACCTGGGACAGT |
| LSPX2 promoter | AGGCTCAGAGGCACACAGGAGTTTCTGGGCTCACCCTGCCCCC |
| (synthetic, | TTCCAACCCCTCAGTTCCCATCCTCCAGCAGCTGTTTGTGTGC |
| liver-specific) | TGCCTCTGAAGTCCACACTGAACAAACTTCAGCCTACTCATGT |
| (SEQ ID NO: 76) | CCCTAAAATGGGCAAACATTGCAAGCAGCAAACAGCAAACACA |
| | CAGCCCTCCCTGCCTGCTGACCTTGGAGCTGGGGCAGAGGTCA |
| | GAGACCTCTCTGGGCCCATGCCACCTCCAACATCCACTCGACC |
| | CCTTGGAATTTCGGTGGAGAGGAGCAGAGGTTGTCCTGGCGTG |
| | GTTTAGGTAGTGTGAGAGGGTCTAGAAGGCTCAGAGGCACACA |
| | GGAGTTTCTGGGCTCACCCTGCCCCCTTCCAACCCCTCAGTTC |
| | CCATCCTCCAGCAGCTGTTTGTGTGCTGCCTCTGAAGTCCACA |
| | CTGAACAAACTTCAGCCTACTCATGTCCCTAAAATGGGCAAAC |
| | ATTGCAAGCAGCAAACAGCAAACACACAGCCCTCCCTGCCTGC |
| | TGACCTTGGAGCTGGGGCAGAGGTCAGAGACCTCTCTGGGCCC |
| | ATGCCACCTCCAACATCCACTCGACCCCTTGGAATTTCGGTGG |
| | AGAGGAGCAGAGGTTGTCCTGGCGTGGTTTAGGTAGTGTGAGA |
| | GGGGTACCCGGGGATCTTGCTACCAGTGGAACAGCCACTAAGG |
| | ATTCTGCAGTGAGAGCAGAGGGCCAGCTAAGTGGTACTCTCCC |
| | AGAGACTGTCTGACTCACGCCACCCCCTCCACCTTGGACACAG |
| | GACGCTGTGGTTTCTGAGCCAGGTACAATGACTCCTTTCGGTA |
| | AGTGCAGTGGAAGCTGTACACTGCCCAGGCAAAGCGTCCGGGC |
| | AGCGTAGGCGGGCGACTCAGATCCCAGCCAGTGGACTTAGCCC |
| | CTGTTTGCTCCTCCGATAACTGGGGTGACCTTGGTTAATATTC |
| | ACCAGCAGCCTCCCCCGTTGCCCCTCTGGATCCACTGCTTAAA |
| | TACGGACGAGGACAGGGCCCTGTCTCCTCAGCTTCAGGCACCA |
| | CCACTGACCTGGGACAGT |
| LTP1 promoter | AGGTTAATTTTTAAAAAGCAGTCAAAAGTCCAAGTGGCCCTTG |
| (synthetic, | GCAGCATTTACTCTCTCTGTTTGCTCTGGTTAATAATCTCAGG |
| liver-specific) | AGCACAAACATTCCAGATCCAGGTTAATTTTTAAAAAGCAGTC |
| (SEQ ID NO: 77) | AAAAGTCCAAGTGGCCCTTGGCAGCATTTACTCTCTCTGTTTG |
| | CTCTGGTTAATAATCTCAGGAGCACAAACATTCCAGATCCGGC |
| | GCGCCAGGGCTGGAAGCTACCTTTGACATCATTTCCTCTGCGA |
| | ATGCATGTATAATTTCTACAGAACCTATTAGAAAGGATCACCC |
| | AGCCTCTGCTTTTGTACAACTTTCCCTTAAAAAACTGCCAATT |
| | CCACTGCTGTTTGGCCCAATAGTGAGAACTTTTTCCTGCTGCC |
| | TCTTGGTGCTTTTGCCTATGGCCCCTATTCTGCCTGCTGAAGA |
| | CACTCTTGCCAGCATGGACTTAAACCCCTCCAGCTCTGACAAT |
| | CCTCTTTCTCTTTTGTTTTACATGAAGGGTCTGGCAGCCAAAG |
| | CAATCACTCAAAGTTCAAACCTTATCATTTTTTGCTTTGTTCC |
| | TCTTGGCCTTGGTTTTGTACATCAGCTTTGAAAATACCATCCC |
| | AGGGTTAATGCTGGGGTTAATTTATAACTAAGAGTGCTCTAGT |
| | TTTGCAATACAGGACATGCTATAAAAATGGAAAGATGTTGCTT |
| | TCTGAGAGGATCTTGCTACCAGTGGAACAGCCACTAAGGATTC |
| | TGCAGTGAGAGCAGAGGGCCAGCTAAGTGGTACTCTCCCAGAG |
| | ACTGTCTGACTCACGCCACCCCCTCCACCTTGGACACAGGACG |
| | CTGTGGTTTCTGAGCCAGGTACAGTGACTCCTTTCGGTAAGTG |
| | CAGTGGAAGCTGTACACTGCCCAGGCAAAGCGTCCGGGCAGCG |
| | TAGGCGGGCGACTCAGATCCCAGCCAGTGGACTTAGCCCCTGT |
| | TTGCTCCTCCGATAACTGGGGTGACCTTGGTTAATATTCACCA |
| | GCAGCCTCCCCCGTTGCCCCTCTGGATCCACTGCTTAAATACG |
| | GACGAGGACAGGGCCCTGTCTCCTCAGCTTCAGGCACCACCAC |
| | TGACCTGGGACAGT |
| LTP2 promoter | AGGCTCAGAGGCACACAGGAGTTTCTGGGCTCACCCTGCCCCC |
| (synthetic, | TTCCAACCCCTCAGTTCCCATCCTCCAGCAGCTGTTTGTGTGC |
| liver-specific) | TGCCTCTGAAGTCCACACTGAACAAACTTCAGCCTACTCATGT |
| (SEQ ID NO: 78) | CCCTAAAATGGGCAAACATTGCAAGCAGCAAACAGCAAACACA |
| | CAGCCCTCCCTGCCTGCTGACCTTGGAGCTGGGGCAGAGGTCA |
| | GAGACCTCTCTGGGCCCATGCCACCTCCAACATCCACTCGACC |
| | CCTTGGAATTTCGGTGGAGAGGAGCAGAGGTTGTCCTGGCGTG |
| | GTTTAGGTAGTGTGAGAGGGTCTAGAGCCCTTAAGCTAGCAGG |
| | TTAATTTTTAAAAAGCAGTCAAAAGTCCAAGTGGCCCTTGGCA |
| | GCATTTACTCTCTCTGTTTGCTCTGGTTAATAATCTCAGGAGC |
| | ACAAACATTCCAGATCCAGGTTAATTTTTAAAAAGCAGTCAAA |
| | AGTCCAAGTGGCCCTTGGCAGCATTTACTCTCTCTGTTTGCTC |
| | TGGTTAATAATCTCAGGAGCACAAACATTCCAGATCCGGCGCG |
| | CCAGGGCTGGAAGCTACCTTTGACATCATTTCCTCTGCGAATG |
| | CATGTATAATTTCTACAGAACCTATTAGAAAGGATCACCCAGC |
| | CTCTGCTTTTGTACAACTTTCCCTTAAAAAACTGCCAATTCCA |
| | CTGCTGTTTGGCCCAATAGTGAGAACTTTTTCCTGCTGCCTCT |
| | TGGTGCTTTTGCCTATGGCCCCTATTCTGCCTGCTGAAGACAC |
| | TCTTGCCAGCATGGACTTAAACCCCTCCAGCTCTGACAATCCT |
| | CTTTCTCTTTTGTTTTACATGAAGGGTCTGGCAGCCAAAGCAA |
| | TCACTCAAAGTTCAAACCTTATCATTTTTTGCTTTGTTCCTCT |
| | TGGCCTTGGTTTTGTACATCAGCTTTGAAAATACCATCCCAGG |
| | GTTAATGCTGGGGTTAATTTATAACTAAGAGTGCTCTAGTTTT |
| | GCAATACAGGACATGCTATAAAAATGGAAAGATGTTGCTTTCT |
| | GAGAGGATCTTGCTACCAGTGGAACAGCCACTAAGGATTCTGC |
| | AGTGAGAGCAGAGGGCCAGCTAAGTGGTACTCTCCCAGAGACT |
| | GTCTGACTCACGCCACCCCCTCCACCTTGGACACAGGACGCTG |
| | TGGTTTCTGAGCCAGGTACAGTGACTCCTTTCGGTAAGTGCAG |
| | TGGAAGCTGTACACTGCCCAGGCAAAGCGTCCGGGCAGCGTAG |
| | GCGGGCGACTCAGATCCCAGCCAGTGGACTTAGCCCCTGTTTG |
| | CTCCTCCGATAACTGGGGTGACCTTGGTTAATATTCACCAGCA |
| | GCCTCCCCCGTTGCCCCTCTGGATCCACTGCTTAAATACGGAC |
| | GAGGACAGGGCCCTGTCTCCTCAGCTTCAGGCACCACCACTGA |
| | CCTGGGACAGT |
| LTP3 promoter | AGGTTAATTTTTAAAAAGCAGTCAAAAGTCCAAGTGGCCCTTG |
| (synthetic, | GCAGCATTTACTCTCTCTGTTTGCTCTGGTTAATAATCTCAGG |
| liver-specific) | AGCACAAACATTCCAGATCCAGGTTAATTTTTAAAAAGCAGTC |
| (SEQ ID NO: 79) | AAAAGTCCAAGTGGCCCTTGGCAGCATTTACTCTCTCTGTTTG |
| | CTCTGGTTAATAATCTCAGGAGCACAAACATTCCAGATCCGGC |
| | GCGCCAGGGCTGGAAGCTACCTTTGACATCATTTCCTCTGCGA |
| | ATGCATGTATAATTTCTACAGAACCTATTAGAAAGGATCACCC |
| | AGCCTCTGCTTTTGTACAACTTTCCCTTAAAAAACTGCCAATT |
| | CCACTGCTGTTTGGCCCAATAGTGAGAACTTTTTCCTGCTGCC |
| | TCTTGGTGCTTTTGCCTATGGCCCCTATTCTGCCTGCTGAAGA |
| | CACTCTTGCCAGCATGGACTTAAACCCCTCCAGCTCTGACAAT |
| | CCTCTTTCTCTTTTGTTTTACATGAAGGGTCTGGCAGCCAAAG |
| | CAATCACTCAAAGTTCAAACCTTATCATTTTTTGCTTTGTTCC |
| | TCTTGGCCTTGGTTTTGTACATCAGCTTTGAAAATACCATCCC |
| | AGGGTTAATGCTGGGGTTAATTTATAACTAAGAGTGCTCTAGT |
| | TTTGCAATACAGGACATGCTATAAAAATGGAAAGATGTTGCTT |
| | TCTGAGAGGATCTTGCTACCAGTGGAACAGCCACTAAGGATTC |
| | TGCAGTGAGAGCAGAGGGCCAGCTAAGTGGTACTCTCCCAGAG |
| | ACTGTCTGACTCACGCCACCCCCTCCACCTTGGACACAGGACG |
| | CTGTGGTTTCTGAGCCAGGTACAGTGACTCCTTTCGGTAAGTG |
| | CAGTGGAAGCTGTACACTGCCCAGGCAAAGCGTCCGGGCAGCG |
| | TAGGCGGGCGACTCAGATCCCAGCCAGTGGACTTAGCCCCTGT |
| | TTGCTCCTCCGATAACTGGGGTGACCTTGGTTAATATTCACCA |
| | GCAGCCTCCCCCGTTGCCCCTCTGGATCCACTGCTTAAATACG |
| | GACGAGGACAGGGCCCTGTCTCCTCAGCTTCAGGCACCACCAC |
| | TGACCTGGGACAGTAAAACAGGTAAGTCCGCTGTTTGTGTGCT |
| | GCCTCTGAAGTCCACACTGAACAAACTTCAGCCTACTCATGTC |
| | CCTAAAATGGGCAAACATTGCAAGCAGCAAACAGCAAACACAC |
| | AGCCCTCCCTGCCTGCTGACCTTGGAGCTGGGGCAGAGGTCAG |
| | AGACCTCTCTGGCCTCTACTAACCATGTTCATGTTTTCTTTTT |
| | TTTTCTACAGGTCCTGGGTGACGAACAG |
| LMTP6 promoter | AGGCTCAGAGGCACACAGGAGTTTCTGGGCTCACCCTGCCCCC |
| (synthetic, | TTCCAACCCCTCAGTTCCCATCCTCCAGCAGCTGTTTGTGTGC |
| liver-muscle | TGCCTCTGAAGTCCACACTGAACAAACTTCAGCCTACTCATGT |
| tandem) | CCCTAAAATGGGCAAACATTGCAAGCAGCAAACAGCAAACACA |
| (SEQ ID NO: 80) | CAGCCCTCCCTGCCTGCTGACCTTGGAGCTGGGGCAGAGGTCA |
| | GAGACCTCTCTGGGCCCATGCCACCTCCAACATCCACTCGACC |
| | CCTTGGAATTTCGGTGGAGAGGAGCAGAGGTTGTCCTGGCGTG |
| | GTTTAGGTAGTGTGAGAGGGCCACTACGGGTTTAGGCTGCCCA |
| | TGTAAGGAGGCAAGGCCTGGGGACACCCGAGATGCCTGGTTAT |
| | AATTAACCCAGACATGTGGCTGCCCCCCCCCCCCCCAACACCT |
| | GCTGCCTCTAAAAATAACCCTGTCCCTGGTGGATCCCACTACG |
| | GGTTTAGGCTGCCCATGTAAGGAGGCAAGGCCTGGGGACACCC |
| | GAGATGCCTGGTTATAATTAACCCAGACATGTGGCTGCCCCCC |
| | CCCCCCCCAACACCTGCTGCCTCTAAAAATAACCCTGTCCCTG |
| | GTGGATCCCACTACGGGTTTAGGCTGCCCATGTAAGGAGGCAA |
| | GGCCTGGGGACACCCGAGATGCCTGGTTATAATTAACCCAGAC |
| | ATGTGGCTGCCCCCCCCCCCCCCAACACCTGCTGCCTCTAAAA |
| | ATAACCCTGTCCCTGGTGGATCCCCTGCATGCGAAGATCTTCG |
| | AACAAGGCTGTGGGGGACTGAGGGCAGGCTGTAACAGGCTTGG |
| | GGGCCAGGGCTTATACGTGCCTGGGACTCCCAAAGTATTACTG |
| | TTCCATGTTCCCGGCGAAGGGCCAGCTGTCCCCCGCCAGCTAG |
| | ACTCAGCACTTAGTTTAGGAACCAGTGAGCAAGTCAGCCCTTG |
| | GGGCAGCCCATACAAGGCCATGGGGCTGGGCAAGCTGCACGCC |
| | TGGGTCCGGGGTGGGCACGGTGCCCGGGCAACGAGCTGAAAGC |
| | TCATCTGCTCTCAGGGGCCCCTCCCTGGGGACAGCCCCTCCTG |
| | GCTAGTCACACCCTGTAGGCTCCTCTATATAACCCAGGGGCAC |
| | AGGGGCTGCCCTCATTCTACCACCACCTCCACAGCACAGACAG |
| | ACACTCAGGAGCCAGCCAGCGTCGAGATCTTGCTACCAGTGGA |
| | ACAGCCACTAAGGATTCTGCAGTGAGAGCAGAGGGCCAGCTAA |
| | GTGGTACTCTCCCAGAGACTGTCTGACTCACGCCACCCCCTCC |
| | ACCTTGGACACAGGACGCTGTGGTTTCTGAGCCAGGTACAGTG |
| | ACTCCTTTCGGTAAGTGCAGTGGAAGCTGTACACTGCCCAGGC |
| | AAAGCGTCCGGGCAGCGTAGGCGGGCGACTCAGATCCCAGCCA |
| | GTGGACTTAGCCCCTGTTTGCTCCTCCGATAACTGGGGTGACC |
| | TTGGTTAATATTCACCAGCAGCCTCCCCCGTTGCCCCTCTGGA |
| | TCCACTGCTTAAATACGGACGAGGACAGGGCCCTGTCTCCTCA |
| | GCTTCAGGCACCACCACTGACCTGGGACAGT |
| LMTP13 promoter | AGGCTCAGAGGCACACAGGAGTTTCTGGGCTCACCCTGCCCCC |
| (synthetic, | TTCCAACCCCTCAGTTCCCATCCTCCAGCAGCTGTTTGTGTGC |
| liver-muscle | TGCCTCTGAAGTCCACACTGAACAAACTTCAGCCTACTCATGT |
| tandem) | CCCTAAAATGGGCAAACATTGCAAGCAGCAAACAGCAAACACA |
| (SEQ ID NO: 81) | CAGCCCTCCCTGCCTGCTGACCTTGGAGCTGGGGCAGAGGTCA |
| | GAGACCTCTCTGGGCCCATGCCACCTCCAACATCCACTCGACC |
| | CCTTGGAATTTCGGTGGAGAGGAGCAGAGGTTGTCCTGGCGTG |
| | GTTTAGGTAGTGTGAGAGGGGTACCCGGGGATCTTGCTACCAG |
| | TCTAGAGGCCGTCCGCCCTCGGCACCATCCTCACGACACCCAA |
| | ATATGGCGACGGGTGAGGAATGGTGGGGAGTTATTTTTAGAGC |
| | GGTGAGGAAGGTGGGCAGGCAGCAGGTGTTGGCGCTCTAAAAA |
| | TAACTCCCGGGAGTTATTTTTAGAGCGGAGGAATGGTGGACAC |
| | CCAAATATGGCGACGGTTCCTCACCCGTCGCCATATTTGGGTG |
| | TCCGCCCTCGGCCGGGGCCGCATTCCTGGGGGCCGGGCGGTGC |
| | TCCCGCCCGCCTCGATAAAAGGCTCCGGGGCCGGCGGCGGCCC |
| | ACGAGCTACCCGGAGGAGCGGGAGGCGCCAAGCGTGAGTATCG |
| | ATCTTGCTACCAGTGGAACAGCCACTAAGGATTCTGCAGTGAG |
| | AGCAGAGGGCCAGCTAAGTGGTACTCTCCCAGAGACTGTCTGA |
| | CTCACGCCACCCCCTCCACCTTGGACACAGGACGCTGTGGTTT |
| | CTGAGCCAGGTACAGTGACTCCTTTCGGTAAGTGCAGTGGAAG |
| | CTGTACACTGCCCAGGCAAAGCGTCCGGGCAGCGTAGGCGGGC |
| | GACTCAGATCCCAGCCAGTGGACTTAGCCCCTGTTTGCTCCTC |
| | CGATAACTGGGGTGACCTTGGTTAATATTCACCAGCAGCCTCC |
| | CCCGTTGCCCCTCTGGATCCACTGCTTAAATACGGACGAGGAC |
| | AGGGCCCTGTCTCCTCAGCTTCAGGCACCACCACTGACCTGGG |
| | ACAGT |
| LMTP14 promoter | GAATGGTGGACACCCAAATATGGCGACGGTTCCTCACCCGTCG |
| (synthetic, | CCATATTTGGGTGTCCGCCCTCGGCCGGGGCCGCATTCCTGGG |
| liver-muscle | GGCCGGGCGGTGCTCCCGCCCGCCTCGATAAAAGGCTCCGGGG |
| tandem) | CCGGCGGCGGCCCACGAGCTACCCGGAGGAGCGGGAGGCGCCA |
| (SEQ ID NO: 82) | AGCGATCTTGCTACCAGTGGAACAGCCACTAAGGATTCTGCAG |
| | TGAGAGCAGAGGGCCAGCTAAGTGGTACTCTCCCAGAGACTGT |
| | CTGACTCACGCCACCCCCTCCACCTTGGACACAGGACGCTGTG |
| | GTTTCTGAGCCAGGTACAGTGACTCCTTTCGGTAAGTGCAGTG |
| | GAAGCTGTACACTGCCCAGGCAAAGCGTCCGGGCAGCGTAGGC |
| | GGGCGACTCAGATCCCAGCCAGTGGACTTAGCCCCTGTTTGCT |
| | CCTCCGATAACTGGGGTGACCTTGGTTAATATTCACCAGCAGC |
| | CTCCCCCGTTGCCCCTCTGGATCCACTGCTTAAATACGGACGA |
| | GGACAGGGCCCTGTCTCCTCAGCTTCAGGCACCACCACTGACC |
| | TGGGACAGT |
| LMTP15 promoter | AGGCTCAGAGGCACACAGGAGTTTCTGGGCTCACCCTGCCCCC |
| (synthetic, | TTCCAACCCCTCAGTTCCCATCCTCCAGCAGCTGTTTGTGTGC |
| liver-muscle | TGCCTCTGAAGTCCACACTGAACAAACTTCAGCCTACTCATGT |
| tandem) | CCCTAAAATGGGCAAACATTGCAAGCAGCAAACAGCAAACACA |
| (SEQ ID NO: 83) | CAGCCCTCCCTGCCTGCTGACCTTGGAGCTGGGGCAGAGGTCA |
| | GAGACCTCTCTGGGCCCATGCCACCTCCAACATCCACTCGACC |
| | CCTTGGAATTTCGGTGGAGAGGAGCAGAGGTTGTCCTGGCGTG |
| | GTTTAGGTAGTGTGAGAGGGTCTAGAGAATGGTGGACACCCAA |
| | ATATGGCGACGGTTCCTCACCCGTCGCCATATTTGGGTGTCCG |
| | CCCTCGGCCGGGGCCGCATTCCTGGGGGCCGGGCGGTGCTCCC |
| | GCCCGCCTCGATAAAAGGCTCCGGGGCCGGCGGCGGCCCACGA |
| | GCTACCCGGAGGAGCGGGAGGCGCCAAGCGATCTTGCTACCAG |
| | TGGAACAGCCACTAAGGATTCTGCAGTGAGAGCAGAGGGCCAG |
| | CTAAGTGGTACTCTCCCAGAGACTGTCTGACTCACGCCACCCC |
| | CTCCACCTTGGACACAGGACGCTGTGGTTTCTGAGCCAGGTAC |
| | AGTGACTCCTTTCGGTAAGTGCAGTGGAAGCTGTACACTGCCC |
| | AGGCAAAGCGTCCGGGCAGCGTAGGCGGGCGACTCAGATCCCA |
| | GCCAGTGGACTTAGCCCCTGTTTGCTCCTCCGATAACTGGGGT |
| | GACCTTGGTTAATATTCACCAGCAGCCTCCCCCGTTGCCCCTC |
| | TGGATCCACTGCTTAAATACGGACGAGGACAGGGCCCTGTCTC |
| | CTCAGCTTCAGGCACCACCACTGACCTGGGACAGT |
| LMTP18 promoter | AGGCTCAGAGGCACACAGGAGTTTCTGGGCTCACCCTGCCCCC |
| (synthetic, | TTCCAACCCCTCAGTTCCCATCCTCCAGCAGCTGTTTGTGTGC |
| liver-muscle | TGCCTCTGAAGTCCACACTGAACAAACTTCAGCCTACTCATGT |
| tandem) | CCCTAAAATGGGCAAACATTGCAAGCAGCAAACAGCAAACACA |
| (SEQ ID NO: 84) | CAGCCCTCCCTGCCTGCTGACCTTGGAGCTGGGGCAGAGGTCA |
| | GAGACCTCTCTGGGCCCATGCCACCTCCAACATCCACTCGACC |
| | CCTTGGAATTTCGGTGGAGAGGAGCAGAGGTTGTCCTGGCGTG |
| | GTTTAGGTAGTGTGAGAGGGCCACTACGGGTTTAGGCTGCCCA |
| | TGTAAGGAGGCAAGGCCTGGGGACACCCGAGATGCCTGGTTAT |
| | AATTAACCCAGACATGTGGCTGCCCCCCCCCCCCCCAACACCT |
| | GCTGCCTCTAAAAATAACCCTGTCCCTGGTGGATCCCCTGCAT |
| | GCGAAGATCTTCGAACAAGGCTGTGGGGGACTGAGGGCAGGCT |
| | GTAACAGGCTTGGGGGCCAGGGCTTATACGTGCCTGGGACTCC |
| | CAAAGTATTACTGTTCCATGTTCCCGGCGAAGGGCCAGCTGTC |
| | CCCCGCCAGCTAGACTCAGCACTTAGTTTAGGAACCAGTGAGC |
| | AAGTCAGCCCTTGGGGCAGCCCATACAAGGCCATGGGGCTGGG |
| | CAAGCTGCACGCCTGGGTCCGGGGTGGGCACGGTGCCCGGGCA |
| | ACGAGCTGAAAGCTCATCTGCTCTCAGGGGCCCCTCCCTGGGG |
| | ACAGCCCCTCCTGGCTAGTCACACCCTGTAGGCTCCTCTATAT |
| | AACCCAGGGGCACAGGGGCTGCCCTCATTCTACCACCACCTCC |
| | ACAGCACAGACAGACACTCAGGAGCCAGCCAGCGTCGAGATCT |
| | TGCTACCAGTGGAACAGCCACTAAGGATTCTGCAGTGAGAGCA |
| | GAGGGCCAGCTAAGTGGTACTCTCCCAGAGACTGTCTGACTCA |
| | CGCCACCCCCTCCACCTTGGACACAGGACGCTGTGGTTTCTGA |
| | GCCAGGTACAGTGACTCCTTTCGGTAAGTGCAGTGGAAGCTGT |
| | ACACTGCCCAGGCAAAGCGTCCGGGCAGCGTAGGCGGGCGACT |
| | CAGATCCCAGCCAGTGGACTTAGCCCCTGTTTGCTCCTCCGAT |
| | AACTGGGGTGACCTTGGTTAATATTCACCAGCAGCCTCCCCCG |
| | TTGCCCCTCTGGATCCACTGCTTAAATACGGACGAGGACAGGG |
| | CCCTGTCTCCTCAGCTTCAGGCACCACCACTGACCTGGGACAG |
| | T |
| LMTP19 promoter | AGGCTCAGAGGCACACAGGAGTTTCTGGGCTCACCCTGCCCCC |
| (synthetic, | TTCCAACCCCTCAGTTCCCATCCTCCAGCAGCTGTTTGTGTGC |
| liver-muscle | TGCCTCTGAAGTCCACACTGAACAAACTTCAGCCTACTCATGT |
| tandem) | CCCTAAAATGGGCAAACATTGCAAGCAGCAAACAGCAAACACA |
| (SEQ ID NO: 85) | CAGCCCTCCCTGCCTGCTGACCTTGGAGCTGGGGCAGAGGTCA |
| | GAGACCTCTCTGGGCCCATGCCACCTCCAACATCCACTCGACC |
| | CCTTGGAATTTCGGTGGAGAGGAGCAGAGGTTGTCCTGGCGTG |
| | GTTTAGGTAGTGTGAGAGGGCCCTGCATGCGAAGATCTTCGAA |
| | CAAGGCTGTGGGGGACTGAGGGCAGGCTGTAACAGGCTTGGGG |
| | GCCAGGGCTTATACGTGCCTGGGACTCCCAAAGTATTACTGTT |
| | CCATGTTCCCGGCGAAGGGCCAGCTGTCCCCCGCCAGCTAGAC |
| | TCAGCACTTAGTTTAGGAACCAGTGAGCAAGTCAGCCCTTGGG |
| | GCAGCCCATACAAGGCCATGGGGCTGGGCAAGCTGCACGCCTG |
| | GGTCCGGGGTGGGCACGGTGCCCGGGCAACGAGCTGAAAGCTC |
| | ATCTGCTCTCAGGGGCCCCTCCCTGGGGACAGCCCCTCCTGGC |
| | TAGTCACACCCTGTAGGCTCCTCTATATAACCCAGGGGCACAG |
| | GGGCTGCCCTCATTCTACCACCACCTCCACAGCACAGACAGAC |
| | ACTCAGGAGCCAGCCAGCGTCGAGATCTTGCTACCAGTGGAAC |
| | AGCCACTAAGGATTCTGCAGTGAGAGCAGAGGGCCAGCTAAGT |
| | GGTACTCTCCCAGAGACTGTCTGACTCACGCCACCCCCTCCAC |
| | CTTGGACACAGGACGCTGTGGTTTCTGAGCCAGGTACAGTGAC |
| | TCCTTTCGGTAAGTGCAGTGGAAGCTGTACACTGCCCAGGCAA |
| | AGCGTCCGGGCAGCGTAGGCGGGCGACTCAGATCCCAGCCAGT |
| | GGACTTAGCCCCTGTTTGCTCCTCCGATAACTGGGGTGACCTT |
| | GGTTAATATTCACCAGCAGCCTCCCCCGTTGCCCCTCTGGATC |
| | CACTGCTTAAATACGGACGAGGACAGGGCCCTGTCTCCTCAGC |
| | TTCAGGCACCACCACTGACCTGGGACAGT |
| LMTP20 promoter | AGGCTCAGAGGCACACAGGAGTTTCTGGGCTCACCCTGCCCCC |
| (synthetic, | TTCCAACCCCTCAGTTCCCATCCTCCAGCAGCTGTTTGTGTGC |
| liver-muscle | TGCCTCTGAAGTCCACACTGAACAAACTTCAGCCTACTCATGT |
| tandem) | CCCTAAAATGGGCAAACATTGCAAGCAGCAAACAGCAAACACA |
| (SEQ ID NO: 86) | CAGCCCTCCCTGCCTGCTGACCTTGGAGCTGGGGCAGAGGTCA |
| | GAGACCTCTCTGGGCCCATGCCACCTCCAACATCCACTCGACC |
| | CCTTGGAATTTCGGTGGAGAGGAGCAGAGGTTGTCCTGGCGTG |
| | GTTTAGGTAGTGTGAGAGGGCCCTTCAGATTAAAAATAACTGA |
| | GGTAAGGGCCTGGGTAGGGGAGGTGGTGTGAGACGCTCCTGTC |
| | TCTCCTCTATCTGCCCATCGGCCCTTTGGGGAGGAGGAATGTG |
| | CCCAAGGACTAAAAAAAGGCCATGGAGCCAGAGGGGCGAGGGC |
| | AACAGACCTTTCATGGGCAAACCTTGGGGCCCTGCTGAAGCTT |
| | TGGCCCACTACGGGTTTAGGCTGCCCATGTAAGGAGGCAAGGC |
| | CTGGGGACACCCGAGATGCCTGGTTATAATTAACCCAGACATG |
| | TGGCTGCCCCCCCCCCCCCCAACACCTGCTGCCTCTAAAAATA |
| | ACCCTGTCCCTGGTGGATCCCCTGCATGCGAAGATCTTCGAAC |
| | AAGGCTGTGGGGGACTGAGGGCAGGCTGTAACAGGCTTGGGGG |
| | CCAGGGCTTATACGTGCCTGGGACTCCCAAAGTATTACTGTTC |
| | CATGTTCCCGGCGAAGGGCCAGCTGTCCCCCGCCAGCTAGACT |
| | CAGCACTTAGTTTAGGAACCAGTGAGCAAGTCAGCCCTTGGGG |
| | CAGCCCATACAAGGCCATGGGGCTGGGCAAGCTGCACGCCTGG |
| | GTCCGGGGTGGGCACGGTGCCCGGGCAACGAGCTGAAAGCTCA |
| | TCTGCTCTCAGGGGCCCCTCCCTGGGGACAGCCCCTCCTGGCT |
| | AGTCACACCCTGTAGGCTCCTCTATATAACCCAGGGGCACAGG |
| | GGCTGCCCTCATTCTACCACCACCTCCACAGCACAGACAGACA |
| | CTCAGGAGCCAGCCAGCGTCGAGATCTTGCTACCAGTGGAACA |
| | GCCACTAAGGATTCTGCAGTGAGAGCAGAGGGCCAGCTAAGTG |
| | GTACTCTCCCAGAGACTGTCTGACTCACGCCACCCCCTCCACC |
| | TTGGACACAGGACGCTGTGGTTTCTGAGCCAGGTACAGTGACT |
| | CCTTTCGGTAAGTGCAGTGGAAGCTGTACACTGCCCAGGCAAA |
| | GCGTCCGGGCAGCGTAGGCGGGCGACTCAGATCCCAGCCAGTG |
| | GACTTAGCCCCTGTTTGCTCCTCCGATAACTGGGGTGACCTTG |
| | GTTAATATTCACCAGCAGCCTCCCCCGTTGCCCCTCTGGATCC |
| | ACTGCTTAAATACGGACGAGGACAGGGCCCTGTCTCCTCAGCT |
| | TCAGGCACCACCACTGACCTGGGACAGT |
| LBTP1 promoter | AGGTTAATTTTTAAAAAGCAGTCAAAAGTCCAAGTGGCCCTTG |
| (synthetic, | GCAGCATTTACTCTCTCTGTTTGCTCTGGTTAATAATCTCAGG |
| liver-bone | AGCACAAACATTCCAGATCCAGGTTAATTTTTAAAAAGCAGTC |
| tandem) | AAAAGTCCAAGTGGCCCTTGGCAGCATTTACTCTCTCTGTTTG |
| (SEQ ID NO: 87) | CTCTGGTTAATAATCTCAGGAGCACAAACATTCCAGATCCGGC |
| | GCGCCAGGGCTGGAAGCTACCTTTGTCTAGAAGGCTCAGAGGC |
| | ACACAGGAGTTTCTGGGCTCACCCTGCCCCCTTCCAACCCCTC |
| | AGTTCCCATCCTCCAGCAGCTGTTTGTGTGCTGCCTCTGAAGT |
| | CCACACTGAACAAACTTCAGCCTACTCATGTCCCTAAAATGGG |
| | CAAACATTGCAAGCAGCAAACAGCAAACACACAGCCCTCCCTG |
| | CCTGCTGACCTTGGAGCTGGGGCAGAGGTCAGAGACCTCTCTG |
| | GGCCCATGCCACCTCCAACATCCACTCGACCCCTTGGAATTTC |
| | GGTGGAGAGGAGCAGAGGTTGTCCTGGCGTGGTTTAGGTAGTG |
| | TGAGAGGGGTACCCGGGGATCTTGCTACCAGTGGAACAGCCAC |
| | TAAGGATTCTGCAGTGAGAGCAGAGGGCCAGCTAAGTGGTACT |
| | CTCCCAGAGACTGTCTGACTCACGCCACCCCCTCCACCTTGGA |
| | CACAGGACGCTGTGGTTTCTGAGCCAGGTACAATGACTCCTTT |
| | CGGTAAGTGCAGTGGAAGCTGTACACTGCCCAGGCAAAGCGTC |
| | CGGGCAGCGTAGGCGGGCGACTCAGATCCCAGCCAGTGGACTT |
| | AGCCCCTGTTTGCTCCTCCGATAACTGGGGTGACCTTGGTTAA |
| | TATTCACCAGCAGCCTCCCCCGTTGCCCCTCTGGATCCACTGC |
| | TTAAATACGGACGAGGACAGGGCCCTGTCTCCTCAGCTTCAGG |
| | CACCACCACTGACCTGGGACAGT |
| LBTP2 promoter | AGGCTCAGAGGCACACAGGAGTTTCTGGGCTCACCCTGCCCCC |
| (synthetic, | TTCCAACCCCTCAGTTCCCATCCTCCAGCAGCTGTTTGTGTGC |
| liver-bone | TGCCTCTGAAGTCCACACTGAACAAACTTCAGCCTACTCATGT |
| tandem) | CCCTAAAATGGGCAAACATTGCAAGCAGCAAACAGCAAACACA |
| (SEQ ID NO: 88) | CAGCCCTCCCTGCCTGCTGACCTTGGAGCTGGGGCAGAGGTCA |
| | GAGACCTCTCTGGGCCCATGCCACCTCCAACATCCACTCGACC |
| | CCTTGGAATTTCGGTGGAGAGGAGCAGAGGTTGTCCTGGCGTG |
| | GTTTAGGTAGTGTGAGAGGGCACACATACACGAACACACATAC |
| | ATATACATACATTCACATATATGCACACACATACACATATACA |
| | CGCATACACGTACACACAAATGCACACTCACACATGCACACAC |
| | ATACATAATATACACACTCTCACACATGCACATACACACATAC |
| | ATACACATACATGTGCATGCACACACACAAATACACATGCATA |
| | CATCCACATTCACACAGATGCAGACACAAATGCACACACACAC |
| | ACACACACACACACACACACACACACACACACGCACACTGCCA |
| | CCCTGAACTAGTGGTGGCTAAATGAACAATAAGTCTCCATCAC |
| | CAGCTTGGGGGGAGGTAGGTGGTAGTGTAGGTGCCCCCATTGT |
| | GTGATCATGTTCATTGTATGAGTTTGTCTGTGTTCATTCATCA |
| | TAGTGACAGTCCCCATGTGGGTAGCAGAGAGTACGTGTGCATG |
| | CATCATCTCCGTGTTTGCTCATGAGTGTGTATGTCAGTGTGTT |
| | CCAGTCTTTCTGTGTGAGTGTCGTCCCCAATCCCCCATCCCCC |
| | CCCCCAGATCTCTAATTAGTGGTTTGGGGTTTGTTCCTTTTCC |
| | CTCCTGTTCCTTTCCTCAGCAGCGCGGCAGCAGCGGCGGCAGC |
| | CTCGGTGGTAGCAGCAGCAGCAGCAGCAGCAGCAGCAGCAGCA |
| | GCAGCAGCAGCAGCAACAGAAGCTGCCGCGCCGCTGAGTAGCA |
| | GCAAGGACTCCGAGTCAAGAGTAGGATTGTAGGATTGGATCTG |
| | AGTGGGAACAAGAGTGAGCTGGCCTGAGAGAGGAGCAGATGCC |
| | TCCCAGCGCCCTCAGGCCACCCATTGCCAGTAATCTTCAAGCC |
| | AGACCTCTTGAGAGGAGACGGGACAGCCAACCCTAGCCTACCC |
| | AGGTACAGACACTGGGCAGTTCTGGGGGACTGCCCACAGATGC |
| | CTATTGGATTCCTGGGGTATGTAGGACTCCCGGGTCTACCAGC |
| | CCTTTTCACCTTTCCCCATAGCACCCCCAAGGAAGCTCTGACA |
| | ACTTGCCCATATTCCTGTTTCCCACCCGTCCCCTGGGCACCCC |
| | CTTTTCTTCTCTCCCTCCCAGATCCCTTCTTTGGGGAGCTCAG |
| | CAAATGGAGCAGGAAATTTGGACCCTCTGCCTCCCTCTCTCGC |
| | CTTCCTCATTGGATCCGGAGTCTTCTCCGCTGGGAAAGCTGTA |
| | ATTAGAGGGTGGATCCCTACAGACAGAGAGCAGCCCCCCCACC |
| | CCCACCCCCCAGTCCCTTCTAACTTTAGATCTCTTCTCTCCCA |
| | TTCTCCCATTCTCCCTCCCTCTCCCTTCTCCCTCTCCCACTGG |
| | CTCCTCGGTTCTCTCCATCTGCCTGACTCCTTGGGACCCGGTC |
| | CCCAGATCTTGCTACCAGTGGAACAGCCACTAAGGATTCTGCA |
| | GTGAGAGCAGAGGGCCAGCTAAGTGGTACTCTCCCAGAGACTG |
| | TCTGACTCACGCCACCCCCTCCACCTTGGACACAGGACGCTGT |
| | GGTTTCTGAGCCAGGTACAGTGACTCCTTTCGGTAAGTGCAGT |
| | GGAAGCTGTACACTGCCCAGGCAAAGCGTCCGGGCAGCGTAGG |
| | CGGGCGACTCAGATCCCAGCCAGTGGACTTAGCCCCTGTTTGC |
| | TCCTCCGATAACTGGGGTGACCTTGGTTAATATTCACCAGCAG |
| | CCTCCCCCGTTGCCCCTCTGGATCCACTGCTTAAATACGGACG |
| | AGGACAGGGCCCTGTCTCCTCAGCTTCAGGCACCACCACTGAC |
| | CTGGGACAGT |
| hAAT promoter | GGATCTTGCTACCAGTGGAACAGCCACTAAGGATTCTGCAGTG |
| (liver-specific) | AGAGCAGAGGGCCAGCTAAGTGGTACTCTCCCAGAGACTGTCT |
| (SEQ ID NO: 89) | GACTCACGCCACCCCCTCCACCTTGGACACAGGACGCTGTGGT |
| | TTCTGAGCCAGGTACAATGACTCCTTTCGGTAAGTGCAGTGGA |
| | AGCTGTACACTGCCCAGGCAAAGCGTCCGGGCAGCGTAGGCGG |
| | GCGACTCAGATCCCACCAGTGGACTTAGCCCCTGTTTGCTCCT |
| | CCGATAACTGGGGTGACCTTGGTTAATATTCACCAGCAGCCTC |
| | CCCCGTTGCCCCTCTGGATCCACTGCTTAAATACGGACGAGGA |
| | CAGGGCCCTGTCTCCTCAGCTTCAGGCACCACCACTGACCTGG |
| | GACAGTGAATGATCCCCCTGATCTGCGGCC |
| ApoE.hAAT | AGGCTCAGAGGCACACAGGAGTTTCTGGGCTCACCCTGCCCCC |
| promoter | TTCCAACCCCTCAGTTCCCATCCTCCAGCAGCTGTTTGTGTGC |
| (liver-specific) | TGCCTCTGAAGTCCACACTGAACAAACTTCAGCCTACTCATGT |
| (SEQ ID NO: 90) | CCCTAAAATGGGCAAACATTGCAAGCAGCAAACAGCAAACACA |
| | CAGCCCTCCCTGCCTGCTGACCTTGGAGCTGGGGCAGAGGTCA |
| | GAGACCTCTCTGGGCCCATGCCACCTCCAACATCCACTCGACC |
| | CCTTGGAATTTCGGTGGAGAGGAGCAGAGGTTGTCCTGGCGTG |
| | GTTTAGGTAGTGTGAGAGGGGTACCCGGGGATCTTGCTACCAG |
| | TGGAACAGCCACTAAGGATTCTGCAGTGAGAGCAGAGGGCCAG |
| | CTAAGTGGTACTCTCCCAGAGACTGTCTGACTCACGCCACCCC |
| | CTCCACCTTGGACACAGGACGCTGTGGTTTCTGAGCCAGGTAC |
| | AATGACTCCTTTCGGTAAGTGCAGTGGAAGCTGTACACTGCCC |
| | AGGCAAAGCGTCCGGGCAGCGTAGGCGGGCGACTCAGATCCCA |
| | GCCAGTGGACTTAGCCCCTGTTTGCTCCTCCGATAACTGGGGT |
| | GACCTTGGTTAATATTCACCAGCAGCCTCCCCCGTTGCCCCTC |
| | TGGATCCACTGCTTAAATACGGACGAGGACAGGGCCCTGTCTC |
| | CTCAGCTTCAGGCACCACCACTGACCTGGGACAGT |
| TBG promoter | GGGCTGGAAGCTACCTTTGACATCATTTCCTCTGCGAATGCAT |
| (liver-specific) | GTATAATTTCTACAGAACCTATTAGAAAGGATCACCCAGCCTC |
| (SEQ ID NO: 91) | TGCTTTTGTACAACTTTCCCTTAAAAAACTGCCAATTCCACTG |
| | CTGTTTGGCCCAATAGTGAGAACTTTTTCCTGCTGCCTCTTGG |
| | TGCTTTTGCCTATGGCCCCTATTCTGCCTGCTGAAGACACTCT |
| | TGCCAGCATGGACTTAAACCCCTCCAGCTCTGACAATCCTCTT |
| | TCTCTTTTGTTTTACATGAAGGGTCTGGCAGCCAAAGCAATCA |
| | CTCAAAGTTCAAACCTTATCATTTTTTGCTTTGTTCCTCTTGG |
| | CCTTGGTTTTGTACATCAGCTTTGAAAATACCATCCCAGGGTT |
| | AATGCTGGGGTTAATTTATAACTAAGAGTGCTCTAGTTTTGCA |
| | ATACAGGACATGCTATAAAAATGGAAAGAT |
| CK8 promoter | CCACTACGGGTTTAGGCTGCCCATGTAAGGAGGCAAGGCCTGG |
| (muscle specific) | GGACACCCGAGATGCCTGGTTATAATTAACCCAGACATGTGGC |
| (SEQ ID NO: 92) | TGCCCCCCCCCCCCCCAACACCTGCTGCCTCTAAAAATAACCC |
| | TGTCCCTGGTGGATCCCACTACGGGTTTAGGCTGCCCATGTAA |
| | GGAGGCAAGGCCTGGGGACACCCGAGATGCCTGGTTATAATTA |
| | ACCCAGACATGTGGCTGCCCCCCCCCCCCCCAACACCTGCTGC |
| | CTCTAAAAATAACCCTGTCCCTGGTGGATCCCACTACGGGTTT |
| | AGGCTGCCCATGTAAGGAGGCAAGGCCTGGGGACACCCGAGAT |
| | GCCTGGTTATAATTAACCCAGACATGTGGCTGCCCCCCCCCCC |
| | CCCAACACCTGCTGCCTCTAAAAATAACCCTGTCCCTGGTGGA |
| | TCCCCTGCATGCGAAGATCTTCGAACAAGGCTGTGGGGGACTG |
| | AGGGCAGGCTGTAACAGGCTTGGGGGCCAGGGCTTATACGTGC |
| | CTGGGACTCCCAAAGTATTACTGTTCCATGTTCCCGGCGAAGG |
| | GCCAGCTGTCCCCCGCCAGCTAGACTCAGCACTTAGTTTAGGA |
| | ACCAGTGAGCAAGTCAGCCCTTGGGGCAGCCCATACAAGGCCA |
| | TGGGGCTGGGCAAGCTGCACGCCTGGGTCCGGGGTGGGCACGG |
| | TGCCCGGGCAACGAGCTGAAAGCTCATCTGCTCTCAGGGGCCC |
| | CTCCCTGGGGACAGCCCCTCCTGGCTAGTCACACCCTGTAGGC |
| | TCCTCTATATAACCCAGGGGCACAGGGGCTGCCCTCATTCTAC |
| | CACCACCTCCACAGCACAGACAGACACTCAGGAGCCAGCCAGC |
| | GTCGA |
| SPc5-12 promoter | GGCCGTCCGCCCTCGGCACCATCCTCACGACACCCAAATATGG |
| (muscle specific) | CGACGGGTGAGGAATGGTGGGGAGTTATTTTTAGAGCGGTGAG |
| (SEQ ID NO: 93) | GAAGGTGGGCAGGCAGCAGGTGTTGGCGCTCTAAAAATAACTC |
| | CCGGGAGTTATTTTTAGAGCGGAGGAATGGTGGACACCCAAAT |
| | ATGGCGACGGTTCCTCACCCGTCGCCATATTTGGGTGTCCGCC |
| | CTCGGCCGGGGCCGCATTCCTGGGGGCCGGGCGGTGCTCCCGC |
| | CCGCCTCGATAAAAGGCTCCGGGGCCGGCGGCGGCCCACGAGC |
| | TACCCGGAGGAGCGGGAGGCGCCAAGC |
| MCK7 promoter | CTAGAAGCTGCATGTCTAAGCTAGACCCTTCAGATTAAAAATA |
| (muscle specific) | ACTGAGGTAAGGGCCTGGGTAGGGGAGGTGGTGTGAGACGCTC |
| (SEQ ID NO: 94) | CTGTCTCTCCTCTATCTGCCCATCGGCCCTTTGGGGAGGAGGA |
| | ATGTGCCCAAGGACTAAAAAAAGGCCATGGAGCCAGAGGGGCG |
| | AGGGCAACAGACCTTTCATGGGCAAACCTTGGGGCCCTGCTGT |
| | CTAGCATGCCCCACTACGGGTCTAGGCTGCCCATGTAAGGAGG |
| | CAAGGCCTGGGGACACCCGAGATGCCTGGTTATAATTAACCCA |
| | GACATGTGGCTGCCCCCCCCCCCCCAACACCTGCTGCCTCTAA |
| | AAATAACCCTGTCCCTGGTGGATCCCCTGCATGCGAAGATCTT |
| | CGAACAAGGCTGTGGGGGACTGAGGGCAGGCTGTAACAGGCTT |
| | GGGGGCCAGGGCTTATACGTGCCTGGGACTCCCAAAGTATTAC |
| | TGTTCCATGTTCCCGGCGAAGGGCCAGCTGTCCCCCGCCAGCT |
| | AGACTCAGCACTTAGTTTAGGAACCAGTGAGCAAGTCAGCCCT |
| | TGGGGCAGCCCATACAAGGCCATGGGGCTGGGCAAGCTGCACG |
| | CCTGGGTCCGGGGTGGGCACGGTGCCCGGGCAACGAGCTGAAA |
| | GCTCATCTGCTCTCAGGGGCCCCTCCCTGGGGACAGCCCCTCC |
| | TGGCTAGTCACACCCTGTAGGCTCCTCTATATAACCCAGGGGC |
| | ACAGGGGCTGCCCTCATTCTACCACCACCTCCACAGCAC |
| truncatedMCK | CCACTACGGGTCTAGGCTGCCCATGTAAGGAGGCAAGGCCTGG |
| (IMCK) promoter | GGACACCCGAGATGCCTGGTTATAATTAACCCCAACACCTGCT |
| (muscle specific) | GCCCCCCCCCCCCCAACACCTGCTGCCTGAGCCTGAGCGGTTA |
| (SEQ ID NO: 95) | CCCCACCCCGGTGCCTGGGTCTTAGGCTCTGTACACCATGGAG |
| | GAGAAGCTCGCTCTAAAAATAACCCTGTCCCTGGTGGATCCAC |
| | TACGGGTCTATGCTGCCCATGTAAGGAGGCAAGGCCTGGGGAC |
| | ACCCGAGATGCCTGGTTATAATTAACCCCAACACCTGCTGCCC |
| | CCCCCCCCCCAACACCTGCTGCCTGAGCCTGAGCGGTTACCCC |
| | ACCCCGGTGCCTGGGTCTTAGGCTCTGTACACCATGGAGGAGA |
| | AGCTCGCTCTAAAAATAACCCTGTCCCTGGTGGACCACTACGG |
| | GTCTAGGCTGCCCATGTAAGGAGGCAAGGCCTGGGGACACCCG |
| | AGATGCCTGGTTATAATTAACCCCAACACCTGCTGCCCCCCCC |
| | CCCCAACACCTGCTGCCTGAGCCTGAGCGGTTACCCCACCCCG |
| | GTGCCTGGGTCTTAGGCTCTGTACACCATGGAGGAGAAGCTCG |
| | CTCTAAAAATAACCCTGTCCCTGGTCCTCCCTGGGGACAGCCC |
| | CTCCTGGCTAGTCACACCCTGTAGGCTCCTCTATATAACCCAG |
| | GGGCACAGGGGCTGCCCCCGGGTCAC |
| Mouse skeletal | GGGGTGATGTGTGTCAGATCTCTGGATTGGGGGAGCTTCAAAG |
| muscle alpha | TGGGAAAGAAAATGGAGTTCAAATGTGGGGCTTATTTTCCATC |
| actin actal | CCTACCTGGAGCCCATGACTCCTCCCGGCTCACCTGACCACAG |
| promoter | GGCTACCTCCCCTGAGCTTAAGCATCAAGGCTTAGTAGTCTGA |
| (muscle specific) | GTTAAGJAACCCATAAATGGGGTGCATTGTGGCAGGTCAGCAA |
| (SEQ ID NO: 96) | TCGTGTGTCCAGGTGGGCAGAACTGGGGAGACCTTTCAAACAG |
| | GTAAATCTTGGGAAGTACAGACCAGCAGTCTGCAAAGCAGTGA |
| | CCTTTGGCCCAGCACAGCCCTTCCGTGAGCCTTGGAGCCAGTT |
| | GGGAGGGGCAGACAGCTGGGGATACTCTCCATATACGGCCTGG |
| | TCCGGTCCTAGCTACCTGGGCCAGGGCCAGTCCTCTCCTTCTT |
| | TGGTCAGTGCAGGAGACCCGGGCGGGGACCCAGGCTGAGAACC |
| | AGCCGAAGGAAGGGACTCTAGTGCCCGACACCCAAATATGGCT |
| | TGGGAAGGGCAGCAACATTCCTTCGGGGCGGTGTGGGGAGAGC |
| | TCCCGGGACTATATAAAAACCTGTGCAAGGGGACAGGCGGTC |
| Human muscle | AGCCAGCCTCAGTTTCCCCTCCACTCAGTCCCTAGGAGGAAGG |
| creatine | GGCGCCCAAGCGCGGGTTTCTGGGGTTAGACTGCCCTCCATTG |
| kinase (MCK) | CAATTGGTCCTTCTCCCGGCCTCTGCTTCCTCCAGCTCACAGG |
| promoter | GTATCTGCTCCTCCTGGAGCCACACCTTGGTTCCCCGAGGTGC |
| (muscle specific) | CGCTGGGACTCGGGTAGGGGTGAGGGCCCAGGGGGCACAGGGG |
| (SEQ ID NO: 97) | GAGCCGAGGGCCACAGGAAGGGCTGGTGGCTGAAGGAGACTCA |
| | GGGGCCAGGGGACGGTGGCTTCTACGTGCTTGGGACGTTCCCA |
| | GCCACCGTCCCATGTTCCCGGCGGGGGGCCAGCTGTCCCCACC |
| | GCCAGCCCAACTCAGCACTTGGTCAGGGTATCAGCTTGGTGGG |
| | GGGGCGTGAGCCCAGCCCCTGGGGCGGCTCAGCCCATACAAGG |
| | CCATGGGGCTGGGCGCAAAGCATGCCTGGGTTCAGGGTGGGTA |
| | TGGTGCGGGAGCAGGGAGGTGAGAGGCTCAGCTGCCCTCCAGA |
| | ACTCCTCCCTGGGGACAACCCCTCCCAGCCAATAGCACAGCCT |
| | AGGTCCCCCTATATAAGGCCACGGCTGCTGGCCCTTCCTTT |
| Human desmin | CTGAGGCTCAGGGCTAGCTCGCCCATAGACATACATGGCAGGC |
| promoter | AGGCTTTGGCCAGGATCCCTCCGCCTGCCAGGCGTCTCCCTGC |
| (NCBI sequence | CCTCCCTTCCTGCCTAGAGACCCCCACCCTCAAGCCTGGCTGG |
| ID No. 1674) | TCTTTGCCTGAGACCCAAACCTCTTCGACTTCAAGAGAATATT |
| (muscle specific) | TAGGAACAAGGTGGTTTAGGGCCTTTCCTGGGAACAGGCCTTG |
| (SEQ ID NO: 98) | ACCCTTTAAGAAATGACCCAAAGTCTCTCCTTGACCAAAAAGG |
| | GGACCCTCAAACTAAAGGGAAGCCTCTCTTCTGCTGTCTCCCC |
| | TGACCCCACTCCCCCCCACCCCAGGACGAGGAGATAACCAGGG |
| | CTGAAAGAGGCCCGCCTGGGGGCTGCAGACATGCTTGCTGCCT |
| | GCCCTGGCGAAGGATTGGCAGGCTTGCCCGTCACAGGACCCCC |
| | GCTGGCTGACTCAGGGGCGCAGGCCTCTTGCGGGGGAGCTGGC |
| | CTCCCCGCCCCCACGGCCACGGGCCGCCCTTTCCTGGCAGGAC |
| | AGCGGGATCTTGCAGCTGTCAGGGGAGGGGAGGCGGGGGCTGA |
| | TGTCAGGAGGGATACAAATAGTGCCGACGGCTGGGGGCCCT |
| Human desmin 2 | CTGCAGACATGCTTGCTGCCTGCCCTGGCGTGCCCTGGCGAGG |
| promoter | CTTGCCGTCACAGGACCCCCGCTGGCTGACTCAGGGGCGCAGG |
| (muscle specific) | CTCTTGCGGGGGAGCTGGCCTCCCGCCCCCACGGCCACGGGCC |
| (SEQ ID NO: 99) | CTTTCCTGGCAGGACAGCGGGATCTTGCAGCTGTCAGGGGAGG |
| | GGATGACGGGGGACTGATGTCAGGAGGGGATACAAATAGTGCC |
| | GAACAAGGACCGGATTAGATCTACC |
| Human skeletal | GGAGTTCCAGGGGCGTAAAGGAGAGGGAGTTCGCCTTCCTTCC |
| muscle alpha | CTTCCTGAGACTCAGGAGTGACTGCTTCTCCAATCCTCCCAAG |
| actin actal | CCCACCACTCCACACGACTCCCTCTTCCCGGTAGTCGCAAGTG |
| promoter | GGAGTTTGGGGATCTGAGCAAAGAACCCGAAGAGGAGTTGAAA |
| (NCBI sequence | TATTGGAAGTCAGCAGTCAGGCACCTTCCCGAGCGCCCAGGGC |
| ID No. 58) | GCTCAGAGTGGACATGGTTGGGGAGGCCTTTGGGACAGGTGCG |
| (muscle specific) | GTTCCCGGAGCGCAGGCGCACACATGCACCCACCGGCGAACGC |
| (SEQ ID NO: 100) | GGTGACCCTCGCCCCACCCCATCCCCTCCGGCGGGCAACTGGG |
| | TCGGGTCAGGAGGGGCAAACCCGCTAGGGAGACACTCCATATA |
| | CGGCCCGGCCCGCGTTACCTGGGACCGGGCCAACCCGCTCCTT |
| | CTTTGGTCAACGCAGGGGACCCGGGGGGGGGCCCAGGCCGCGA |
| | ACCGGCCGAGGGAGGGGGCTCTAGTGCCCAACACCCAAATATG |
| | GCTCGAGAAGGGCAGCGACATTCCTGCGGGGGGCGCGGAGGGA |
| | ATGCCCGCGGGCTATATAAAACCTGAGCAGAGGGACAAGC |
| Mouse muscle | AGAAACCTGTGGTCTAGAGGGGGGCGGGGCCGATGGAGGCAAC |
| creatine kinase | GCACGCCCCCGCAGGCGCCCAGGCCACGCCCTCTGCCGCAGCA |
| (MCK) promoter | TTCGGTGAAACCTGCGTTCCGAGAACTTCTGAAAACTTTATCT |
| (NCBI sequence | GGGGGCCTTCGAGAAGGCTCAGACAGTAAGGGTGCATGCTGCC |
| ID No. 12715) | AATCCTGAGGAGCTGAGTTCGATCCCTGAGACCTTCAGGGTGG |
| (muscle specific) | ACAGAGACGGACTCCCACATGTTGTTTTCTGACTTCTACATGT |
| (SEQ ID NO: 101) | GTCCAGTCATACATACACAAATATGGAATAAACAGATGGCTCA |
| | TCAGGTAAGAGTGCTGGCTGCTTTTGCAGAGGACCCAGGTTCG |
| | ATTTCCAGAACCCACATGTCGGCTCAAAATCATCTGTAATTCC |
| | AGTTCCAGGGAGATCCAGCACTTTCTTCCAGGGCCTCCACAGA |
| | CACACATAAAATAAAGATAAAAATCTCCAAAAAATATTGTTTT |
| | AATAATTACAACCTGAAGACCTTGCACAACTATTCCTGGCTGA |
| | GAAGATGGTAAGGGCGCTAGCTGCCAAGCTTGACAGCCTGAGT |
| | TTCATCTCCAAGAACCATGAAAACTGACTCCTGGGAATTA |
| Mouse desmin | GGAAGCAGAAGGCCAACATTCCTCCCAAGGGAAACTGAGGCTC |
| promoter | AGAGTTAAAACCCAGGTATCAGTGATATGCATGTGCCCCGGCC |
| (NCBI sequence | AGGGTCACTCTCTGACTAACCGGTACCTACCCTACAGGCCTAC |
| ID No. 13346) | CTAGAGACTCTTTTGAAAGGATGGTAGAGACCTGTCCGGGCTT |
| (muscle specific) | TGCCCACAGTCGTTGGAAACCTCAGCATTTTCTAGGCAACTTG |
| (SEQ ID NO: 102) | TGCGAATAAAACACTTCGGGGGTCCTTCTTGTTCATTCCAATA |
| | ACCTAAAACCTCTCCTCGGAGAAAATAGGGGGCCTCAAACAAA |
| | CGAAATTCTCTAGCCCGCTTTCCCCAGGATAAGGCAGGCATCC |
| | AAATGGAAAAAAAGGGGCCGGCCGGGGGTCTCCTGTCAGCTCC |
| | TTGCCCTGTGAAACCCAGCAGGCCTGCCTGTCTTCTGTCCTCT |
| | TGGGGCTGTCCAGGGGCGCAGGCCTCTTGCGGGGGAGCTGGCC |
| | TCCCCGCCCCCTCGCCTGTGGCCGCCCTTTTCCTGGCAGGACA |
| | GAGGGATCCTGCAGCTGTCAGGGGAGGGGCGCCGGGGGGTGAT |
| | GTCAGGAGGGCTACAAATAGTGCAGACAGCTAAGGGGCTCC |
| CXCL10 promoter | TCAAGGAGGACTGTCCAGGTAAATCACTGTTCTAATAATCAGG |
| (synovial | CACAACTTGCTGTTACCAAAAAATTAGGTTTACCTATAAAGGA |
| specific) | TGAAAAATTTCTATTACTGGGGATATTTTGAAAAATAACCTCT |
| (See Broeren | GAAAGTAACTGTAAAAGTTTAAGTGTTGAGAAAAAGCATTATA |
| MGA et al., | GTTAGAATGGATTGCAACCTTTGTTTTTTTCTATATGCAATGA |
| Hum Gene Ther. | AGTTCTTTTTTTCAAGAAACAGTTCATGTTTTGGAAAGTGAAA |
| 2016;27(3): | CCTAATTCACTATTACCAAAAAAAGAGGAGCAGAGGGAAATTC |
| 244-54) | CGTAACTTGGAGGCTACAATAAATAATACCTTCGAGTCTGCAA |
| (SEQ ID NO: 103) | CATGGGACTTCCCCAGGAACAGCCAGCAGGTTTTGCTAAGTCA |
| | ACTGTAATGCCCTTATCCAATCAGAATTAGGGAGGGAAAATGG |
| | CTTTGCAGATAAATATGGCACACTAGCCCCACGTTTTCTGAGA |
| | CATTCCTCAATTGCTTAGACATATTCTGAGCCTACAGCAGAGG |
| | AACCTCCAGTCTCAGCACC |

Table 10 provides examples and nucleotide sequences for ocular promoters for use herein.

TABLE 10
Representative Ocular-Specific Promoters

| Promoter | Sequence |
|---|---|
| RPE65 promoter | AAATCAAAAGTCCTCAACCTGGTTGGAAGAATATTGGCACTGA |
| (SEQ ID NO: 104) | ATGGTATCAATAAGGTTGCTAGAGAGGGTTAGAGGTGCACAAT |
| Derived from | GTGCTTCCATAACATTTTATACTTCTCCAATCTTAGCACTAAT |
| GenBank: | CAAACATGGTTGAATACTTTGTTTACTATAACTCTTACAGAGT |
| AF304008.1 | TATAAGATCTGTGAAGACAGGGACAGGGACAATACCCATCTCT |
| | GTCTGGTTCATAGGTGGTATGTAATAGATATTTTTAAAAATAA |
| | GTGAGTTAATGAATGAGGGTGAGAATGGAGGCACAGAGGTATT |
| | AGGGGGAGGTGGGCCCCAGAGAATGGTGCCAAGGTCCAGTGGG |
| | GTGACTGGGATCAGCTCAGGCCTGACGCTGGCCACTCCCACCT |
| | AGCTCCTTTCTTTCTAATCTGTTCTCATTCTCCTTGGGAAGGA |
| | TTGAGGTCTCTGGAAAACAGCCAAACAACTGTTATGGGAACAG |
| | CAAGCCCAAATAAAGCCAAGCATCAGGGGGATCTGAGAGCTGA |
| | AAGCAACTTCTGTTCCCCCTCCCTCAGCTGAAGGGGTGGGGAA |
| | GGGCTCCCAAAGCCATAACTCCTTTTAAGGGATTTAGAAGGCA |
| | TAAAAAGGCCCCTGGCTGAGAACT |
| Red cone opsin | AGCCATCGGCTGTTAGTGACAAAGCCCCTGAGTCAAGATGACA |
| promoter | GCAGCCCCCATAACTCCTAATCGGCTCTCCCGCGTGGAGTCAT |
| (SEQ ID NO: 105) | TTAGGAGTAGTCGCATTAGAGACAAGTCCAACATCTAATCTTC |
| GenBank: | CACCCTGGCCAGGGCCCCAGCTGGCAGCGAGGGTGGGAGACTC |
| KT886396.1 | CGGGCAGAGCAGAGGGCGCTGACATTGGGGCCCGGCCTGGCTT |
| | GGGTCCCTCTGGCCTTTCCCCAGGGGCCCTCTTTCCTTGGGGC |
| | TTTCTTGGGCCGCCACTGCTCCCGCTCCTCTCCCCCCATCCCA |
| | CCCCCTCACCCCCTCGTTCTTCATATCCTTCTCTAGTGCTCCC |
| | TCCACTTTCATCCACCCTTCTGCAAGAGTGTGGGACCACAAAT |
| | GAGTTTTCACCTGGCCTGGGGACACACGTGCCCCCACAGGTGC |
| | TGAGTGACTTTCTAGGACAGTAATCTGCTTTAGGCTAAAATGG |
| | GACTTGATCTTCTGTTAGCCCTAATCATCAATTAGCAGAGCCG |
| | GTGAAGGTGCAGAACCTACCGCCTTTCCAGGCCTCCTCCCACC |
| | TCTGCCACCTCCACTCTCCTTCCTGGGATGTGGGGGCTGGCAC |
| | ACGTGTGGCCCAGGGCATTGGTGGGATTGCACTGAGCTGGGTC |
| | ATTAGCGTAATCCTGGACAAGGGCAGACAGGGCGAGCGGAGGG |
| | CCAGCTCCGGGGCTCAGGCAAGGCTGGGGGCTTCCCCCAGACA |
| | CCCCACTCCTCCTCTGCTGGACCCCCACTTCATAGGGCACTTC |
| | GTGTTCTCAAAGGGCTTCCAAATAGCATGGTGGCCTTGGATGC |
| | CCAGGGAAGCCTCAGAGTTGCTTATCTCCCTCTAGACAGAAGG |
| | GGAATCTCGGTCAAGAGGGAGAGGTCGCCCTGTTCAAGGCCAC |
| | CCAGCCAGCTCATGGCGGTAATGGGACAAGGCTGGCCAGCCAT |
| | CCCACCCTCAGAAGGGACCCGGTGGGGCAGGTGATCTCAGAGG |
| | AGGCTCACTTCTGGGTCTCACATTCTTGGATCCGGTTCCAGGC |
| | CTCGGCCCTAAATAGTCTCCCTGGGCTTTCAAGAGAACCACAT |
| | GAGAAAGGAGGATTCGGGCTCTGAGCAGTTTCACCACCCACCC |
| | CCCAGTCTGCAAATCCTGACCCGTGGGTCCACCTGCCCCAAAG |
| | GCGGACGCAGGACAGTAGAAGGGAACAGAGAACACATAAACAC |
| | AGAGAGGGCCACAGCGGCTCCCACAGTCACCGCCACCTTCCTG |
| | GCGGGGATGGGTGGGGCGTCTGAGTTTGGTTCCCAGCAAATCC |
| | CTCTGAGCCGCCCTTGCGGGCTCGCCTCAGGAGCAGGGGAGCA |
| | AGAGGTGGGAGGAGGAGGTCTAAGTCCCAGGCCCAATTAAGAG |
| | ATCAGGTAGTGTAGGGTTTGGGAGCTTTTAAGGTGAAGAGGCC |
| | CGGGCTGATCCCACAGGCCAGTATAAAGCGCCGTGACCCTCAG |
| | GTGATGCGCCAGGGCCGGCTGCCGTCGGGGACAGGGCTTTCCA |
| | TAGCCATG |
| BST1 promoter | GTCGACAGAATTCTGTCATTTTACTAGGGTGATGAAATTCCCA |
| (SEQ ID NO: 106) | AGCAACACCATCCTTTTCAGATAAGGGCACTGAGGCTGAGAGA |
| | GGAGCTGAAACCTACCCGGGGTCACCACACACAGGTGGCAAGG |
| | CTGGGACCAGAAACCAGGACTGTTGACTGCAGCCCGGTATTCA |
| | TTCTTTCCATAGCCCACAGGGCTGTCAAAGACCCCAGGGCCTA |
| | GTCAGAGGCTCCTCCTTCCTGGAGAGTTCCTGGCACAGAAGTT |
| | GAAGCTCAGCACAGCCCCCTAACCCCCAACTCTCTCTGCAAGG |
| | CCTCAGGGGTCAGAACACTGGTGGAGCAGATCCTTTAGCCTCT |
| | GGATTTTAGGGCCATGGTAGAGGGGGTGTTGCCCTAAATTCCA |
| | GCCCTGGTCTCAGCCCAACACCCTCCAAGAAGAAATTAGAGGG |
| | GCCATGGCCAGGCTGTGCTAGCCGTTGCTTCTGAGCAGATTAC |
| | AAGAAGGGACTAAGACAAGGACTCCTTTGTGGAGGTCCTGGCT |
| | TAGGGAGTCAAGTGACGGCGGCTCAGCACTCACGTGGGCAGTG |
| | CCAGCCTCTAAGAGTGGGCAGGGGCACTGGCCACAGAGTCCCA |
| | GGGAGTCCCACCAGCCTAGTCGCCAGAGAATTC |

### Other Regulatory Cassette Elements, Including Introns, ITRs, and UTRs

In certain embodiments, the viral vectors provided herein comprise one or more regulatory/cassette elements other than a promoter (e.g., introns, inverted terminal repeats (ITRs), and/or untranslated regions (UTRs)). In certain embodiments, the viral vectors provided herein comprise an enhancer. In certain embodiments, the viral vectors provided herein comprise a repressor. In certain embodiments, the viral vectors provided herein comprise an intron (e.g. VH4 intron (SEQ ID NO: 108) or a chimeric intron (SEQ ID NO: 107). In certain embodiments, the viral vectors provided herein comprise a polyadenylation sequence (e.g. Rabbit β-globin poly A (SEQ ID NO: 109) and β-globin PolyA signal (SEQ ID NO: 110)). In certain embodiments, the viral vectors provided herein comprise a 5′ ITR or a 3′ ITR (e.g., SEQ ID NOs: 111-113 and 183). Table 11 provides examples and sequences of regulatory elements.

In certain embodiments, the viral vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the mRNA half-life of the transgene (DN-TNFα). In certain embodiments, the UTRs are optimized for the stability of the mRNA of the transgene (DN-TNFα). In certain embodiments, the UTRs are optimized for the secondary structure of the mRNA of the transgene (DN-TNFα).

In certain embodiments, the viral vectors provided herein comprise one or more inverted terminal repeat (ITR) sequences. ITR sequences may be used for packaging the recombinant gene expression cassette into the virion of the viral vector. In certain embodiments, the ITR is from an AAV, e.g., AAV8 or AAV2 (see, e.g., Yan Z et al., 2005, J. Virol., 79(1): 364-379; U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety). In some embodiments, nucleotide sequences encoding the ITRs may, for example, comprise any one of the nucleotide sequences of SEQ ID NOs: 111-112 (5′-ITR) and any one of the nucleotide sequences of SEQ ID NOs: 113 and 183 (3′-ITR). In certain embodiments, the modified ITRs used to produce self-complementary vectors, e.g., scAAV, may be used (see, e.g., Wu J. et al., 2007, Human Gene Therapy, 18 (2): 171-82, McCarty D M, et al., Gene Ther. 2001 August; 8 (16): 1248-54; and U.S. Pat. Nos. 6,596,535; 7,125,717; and 7,456,683, each of which is incorporated herein by reference in its entirety) . . . . Table 11 provides representative regulatory/cassette elements and their nucleotide sequences.

TABLE 11
Representative Regulatory/Cassette Elements

| Regulatory/ | |
|---|---|
| Cassette Element | Sequence |
| Chimeric Intron | GTAAGTATCAAGGTTACAAGACAGGTTTAAGGAGACCAATAGAA |
| (SEQ ID NO: 107) | ACTGGGCTTGTCGAGACAGAGAAGACTCTTGCGTTTCTGATAGG |
| | CACCTATTGGTCTTACTGACATCCACTTTGCCTTTCTCTCCACA |
| | G |
| VH4 Intron | GTGAGTATCTCAGGGATCCAGACATGGGGATATGGGAGGTGCCT |
| (SEQ ID NO: 108) | CTGATCCCAGGGCTCACTGTGGGTCTCTCTGTTCACAG |
| Rabbit β-globin | GATCTTTTTCCCTCTGCCAAAAATTATGGGGACATCATGAAGCC |
| polyA | CCTTGAGCATCTGACTTCTGGCTAATAAAGGAAATTTATTTTCA |
| (SEQ ID NO: 109) | TTGCAATAGTGTGTTGGAATTTTTTGTGTCTCTCACTCG |
| β-globin PolyA | ATAAAGGAAATTTATTTTCATTGCAATAGTGTGTTGGAATTTTT |
| signal | TGTGTCTCTCA |
| (SEQ ID NO: 110) | |
| 5′ ITR-A | CTGCGCGCTCGCTCGCTCACTGAGGCCGCCCGGGCAAAGCCCGG |
| (SEQ ID NO: 111) | GCGTCGGGCGACCTTTGGTCGCCCGGCCTCAGTGAGCGAGCGAG |
| | CGCGCAGAGAGGGAGTGGCCAACTCCATCACTAGGGGTTCCT |
| 5′ ITRm (deleted | CTGCGCGCTCGCTCGCTCACTGAGGCCGCCCGGGCAAAGCCCGG |
| D sequence) or | GCGTCGGGCGACCTTTGGTCGCCCGGCCTCAGTGAGCGAGCGAG |
| 5′ ITR-B | CGCGCAGAGAGGGAGTGG |
| (SEQ ID NO: 112) | |
| 3′-ITR AAV | AGGAACCCCTAGTGATGGAGTTGGCCACTCCCTCTCTGCGCGCT |
| (SEQ ID NO: 113) | CGCTCGCTCACTGAGGCCGGGCGACCAAAGGTCGCCCGACGCCC |
| | GGGCTTTGCCCGGGGGGCCTCAGTGAGCGAGCGAGCGCGCAG |
| Alternative 3′TTR | GAACCCCTAGTGATGGAGTTGGCCACTCCCTCTCTGCGCGCTCG |
| (SEQ ID NO: 183) | CTCGCTCACTGAGGCCGCCCGGGCAAAGCCCGGGCGTCGGGCGA |
| | CCTTTGGTCGCCCGGCCTCAGTGAGCGAGCGAGCGCGCAGAGAG |
| | GGAGTGGCCAA |
| BGH polyA | TAGCAGGCATGCTGGGGAG |
| (SEQ ID NO: 114) | |
| 5′-ITR1 (GenBank | TTGCCCACTCCCTCTCTGCGCGCTCGCTCGCTCGGTGGGGCCTG |
| NC_002077.1) (SEQ | CGGACCAAAGGTCCGCAGACGGCAGAGCTCTGCTCTGCCGGCCC |
| ID NO: 184) | CACCGAGCGAGCGAGCGCGCAGAGAGGGAGTGGGCAACTCCATC |
| | ACTAGGGGTAA |
| 5′-ITR2 (GenBank | TTGGCCACTCCCTCTCTGCGCGCTCGCTCGCTCACTGAGGCCGG |
| NC_001401.2) (SEQ | GCGACCAAAGGTCGCCCGACGCCCGGGCTTTGCCCGGGGGGCCT |
| ID NO: 185) | CAGTGAGCGAGCGAGCGCGCAGAGAGGGAGTGGCCAACTCCATC |
| | ACTAGGGGTTCCT |
| 5′-ITR3 (GenBank | TTGGCCACTCCCTCTATGCGCACTCGCTCGCTCGGTGGGGCCTG |
| NC_JB292182.1) | GCGACCAAAGGTCGCCAGACGGACGTGCTTTGCACGTCCGGCCC |
| (SEQ ID NO: 186) | CACCGAGCGAGCGAGTGCGCATAGAGGGAGTGGCCAACTCCATC |
| | ACTAGAGGTAT |
| 5′-ITR4 (GenBank | TTGGCCACTCCCTCTATGCGCGCTCGCTCACTCACTCGGCCCTG |
| NC_001829.1) (SEQ | GAGACCAAAGGTCTCCAGACTGCCGGCCTCTGGCCGGCAGGGCC |
| ID NO: 187) | GAGTGAGTGAGCGAGCGCGCATAGAGGGAGTGGCCAACTCCATC |
| | ATCTAGGTTTGCCC |
| 5′-ITR7 (GenBank | TTGGCCACTCCCTCTATGCGCGCTCGCTCGCTCGGTGGGGCCTG |
| NC_006260.1) (SEQ | CGGACCAAAGGTCCGCAGACGGCAGAGCTCTGCTCTGCCGGCCC |
| ID NO: 188) | CACCGAGCGAGCGAGCGCGCATAGAGGGAGTGGCCAACTCCATC |
| | ACTAGGGGTACCG |
| 3′-ITR2 (GenBank | AGGAACCCCTAGTGATGGAGTTGGCCACTCCCTCTCTGCGCGCT |
| NC_001401.2) (SEQ | CGCTCGCTCACTGAGGCCGGGCGACCAAAGGTCGCCCGACGCCC |
| ID NO: 189) | GGGCTTTGCCCGGGCGGCCTCAGTGAGCGAGCGAGCGCGCAGAG |
| | AGGGAGTGGCCAA |

### Signal Peptides

In certain embodiments, the vectors provided herein comprise components that modulate protein delivery. In certain embodiments, the viral vectors provided herein comprise one or more signal peptides (also referred to herein as “signal sequences”). Signal peptides are also referred to herein as “leader sequences” or “leader peptides.” In certain embodiments, the signal peptides allow for the transgene product to achieve the proper packaging (e.g., glycosylation) in the cell. In certain embodiments, the signal peptides allow for the transgene product to achieve the proper localization in the cell. In certain embodiments, the signal peptides allow for the transgene product to achieve secretion from the cell. Secretory signal peptides (SSPs) are sequence motifs targeting proteins for translocation across the endoplasmic reticulum membrane and secretion into the extracellular environment.

There are two general approaches to select a signal peptide for protein production in a gene therapy context or in cell culture. One approach is to use a signal peptide from proteins homologous to the protein being expressed. For example, a human antibody signal peptide may be used to express IgGs in human cells, but may also express in other mammalian cells such as CHO cells. Another approach is to identify signal peptides that work best for the particular host cells used for expression. Signal peptides may be interchanged between different proteins or even between proteins of different organisms, and in some instances, the signal peptides of the most abundant secreted proteins of that cell type are used for protein expression.

For example, the signal peptide of human albumin, the most abundant protein in plasma, was found to substantially increase protein production yield in CHO cells. However, certain signal peptides may retain function and exert activity after being cleaved from the expressed protein as “post-targeting functions.” Thus, in specific embodiments, the signal peptide is selected from signal peptides of the most abundant proteins secreted by the cells used for expression to avoid the post-targeting functions.

A representative sequence is MYRMQLLLLIALSLALVTNS (SEQ ID NO: 171), which can be encoded by a nucleotide sequence of SEQ ID NO: 116 or 117. In some embodiments, the signal peptide is a heterologous signal peptide that controls the secretion of the protein encoded by the transgene (e.g., DN-TNFα). A heterologous signal sequence is one that is connected to a protein that is not naturally present or not its cognate protein in a given organism. “Secretion” includes release of the protein into the extracellular space, such as through the endoplasmic reticulum (Owji, H, et al European Journal of Cell Biology 97 (2018) 422-441; Zhang, L., et al. J Gene Med 2005; 7:354-365). In some embodiments, the signal sequence is not a native signal sequence. In some embodiments, the signal sequence is not a native tumor necrosis factor alpha (TNF) signal sequence. In some embodiments, the signal sequence does not comprise a TNF-alpha-converting enzyme (TACE) recognition sequence. In some embodiments, the signal sequence is a secretory signal sequence targeting protein for translocation across the endoplasmic reticulum membrane. Alternatively, signal sequences that are appropriate for expression, and may cause selective expression or directed expression and secretion of DN-TNFα in CNS, eye, muscle, or liver cells are provided in Tables 12, 13 and 14, respectively, below.

TABLE 12
Representative Signal Peptides for
Expression in Eye/CNS Tissue

| | SEQ | |
|---|---|---|
| Signal Peptide | ID | |
| Origin | NO: | Sequence |
| Mutant interleukin 2 | 115 | MYRMQLLLLIALSLALVINS |
| signal peptide | | |
| Mutant interleukin 2 | 116 | ATGTATAGGATGCAACTGCTCC |
| signal peptide coding | | TCCTGATTGCTCTGAGCCTGGC |
| sequence (1) | | TCTTGTGACCAACTCT |
| Mutant interleukin 2 | 117 | ATGTATAGAATGCAGCTGCTGC |
| signal peptide coding | | TGCTGATTGCTCTGTCCCTGGC |
| sequence (2) | | TCTGGTGACCAACTCT |
| VEGF-A signal | 118 | MNFLLSWVHWSLALLLYLHHAK |
| peptide | | WSQA |
| Fibulin-1 signal | 119 | MERAAPSRRVPLPLLLLGGLAL |
| peptide | | LAAGVDA |
| Vitronectin signal | 120 | MAPLRPLLILALLAWVALA |
| peptide | | |
| Complement Factor H | 121 | MRLLAKIICLMLWAICVA |
| signal peptide | | |
| Opticin signal | 122 | MRLLAFLSLLALVLQETGT |
| peptide | | |
| Albumin signal | 123 | MKWVTFISLLFLFSSAYS |
| peptide | | |
| Chymotrypsinogen | 124 | MAFLWLLSCWALLGTTFG |
| signal peptide | | |
| Interleukin-2 signal | 125 | MYRMQLLSCIALILALVINS |
| peptide | | |
| Trypsinogen-2 signal | 126 | MNLLLILTFVAAAVA |
| peptide | | |

TABLE 13
Representative Signal Peptides for
Expression in Muscle Cells

| | SEQ | |
|---|---|---|
| Signal Peptide | ID | |
| Origin | NO: | Sequence |
| Human SPARC | 127 | MRAWIFFLLCLAGRALA |
| Human Collagen | 128 | MFSFVDLRLLLLLAATALLTHG |
| alpha-1(I) chain | | |
| Human | 129 | MKLVFLVLLFLGALGLCLA |
| Lactotransferrin | | |
| Human Complement | 130 | MGPTSGPSLLLLLLTHLPLALG |
| C3 | | |
| Human Lumican | 131 | MSLSAFTLFLALIGGTSG |
| Human Gelsolin | 132 | MAPHRPAPALLCALSLALCALS |
| isoform 1 | | LPVRA |
| Human Pro- | 133 | MWATLPLLCAGAWLLGVPVCGA |
| cathepsin H | | |
| Human SERPINF1 | 134 | MQALVLLLCIGALLGHSSC |
| Human SERPINE1 | 135 | MQMSPALTCLVLGLALVFGEGS |
| | | A |
| Human Cathepsin D | 136 | MQPSSLLPLALCLLAAPASA |
| Human TIMP1 | 137 | MAPFEPLASGILLLLWLIAPSR |
| | | A |
| Human Fibronectin | 138 | MLRGPGPGLLLLAVQCLGTAVP |
| | | STGASKSKR |
| Human Complement | 139 | MWCIVLFSLLAWVYA |
| C1s subcomponent | | |
| Human Cathepsin L1 | 140 | MNPTLILAAFCLGIASA |
| Human Cathepsin B | 141 | MWQLWASLCCLLVLANA |
| Human Salivary | 142 | MLLILLSVALLAFSSA |
| acidic proline- | | |
| rich phospho- | | |
| protein ½ | | |
| Human Follistatin- | 143 | MWKRWLALALALVAVAWVRA |
| related protein 1 | | |

TABLE 14
Representative Signal Peptides for
Expression in Liver Cells

| | SEQ | |
|---|---|---|
| Signal Peptide | ID | |
| Origin | NO: | Sequence |
| Human Serum | 144 | MKWVTFISLLFLFSSAYS |
| albumin | | |
| Human α-1 | 145 | MPSSVSWGILLLAGLCCLVPVSLA |
| Antitrypsin | | |
| (SERPINA1) | | |
| Human Apoli- | 146 | MKAAVLTLAVLFLTGSQA |
| poprotein A-1 | | |
| Human Apoli- | 147 | MKLLAATVLLLTICSLEG |
| poprotein A-2 | | |
| Human Apoli- | 148 | MDPPRPALLALLALPALLLLLLAGAR |
| poprotein B-100 | | A |
| Human Coagulation | 149 | MQRVNMIMAESPGLITICLLGYLLSA |
| Factor IX | | EC |
| Human Complement | 150 | MGPLMVLFCLLFLYPGLADS |
| C2 | | |
| Human Complement | 151 | MWLLVSVILISRISSVGG |
| Factor H-related | | |
| Protein 2 (CFHR2) | | |
| Human Complement | 152 | MLLLFSVILISWVSTVGG |
| Factor H-related | | |
| Protein 5 (CFHR5) | | |
| Human Fibrinogen | 153 | MFSMRIVCLVLSVVGTAWT |
| α-chain (FGA) | | |
| Human Fibrinogen | 154 | MKRMVSWSFHKLKTMKHLLLLLLCVF |
| β-chain (FGB) | | LVKS |
| Human Fibrinogen | 155 | MSWSLHPRNLILYFYALLFLSSTCVA |
| γ-chain (FGG) | | |
| Human α-2-HS- | 156 | MKSLVLLLCLAQLWGCHS |
| Glycoprotein | | |
| (AHSG) | | |
| Human Hemopexin | 157 | MARVLGAPVALGLWSLCWSLAIA |
| (HPX) | | |
| Human Kininogen-1 | 158 | MKLITILFLCSRLLLSLT |
| Human Mannose- | 159 | MSLFPSLPLLLLSMVAASYS |
| binding protein C | | |
| (MBL2) | | |
| Human Plasminogen | 160 | MEHKEVVLLLLLFLKSGQG |
| (PLMN) | | |
| Human Prothrombin | 161 | MAHVRGLOLPGCLALAALCSLVHS |
| (Coagulation | | |
| Factor II) | | |
| Human Secreted | 162 | MISRMEKMTMMMKILIMFALGMNYWS |
| Phosphoprotein 24 | | CSG |
| Human Anti- | 163 | MYSNVIGTVTSGKRKVYLLSLLLIGF |
| thrombin-III | | WDCVTC |
| (SERPINC1) | | |
| Human Serotrans- | 164 | MRLAVGALLVCAVLGLCLA |
| ferrin (TF) | | |

Methods for Producing and Isolating rAAV Particles

The viral vectors provided herein can be manufactured using host cells. The viral vectors provided herein may be manufactured using mammalian host cells, for example, A549, WEHI, 10T1/2, BHK, MDCK, COSI, COS7, BSC 1, BSC 40, BMT 10, VERO, W138, HeLa, HEK293, Saos, C2C12, L, HT1080, HepG2, primary fibroblast, hepatocyte, and myoblast cells. The viral vectors provided herein can be manufactured using host cells from human, monkey, mouse, rat, rabbit, or hamster.

The host cells are stably transformed with the sequences encoding the transgene and associated elements (e.g., the vector genome), and the means of producing viruses in the host cells, for example, the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation.

Alternatively, baculovirus expression systems in insect cells can be used to produce AAV vectors. For a review, see Aponte-Ubillus J J et al., 2018, Appl. Microbiol. Biotechnol. 102:1045-1054 which is incorporated by reference herein in its entirety for manufacturing techniques.

In vitro assays, e.g., cell culture assays, can be used to measure transgene expression from a vector described herein, thus indicating, e.g., potency of the vector. For example, the SH-SY5Y human neuroblastoma cell line (ATCC CRL-2266), or a cell line derived from human retinal pigment epithelial (RPE) cells, e.g., the cell line hTERT RPE-1 (available from ATCC®), can be used to assess transgene expression. Once expressed in a cell line, expressed product can be isolated and characteristics of the expressed product can be determined. Expressed product may also be expressed ex vivo or in vivo, isolated from cells or tissues and characteristics of the expressed product, such as protein structure or function, can be determined by various assays as described elsewhere in this disclosure.

In some embodiments, the disclosure provides methods for producing a composition comprising isolated rAAV particles, comprising isolating rAAV particles from a feed comprising an impurity (for example, rAAV production culture). In some embodiments, a method for producing a formulation comprising isolated recombinant adeno-associated virus (rAAV) particles disclosed herein comprises (a) isolating rAAV particles from a feed comprising an impurity (for example, rAAV production culture), and (b) formulating the isolated rAAV particles to produce the formulation.

In some embodiments, the disclosure further provides methods for producing a pharmaceutical unit dosage of a formulation comprising isolated recombinant adeno-associated virus (rAAV) particles, comprising isolating rAAV particles from a feed comprising an impurity (for example, rAAV production culture), and formulating the isolated rAAV particles.

Isolated rAAV particles can be isolated using methods known in the art. In some embodiments, methods of isolating rAAV particles comprises downstream processing such as, for example, harvest of a cell culture, clarification of the harvested cell culture (e.g., by centrifugation or depth filtration), tangential flow filtration, affinity chromatography, anion exchange chromatography, cation exchange chromatography, size exclusion chromatography, hydrophobic interaction chromatography, hydroxyapatite chromatography, sterile filtration, or any combination(s) thereof. In some embodiments, downstream processing includes at least 2, at least 3, at least 4, at least 5, or at least 6 of: harvest of a cell culture, clarification of the harvested cell culture (e.g., by centrifugation or depth filtration), tangential flow filtration, affinity chromatography, anion exchange chromatography, cation exchange chromatography, size exclusion chromatography, hydrophobic interaction chromatography, hydroxyapatite chromatography, and sterile filtration. In some embodiments, downstream processing comprises harvest of a cell culture, clarification of the harvested cell culture (e.g., by depth filtration), sterile filtration, tangential flow filtration, affinity chromatography, and anion exchange chromatography.

In some embodiments, downstream processing comprises clarification of a harvested cell culture, sterile filtration, tangential flow filtration, affinity chromatography, and anion exchange chromatography. In some embodiments, downstream processing comprises clarification of a harvested cell culture by depth filtration, sterile filtration, tangential flow filtration, affinity chromatography, and anion exchange chromatography. In some embodiments, clarification of the harvested cell culture comprises sterile filtration. In some embodiments, downstream processing does not include centrifugation. In certain embodiments, the rAAV particles comprise a capsid protein of the AAV8 serotype. In some embodiments, the rAAV particles comprise a capsid protein of the AAV9 serotype.

In some embodiments, a method of isolating rAAV particles produced according to a method disclosed herein comprises harvest of a cell culture, clarification of the harvested cell culture (e.g., by depth filtration), a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles disclosed herein comprises harvest of a cell culture, clarification of the harvested cell culture (e.g., by depth filtration), a first sterile filtration, affinity chromatography, anion exchange chromatography (e.g., monolith anion exchange chromatography or AEX chromatography using a quaternary amine ligand), a tangential flow filtration, and a second sterile filtration. In some embodiments, a method of isolating rAAV particles produced according to a method disclosed herein comprises clarification of a harvested cell culture, a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles disclosed herein comprises clarification of a harvested cell culture, a first sterile filtration, affinity chromatography, anion exchange chromatography (e.g., monolith anion exchange chromatography or AEX chromatography using a quaternary amine ligand), tangential flow filtration, and a second sterile filtration.

In some embodiments, a method of isolating rAAV particles produced according to a method disclosed herein comprises clarification of a harvested cell culture by depth filtration, a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles disclosed herein comprises clarification of a harvested cell culture by depth filtration, a first sterile filtration, affinity chromatography, anion exchange chromatography (e.g., monolith anion exchange chromatography or AEX chromatography using a quaternary amine ligand), tangential flow filtration, and a second sterile filtration. In some embodiments, the method does not include centrifugation. In certain embodiments, clarification of the harvested cell culture comprises sterile filtration. In some embodiments, the rAAV particles comprise a capsid protein of the AAV8 serotype. In some embodiments, the rAAV particles comprise a capsid protein of the AAV9 serotype.

Numerous methods are known in the art for producing rAAV particles, including transfection, stable cell line production, and infectious hybrid virus production systems which include adenovirus-AAV hybrids, herpesvirus-AAV hybrids and baculovirus-AAV hybrids. rAAV production cultures for the production of rAAV virus particles all require: (1) suitable host cells, including, for example, human-derived cell lines such as HeLa, A549, or HEK293 cells and their derivatives (HEK293T cells, HEK293F cells), mammalian cell lines such as Vero, or insect-derived cell lines such as SF-9 in the case of baculovirus production systems; (2) suitable helper virus function, provided by wild type or mutant adenovirus (such as temperature sensitive adenovirus), herpes virus, baculovirus, or a plasmid construct providing helper functions; (3) AAV rep and cap genes and gene products; (4) a transgene (such as a therapeutic transgene) flanked by AAV ITR sequences; and (5) suitable media and media components to support rAAV production. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), and Sf-900 II SFM media as described in U.S. Pat. No. 6,723,551, which is incorporated herein by reference in its entirety.

rAAV production cultures can routinely be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, RK cells, Per C6 cells, chicken embryo cells, or SF-9 cells, which can be cultured in a variety of ways including, for example, spinner flasks, stirred tank bioreactors, and disposable systems such as the Wave bag system. In some embodiments, the cells are HEK293 cells. In some embodiments, the cells are HEK293 cells adapted for growth in suspension culture. Numerous suspension cultures are known in the art for production of rAAV particles, including for example, the cultures disclosed in U.S. Pat. Nos. 6,995,006, 9,783,826, and in U.S. Pat. Appl. Pub. No. 20120122155, each of which is incorporated herein by reference in its entirety.

In some embodiments, the rAAV production culture comprises a high density cell culture. In some embodiments, the culture has a total cell density of between about 1×10E+06 cells/ml and about 30×10E+06 cells/ml. In certain embodiments, more than about 50% of the cells are viable cells. In some embodiments, the cells are HeLa cells, HEK293 cells, HEK293 derived cells (e.g., HEK293T cells, HEK293F cells), Vero cells, or SF-9 cells. In further embodiments, the cells are HEK293 cells. In further embodiments, the cells are HEK293 cells adapted for growth in suspension culture.

In additional embodiments of the provided methods, the rAAV production culture comprise a suspension culture comprising rAAV particles. Numerous suspension cultures are known in the art for production of rAAV particles, including for example, the cultures disclosed in U.S. Pat. Nos. 6,995,006, 9,783,826, and in U.S. Pat. Appl. Pub. No. 20120122155, each of which is incorporated herein by reference in its entirety. In some embodiments, the suspension culture comprises a culture of mammalian cells or insect cells. In some embodiments, the suspension culture comprises a culture of Hela cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, RK cells, Per.C6 cells, chicken embryo cells or SF-9 cells. In some embodiments, the suspension culture comprises a culture of HEK293 cells.

In some embodiments, methods for the production of rAAV particles encompasses providing a cell culture comprising a cell capable of producing rAAV; adding to the cell culture a histone deacetylase (HDAC) inhibitor to a final concentration between about 0.1 mM and about 20 mM; and maintaining the cell culture under conditions that allows production of the rAAV particles. In some embodiments, the HDAC inhibitor comprises a short-chain fatty acid or salt thereof. In some embodiments, the HDAC inhibitor comprises butyrate (e.g., sodium butyrate), valproate (e.g., sodium valproate), propionate (e.g., sodium propionate), or a combination thereof.

In some embodiments, rAAV particles are produced as disclosed in WO 2020/033842, which is incorporated herein by reference in its entirety.

Recombinant AAV particles can be harvested from rAAV production cultures by harvest of the production culture comprising host cells or by harvest of the spent media from the production culture, provided the cells are cultured under conditions known in the art to cause release of rAAV particles into the media from intact host cells. Recombinant AAV particles can also be harvested from rAAV production cultures by lysis of the host cells of the production culture. Suitable methods of lysing cells are also known in the art and include for example multiple freeze/thaw cycles, sonication, microfluidization, and treatment with chemicals, such as detergents and/or proteases.

At harvest, rAAV production cultures can contain one or more of the following: (1) host cell proteins; (2) host cell DNA; (3) plasmid DNA; (4) helper virus; (5) helper virus proteins; (6) helper virus DNA; and (7) media components including, for example, serum proteins, amino acids, transferrins, and other low molecular weight proteins. rAAV production cultures can further contain product-related impurities, for example, inactive vector forms, empty viral capsids, aggregated viral particles or capsids, mis-folded viral capsids, degraded viral particle. In some embodiments, the rAAV production culture harvest is clarified to remove host cell debris. In certain embodiments, the production culture harvest is clarified by filtration through a series of depth filters. Clarification can also be achieved by a variety of other standard techniques known in the art, such as, centrifugation or filtration through any cellulose acetate filter of 0.2 mm or greater pore size known in the art. In some embodiments, clarification of the harvested cell culture comprises sterile filtration. In some embodiments, the production culture harvest is clarified by centrifugation. In certain embodiments, clarification of the production culture harvest does not included centrifugation.

In some embodiments, harvested cell culture is clarified using filtration. In some embodiments, clarification of the harvested cell culture comprises depth filtration. In certain embodiments, clarification of the harvested cell culture further comprises depth filtration and sterile filtration. In some embodiments, harvested cell culture is clarified using a filter train comprising one or more different filtration media. In some embodiments, the filter train comprises a depth filtration media. In some embodiments, the filter train comprises one or more depth filtration media. In some embodiments, the filter train comprises two depth filtration media. In some embodiments, the filter train comprises a sterile filtration media. In certain embodiments, the filter train comprises two depth filtration media and a sterile filtration media. In some embodiments, the depth filter media is a porous depth filter. In some embodiments, the filter train comprises Clarisolve® 20 MS, Millistak+® COHC, and a sterilizing grade filter media. In some embodiments, the filter train comprises Clarisolve® 20 MS, Millistak+® COHC, and Sartopore® 2 XLG 0.2 μm. In some embodiments, the harvested cell culture is pretreated before contacting it with the depth filter. In certain embodiments, the pretreating comprises adding a salt to the harvested cell culture. In some embodiments, the pretreating comprises adding a chemical flocculent to the harvested cell culture. In some embodiments, the harvested cell culture is not pre-treated before contacting it with the depth filter.

In some embodiments, the production culture harvest is clarified by filtration are disclosed in WO 2019/212921, which is incorporated herein by reference in its entirety. In some embodiments, the rAAV production culture harvest is treated with a nuclease (e.g., Benzonase®) or endonuclease (e.g., endonuclease from Serratia marcescens) to digest high molecular weight DNA present in the production culture. The nuclease or endonuclease digestion can routinely be performed under standard conditions known in the art. For example, nuclease digestion is performed at a final concentration of 1-2.5 units/ml of Benzonase® at a temperature ranging from ambient to 37° C. for a period of 30 minutes to several hours.

Sterile filtration encompasses filtration using a sterilizing grade filter media. In some embodiments, the sterilizing grade filter media is a 0.2 or 0.22 μm pore filter. In some embodiments, the sterilizing grade filter media comprises polyethersulfone (PES). In some embodiments, the sterilizing grade filter media comprises polyvinylidene fluoride (PVDF). In some embodiments, the sterilizing grade filter media has a hydrophilic heterogeneous double layer design. In some embodiments, the sterilizing grade filter media has a hydrophilic heterogeneous double layer design of a 0.8 μm pre-filter and 0.2 μm final filter membrane. In some embodiments, the sterilizing grade filter media has a hydrophilic heterogeneous double layer design of a 1.2 μm pre-filter and 0.2 μm final filter membrane. In some embodiments, the sterilizing grade filter media is a 0.2 or 0.22 μm pore filter. In further embodiments, the sterilizing grade filter media is a 0.2 μm pore filter. In some embodiments, the sterilizing grade filter media is a Sartopore® 2 XLG 0.2 μm, Durapore™ PVDF Membranes 0.45 μm, or Sartoguard® PES 1.2 μm+0.2 μm nominal pore size combination. In some embodiments, the sterilizing grade filter media is a Sartopore® 2 XLG 0.2 μm.

In some embodiments, the clarified feed is concentrated via tangential flow filtration (“TFF”) before being applied to a chromatographic medium, for example, affinity chromatography medium. Large scale concentration of viruses using TFF ultrafiltration has been described by Paul R W et al., Human Gene Therapy 4:609-615 (1993). TFF concentration of the clarified feed enables a technically manageable volume of clarified feed to be subjected to chromatography and allows for more reasonable sizing of columns without the need for lengthy recirculation times. One of ordinary skill in the art will also recognize that TFF can also be used to remove small molecule impurities (e.g., cell culture contaminants comprising media components, serum albumin, or other serum proteins) form the clarified feed via diafiltration. In some embodiments, the clarified feed is subjected to diafiltration to remove small molecule impurities. In some embodiments, the diafiltration comprises the use of between about 3 and about 10 diafiltration volume of buffer. In certain embodiments, the diafiltration comprises the use of about 5 diafiltration volume of buffer. One of ordinary skill in the art will also recognize that TFF can also be used at any step in the purification process where it is desirable to exchange buffers before performing the next step in the purification process. In some embodiments, the methods for isolating rAAV from the clarified feed disclosed herein comprise the use of TFF to exchange buffers.

Affinity chromatography can be used to isolate rAAV particles from a composition. In some embodiments, affinity chromatography is used to isolate rAAV particles from the clarified feed. In some embodiments, affinity chromatography is used to isolate rAAV particles from the clarified feed that has been subjected to tangential flow filtration. Suitable affinity chromatography media are known in the art and include without limitation, AVB Sepharose™ POROS™ CaptureSelect™ AAVX affinity resin, POROS™ CaptureSelect™ AAV9 affinity resin, and POROS™ CaptureSelect™ AAV8 affinity resin. In certain embodiments, the affinity chromatography media is POROS™ CaptureSelect™ AAV9 affinity resin. In some embodiments, the affinity chromatography media is POROS™ CaptureSelect™ AAV8 affinity resin. In some embodiments, the affinity chromatography media is POROS™ CaptureSelect™ AAVX affinity resin.

Anion exchange chromatography can be used to isolate rAAV particles from a composition. In some embodiments, anion exchange chromatography is used after affinity chromatography as a final concentration and polish step. Suitable anion exchange chromatography media are known in the art and include without limitation, UNOsphere™ Q (Biorad, Hercules, Calif.), and N-charged amino or imino resins such as e.g., POROS™ 50 PI, or any DEAE, TMAE, tertiary or quaternary amine, or PEI-based resins known in the art (U.S. Pat. No. 6,989,264; Brument N. et al., Mol. Therapy 6 (5): 678-686 (2002); Gao G et al., Hum. Gene Therapy 11:2079-2091 (2000)). In certain embodiments, the anion exchange chromatography media comprises a quaternary amine. In some embodiments, the anion exchange media is a monolith anion exchange chromatography resin. In some embodiments, the monolith anion exchange chromatography media comprises glycidylmethacrylate-ethylenedimethacrylate or styrene-divinylbenzene polymers.

In some embodiments, the monolith anion exchange chromatography media is selected from the group consisting of CIMmultus™ QA-1 Advanced Composite Column (Quaternary amine), CIMmultus™ DEAE-1 Advanced Composite Column (Diethylamino), CIM® QA Disk (Quaternary amine), CIM® DEAE, and CIM® EDA Disk (Ethylene diamino). In some embodiments, the monolith anion exchange chromatography media is CIMmultus™ QA-1 Advanced Composite Column (Quaternary amine). In some embodiments, the monolith anion exchange chromatography media is CIM® QA Disk (Quaternary amine). In some embodiments, the anion exchange chromatography media is CIM QA (BIA Separations, Slovenia). In certain embodiments, the anion exchange chromatography media is BIA CIM® QA-80 (Column volume is 80 mL). One of ordinary skill in the art can appreciate that wash buffers of suitable ionic strength can be identified such that the rAAV remains bound to the resin while impurities, including without limitation impurities which may be introduced by upstream purification steps are stripped away.

In some embodiments, anion exchange chromatography is performed according to a method disclosed in WO 2019/241535, which is incorporated herein by reference in its entirety.

In cewrtain embodiments, a method of isolating rAAV particles comprises determining the vector genome titer, capsid titer, and/or the ratio of full to empty capsids in a composition comprising the isolated rAAV particles. In some embodiments, the vector genome titer is determined by quantitative PCR (qPCR) or digital PCR (dPCR) or droplet digital PCR (ddPCR) In some embodiments, the capsid titer is determined by serotype-specific ELISA. In certain embodiments, the ratio of full to empty capsids is determined by Analytical Ultracentrifugation (AUC) or Transmission Electron Microscopy (TEM).

In some embodiments, the vector genome titer, capsid titer, and/or the ratio of full to empty capsids is determined by spectrophotometry, for example, by measuring the absorbance of the composition at 260 nm; and measuring the absorbance of the composition at 280 nm. In certain embodiments, the rAAV particles are not denatured prior to measuring the absorbance of the composition. In some embodiments, the rAAV particles are denatured prior to measuring the absorbance of the composition. In some embodiments, the absorbance of the composition at 260 nm and 280 nm is determined using a spectrophotometer. In some embodiments, the absorbance of the composition at 260 nm and 280 nm is determined using a HPLC. In certain embodiments, the absorbance is peak absorbance. Several methods for measuring the absorbance of a composition at 260 nm and 280 nm are known in the art. Methods of determining vector genome titer and capsid titer of a composition comprising the isolated recombinant rAAV particles are disclosed in WO 2019/212922, which is incorporated herein by reference in its entirety.

In additional embodiments the disclosure provides compositions comprising isolated rAAV particles produced according to a method disclosed herein. In some embodiment, the composition is a pharmaceutical composition comprising a pharmaceutically acceptable carrier.

As used herein the term “pharmaceutically acceptable” means a biologically acceptable formulation, gaseous, liquid or solid, or mixture thereof, which is suitable for one or more routes of administration, in vivo delivery or contact. A “pharmaceutically acceptable” composition is a material that is not biologically or otherwise undesirable, e.g., the material may be administered to a subject without causing substantial undesirable biological effects. Thus, such a pharmaceutical composition may be used, for example in administering rAAV isolated according to the disclosed methods to a subject. Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes. Pharmaceutical compositions and delivery systems appropriate for rAAV particles and methods and uses of the invention are known in the art (see, e.g., Remington: The Science and Practice of Pharmacy (2003) 20th ed., Mack Publishing Co., Easton, Pa.; Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11th ed., Lippincott Williams & Wilkins, Baltimore, Md.; and Poznansky et al., Drug Delivery Systems (1980), R. L. Juliano, ed., Oxford, N.Y., pp. 253-315).

In some embodiments, the composition is a pharmaceutical unit dose. A “unit dose” refers to a physically discrete unit suited as a unitary dosage for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dose forms may be within, for example, ampules and vials, which can include a liquid composition, or a composition in a freeze-dried or lyophilized state; a sterile liquid carrier, for example, can be added prior to administration or delivery in vivo. Individual unit dose forms can be included in multi-dose kits or containers.

Recombinant vector (e.g., AAV) sequences, plasmids, vector genomes, and recombinant virus particles, and pharmaceutical compositions thereof can be packaged in single or multiple unit dose form for ease of administration and uniformity of dosage. In some embodiments, the composition comprises rAAV particles comprising an AAV capsid protein from an AAV capsid serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the AAV capsid serotype is AAV8. In some embodiments, the AAV capsid serotype is AAV9.

### Methods for Isolating and Characterizing DN-TNFα

The DN-TNFα produced by the methods of the disclosure product may be expressed ex vivo or in vivo, isolated from cells or tissues and characteristics of the expressed product, such as protein structure or function, can be determined by various assays. A recombinant DN-TNFα protein of the disclosure is made using recombinant techniques, i.e. through the expression of a recombinant nucleic acid described elsewhere in this application. The recombinant protein is distinguished from naturally occurring protein by at least one or more characteristics. In one embodiment, a recombinant nucleotide expression vector comprising a DN-TNFα transgene, operably linked to a secretory signal peptide and one or more regulatory sequences is transduced into a target human cell or tissue so that a depot is formed that releases a human post-translationally modified (HuP™) form of the expressed DN-TNFα. The production of HuP™ DN-TNFα by such human cells should result in a “biobetter” molecule for the treatment of disease accomplished via gene therapy—e.g., by administering a viral vector or other DNA expression construct encoding a full-length HuP™ DN-TNFα to a patient (human subject) diagnosed with a disease indication, to create a permanent depot in the subject that continuously supplies the human-glycosylated and/or sulfated transgene product produced by the subject's transduced cells. The cDNA construct for the HuP™ DN-TNFα includes a signal peptide that ensures proper co- and post-translational processing (glycosylation and protein sulfation) by the transduced human cells by transporting the expressed protein to the endoplasmic reticulum. Furthermore, the DN-TNFα protein outlined herein is in a form not normally found in nature, as it contains amino acid substitutions. In particular, the DN-TNFα protein of the disclosure has two substitutions at amino acid residue 87 and 145 (Y87H/A145R) as compared to wild type TNFα protein (SEQ ID NO: 1) and are dominant negative TNFα (DN-TNFα) proteins. The DN-TNFα protein of the disclosure mimics soluble TNF (sTNF) by displacing one or more sTNF molecules and forming trimeric clusters (heterotrimers 1:2 or 2:1 TNF:DN-TNF, or homotrimers consisting of three DN-TNF molecules). The trimers disrupt TNFR1 signaling that normally activate inflammatory pathways. However prosurvival and homeostatic TNFR2 signaling remains intact in the presence of DN-TNF-containing trimers. Thus, the DN-TNFα protein of the disclosure is an antagonist of wild type TNFα, in that it inhibits or significantly decreases at least one biological activity of wild-type TNFα, but it does not significantly antagonize transmembrane TNFα. The DN-TNFα protein inhibits signaling by soluble TNFα, but not transmembrane TNFα. By “inhibits the activity of TNFα” and grammatical equivalents is meant at least a 10%, at least a 20%, at least a 30%, at least a 40%, at least a 50%, at least a 60%, at least a 70%, at least a 80%, or at least a 90% reduction in wild-type, soluble TNFα activity. In some embodiments, there is an inhibition in wild-type soluble TNFα activity in the absence of reduced signaling by transmembrane TNFα. For example, the activity of soluble TNFα is inhibited while the activity of transmembrane TNFα is substantially completely maintained.

The DN-TNFα protein of the disclosure has modulated activity as compared to wild type TNFα protein. In one embodiment, DN-TNFα proteins exhibit decreased biological activity (e.g. antagonism) as compared to wild type TNFα, including, but not limited to, decreased binding to a receptor (TNFR1, TNFR2 or both), decreased activation and/or ultimately a loss of cytotoxic activity. “Cytotoxic activity” herein refers to the ability of DN-TNFα to selectively kill or inhibit cells. N some embodiments, DN-TNFα proteins exhibit less than 50% biological activity as compared to wild type TNFα protein. For example, the DN-TNFα protein can exhibit less than about 30%, (e.g., less than 25%, less than 20%, or less than 10%) of a biological activity of wild-type TNFα. Suitable assays include, but are not limited to, caspase assays, TNFα cytotoxicity assays, DNA binding assays, transcription assays (using reporter constructs), size exclusion chromatography assays and radiolabeling/immuno-precipitation, and stability assays (including the use of circular dichroism (CD) assays and equilibrium studies), according to methods know in the art.

In one embodiment, at least one property critical for binding affinity of the DN-TNFα protein is altered when compared to the same property of wild type TNFα and in particular, the DN-TNFα protein has an altered receptor affinity. The DN-TNFα protein has altered affinity toward oligomerization to wild type TNFα. Thus, the disclosure provides a DN-TNFα protein with an altered binding affinity such that the DN-TNFα protein will preferentially oligomerize with wild type TNFα, but does not substantially interact with wildtype TNF receptors, e.g., TNFR1, TNFR2. “Preferentially” in this case means that given equal amounts of DN-TNFα monomers and wild type TNFα monomers, at least about 25% (e.g., at least 30%, at least 40%, at least 50%, at least 60%, at least 70%, or at least 80%, or at least 90%) of the resulting trimers are mixed trimers of DN-TNFα and wild type TNFα. “Do not substantially interact with TNF receptors” means that the DN-TNFα protein will not be able to associate with either the p55 or p75 receptors to significantly activate the receptor and initiate the TNF signaling pathway(s). In some embodiments, at least a 50% decrease in receptor activation is seen.

In some embodiments, the DN-TNFα protein selectively inhibits soluble TNFα but not transmembrane TNFα. In some embodiments, the biological activity of the heterotrimers containing one or more DN-TNFα proteins of the disclosure reduces by at least 10% (e.g., at least 20%, at least 30%, at least 40%, at least 50%, at least 60%, or at least 70%, or at least 80%). In these embodiments, at least about 80%, (e.g., 85%, 90%, 95%, 98%, 99% or 100%) of the transmembrane TNFα activity is maintained. The DN-TNFα protein of the disclosure substantially inhibits or eliminates soluble TNFα activity (for example by exchanging with homotrimeric wild-type TNFα to form heterotrimers that do not bind to TNFα receptors or that bind but do not activate receptor signaling) but does not significantly affect or does not alter transmembrane TNFα activity.

Without being bound by theory, the DN-TNFα protein of the disclosure exhibiting such differential inhibition allows the decrease of inflammation or when in the context of the appropriate disorder, ameliorates or treats a pathological condition disclosed herein (e.g., a neuroinflammatory condition, an ocular condition, or a systemic autoimmune condition).

In one embodiment, the affected biological activity of the DN-TNFα proteins is the activation of receptor signaling by wild type TNFα proteins. In one example, the DN-TNFα proteins interact with the wild type TNFα protein such that the complex comprising the DN-TNFα proteins and wild type TNFα has reduced capacity to activate (as outlined above for “substantial inhibition”), and in some embodiments, is incapable of activating, one or both of the TNF receptors, e.g., TNFR1 or TNFR2. In some cases, the DN-TNFα protein functions as an antagonist of wild type TNFα. In some embodiments, the DN-TNFα protein interacts with wild type TNFα to form mixed trimers with the wild type protein, such that receptor binding does not significantly occur and/or TNFα signaling is not initiated. In such mixed trimers, monomers of wild type and variant TNFα proteins interact to form heterotrimeric TNFα, as well as homotrimeric clusters of three DN-TNF. Mixed trimers (heterotrimers) may comprise 1 DN-TNFα protein: 2 wildtype TNFα proteins and/or 2 DN-TNFα proteins: 1 wild type TNFα protein. In some embodiments, trimers may be formed comprising only DN-TNFα proteins (DN-TNF homotrimers).

The DN-TNFα antagonist proteins of the disclosure are highly specific for TNFα antagonism relative to TNF-beta antagonism. Additional characteristics include improved stability, pharmacokinetics, and high affinity for wild type TNFα. DN-TNFα proteins, for example are experimentally tested and validated in in vivo and in in vitro assays. Suitable assays include, but are not limited to, activity assays and binding assays. For example, TNFα activity assays, such as detecting apoptosis via caspase activity can be used to determine antagonistic activity levels of the DN-TNFα protein of this disclosure. Other assays include using the Sytox green nucleic acid stain to detect TNF-induced cell permeability in an Actinomycin-D sensitized cell line. An example of an NF kappaB assay is presented in Example 7 of U.S. Pat. No. 7,446,174, incorporated by reference herein in its entirety.

In some embodiments, the binding affinity of DN-TNFα proteins as compared to wild type TNFα proteins for naturally occurring TNFα and TNF receptor proteins such as p55 and p75 are determined. Suitable assays include, but are not limited to, e.g., quantitative comparisons comparing kinetic and equilibrium binding constants, as are known in the art. Examples of binding assays are described in Example 6 of U.S. Pat. No. 7,446,174, incorporated by reference herein in its entirety.

### Neuroinflammatory Conditions

The compositions and methods of this disclosure can be used to treat neuroinflammatory conditions, including, but not limited to, Alzheimer's Disease (AD), frontotemporal dementia (FD), tauopathies, progressive supranuclear palsy, chronic traumatic encephalopathy, Pick's Complex, and primary age-related tauopathy, Huntington's disease, juvenile Huntington's disease, Parkinson's disease, synucleinopathies, Amyotrophic lateral sclerosis (ALS), migraines, cluster headaches, as well as conditions with elevated TNFα, including but not limited to stroke, depression, post-traumatic stress disorder (PTSD) and traumatic brain injury (TBI).

Treatment efficacy in AD with the disclosed compositions and methods can be measured by a number of methods, including, but not limited to, measuring levels of Aβ accumulation and amyloid plaques and Tau neuroinflammatory markers in AD, measuring levels of white matter free water (WMFW); and levels of inflammatory markers including, but not limited to, levels of CCL8, OLR1, IL2, CXCL9, TGFA, IL6, TNFSF12, CCL11, HGF, FLT3LG, IL17F, IL7, IL18, CCL13, TNFSF10, CXCL10, IFNG, IL10, 1L15, CCL3, CXCL8, MMP12, CSF2, VEGFA, IL17C, CCL2, IL17A, OSM, CSF1, CCL4, CXCL11, LTA, CCL7, and MMP1. In some embodiments, treatment efficacy can be determined by tests that measure cognitive abilities and the level of microglial phagocytosis.

Measurement of the risk, existence, severity, and progression of a neuroinflammatory condition of this disclosure (e.g., AD) can be determined by clinical diagnosis over time; assessment of the global functional level of the patient; evaluation of the daily living capacities or behavioral deficits; volumetric analysis of brain structures; in vivo measurement of pathological deposits of abnormal proteins in brain (e.g. PET beta-amyloid imaging), and/or biochemical variables in body fluids (e.g. tau proteins or amyloid beta peptides); and by comparison to the natural course/history of the disease. In some embodiments, one or more of the following clinical assessments can be employed in determining the stage of Alzheimer's disease in the patient: Clinical Dementia Rating (CDR), the Free and Cued Selective Reminding Test (FCSRT), Neuropsychiatry Inventory-Questionnaire (NPI-Q), and a neuropsychological test battery comprising Rey Auditory Verbal Learning Test (RA VLT) Immediate and Delayed Recall, Wechsler Memory Scale (WMS) Verbal Pair Associate Learning Test Immediate and Delayed Recall, Delis-Kaplan Executive Function System Verbal Fluency Conditions 1 and 2, and the Wechsler Adult Intelligence Scale Fourth Edition Symbol Search and Coding Subsets; and the Cognitive Drug Research computerized test battery.

In some embodiments, biomarkers can be used for defining AD and for staging of the disease along its spectrum. Biomarkers of AD include, but are not limited to, ApoE isotype, amyloid PET, total Tau, phospho-Tau, pyroglutamate-Aβ, Aβ40, and Aβ42 in blood or CSF, and hippocampal volumetric (HCV) MRI.

The amyloid plaque burden in the brain can be measured by ¹⁸F-AV-45 PET. ¹⁸F-AV-45 is an amyloid ligand developed by Avid Radiopharmaceuticals (Philadelphia, Pennsylvania) that binds to fibrillar Aβ with a high affinity (Kd=3.1 nM). Results with ¹⁸F-AV-45 PET imaging have shown that patients with AD have selective retention of tracer in cortical areas expected to be high in amyloid deposition, whereas healthy controls have shown rapid washout from these areas, with only minimal cortical tracer retention. A significant difference in mean uptake of ¹⁸F-AV-45 has been observed between AD and age-matched control subjects. Test-retest variance of ¹⁸F-AV-45 PET imaging is low (less than 5%) in both AD patients and cognitively healthy controls. Visual interpretation of the ¹⁸F-AV-45 PET images and mean quantitative estimates of cortical uptake correlate with presence and quantity of amyloid pathology at autopsy as measured by immunohistochemistry and silver stain neuritic plaque score (Clark C M, et al., JAMA. 2011 Jan. 19; 305 (3): 275-83).

Morphometry MRI measures can also aid in the assessment of AD. These include whole brain volume, hippocampal volume, ventricle volume, and cortical gray matter volume. Cerebral blood flow as measured by ASL-MRI and functional connectivity as measured by tf-fMRI can be included in the assessment protocols. See, e.g., WO2016087944A2 for further description of PET imaging and other techniques associated with aiding in the assessment of AD. Cognitive function may also be measured using imaging techniques such as Positron Emission Tomography (PET), functional magnetic resonance imaging (fMRI), Single Photon Emission Computed Tomography (SPECT), or any other imaging technique that allows one to measure brain function. In animals, cognitive function may also be measured with electrophysiological techniques.

There are various tests known in the art for assessing cognitive function in humans, for example and without limitation, the Alzheimer's Disease Assessment Scale-cognitive subscale (ADAS-cog); clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB); the Sandoz Clinical Assessment-Geriatric (SCAG), the Buschke Selective Reminding Test (Buschke and Fuld, 1974); the Verbal Paired Associates subtest; the Logical Memory subtest; and the explicit 3-alternative forced choice task (see, e.g., WO2012109491); the Visual Reproduction subtest of the Wechsler Memory Scale-Revised (WMS-R) (Wechsler, 1997); the Benton Visual Retention Test. See Folstein M F et al., J Psychiatric Res 12:189-98, (1975); Robbins T W et al., Dementia 5:266-81, (1994); Rey, L'examen clinique en psychologie, (1964); Luger et al., J Geriatric Psychiatry Neurol 12:168-79, (1999); Marquis S et al., Archives of Neurology. 2002; 59:601-606 and Masur D M, et al., Neurology. 1994 August; 44 (8): 1427-32.

Use of the rAAV-DN-TNFα composition of the disclosure for the treatment of Alzheimer's disease results in an improvement in one or more of the following parameters: reduction in levels of Aβ accumulation, amyloid plaques, and/or Tau neuroinflammatory markers, enhanced cognitive function, etc. over baseline measurements, or at least prevents or slows the progression of AD from one stage to the next stage. In some embodiments, myelination of the neurons is protected and/or improved with treatment of the methods of this disclosure.

### Systemic and Peripheral Inflammatory Conditions

The compositions and methods of this disclosure can be used to treat systemic and peripheral inflammatory conditions including, but not limited to, Rheumatoid Arthritis (RA), Crohn's Disease and other inflammatory conditions of the peripheral tissues.

Rheumatoid arthritis is an autoimmune and inflammatory disease of the joints affecting 1.5 million Americans. To date, there is no cure for RA. Patients are usually treated with systemic steroids or disease-modifying antirheumatic drugs (DMARDs). Treatment efficacy in RA with the disclosed compositions and methods can be measured by a number of methods, including but not limited to, a reduction in the American College of Rheumatology (ACR) score, a reduction in the Disease Activity Score 28 (DAS28), a reduction in total joint score progression, a reduction in serum C-reactive protein and a reduction in circulating soluble TNF receptors. RA Disease Activity Measures are well known in the art and include the American College of Rheumatology core set of measurements. Other measures include tender joint count, swollen joint count, function, pain, patient global assessment, physician global assessment, and laboratory marker for inflammation (erythrocyte sedimentation rate [ESR] or C-reactive protein [CRP] levels. See, e.g., Anderson J, et al., Arthritis Care Res (Hoboken). 2012 May; 64 (5):640-7. and Buzatu C, Moots RJ. Expert Rev Clin Immunol. 2019 February; 15 (2):135-145.

In some embodiments, treatment efficacy can be measured by performing one or more tests to evaluate the level of inflammation, for example, by measuring a level of a marker of inflammation in the subject with rheumatoid arthritis. Examples of markers of inflammation include but are not limited to CRP, soluble intercellular adhesion molecule (sICAM-1), ICAM 3, BL-CAM, LFA-2, VCAM-1, NCAM, PECAM, fibrinogen, serum amyloid A (SAA), lipoprotein associated phospholipase A2 (LpPIA2), sCD40 ligand (sCD40L), myeloperoxidase, Interleukin-1β (IL-1β), Interleukin-6 (IL-6), Interleukin-8 (IL8), Interleukin-17A (IL-17A), interferon-γ (IFN-γ). See, e.g., Shrivastava A K, et al., Allergol Immunopathol (Madr). 2015 January-February; 43(1):81-7; and Lin Y J, et al., Cells. 2020; 9(4):880. Published 2020 Apr. 3.

Inflammation is associated with neuromuscular conditions, such as dystrophinopathies, particularly Duchenne muscular dystrophy (DMD). A method of decreasing inflammation and/or muscle degeneration in a muscle of a subject in need thereof is provided, comprising administering a AAV-DN-TNFα pharmaceutical composition as disclosed herein. The AAV-DN-TNFα pharmaceutical composition can be administered in combination with, e.g., sequentially or concomitantly with, an agent capable of restoring an active fragment of dystrophin, such as AAV-microdystrophin or an exon-skipping therapy.

The AAV-DN-TNFα as disclosed herein is administered therapeutically to ameliorate or stop molecular signals that mediate inflammation and fibrosis (tissue scaring) mechanisms in muscle, including the heart, triggered by the loss of dystrophin protein—the hallmark feature of DMD. Inflammation in muscle may be assessed, for example, based on Hematoxylin and Eosin (H&E) staining and microscopically examining representative areas of inflammatory foci within the tissue. Percent inflammation is subsequently measured. Regenerating fibers in the muscle may be examined by anti-eMHC staining, a marker of regeneration to identify areas of positive degenerating fibers.

The protocol described in any of the Examples can be used for administration of AAV-DN-TNF. The dosing for a second therapeutic used, such as a microdystrophin, can be any of the clinical protocols known for the second therapeutic. The combination treatments can last for at least one or more months, e.g., 6 months, one year, two years, three years, four years, five years, or up to at least 10 years. The combination can provide a synergistic, greater than additive, therapeutic benefit for one or more of the monitored clinical endpoints as compared to each therapeutic on its own or, alternatively, the therapeutics may each ameliorate a different set of therapeutic end points such that the therapeutic benefit is greater than each therapeutic administered on its own. The combination can be a combination of DN-TNF vector and microdystrophin vector. DN-TNF vector and casimersen, DN-TNF vector and eteplirsen, DN-TNF vector and golodirsen, DN-TNF vector and viltolarsen, DN-TNF vector and ataluren, DN-TNF vector and prednisone, or DN-TNF vector and deflazacort. Microdystrophin vectors are disclosed in International Application PCT/US2020/062484, filed Nov. 27, 2020 (WO2021108755A2), which is hereby incorporated by reference in its entirety.

### Ocular Conditions

The compositions and methods of this disclosure can be used to treat ocular conditions, including, but not limited to, non-infectious uveitis, retinal disorders including diabetic retinopathy, myopic choroidal neovascularization (mCNV), macular degeneration (e.g., neovascular (wet) or dry age-related macular degeneration (nAMD)), macular edema (e.g., macular edema following a retinal vein occlusion (RVO) or diabetic macular edema (DME)), retinal vein occlusion, diabetic retinopathy (DR), glaucoma, and abnormal vascularization of the retina.

Non-infectious posterior uveitis is a form of ocular inflammation that affects the retina and choroid of the eye and leads to blindness. It afflicts approximately 38,000 Americans per year. Patients are usually treated with systemic steroids or corticosteroids therapy, which results in high risks of systemic complications. The term “uveitis” as used herein refers to inflammation of the eye that may affect the uvea, or middle layer of the eye but also the lens, retina, optic nerve, and vitreous chamber. Uveitis may involve the full eye (panuveitis) or a segment of the eye (anterior, intermediate or posterior). Examples of uveitis include, but are not limited to, anterior uveitis (comprising iritis, iridiocyclitis, and anterior cylitis), intermediate uveitis (comprising pars planitis, posterior cyclitis, and hyalitis), posterior uveitis (comprising focal, multifocal or diffuse choroiditis, chorioretinitis, retinochoroiditis, retinitis, and neuroretinitis), panuveitis, acute uveitis, recurring uveitis and chronic uveitis. In one embodiment, uveitis is non-infectious uveitis. Examples of causes of non-infectious uveitis include, but are not limited to, systemic autoimmune disorders (such as, for example Behcet's disease and Vogt-Koyanagi-Harada (VKH) disease); trauma and surgery. In one embodiment, non-infectious uveitis is idiopathic non-infectious uveitis.

The term “treatment” or “treating” in the context of ocular conditions such as uveitis as used herein generally refers to any improvement in the clinical symptoms of uveitis, as well as any improvement in the well-being of the patients, in particular an improvement manifested by at least one of the following: reduction of visual haze, maintenance or improvement of vision, therapeutic response that may be assessed by dilated fundus examination or by other assessment method aiming at observing for example, healing or decrease of inflammatory lesions, tissue destruction, biomarkers of autoimmunity and/or inflammation, vasculitis, disruption of the retina blood barrier, reduction in cellular infiltration, reduction in edema, or renewal of tissues, reduction in the presence of retinal folds or retinal detachment.

In some embodiments, treatment of uveitis may correspond to reduction of the grade for anterior chamber cells, as described in Zierhut M. et al., European Ophthalmic Review, Touch Briefings, 2007:77-78). In this grading scheme, the number of cells in a field having a size of 1×1 mm slit beam is measured, and grade O corresponds to <1 cells in field, grade 0.5+ corresponds to 1-5 cells in field, grade 1+ corresponds to 6-15 cells in field, grade 2+ corresponds to 16-25 cells in field, grade 3+ corresponds to 26-50 cells in field, and grade 4+ corresponds to >50 cells in field.

In some embodiments, treatment of uveitis may correspond to reduction of the grade for anterior chamber flare, as described in Zierhut M. et al., (European Ophthalmic Review, Touch Briefings, 2007:77-78). In this grading scheme, the flair is evaluated, with grade 0 corresponding to the absence of flare, grade 1+ corresponding to faint flare, grade 2+ corresponding to moderate flare (iris and lens details clear), grade 3+ corresponding to marked flare (iris and lens details hazy), and grade 4+ corresponding to intense flare (fibrin or plastic aqueous).

In some embodiments, treatment of uveitis may correspond to reduction of vitreous haze, as described in Zierhut M. et al., (European Ophthalmic Review, Touch Briefings, 2007:77-78). In this grading scheme, vitreous haze is analyzed, with score 0 (Nil) corresponding to no clinical finding, score 1 (minimal) corresponding to a posterior pole clearly visible, score 2 (mild) corresponding to posterior pole details slightly hazy, score 3 (moderate) corresponding to posterior pole details very hazy, score 4 (marked) corresponding to posterior pole details barely visible and score 5 (severe) corresponding to a stage where fundal details are not visible.

In certain embodiments, maintenance or improvement of vision may be assessed by scores of visual acuity, which are well known by the skilled artisan.

In some embodiments, treatment efficacy can be measured by performing one or more tests to evaluate the level of inflammation, for example, by measuring a level of a marker of inflammation in the subject with uveitis. Examples of markers of inflammation include but are not limited to IL-17, IL-23, IL-6, IL-22, and IL-10. In some embodiments, treatment with the methods of this disclosure decrease the levels of IL-17, IL-23, IL-6, and IL22 in the eye(s) of the subject with uveitis. In some embodiments, treatment with the methods of this disclosure increase the levels of IL-10 in the eye(s) of the subject with uveitis. See, e.g., Weinstein J E, Pepple K L. Curr Opin Ophthalmol. 2018; 29(3):267-274.

### Pharmaceutical Compositions and Formulations

Pharmaceutical compositions suitable for administration to human subjects comprise a suspension of the recombinant AAV construct of the disclosure in a formulation buffer comprising a physiologically compatible aqueous buffer, a surfactant and optional excipients. Such formulation buffer can comprise one or more of a polysaccharide, a surfactant, polymer, or oil. In some embodiments, the pharmaceutical composition comprises rAAV combined with a pharmaceutically acceptable carrier for administration to a subject.

In one embodiment, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™ as known in the art. The pharmaceutical composition of the present disclosure can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

### Methods of Treatment

Provided herein are methods of treating human subjects with a neuroinflammatory condition, a systemic inflammatory condition or an ocular inflammatory condition using an rAAV-DN-TNFα construct.

### Methods of Treatment for Neuroinflammatory Conditions

In certain embodiments, the subject has been diagnosed with and/or has symptoms associated with AD, or prodromal AD, e.g., a mild cognitive impairment associated with early AD or even pre-AD. Recombinant vectors used for delivering the DN-TNFα transgene to the human central nervous system (CNS) are described herein. Such vectors are designed to have a tropism for human CNS cells (e.g., neurons and/or glia) and can include non-replicating rAAV, particularly those bearing an AAV9 or AAVrh10 capsid. The recombinant vectors can be administered in any manner such that the recombinant vector enters the CNS, e.g., by introducing the recombinant vector into the cerebral spinal fluid (CSF). In some embodiments, the rAAV vector is administered intravenous, intracerebral, intraparenchymal, intrastriatal, intracerebroventricular (ICV), intracisternal (IC), intraventricular, lumbar intrathecal (IT), or by a brain implant.

Therapeutically effective doses of a recombinant vector of the disclosure should be administered in any manner such that the recombinant vector enters the CNS, e.g. intracerebrally, intraparenchymally, intrastriatally, intracisternally, lumbar intrathecally, intracerebroventricularly, intraventricularly, or by a brain implant. In specific, embodiments, the vector is administered intrathecally, specifically intracisternally (such as to the cisterna magna) or, alternatively, lumbar delivery. Alternatively, the recombinant vector may be administered intravenously. Intrathecal, including intracisternal or lumbar administration, or intravenous administration should result in expression of the soluble DN-TNFα in cells of the CNS. The expression of the DN-TNFα protein results in delivery and maintenance of DN-TNFα in the CNS. Because the DN-TNFα is continuously produced, maintenance of lower concentrations can be effective. The concentration of DN-TNFα can be measured in patient samples of the CSF. The composition of the disclosure is administered to the subject in need thereof at an appropriate dose as determined by a person of skill in the art.

In some embodiments, the AAV vectors of the disclosure may be delivered to the CNS by any of the methods at any of the relevant doses described in International Patent Application Nos. PCT/US2017/014914 and PCT/US2019/042205, incorporated by reference herein in their entireties. Briefly, the AAV vector can be delivered to the CNS by injection into the CSF via an intracerebroventricular, intrathecal cisternal, or intrathecal lumbar route. This method can deliver the AAV vector to neurons, Purkinje neurons and/or astrocytes. In some embodiments, a total dose of about 5×10¹² to about 2×10¹⁴ genome copies (GC), or about 2×10¹³ to about 7×10¹⁴ GC of the AAV vector can be administered to an adult subject.

In specific embodiments, provided are constructs for gene therapy administration to a human subject, comprising an AAV vector, which comprises a viral capsid that is at least 95% identical to the amino acid sequence of an AAV8 capsid (SEQ ID NO: 11); and a viral or artificial genome comprising an expression cassette flanked by AAV inverted terminal repeats (ITRs) wherein the expression cassette comprises a transgene encoding DN-TNFα, operably linked to one or more regulatory sequences that control expression of the transgene in human cells that express and deliver DN-TNFα in a therapeutically appropriate manner as disclosed herein, particularly expressed from CNS cells. In certain embodiments, the encoded AAV8 capsid has the sequence of SEQ ID NO: 11 with 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions, particularly substitutions with amino acid residues found in the corresponding position in other AAV capsids, for example, in the SUBS row of FIG. 6, which provides a comparison of the amino acid sequences of the capsid sequences of various AAVs, highlighting amino acids appropriate for substitution at different positions within the capsid sequence. As such, provided are viral vectors in which the capsid protein is a variant of the AAV8 capsid protein (SEQ ID NO: 11), and the capsid protein is e.g., at least 95%, 96%, 97%, 98%, 99% or 99.9% identical to the amino acid sequence of the AAV8 capsid protein (SEQ ID NO: 11), while retaining a biological function of the native capsid.

Other specific embodiments provide constructs for gene therapy administration to a human subject that comprise an AAV vector, which, in turn, comprises a viral capsid that is at least 95% identical to the amino acid sequence of an AAV9 capsid (SEQ ID NO: 5); and a viral or artificial genome comprising an expression cassette flanked by AAV inverted terminal repeats (ITRs) wherein the expression cassette comprises a transgene encoding the DN-TNFα, operably linked to one or more regulatory sequences that control expression of DN-TNFα in human cells that express and deliver the DN-TNFα in a therapeutically appropriate manner as disclosed herein, particularly from CNS cells. In certain embodiments, the encoded AAV9 capsid has the sequence of SEQ ID NO: 5 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions, particularly substitutions with amino acid residues found in the corresponding position in other AAV capsids, for example, in the SUBS row of FIG. 6, which provides a comparison of the amino acid sequences of the capsid sequences of various AAVs, highlighting amino acids appropriate for substitution at different positions within the capsid sequence. As such, provided are viral vectors in which the capsid protein is a variant of the AAV9 capsid protein (SEQ ID NO: 5), and the capsid protein is e.g., at least 95%, 96%, 97%, 98%, 99% or 99.9% identical to the amino acid sequence of the AAV9 capsid protein (SEQ ID NO: 5), while retaining a biological function of the native capsid.

Other specific embodiments provide constructs for gene therapy administration to a human subject that comprise an AAV vector, which, in turn, comprises a viral capsid that is at least 95% identical to the amino acid sequence of an AAVrh10 capsid (SEQ ID NO: 16); and a viral or artificial genome comprising an expression cassette flanked by AAV inverted terminal repeats (ITRs) wherein the expression cassette comprises a transgene encoding the DN-TNFα, operably linked to one or more regulatory sequences that control expression of DN-TNFα in human cells that express and deliver the DN-TNFα in a therapeutically appropriate manner as disclosed herein, particularly from CNS cells. In certain embodiments, the encoded AAVrh10 capsid has the sequence of SEQ ID NO: 16 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions, particularly substitutions with amino acid residues found in the corresponding position in other AAV capsids, for example, in the SUBS row of FIG. 6, which provides a comparison of the amino acid sequences of the capsid sequences of various AAVs, highlighting amino acids appropriate for substitution at different positions within the capsid sequence. As such, provided are viral vectors in which the capsid protein is a variant of the AAVrh10 capsid protein (SEQ ID NO: 16), and the capsid protein is e.g., at least 95%, 96%, 97%, 98%, 99% or 99.9% identical to the amino acid sequence of the AAVrh10 capsid protein (SEQ ID NO: 16), while retaining a biological function of the native capsid.

Other specific embodiments provide constructs for gene therapy administration to a human subject that comprise an AAV vector, which, in turn, comprises a viral capsid that is at least 95% identical to any of the amino acid sequences of the capsids in Table 1 (SEQ ID NOS: 5-24); and a viral or artificial genome comprising an expression cassette flanked by AAV inverted terminal repeats (ITRs) wherein the expression cassette comprises a transgene encoding the DN-TNFα, operably linked to one or more regulatory sequences that control expression of DN-TNFα in human cells that express and deliver the DN-TNFα in a therapeutically appropriate manner as disclosed herein, particularly from CNS cells.

In some embodiments, the DN-TNFα transgene should be controlled by appropriate expression control elements for expression of the DN-TNFα transgene in human CNS cells, including, but not limited to, any of the promoters in Tables 4 and 5. For example, the promoter can be the CB7 promoter (a chicken β-actin promoter and CMV enhancer), RSV promoter, GFAP promoter (glial fibrillary acidic protein), MBP promoter (myelin basic protein), MMT promoter, EF-1α, U86 promoter, RPE65 promoter or opsin promoter, an inducible promoter, for example, a hypoxia-inducible promoter or a drug inducible promoter, such as a promoters induced by rapamycin and related agents, and other expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 late splice donor/splice acceptor (19S/16S) intron, and hybrid adenovirus splice donor/IgG splice acceptor intron and poly A signals such as the rabbit β-globin polyA signal, human growth hormone (hGH) polyA signal, SV40 late polyA signal, synthetic polyA (SPA) signal, and bovine growth hormone (bGH) polyA signal). See, e.g., Powell S K, et al., Discov Med. 2015 January; 19 (102): 49-57. In one specific embodiment, the constructs described herein comprise the following components: (1) AAV2 inverted terminal repeats that flank the expression cassette; (2) Control elements, which include a) the CAG promoter, and b) a rabbit β-globin poly A signal; and (3) nucleic acid sequences coding for DN-TNFα. A representative construct is provided in FIG. 1.

Other specific embodiments provide AAV vectors that comprise a viral capsid that is at least 95% identical to the amino acid sequence of an AAV9 (SEQ ID NO: 5) or AAVrh10 (SEQ ID NO: 16) capsid; and an artificial genome comprising an expression cassette flanked by AAV inverted terminal repeats (ITRs), wherein the expression cassette comprises a transgene encoding DN-TNFα, operably linked to one or more regulatory sequences that control expression of DN-TNFα in human CNS cells.

Pharmaceutical compositions suitable for intravenous, intracerebral, intraparenchymal, instrastriatal, intracerebroventricular (ICV), intracisternal (IC), intraventricular, lumbar intrathecal (IT) delivery, or administration by a brain implant comprise a suspension of the recombinant vector comprising the DN-TNFα transgene in a formulation buffer comprising a physiologically compatible aqueous buffer. The formulation buffer can comprise one or more of a polysaccharide, a surfactant, polymer, or oil.

In some embodiments, therapeutically effective doses of the recombinant vector are administered to the CSF via intrathecal administration (i.e., injection into the subarachnoid space so that the recombinant vectors distribute through the CSF and transduce cells in the CNS). This can be accomplished in a number of ways—e.g., by intracranial (cisternal or ventricular) injection, or injection into the lumbar cistern. For example intracistemal (IC) injection (into the cisterna magna) can be performed by CT-guided suboccipital puncture; or injection into the subarachnoid space can be performed via a Cl −2 puncture when feasible for the patient; or lumbar puncture (typically diagnostic procedures performed in order to collect a sample of CSF) can be used to access the CSF. Alternatively, intracerebroventricular (ICV) administration (a more invasive technique used for the introduction of anti-infective or anti-cancer drugs that do not penetrate the blood-brain barrier) can be used to instill the recombinant vectors directly into the ventricles of the brain. Alternatively, intranasal administration may be used to deliver the recombinant vector to the CNS. Doses that maintain a CSF concentration of DN-TNFα at a minimum of at least 5 μg/mL or concentrations ranging from 5 to 500 μg/mL may be used.

### Methods of Treatment for Systemic Conditions

In certain embodiments, the subject has been diagnosed with and/or has symptoms associated with systemic conditions, such as rheumatoid arthritis. Recombinant vectors used for delivering the DN-TNFα transgene systemically are described herein. Such vectors should be designed to have a tropism for human muscle cells, liver cells, and/or synovial cells and can include non-replicating rAAV, particularly those bearing an AAV8 or AAV9 capsid. The recombinant vectors can be administered in any manner such that the recombinant vector enters the muscle cells, liver cells, and/or synovial cells, e.g., by introducing the recombinant vector into the cerebral spinal fluid (CSF). In some embodiments, the rAAV vector is administered by intravenous, intramuscular, intrasynovial, intra-articular, or peri-articular delivery.

Therapeutically effective doses of a recombinant vector of the disclosure should be administered in any manner such that the recombinant vector enters the muscle, liver, and/or synovial tissue, e.g. by intravenous, intramuscular, intrasynovial, intra-articular, or peri-articular delivery. Successful administration should result in expression of the soluble DN-TNFα in cells of the liver, muscle, and/or synovial tissue. The expression of the DN-TNFα protein results in delivery and maintenance of DN-TNFα in the liver, muscle and/or synovial tissue. Because the DN-TNFα is continuously produced, maintenance of lower concentrations can be effective. The concentration of DN-TNFα can be measured in patient samples of the plasma. The composition of the disclosure is administered at an appropriate dose as determined by a person of skill in the art.

In some embodiments, the AAV vectors of the disclosure are delivered systemically by any of the methods at any of the relevant doses described in International Patent Application Nos. PCTUS2019043631, incorporated herein in its entirety. In certain embodiments, dosages are measured by the number of genome copies administered to the human subject via AAVs provided herein. In some embodiments, 1×10¹⁰ to 1×10¹⁶ genome copies of the AAV vector are administered.

Other specific embodiments provide constructs for gene therapy administration to a human subject, that comprise an AAV vector, which, in turn, comprises a viral capsid that is at least 95% identical to any of the amino acid sequences of the capsids in Table 2 (SEQ ID NOs: 5, 11, and 25-29); and a viral or artificial genome comprising an expression cassette flanked by AAV inverted terminal repeats (ITRs) wherein the expression cassette comprises a transgene encoding the DN-TNFα, operably linked to one or more regulatory sequences that control expression of DN-TNFα in human cells that express and deliver DN-TNFα in a therapeutically appropriate manner as disclosed herein, particularly from muscle, liver, and/or synovial cells.

In some embodiments, the DN-TNFα transgene are controlled by appropriate expression control elements for expression of the DN-TNFα transgene in human muscle, liver, or synovial cells, including, but not limited to any of the promoters in Tables 7 and 9.

### Methods of Treatment for Ocular Conditions

In certain embodiments, the subject has been diagnosed with and/or has symptoms associated with ocular conditions. Recombinant vectors used for delivering the DN-TNFα transgene systemically are described herein. Such vectors should be designed to have a tropism for human eye cell types (e.g., ocular cells, retina cells, retinal pigment cells) and can include non-replicating rAAV, particularly those bearing an AAV8, or AAV9 capsid. The recombinant vectors can be administered in any manner such that the recombinant vector enters the eye cells, e.g., by introducing the recombinant vector into the ocular fluid (e.g., aqueous or vitreous humor). In some embodiments, the rAAV vector is administered by subretinal, intravitreal, suprachoroidal or intracameral delivery.

Therapeutically effective doses of a recombinant vector of the disclosure should be administered in any manner such that the recombinant vector enters the ocular tissue, e.g. by intravenous, intramuscular, intrasynovial, intra-articular, or peri-articular delivery. Successful administration should result in expression of the soluble DN-TNFα in cells of the ocular tissue. The expression of the DN-TNFα protein results in delivery and maintenance of DN-TNFα in the ocular tissue. Because the DN-TNFα is continuously produced, maintenance of lower concentrations can be effective. The concentration of DN-TNFα can be measured in patient samples of the ocular fluid. The composition of the disclosure is administered to the subject at an appropriate dose as determined by a person of skill in the art. In some embodiments, about 2×10¹⁰ GC to about 6×10¹⁰ GC per eye of the recombinant AAV vector are administered.

Other specific embodiments provide constructs for gene therapy administration to a human subject, that comprise an AAV vector, which, in turn, comprises a viral capsid that is at least 95% identical to any of the amino acid sequences of the capsids in Table 3 (SEQ ID NOs: 5, and 30-47); and a viral or artificial genome comprising an expression cassette flanked by AAV inverted terminal repeats (ITRs) wherein the expression cassette comprises a transgene encoding the DN-TNFα, operably linked to one or more regulatory sequences that control expression of DN-TNFα in human cells that express and deliver DN-TNFα in a therapeutically appropriate manner as disclosed herein, particularly from ocular cells.

In some embodiments, the DN-TNFα transgene should be controlled by appropriate expression control elements for expression of the DN-TNFα transgene in human ocular cells, including, but not limited to any of the promoters in Tables 7 and 10.

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### EXAMPLES

### Example 1: DN-TNF Alpha Vector Genome Construction and rAAV Production

A recombinant AAV (ssAAV.DNTNF.001) was made by first constructing a Cis plasmid (DNTNF.001) having the following components in 5′ to 3′ order: 5′-inverted terminal repeat (ITR), a CAG promoter, Vh4 intron, modified IL-2 leader (signal sequence), dominant-negative tumor necrosis factor alpha (DN-TNFα) coding sequence, poly-adenylation sequence (polyA), and 3′-ITR, using standard molecular biology techniques. The CAG promoter is a robust ubiquitous promoter that includes the cytomegalovirus (CMV) early enhancer, chicken beta-actin promoter, the first exon and the first intron of a chicken beta-actin gene, and the splice acceptor of the rabbit beta-globin gene. The consensus/Kozak sequence GCCACC (SEQ ID NO: 176) was also added upstream of the ATG start codon of DN-TNFα to increase translation. The dominant-negative tumor necrosis factor alpha (DN-TNFα) protein is a TNFα variant containing two mutations at amino acid residue 87 and 145 (Y87H/A145R) (Steed, P M et al., Science, 2003. 301 (5641): 1895-8) as compared to the wild-type TNFα protein. The DN-TNFα nucleotide sequence encoding the DN-TNFα protein (SEQ ID NO: 3) was human codon-optimized and CpG-depleted (GeneArt). The DN-TNFα coding sequence is preceded by a modified human interleukin-2 (IL2) signal peptide (SEQ ID NO: 117) to augment protein secretion.

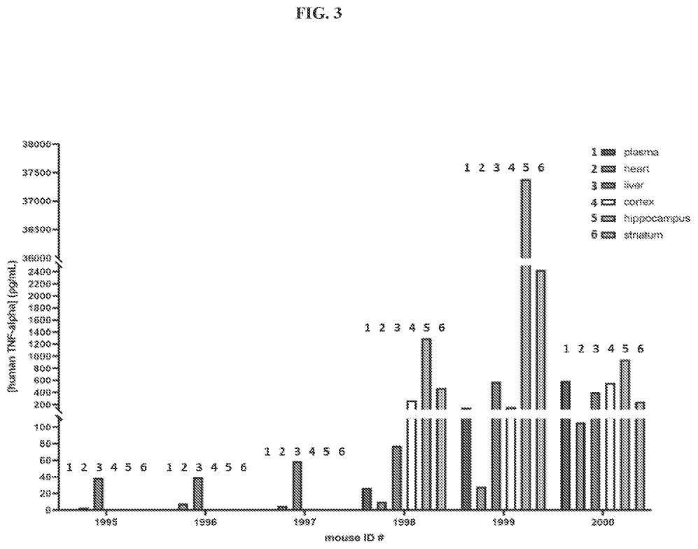

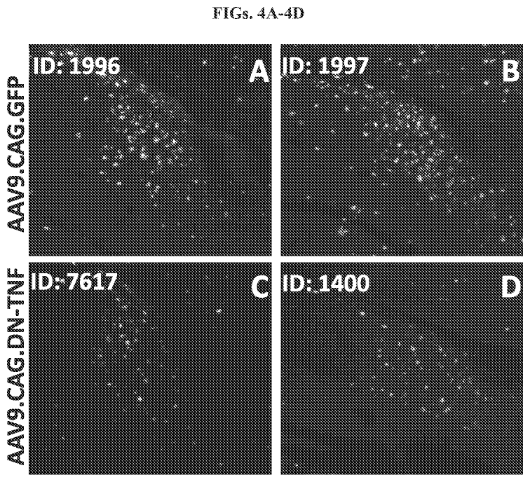

In another AAV vector (scAAV.DNTNF.002), the D sequences and terminal resolution site were deleted on one AAV inverted terminal repeat (ITR) for rendering double-stranded AAV genome packaging, also known as self-complementary AAV (scAAV). Human synapsin (hSyn) promoter (GenBank: MH458079.1) was used for neuron-specific transgene expression of the gene of interest, DN-TNFα, in this vector. The Cis plasmid (DNTNF.002) contains the following components in 5′ to 3′ order: mutant 5′-ITR, hSyn promoter, Vh4 intron, modified IL-2 leader (signal sequence), DN-TNFα coding sequence, polyA, and 3′-ITR. See FIGS. 1 and 3.

Triple transfection of AAV plasmids (cis plasmid, trans (rep/cap) plasmid, and helper plasmid) was implemented for production of AAV particles in HEK293 cells. Briefly, AAV vectors were produced via transient transfection of HEK293 cells, whereas HEK 293 cells were thawed and expanded in culture medium. Shake flasks or bioreactors were seeded with HEK 293 cells at a density of about 1.0-1.2×10⁶ viable cells/mL. At approx. 48-72 hours, Elapsed Culture Duration (ECD), the cells were transfected with a mixture of polyethylenimine (PEI) and three plasmids encoding adenovirus helper functions, transgene (cis plasmid described herein), and AAV rep/cap. The supernatant and cell lysates of cell cultures were harvested at an ECD of 3-5 days post-transfection and rAAV particles were purified using standard methods.

### Example 2: AAV9-DN-TNFα Administration in 5XFAD Mouse Brains Results in Sustained DN-TNFα Expression

A study was conducted to determine the expression following administration of the AAV9-CAG.DN-TNFα (DNTNF.001) vector constructs described in Example 1 when injected directly into the brains in a mouse model of Alzheimer's disease.

5XFAD transgenic mice recapitulate major features of Alzheimer's disease amyloid pathology (Oakley H. J Neurosci. 2006 Oct. 4; 26(40): 10129-10140). 5XFAD transgenic mice overexpress mutant human amyloid beta (A4) precursor protein 695 (APP) with several mutations: the Swedish (K670N, M671L), Florida (1716V), and London (V717I) Familial Alzheimer's Disease (FAD) mutations; as well as human presenilin 1 (PS1) harboring two FAD mutations, M146L and L286V. 5XFAD mice are considered to be a useful model of intraneuronal Abeta-42 induced neurodegeneration and amyloid plaque formation.

To determine whether direct injection of AAV containing DN-TNFα into the central nervous system (CNS) will result in the expression of DN-TNFα in the CNS, AAV9.CAG.GFP (placebo) or AAV9.CAG.DN-TNFα (test) were directly injected intraparenchymally into the subiculum (2 μl 1e10 vg injected bilaterally) of 6 month old 5XFAD transgenic mice. 18 weeks after vector delivery, the mice were sacrificed, and regions of brain were dissected and homogenized. ELISA was performed to determine expression of human TNF protein in the homogenized tissue. The level of TNF was found to be several fold higher in several tissues of mice administered 1e11 vg AAV9-DN-TNFα, including the brain cortex (about 95,000 μg/ml), hippocampus (about 100,000 μg/ml), and striatum (about 93,000 μg/ml) with the highest levels of TNFα in the hippocampus (FIG. 5) compared to DN-TNFα levels (all under about 1500 pg/ml) in other tissues such as cerebellum, heart, liver, and plasma.

These results demonstrate that direct administration of an AAV-DN-TNFα into mouse brains results in high brain expression of DN-TNFα even 18 weeks after vector delivery, indicating that the vector is suitable for long-term expression of protein.

### Example 3: AAV9-DN-TNFα Administration in 5XFAD Mouse Brains Reduces Amyloid Beta Accumulation

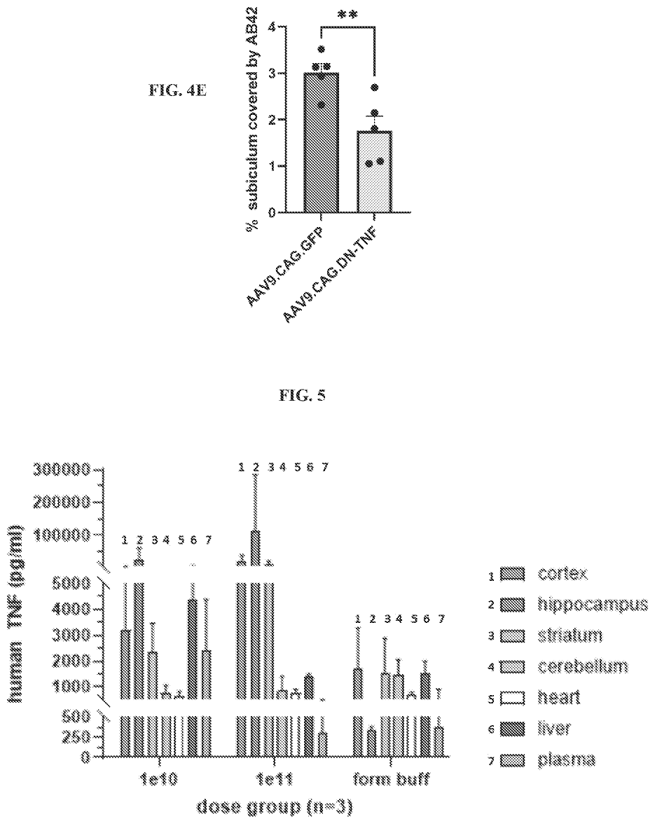

The effects of DN-TNFα injected into 5XFAD mouse brains as described in Example 2 was evaluated after 18 weeks. Briefly, mice were perfused with 30 ml PBS to clear blood from tissue. Left brain hemispheres were collected and drop fixed in 4% paraformaldehyde for 24 hours, then transferred to 30% sucrose for at least 24 hours before histology. Mouse brains were sliced in 50 μm thick sagittal sections, then immunostained with anti-amyloid beta precursor protein, which detects full-length and cleaved, aggregated amyloid beta. Sections were imaged and extracellular aggregates of amyloid beta were quantified using ImageJ in a blinded manner. FIGS. 4A-4D each show one representative image from a mouse injected with either the placebo (AAV9.CAG.GFP) or the test (AAV9.CAG.DN-TNFα/DNTNF.001) construct as indicated, and FIG. 4E shows the quantification of amyloid beta positive aggregates in the subiculum of the 6-month-old 5XFAD mice. As can be seen, the number of amyloid beta puncta is significantly higher in the placebo group compared to that in the test group.

These results demonstrate that AAV9 mediated expression of DN-TNFα in the CNS can reduce accumulation of beta-amyloid aggregates, a major pathological feature of Alzheimer's Disease.

### Example 4: AAV9-DN-TNFα ICV Administration in WT C57B6/J Mouse Brains Results in a Dose-Dependent Increase of DN-TNFα in CNS Tissues

A study was conducted to determine the expression of TNF following administration of AAV9-DN-TNFα constructs injected directly into the brains of wildtype (WT) C57B6/J mice. Six week old C57B6/J mice were administered 1e10 or 1e11 vg of AAV9.CAG.DN-TNF (DNTNF.001) in a 5 μl dose via unilateral intracerebroventricular (ICV) injection into the right hemisphere (n=3 mice per group). One group of mice received the control (formulation control). 4 weeks later, mice were sacrificed and perfused with 30 ml PBS. Right hemispheres were dissected and peripheral tissues were collected and frozen on dry-ice and stored at −80° C. Tissues were homogenized and equal protein concentrations from homogenates were analyzed by ELISA for DN-TNFα expression.

As shown in FIG. 5, the ICV administration of both doses of AAV9.CAG.DN-TNF resulted in elevated DN-TNFα expression in brain tissue (cortex, hippocampus, striatum, and cerebellum), and TNF was measurable in certain peripheral tissues (heart, liver and plasma) comparable to TNF measured in the periphery for animals treated with formulation buffer. Importantly, these results indicate a dose dependent increase of DN-TNFα expression in CNS tissues following ICV administration of gene therapy vector expressing DN-TNF in mice.

### Example 5: AAV-DN-TNFα Construct for Use in Peripheral and Systemic Inflammation

A DN-TNFα cDNA-based AAV vector is constructed comprising a transgene comprising nucleotide sequences encoding the DN-TNFα sequence (SEQ ID NO: 3). The nucleotide sequence coding for DN-TNFα is codon optimized for expression in human liver and/or muscle cells. The transgene also comprises nucleotide sequences that encode a signal peptide, e.g., mIL2 (SEQ ID NO: 115). The vector additionally includes a promoter, such as CAG promoter (SEQ ID NO: 48), an inducible promoter, or a liver and/or muscle tissue specific promoter such as the LSPX1 promoter (SEQ ID NO: 75).

Representative AAV-DN-TNFα plasmids are constructed to include combinations of one of the 5′ ITR, one of the promoters, one of the signal peptides, the codon-optimized & CpG-depleted DN-TNFα coding sequence, one or more of the regulatory elements, and one of the 3′ ITRs listed in Table 15, below.

TABLE 15
Elements Useful for DN-TNFα Constructs
for Use in Peripheral and Systemic Inflammation

| Promoters | Signal peptides | | PolyA |
|---|---|---|---|
| CAG promoter | Mutant IL2 | Codon- | Rabbit β-globin |
| (SEQ ID NO: | signal peptide | optimized | polyA (SEQ ID NO: |
| 48) | (SEQ ID NO: | & CpG- | 109) |
| | 115) | depleted | |
| LSPX1 promoter | VEGF-A signal | DN-TNFα | BGH polyA |
| (SEQ ID NO: | peptide | coding | (SEQ ID NO: 114) |
| 75) | (SEQ ID NO: | sequence | |
| | 118) | (SEQ | |
| LTP1 promoter | Mutant IL2 | ID NO: 3) | Rabbit β-globin |
| (SEQ ID NO: | signal peptide | | polyA (SEQ ID NO: |
| 77) | (SEQ ID NO: | | 109) |
| | 115) | | |
| LMTP6 | Mutant IL2 | | Rabbit β-globin |
| promoter | signal peptide (SEQ | | polyA (SEQ ID NO: |
| (SEQ ID NO: | ID NO: 115) | | 109) |
| 80) | | | |

Any of these constructs can be used to reduce peripheral and systemic inflammation in conditions such as Rheumatoid arthritis or Crohn's Disease.

### Example 6: AAV-DN-TNFα Construct Reduces Inflammation in Rheumatoid Arthritis

A DN-TNFα cDNA-based AAV vector such as any one of those described in Example 5 is used to evaluate the reduction in inflammation in vitro or in vivo (e.g., in a rodent model of Rheumatoid Arthritis or RA). Control AAV particles and AAV-DN-TNFα particles are produced using methods described in Example 1 and see, e.g., methods described in WO2005021768, incorporated herein in its entirety.

For in vitro studies, the ability of AAV-DN-TNFα particles to alter cytokine production and secretion is assessed. Several cytokines are known to be involved in the pathology of RA. See, e.g., Lubberts E, van den Berg W B: Madame Curie Bioscience Database. Austin (TX): Landes Bioscience; 2000-2013), including IL1, IL15, IL17, RANKL and others. Altered cytokine production is measured using in vitro or ex vivo assays, such as enzyme-linked immunosorbent assays (ELISA), enzyme-linked immune absorbent spot (ELISPOT), polymerase chain reaction (PCR), immunoassays, in vivo cytokine capture assays (IVCCA), or cytokine release assays (CRAs) using human or animal cells. Such methods are described in the art. See, e.g., Finkelman F. et al., Current Protocols in Immunology, 2003, 54(1):6.28.1-6.28.10; Favre N et al, Journal of Immunological Methods, 1997, 204(1):57-66; Finco D et al., Cytokine, April 2014, 66 (2): 143-155.

In rats, appropriate doses of control AAV particles or AAV-DN-TNFα particles (e.g., 1×10¹⁰ vg to 10×10¹⁰ viral genomes (vg)) are injected into the ankle joints of rats with adjuvant arthritis (AA) on day 12 after adjuvant immunization. Joints are harvested after 2 weeks and analyzed for DN-TNFα expression by direct in-situ staining of frozen sections, and quantified by digital image analysis and RT-PCR. Thereafter, the number of cells expressing DN-TNFα in synovial tissue are assessed for the control animals and the treated animals. Further, the collected tissue is analyzed for changes in inflammatory activity (such as cytokine production and secretion) between the control and treated groups.

In mice, a suitable mouse model of rheumatoid arthritis (see e.g., Caplazi P, et al., Vet Pathol. 2015 September; 52(5):819-26) is used for the study. In particular, collagen-induced arthritis (CIA) is induced by intradermal injection at the base of the tail with 100 μl of collagen solution at 1 μg/μl at day 0. Bovine type II collagen (bCII) is diluted at 2 mg/ml with acetic acid 50 mM, and emulsified with an equal volume of Freund's complete adjuvant before use. On day 21, animals are boosted with an intradermal injection of 100 μl bCII solution emulsified with an equal volume of Freund's incomplete adjuvant before use. Following arthritis induction paw thickness is measured over time with a micrometer Mitutoyo. On day 28, mice are intraperitoneally injected with 40 μg LPS. When clinical signs for arthritis appear, mice are anaesthetized by intraperitoneal injection of a ketamine (30 mg/Kg) and xylazine (10 mg/Kg) solution. The skin above the knee is shaved, and appropriate doses of control AAV particles or AAV-DN-TNFα particles (2×10¹⁰ vg to 10×10¹⁰ viral genomes (vg) are injected intra-articularly in 5 μl of 0.9% NaCl into the left knee joint, by using a Hamilton syringe with a 30-gauge needle. Thereafter, clinical progression of the arthritis is measured as described in Fischer B D et al, Arthritis Res Ther 19, 146 (2017).

At the day of sacrifice, whole knee joints are collected and frozen in liquid nitrogen for in situ quantification of DN-TNFα staining on frozen sections. Blood samples are taken at various time points before and after vector injection, and stored at −20° C. until tested. At day of sacrifice, left and right patellaes are collected and incubated for 24 hours in RPMI (200 μl). Supernatants are stored at −20° C. and patellaes are stored in liquid nitrogen until tested. The collected tissue is analyzed for changes in inflammatory activity (such as cytokine production and secretion) between the control and treated groups.

Cells or animals treated with AAV-DN-TNFα are expected to show lower levels of pro-inflammatory cytokines such as IL1, IL15, IL17, RANKL. Furthermore, animals treated with AAV-DN-TNFα are expected to have lower clinical scores of joint inflammation, slower disease progression, and higher locomotor activity compared to animals treated with the control AAV vector.

### Example 7: AAV-DN-TNFα Construct for Use in Ocular Inflammatory Conditions

A DN-TNFα cDNA-based AAV vector is constructed as described in the examples above, but comprising a transgene comprising nucleotide sequences encoding the DN-TNFα amino acid sequence (SEQ ID NO: 2). The nucleotide sequence coding for DN-TNFα is codon optimized for expression in human eye cells (e.g., retina cells, RPE cells, photoreceptor cells, etc.) and can be the nucleotide sequence of SEQ ID NO: 3. The transgene also comprises one or more nucleotide sequences that encode a signal peptide, e.g., mIL2 (SEQ ID NO: 115). The vector additionally includes a promoter, such as a CAG promoter (SEQ ID NO: 48), an eye-specific promoter such as RPE65 promoter (SEQ ID NO: 104), or a retina-specific promoter such as a red cone opsin promoter (SEQ ID NO: 105).

Representative AAV-DN-TNFα plasmids are constructed to include combinations of one of the 5′ ITR, one of the promoters, one of the signal peptides, the codon-optimized & CpG-depleted DN-TNFα coding sequence, one or more of the regulatory elements, and one of the 3′ ITRs listed in Table 16.

TABLE 16
Elements Useful for DN-TNFα Constructs
for Use in Ocular Inflammation

| Promoters | Signal peptides | | Regulatory elements |
|---|---|---|---|
| CAG promoter | Mutant IL2 | Codon- | Rabbit β-globin |
| (SEQ ID NO: | signal peptide | optimized & | polyA (SEQ ID NO: |
| 48) | (SEQ ID NO: | CpG-depleted | 109) |
| | 115) | DN-TNFα coding | |
| RPE65 | VEGF-A signal | sequence | BGH polyA |
| promoter | peptide | (SEQ ID NO: 3) | (SEQ ID NO: 114) |
| (SEQ ID NO: | (SEQ ID NO: | | |
| 104) | 118) | | |
| Red cone opsin | Mutant IL2 | | Rabbit β-globin |
| Promoter | signal peptide | | polyA (SEQ ID NO: |
| (SEQ ID NO: | (SEQ ID NO: | | 109) |
| 105) | 115) | | |

These constructs are expected to reduce ocular inflammation in conditions such as non-infectious uveitis, diabetic retinopathy, myopic choroidal neovascularization (mCNV), macular degeneration (e.g., neovascular (wet) or dry age-related macular degeneration (nAMD)), macular edema (e.g., macular edema following a retinal vein occlusion (RVO) or diabetic macular edema (DME)), retinal vein occlusion, diabetic retinopathy (DR), glaucoma, or abnormal vascularization of the retina.

### Example 8: AAV-DN-TNFα Construct Reduces Inflammation in Non-Infectious Posterior Uveitis

In vivo Study 1: In this study, AAV9.CAG.DNTNF and AAV8.CAG.DNTNF are evaluated for AAV-mediated expression of DN-TNF in vivo in rodent ocular tissues via local administration (subretinal, SR). AAV9.CAG.GFP, AAV8.CAG.GFP and vehicle serve as controls.

Young adult C57BL/6 mice (8-10 weeks old) are used for this study. The DNTNF-expressing and GFP-expressing vectors are delivered into the mouse eyes via subretinal (SR) injection at different doses (1×10⁷, 1×10⁸ and 1×10⁹ vg/eye) in 1 μl of formulation buffer. Fundus and OCT imaging are performed at days 6 and 16 after SR injection. Ocular samples are collected at 21 days post administration. Levels of DN-TNF protein expression in ocular tissues (RPE, Retina and Anterior Segment) are quantified by ELISA. Cell type specificity is determined by immunofluorescent staining with various retinal cell markers. Retina structure changes and neuron survival are evaluated by histology and immunofluorescent staining at 6 and 16 days post administration.

Immunofluorescence double staining of tissue sections is used to confirm expression of human DN-TNF (as determined by using an antibody against human TNFα) in the RPE, Retina structure changes and neuron survival are evaluated by histology and immunofluorescent staining post AAV administration.

In vivo Study 2: Pre-injection of exogenous TNFα can induce inflammation in the eye. The ability of human TNFα to induce inflammation in a rodent eye is measured upon examination of the eye per the Clinical Grading of EAU guidelines of Agarwal, R J et al., Methods in Molecular Medicine. 2004: Vol. 102, pp 395-419. Briefly, to evaluate the efficacy of vectorized hDN-TNFα treatment: vector (e.g. AAV8.CAG.DNTNF or control vector) or vehicle is administered subretinally (SR) in both eyes (OU) at a dose of 1.0E+7 to 1.0E+9 GC/eye at day −21 (21 days before TNF-α induction), followed by 50-150 ng hTNFα (induction) administered to rodents (e.g., Lewis rats) by intravitreal (IVT) injection at day 0. Body Weights are measured prior to dosing and at necropsy; Ophthalmic Exams are done at baseline, 4 hours, 24 hours, Day 3, and Day 7, and EAU scores per the Clinical Grading of EAU guidelines of Agarwal (supra) are assessed. Necropsy is performed at Day 7, whereas one eye per animal/group is analyzed for transgene/TNFα levels, and one eye per animal/group is analyzed for histopathology.

### Example 9: AAV-DN-TNFα Dose Study Via Various Routes of Administration

To evaluate AAV-DN-TNFα dose, route of administration (ROA) and vector, brain and peripheral expression relative to vector biodistribution were assessed using ELISA and qPCR following in vivo administration to a mouse model of neurodegeneration, and tissues were also subject to analysis of neuroinflammation and amyloid beta deposition in 5XFAD mice. In this study, 6-8 week-old female C57/B6 mice are injected with either AAV9.CAG.DN-TNF or AAV9.hSyn.DN-TNF using peripheral, intracerebroventricular, or hippocampal routes of administration (ROAs). For peripheral ROA, 50 μL of vector is injected into the retroorbital sinus at three dose levels (1e12, 1e11, 1e10 vg). For ICV and hippocampal ROAs, doses are 1e11, 1e10, and 1e9 vg. For ICV, 2 μL of vector is injected for each hemisphere, while intrahippocampal ROA is performed with 2 injections of 1 μL each for each hemisphere. 3-4 weeks after injection, blood is collected via retroorbital bleeding. All mice are then sacrificed via transcardial perfusion and the liver, heart, cortex, hippocampus, and striatum are collected and snap frozen in dry ice. Biodistribution is assessed via ddPCR in the above-listed tissues. DN-TNF is also measured using several ELISA and other assays.

Per the protocol, 6-8 week-old female C57/B6 mice were injected with either DNTNF.001 (AAV9.CAG.DN-TNF) or DNTNF.002 (AAV9.hSyn.DN-TNF) using peripheral, intracerebroventricular, or hippocampal routes of administration (ROAs). For peripheral ROA, 50 μL of vector was injected into the retro-orbital sinus at three dose levels (1e12, 1e11, 1e10 vg). For ICV and hippocampal ROAs, doses were 1e11, 1e10, and 1e9 vg. For ICV, 2 μL of vector was injected for each hemisphere, while intrahippocampal ROA was performed with 2 injections of 1 uL each for each hemisphere. 3-4 weeks after injection, blood was collected via retro-orbital bleeding. All mice were then sacrificed via transcardial perfusion and the liver, heart, cortex, hippocampus, and striatum were collected and snap frozen in dry ice. Biodistribution was assessed via ddPCR in the above-listed tissues.

Biodistribution results showed no apparent transduction in peripheral tissues, for peripheral ROA, except for liver at high dose (1e12 vg) for each vector.

For ICV at high dose (1e11 vg) for DNTNF.002, 50 GC/cell and 100 GC/cell were found in cortex and hippocampus respectively, while all other vectors/doses showed no detectable GC/cell in these tissues with ICV ROA.

For intrahippocampal ROA, the high dose (1e11 vg) for DNTNF.002 showed comparable GC/cell to ICV results in both cortex and hippocampus, while DNTNF.001 also showed moderate GC/cell, but lower than the hSyn vector in both cortex and hippocampus.

DNTNF protein was also measured using an ELISA kit for hTNF, which measured the human DNTNF that is expressed (while not accounting for endogenous mouse TNF). DN-TNF expression levels were highest in the hippocampus tissue for the hippocampal ROA for both vectors at all the doses tested, with DNTNF-002 (AAV.hSyn.DN-TNF) having surprisingly higher overall expression than that expressed from DNTNF.001 (AAV.CAG.DN-TNF). Also in the hippocampal tissue, ICV ROA yielded higher expression at 1e11 and 1e10 for DNTNF.002 (AAV.hSyn.DN-TNF) than DNTNF.001 (AAV.CAG.DN-TNF), but yielded similar expression in the hippocampus as that for the hippocampal ROA at 1e11.

### Example 10: AAV-DN-TNFα Proof of Concept Study in 5XFAD Mouse Model

An optimal dose, route of administration, and vector were selected from a previous study (see Example 9). Two-month old female mice were injected bilaterally into the subiculum (hippocampus) with 2 μl of either AAV-CAG.DN-TNFα, or with AAV-CAG.GFP at 1e10 vg. Four months after vector delivery, mice were sacrificed. Some mice were perfused with PBS and 4% paraformaldehyde for 24 hours, then transferred to 30% sucrose for at least 24 hours before histology. Some mice were perfused with PBS, and regions of the brain were dissected and homogenized.

For histology, mouse brains were sliced in 50 μm thick sagittal sections, then immunostained with anti-amyloid antibodies. Cellular markers for neuroinflammation, including glial fibrillary acidic protein (GFAP) for astrocytes, ionized calcium-binding adapter molecule 1 (IBA1) for microglia, and CD11b/CD45/CD8 for infiltrating immune cells were also stained (data not shown). Both the anti-amyloid antibodies 6E10 and AB42 were tested for staining the subiculum of the treated 5xFAD mice. However, these antibodies show different labeling patterns because the 6E10 anti-amyloid antibody binds residues 1-16 found in both amyloid precursor protein and amyloid plaques. The background was higher in the 6E10 stained samples making measurement of plaques vs. precursor difficult. Background is reduced with the AB42 anti-amyloid antibody, because it binds residues 1-42 found in only amyloid plaques.

Five slices per mouse were quantified, matched between all mice based on coronal section (based on the Allen Mouse Brain Atlas), based on the following formulae:

$$\frac{total puncta count in all slices}{total subiculum area in all slices , {mm}^{2}} = puncta per mm \hat{} 2$$

$$\frac{total puncta area in all slices}{total subiculum area in all slices} * 100 = \% subiculum covered by A β 42$$

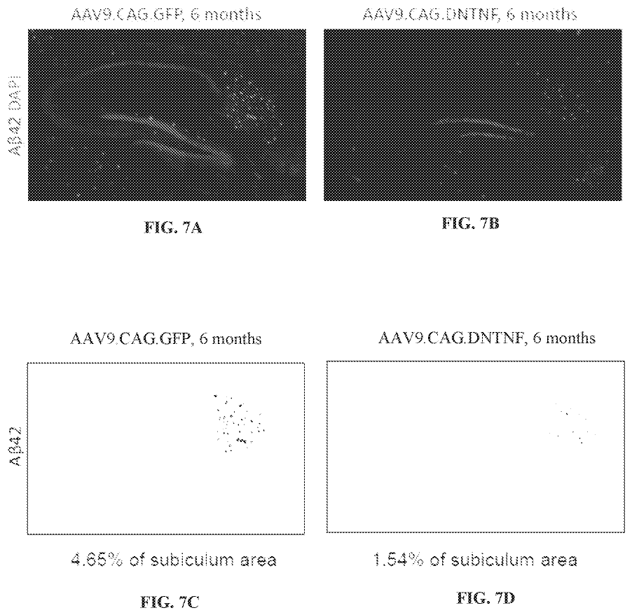

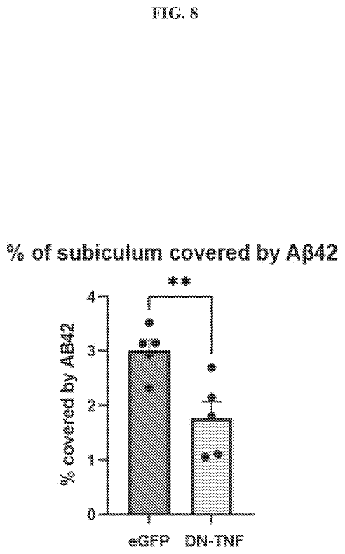

Representative images of the subiculum show puncta staining following anti-AB42 staining (FIGS. 7A-7D). The overall percentage of the subiculum area having puncta representing AB42 plaques was 4.65% in 6 month old 5XFAD mice treated with control (injection of AAV control vector at 2 months). The number of plaques in the subiculum area were reduced significantly (to 1.54% of subiculum area) four months following a single administration of AAV-CAG.DN-TNFα vector to the hippocampus (FIG. 8).

### Example 11: DN-TNF Reduces TNF-Induced Activation of NF-kB in Hela Cells and HEK-Blue-TNFα Cells

TNFα induces pro-inflammatory signaling by causing p65 nuclear localization. Activation of TNFR2 is pro-survival, while TNFR1 signaling can lead to cell death under certain conditions. DN-TNFα selectively inhibits TNFR1 signaling, allowing for transmembrane (tm) TNFα to induce pro-inflammatory and pro-survival signaling, yet reducing the activation of cell death pathways

A. DN-TNF was collected from conditioned media of HEK293T cells transfected with cis plasmid expressing DN-TNFα. The protein concentration of the DN-TNF conditioned media was determined using a hTNF-α ELISA kit, capable of detectin DN-TNF and not TNF.

Briefly, HELA cells were seeded at 150,00 cells per well. Fresh TNFα was added at 20 ng/mL while the DN-TNF added was 30-fold higher. The combined TNFa/DN-TNF mixture was added to HeLa cells plated on chamber slides. After 30 minutes the cells were fixed, blocked and permeabilized before adding the primary (anti-p65) and secondary antibodies. Immuno-fluorescence staining was performed to detect the transcription factor p65, which localizes to the nucleus in response to NF-κB/TNFα activation. A decrease in p65 nuclear fluorescence after DN-TNF treatment compared to untreated controls with TNF only was seen in replicated experiments.

B. Another study utilized the HEK-Blue TNFα cell line in which a SEAP reporter gene is fused to NF-κB. The substrate SEAP was produced in response to TNF-induced activation of NF-κB. The levels of SEAP in the supernatant were determined by adding QUANTI-Blue Solution, which turns the solution purple/blue in the presence of SEAP. Relative values were determined by measuring optical density via plate reader. Here, purple/blue color change indicates the degree of TNF-induced NF-κB activation. To determine if DN-TNF reduced TNF-induced color change in HEK-Blue cells, cell media was replaced with mixtures of pre-incubated DN-TNF (obtained from conditioned media from transfected HEK293T cells) combined with TNFα. The different mixtures of about 150 μL contained 60%, 50%, 45%, 15%, 5%, 1,7% and 0.5% DN-TNF with constant concentration of TNF. The first technical replicate showed a decrease in absorbance/TNFα activity when as low as 5% of the media contained DN-TNF conditioned media. This decrease in TNFα activity was comparable to the effect seen when media was replaced with adulimumab IgG, a well-studied anti-TNF antibody.

Analogously, a transfected L929 cell line (mouse fibroblast cell line) can be utilized in a cell death assay (e.g., phototoxicity) measured via optical density to elucidate reduction of TNF-induced cell death by expression of DN-TNF following transfection of vector in the cells.

### Example: 12. AAV-Delivered DN-TNF to Reduce Inflammation in a Mouse Model of Duchenne Muscular Dystrophy (DMD)

DN-TNF alone, AAV9.CAG.DN-TNF, or AAV9.MusclePromoter.DN-TNF is injected systemically (IV) or locally into muscle of mdx (−/−) mice to reduce inflammation and promote muscle regeneration in a relevant model of Duchenne Muscular Dystrophy. This treatment is combined with other vectors that replace dysfunctional microdystrophin (AAV-microdystrophin) to restore a functional fragment of dystrophin thus restoring muscle function and reducing inflammation associated with muscle degeneration. Readout measures include cytokine assays and muscle function/behavioral tests. Vectorized DN-TNF provides a benefit by reducing the inflammatory symptoms associated with DMD.

What is claimed is:

1. A pharmaceutical composition for treating neuroinflammation in a human subject in need thereof, comprising an adeno-associated virus (AAV) vector comprising:
(a) a viral capsid that is at least 95% identical to the amino acid sequence of AAV9 capsid (SEQ ID NO: 5); AAV.hDyn capsid (SEQ ID NO: 6); AAV.PHP.eB capsid (SEQ ID NO: 7); AAV.PHP.B capsid (SEQ ID NO: 8); AAV.PHP.S capsid (SEQ ID NO: 9); AAV.PHP.SH capsid (SEQ ID NO: 10); AAV8 capsid (SEQ ID NO: 11); AAV8.BBB capsid (SEQ ID NO: 12); AAV8.BBB.LD capsid (SEQ ID NO: 13); AAV9.BBB capsid (SEQ ID NO: 14); AAV9.BBB.LD capsid (SEQ ID NO: 15); AAVrh10 capsid (SEQ ID NO: 16); AAVrh.10.LD capsid (SEQ ID NO: 17); AAV9.496NNN/AAA498 capsid (SEQ ID NO: 18); VOY101 capsid (SEQ ID NO: 19); VOY201 capsid (SEQ ID NO: 20); VOY701 capsid (SEQ ID NO: 21); VOY801 capsid (SEQ ID NO: 22); VOY1101 capsid (SEQ ID NO: 23); or AAV.S454.Tfr3 (SEQ ID NO: 24); and
(b) an artificial genome comprising an expression cassette flanked by AAV Inverted Terminal Repeats (ITRs), wherein the expression cassette comprises a transgene encoding a dominant-negative tumor necrosis factor alpha (DN-TNFα) polypeptide, operably linked to a heterologous signal sequence, and one or more regulatory sequences that control expression of the transgene in human cells;
wherein said AAV vector is formulated for administration to the subject.

2. The pharmaceutical composition of claim 1, wherein the one or more regulatory sequences comprises a promoter selected from Table 7 or Table 8.

3. The pharmaceutical composition of claim 1, wherein the one or more regulatory sequences comprises (a) a promoter selected from Table 7 or Table 8, and (b) a poly A selected from Rabbit β-globin polyA (SEQ ID NO: 109), and β-globin PolyA signal (SEQ ID NO: 110).

4. The pharmaceutical composition of any one of claims 1 to 3, wherein the expression cassette comprises the nucleotide sequence set forth in any one of SEQ ID NOs: 165-170.

5. The pharmaceutical composition of any one of claims 1 to 4, wherein the neuroinflammation is associated with Alzheimer's Disease (AD), frontotemporal dementia (FD), tauopathies, progressive supranuclear palsy, chronic traumatic encephalopathy, Pick's Complex, and primary age-related tauopathy, Huntington's Disease (HD), Juvenile Huntington's Disease, Parkinson's Disease (PD), synucleinopathies, Amyotrophic Lateral Sclerosis (ALS), migraines, cluster headaches, stroke, depression, post-traumatic stress disorder (PTSD), or traumatic brain injury (TBI).

6. The pharmaceutical composition of any one of claims 1 to 5, wherein the transgene encoding the dominant-negative tumor necrosis factor alpha (DN-TNFα) polypeptide comprises the codon optimized nucleotide sequence set forth in SEQ ID NO: 3.

7. The pharmaceutical composition of any one of claims 1 to 6, wherein the transgene encoding the dominant-negative tumor necrosis factor alpha (DN-TNFα) polypeptide comprises the nucleotide sequence set forth in SEQ ID NO: 4.

8. The pharmaceutical composition of any one of claims 1 to 7, wherein the transgene encoding the DN-TNFα polypeptide is preceded by a nucleic acid sequence encoding a signal peptide selected from the group consisting of Mutant interleukin 2 (mIL2) signal peptide (SEQ ID NO: 115), VEGF-A signal peptide (SEQ ID NO: 118), Fibulin-1 signal peptide (SEQ ID NO: 119), Vitronectin signal peptide (SEQ ID NO: 120), Complement Factor H signal peptide (SEQ ID NO: 121), Opticin signal peptide (SEQ ID NO: 114), Albumin signal peptide (SEQ ID NO: 123), Chymotrypsinogen signal peptide (SEQ ID NO: 124), Interleukin-2 signal peptide (SEQ ID NO: 125), and Trypsinogen-2 signal peptide (SEQ ID NO: 126).

9. A pharmaceutical composition for treating Alzheimer's Disease in a human subject in need thereof, comprising an adeno-associated virus (AAV) vector comprising:
(a) a viral capsid that is at least 95% identical to the amino acid sequence of AAV9 capsid (SEQ ID NO: 5); AAV.hDyn capsid (SEQ ID NO: 6); AAV.PHP.eB capsid (SEQ ID NO: 7); AAV.PHP.B capsid (SEQ ID NO: 8); AAV.PHP.S capsid (SEQ ID NO: 9); AAV.PHP.SH capsid (SEQ ID NO: 10); AAV8 capsid (SEQ ID NO: 11); AAV8.BBB capsid (SEQ ID NO: 12); AAV8.BBB.LD capsid (SEQ ID NO: 13); AAV9.BBB capsid (SEQ ID NO: 14); AAV9.BBB.LD capsid (SEQ ID NO: 15); AAVrh10 capsid (SEQ ID NO: 16); AAVrh.10.LD capsid (SEQ ID NO: 17); AAV9.496NNN/AAA498 capsid (SEQ ID NO: 18); VOY101 capsid (SEQ ID NO: 19); VOY201 capsid (SEQ ID NO: 20); VOY701 capsid (SEQ ID NO: 21); VOY801 capsid (SEQ ID NO: 22); VOY1101 capsid (SEQ ID NO: 23); or AAV.S454.Tfr3 (SEQ ID NO: 24); and
(b) an artificial genome comprising an expression cassette flanked by AAV Inverted Terminal Repeats (ITRs), wherein the expression cassette comprises a transgene encoding a dominant-negative tumor necrosis factor alpha (DN-TNFα) polypeptide, operably linked to a heterologous signal sequence, and one or more regulatory sequences that control expression of the transgene in human brain cells;
wherein said AAV vector is formulated for administration to the subject.

10. The pharmaceutical composition of claim 9, wherein the regulatory sequence is one or more nucleic acid sequences selected from the group consisting of Chimeric Intron (SEQ ID NO: 107), VH4 Intron (SEQ ID NO: 108), Rabbit β-globin polyA (SEQ ID NO: 109), β-globin PolyA signal (SEQ ID NO: 110), and any of the promoters in Table 7 and Table 8.

11. The pharmaceutical composition of claim 9 or claim 10, wherein the ITRs are (a) a 5′ ITR selected from 5′ITR-A (SEQ ID NO: 111), and 5′-ITR-B (SEQ ID NO: 112); and (b) a 3′ ITR selected from 3′-ITR AAV (SEQ ID NO: 113), and Alternative 3′-ITR (SEQ ID NO: 183).

12. The pharmaceutical composition of any one of claims 9 to 11, wherein the DN-TNFα polypeptide is a variant sequence relative to the sequence encoding wild-type TNF-α polypeptide.

13. The pharmaceutical composition of any one of claims 9 to 12, wherein the DN-TNFα polypeptide has the amino acid substitution A145R and Y87H.

14. A pharmaceutical composition for delivering dominant-negative tumor necrosis factor alpha (DN-TNFα) to a brain to treat Alzheimer's Disease, prevent or inhibit the onset of AD, or reduce cognitive or functional decline in AD, in a human subject in need or at risk thereof, the composition comprising a recombinant AAV comprising a transgene encoding DN-TNFα operably linked to a heterologous signal sequence, and one or more regulatory sequences that control expression of the transgene in brain cells, wherein the recombinant AAV is administered to the human subject at a dose of about 5×10¹² to about 2×10¹⁴ genome copies to the brain of the human subject.

15. A method of treating Alzheimer's Disease (AD), inhibiting the onset of AD, or reducing cognitive or functional decline in AD, in a human subject in need or at risk thereof, the method comprising administering a recombinant adeno-associated virus (AAV) vector comprising a transgene encoding dominant-negative tumor necrosis factor alpha (DN-TNFα) to the brain of the subject, wherein the transgene encoding the DN-TNFα is operably linked to a heterologous signal sequence, and one or more regulatory sequences that control expression of the transgene in brain cells, wherein the recombinant AAV is administered to the human subject at a dose of about 5×10¹² to about 2×10¹⁴ genome copies to the brain of the human subject.

16. The composition of claim 14, or the method of claim 15, wherein the regulatory sequence in the AAV vector is one or more nucleic acid sequences selected from the group consisting of Chimeric Intron (SEQ ID NO: 107), VH4 Intron (SEQ ID NO: 108), Rabbit β-globin polyA (SEQ ID NO: 109), and β-globin Poly A signal (SEQ ID NO: 110), and any of the promoters in Table 7 and Table 8.

17. The composition of claim 14, or the method of claim 15, wherein the transgene encoding the DN-TNFα polypeptide in the AAV vector is preceded by a nucleic acid sequence encoding a signal peptide selected from the group consisting of Mutant interleukin 2 (mIL2) signal peptide (SEQ ID NO: 115), VEGF-A signal peptide (SEQ ID NO: 118), Fibulin-1 signal peptide (SEQ ID NO: 119), vitronectin signal peptide (SEQ ID NO: 120), Complement Factor H signal peptide (SEQ ID NO: 121), opticin signal peptide (SEQ ID NO: 122), Albumin signal peptide (SEQ ID NO: 123), chymotrypsinogen signal peptide (SEQ ID NO: 124), Interleukin-2 signal peptide (SEQ ID NO: 125), and Trypsinogen-2 signal peptide (SEQ ID NO: 126).

18. The method of any one of claims 15-17, wherein administration of the recombinant AAV results in reduced levels of one or more of the following parameters:
(a) Aβ accumulation;
(b) amyloid plaques;
(c) Tau accumulation;
(d) neuroinflammation;
(e) white matter free water (WMFW); and/or
(f) one or more of CCL8, OLR1, IL2, CXCL9, TGFA, IL6, TNFSF12, CCL11, HGF, FLT3LG, IL17F, IL7, IL18, CCL13, TNFSF10, CXCL10, IFNG, IL10, 1L15, CCL3, CXCL8, MMP12, CSF2, VEGFA, IL17C, CCL2, IL17A, OSM, CSF1, CCL4, CXCL11, LTA, CCL7, and MMP1.

19. The method of any one of claims 15-17, wherein administration of the recombinant AAV results in enhanced cognitive function and/or increased microglial phagocytosis.

20. The method of claim 18, wherein the level(s) of one or more parameters in (a)-(f) is/are lower by at least 10%, as compared to corresponding reference level(s) in the subject or in a control.

21. The method of claim 19, wherein the subject's cognitive function is enhanced by at least about 10%, as measured on one or more tests selected from the group consisting of the Alzheimer's Disease Assessment Scale-cognitive subscale (ADAS-cog); clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB); the Sandoz Clinical Assessment-Geriatric (SCAG), the Buschke Selective Reminding Test; the Verbal Paired Associates subtest; the Logical Memory subtest: the Visual Reproduction subtest of the Wechsler Memory Scale-Revised (WMS-R); the explicit 3-alternative forced choice task; and the Benton Visual Retention Test.

22. The method of any one of claims 15-21, wherein the subject is also treated with one or more agents selected from the group consisting of a cholinesterase inhibitor, an N-methyl-D-aspartate (NMDA) receptor antagonist, a hormone, a vitamin, an antipsychotic, a tricyclic antidepressant, a benzodiazepine, insulin, adeno-associated virus delivery of nerve growth factor (NGF), beta-blocker, human amyloid vaccine, beta or gamma secretase inhibitor, nicotinic or muscarinic agonist, and an antibody.

23. The method of any one of claims 15-22, wherein the cognitive decline is assessed by determining the subject's score before and after administration of said AAV vector comprising the transgene encoding DN-TNFα using an Alzheimer's Disease Assessment Scale-Cognition (ADAS-Cog) test.

24. The method of claim 23, wherein the reduction in cognitive decline as measured by ADAS-Cog is at least 10%, relative to a placebo.

25. The method of any one of claims 15-24, wherein the subject has mild, moderate, or severe AD.

26. The method of any one of claims 15-25, wherein the treatment is prophylactic for completely or partially preventing AD or symptoms thereof in the subject.

27. The method of any one of claims 15-26, wherein the treatment is therapeutic for partially or completely curing AD or symptoms associated with AD in the subject.

28. The method of any one of claims 15-27, wherein the recombinant AAV is administered intravenously (IV), intraparenchymally, intracerebroventricularly (ICV), intracisternally (IC), or by lumbar intrathecal (IT) delivery.

29. The method of claim 28, wherein the intraparenchymal administration is intrastriatal or intrahippocampal.

30. A pharmaceutical composition for delivering a dominant-negative tumor necrosis factor alpha (DN-TNFα) polypeptide into the brain of a human subject in need thereof, comprising an adeno-associated virus (AAV) vector comprising:
(a) an AAV viral capsid that transduces brain cells; and
(b) an artificial genome comprising an expression cassette flanked by AAV Inverted Terminal Repeats (ITRs), wherein the expression cassette comprises a transgene encoding DN-TNFα, operably linked to a heterologous signal sequence, and a promoter that directs expression of the transgene in brain cells.

31. A method of treating Alzheimer's Disease (AD), inhibiting the onset of AD, or reducing cognitive or functional decline in AD, in a human subject in need or at risk thereof, the method comprising administering a dominant-negative tumor necrosis factor alpha (DN-TNFα) polypeptide in the brain of the human subject in need thereof, comprising an adeno-associated virus (AAV) vector comprising:
(a) an AAV viral capsid that transduces brain cells; and
(b) an artificial genome comprising an expression cassette flanked by AAV Inverted Terminal Repeats (ITRs), wherein the expression cassette comprises a transgene encoding a DN-TNFα, operably linked to a heterologous signal sequence, and a promoter that directs expression of the transgene in brain cells.

32. The method of claim 31, wherein the administration of the AAV vector is intravenous, intracerebral, intraparenchymal, intracerebroventricular (ICV), intracisternal (IC), intraventricular, lumbar intrathecal (IT), or by a brain implant.

33. The method of claim 32, wherein the intraparenchymal administration is intrastriatal or intrahippocampal.

34. The composition of claim 30, or the method of claim 31, wherein the promoter in the AAV vector is CAG (SEQ ID NO: 48); CB/CBA promoter (SEQ ID NO: 49); UbC promoter (SEQ ID NO: 50); mU1a (SEQ ID NO: 51); EF-1α (SEQ ID NO: 52); Human Synapsin Promoter 1 (hSyn-1; (SEQ ID NO: 53); Human Synapsin Promoter 2 (hSyn-2) (SEQ ID NO: 54); Human Synapsin Promoter 3 (hSyn-3) (SEQ ID NO: 55); Human Synapsin Promoter 4 (hSyn-4) (SEQ ID NO: 56); Human Synapsin Promoter 5 (hSyn-5) (SEQ ID NO: 57); Mecp2 promoter (SEQ ID NO: 58); hGFAP promoter (SEQ ID NO: 59); Rat NSE/RUS' promoter (SEQ ID NO: 60); NeuN (SEQ ID NO: 61); CaMKII_1 (SEQ ID NO: 62); C1ql2 1 (SEQ ID NO: 63); C1ql2 2 (SEQ ID NO: 64); DRD1 (SEQ ID NO: 65); DRD2 isoform 1 (SEQ ID NO: 66); DRD2 isoform 2 (SEQ ID NO: 67); POMC (SEQ ID NO: 68); PROX1 isoform 1 (SEQ ID NO: 69); PROX1 isoform 2 (SEQ ID NO: 70); MAP1B isoform 1 (SEQ ID NO: 71); MAP1B isoform 2 (SEQ ID NO: 72); MAP1B isoform 3 (SEQ ID NO: 73); or Ta-1/TUBA1A isoform 1 (SEQ ID NO: 74).

35. A pharmaceutical composition for treating Rheumatoid Arthritis in a human subject in need thereof, comprising an adeno-associated virus (AAV) vector comprising:
(a) a viral capsid that is at least 95% identical to the amino acid sequence of AAV8 capsid (SEQ ID NO: 11); AAV9 capsid (SEQ ID NO: 5); AAV.hu37 capsid (SEQ ID NO: 25); AAVrh74 version 1 capsid (SEQ ID NO: 26); AAVrh74 version 2 capsid (SEQ ID NO: 27); AAV.hu.31 capsid (SEQ ID NO: 28); or AAV.hu32 capsid (SEQ ID NO: 29); and
(b) an artificial genome comprising an expression cassette flanked by AAV Inverted Terminal Repeats (ITRs), wherein the expression cassette comprises a transgene encoding a dominant-negative tumor necrosis factor alpha (DN-TNFα) polypeptide, operably linked to a heterologous signal sequence, and one or more regulatory sequences that control expression of the transgene in human muscle, liver and/or synovial cells;
wherein said AAV vector is formulated for administration to the subject.

36. The pharmaceutical composition of claim 35, wherein the transgene encoding the dominant-negative tumor necrosis factor alpha (DN-TNFα) polypeptide comprises the sequence set forth in SEQ ID NO: 3 or SEQ ID NO: 4.

37. The pharmaceutical composition of claim 35 or claim 36, wherein the regulatory sequence is one or more nucleic acid sequences selected from the group consisting of Chimeric Intron (SEQ ID NO: 107), VH4 Intron (SEQ ID NO: 108), Rabbit β-globin polyA (SEQ ID NO: 109), and β-globin PolyA signal (SEQ ID NO: 110), and any of the promoters in Table 7 and Table 9.

38. The pharmaceutical composition of any one of claims 35 to 37, wherein the ITRs are (a) a 5′ ITR selected from 5′ITR-A (SEQ ID NO: 111), and 5′-ITR-B (SEQ ID NO: 112); and (b) a 3′ ITR selected from 3′-ITR AAV (SEQ ID NO: 113), and Alternative 3′-ITR (SEQ ID NO: 183).

39. The pharmaceutical composition of any one of claims 35 to 38, wherein the transgene encoding the DN-TNFα polypeptide is preceded by a nucleic acid sequence encoding a signal peptide selected from the group consisting of Mutant interleukin 2 (mIL2) signal peptide (SEQ ID NO: 115), VEGF-A signal peptide (SEQ ID NO: 118), Fibulin-1 signal peptide (SEQ ID NO: 119), vitronectin signal peptide (SEQ ID NO: 120), Complement Factor H signal peptide (SEQ ID NO: 121), opticin signal peptide (SEQ ID NO: 122), Albumin signal peptide (SEQ ID NO: 123), chymotrypsinogen signal peptide (SEQ ID NO: 124), Interleukin-2 signal peptide (SEQ ID NO: 125), and Trypsinogen-2 signal peptide (SEQ ID NO: 126).

40. The pharmaceutical composition of any one of claims 35 to 39, wherein the DN-TNFα polypeptide is a variant sequence relative to the sequence encoding wild-type TNF-α polypeptide.

41. The pharmaceutical composition of any one of claims 35 to 40, wherein the DN-TNFα polypeptide has the amino acid substitution A145R and Y87H.

42. A pharmaceutical composition for delivering dominant-negative tumor necrosis factor alpha (DN-TNFα) to the human subject to treat Rheumatoid Arthritis (RA), prevent or inhibit the onset of RA, or reduce inflammation in RA, in a human subject in need or at risk thereof, the composition comprising a recombinant AAV comprising a transgene encoding DN-TNFα operably linked to a heterologous signal sequence, and one or more regulatory sequences that control expression of the transgene in muscle, liver and/or synovial cells, wherein the recombinant AAV is administered to the human subject at a dose of 1×10¹⁰ to 1×10¹⁶ genome copies.

43. A method of treating Rheumatoid Arthritis (RA), preventing or inhibiting the onset of RA, or reducing inflammation in RA, in a human subject in need or at risk thereof, the method comprising administering a recombinant adeno-associated virus (AAV) vector comprising a transgene encoding dominant-negative tumor necrosis factor alpha (DN-TNFα) to the subject, wherein the transgene encoding the DN-TNFα is operably linked to a heterologous signal sequence, and one or more regulatory sequences that control expression of the transgene in muscle, liver, and/or synovial cells, wherein the recombinant AAV is administered to the human subject at a dose of 1×10¹⁰ to 1×10¹⁶ genome copies.

44. The composition of claim 42 or method of claim 43, wherein the regulatory sequence in the AAV vector is one or more nucleic acid sequences selected from the group consisting of Chimeric Intron (SEQ ID NO: 107), VH4 Intron (SEQ ID NO: 108), Rabbit β-globin polyA (SEQ ID NO: 109), and β-globin Poly A signal (SEQ ID NO: 110), and any of the promoters in Table 7 and Table 9.

45. The composition of claim 42 or method of claim 43, wherein the ITRs are (a) a 5′ ITR selected from 5′ITR-A (SEQ ID NO: 111), and 5′-ITR-B (SEQ ID NO: 112); and (b) a 3′ ITR selected from 3′-ITR AAV (SEQ ID NO: 113), and Alternative 3′-ITR (SEQ ID NO: 183).

46. The composition of claim 42 or method of claim 43, wherein the transgene encoding the DN-TNFα polypeptide in the AAV vector is preceded by a nucleic acid sequence encoding a signal peptide selected from the group consisting of Mutant interleukin 2 (mIL2) signal peptide (SEQ ID NO: 115), VEGF-A signal peptide (SEQ ID NO: 118), Fibulin-1 signal peptide (SEQ ID NO: 119), Vitronectin signal peptide (SEQ ID NO: 120), Complement Factor H signal peptide (SEQ ID NO: 121), Opticin signal peptide (SEQ ID NO: 122), Albumin signal peptide (SEQ ID NO: 123), Chymotrypsinogen signal peptide (SEQ ID NO: 124), Interleukin-2 signal peptide (SEQ ID NO: 125), and Trypsinogen-2 signal peptide (SEQ ID NO: 126).

47. The method of any one of claims 43-46, wherein administration of the recombinant AAV results in one or more of the following parameters: a reduction in the American College of Rheumatology (ACR) score, a reduction in the Disease Activity Score 28 (DAS28), a reduction in total joint score progression, a reduction in serum C-reactive protein (CRP) and a reduction in circulating soluble TNF receptors.

48. The method of any one of claims 43-46, wherein administration of the recombinant AAV results in an improvement in the Visual Analog Scale (VAS).

49. The method of claim 47, wherein the level(s) of one or more parameters is/are lower by at least 20%, as compared to corresponding reference level(s) in the subject or in a control.

50. The method of any one of claims 43-49, wherein the subject is concurrently treated with one or more agents selected from the group consisting of A Disease Modifying Anti-Rheumatic Drug (DMARD) or a Nonsteroidal Anti-Inflammatory Drug (NSAID) and a steroid.

51. The method of any one of claims 43-50, wherein the treatment is prophylactic for completely or partially preventing RA or symptoms thereof in the subject.

52. The method of any one of claims 43-51, wherein the treatment is therapeutic for partially or completely curing RA or symptoms associated with RA in the subject.

53. The method of any one of claims 43-52, wherein the recombinant AAV is administered by intravenous, intramuscular, intrasynovial, intra-articular, or peri-articular delivery.

54. A pharmaceutical composition for delivery of a dominant-negative tumor necrosis factor alpha (DN-TNFα) polypeptide in the muscle of a human subject in need thereof, comprising an adeno-associated virus (AAV) vector comprising:
(a) an AAV viral capsid that infects muscle, liver, and/or synovial cells; and
(b) an artificial genome comprising an expression cassette flanked by AAV Inverted Terminal Repeats (ITRs), wherein the expression cassette comprises a transgene encoding a DN-TNFα, operably linked to a promoter that directs expression in muscle, liver and/or synovial cells.

55. A method of treating Rheumatoid arthritis (RA), inhibiting the onset of RA, or reducing inflammation in RA, in a human subject in need or at risk thereof, the method comprising administering a dominant-negative tumor necrosis factor alpha (DN-TNFα) polypeptide in the muscle of the human subject in need thereof, comprising an adeno-associated virus (AAV) vector comprising:
(a) an AAV viral capsid that infects muscle, liver and/or synovial cells; and
(b) an artificial genome comprising an expression cassette flanked by AAV Inverted Terminal Repeats (ITRs), wherein the expression cassette comprises a transgene encoding a DN-TNFα, operably linked to a promoter that directs expression in muscle, liver and/or synovial cells.

56. The method of claim 55, wherein the administration of the AAV vector is intravenous, intramuscular, intrasynovial, intra-articular, or peri-articular.

57. The composition of claim 54, or the method of claim 55, wherein the promoter in the AAV vector is CAG (SEQ ID NO: 48); CB/CBA promoter (SEQ ID NO: 49); UbC promoter (SEQ ID NO: 50); mU1a (SEQ ID NO: 51); EF-1α (SEQ ID NO: 52); LSPX1 (SEQ ID NO: 75); LSPX2 (SEQ ID NO: 76); LTP1 (SEQ ID NO: 77); LTP2 (SEQ ID NO: 78); LTP3 (SEQ ID NO: 79); LMTP6 (SEQ ID NO: 80); LMTP13 (SEQ ID NO: 81); LMTP14 (SEQ ID NO: 82); LMTP15 (SEQ ID NO: 83); LMTP18 (SEQ ID NO: 84); LMTP19 (SEQ ID NO: 85); LMTP20 (SEQ ID NO: 86); LBTP1 (SEQ ID NO: 87); LBTP2 (SEQ ID NO: 88); hAAT (SEQ ID NO: 89); ApoE.hAAT (SEQ ID NO: 90); TBG (SEQ ID NO: 91); CK8 (SEQ ID NO: 92); SPc5-12 (SEQ ID NO: 93); MCK7 (SEQ ID NO: 94); truncatedMCK (IMCK) (SEQ ID NO: 95); Mouse skeletal muscle alpha actin acta1 (SEQ ID NO: 96); Human muscle creatine kinase (MCK) (SEQ ID NO: 97); Human desmin (SEQ ID NO: 98); Human desmin 2 (SEQ ID NO: 99); Human skeletal muscle alpha actin acta1 (SEQ ID NO: 100); Mouse muscle creatine kinase (MCK) (SEQ ID NO: 101); Mouse desmin (SEQ ID NO: 102); or CXCL10 (SEQ ID NO: 103).

58. A pharmaceutical composition for treating uveitis in a human subject in need thereof, comprising an adeno-associated virus (AAV) vector comprising:
(a) a viral capsid that is at least 95% identical to the amino acid sequence of AAV8 capsid (SEQ ID NO: 11); AAV9 capsid (SEQ ID NO: 5); AAV2 capsid (SEQ ID NO: 30); AAV3B capsid (SEQ ID NO: 31); AAV2.7m8 capsid (SEQ ID NO: 32); AAV.rh.34 capsid (SEQ ID NO: 33); AAV.hu.31 capsid (SEQ ID NO: 28); AAV.rh.31 capsid (SEQ ID NO: 34); AAV.hu.12 capsid (SEQ ID NO: 35); AAV.hu.13 capsid (SEQ ID NO: 36); AAV.hu.21 capsid (SEQ ID NO: 37); AAV.hu.26 capsid (SEQ ID NO: 38); AAV.hu.53 capsid (SEQ ID NO: 39); AAV.hu.56 capsid (SEQ ID NO: 40); AAV.rh.24 capsid (SEQ ID NO: 41); AAV.hu.38 capsid (SEQ ID NO: 42); AAV.rh.72 capsid (SEQ ID NO: 43); AAV.cy.5 capsid (SEQ ID NO: 44); AAV.cy.6 capsid (SEQ ID NO: 45); AAV.rh.46 capsid (SEQ ID NO: 46); or AAV.rh.2 capsid (SEQ ID NO: 47); and
(b) an artificial genome comprising an expression cassette flanked by AAV Inverted Terminal Repeats (ITRs), wherein the expression cassette comprises a transgene encoding a dominant-negative tumor necrosis factor alpha (DN-TNFα) polypeptide, operably linked to a heterologous signal sequence, and one or more regulatory sequences that control expression of the transgene in human ocular cells, retinal pigment epithelial cells, and/or retinal cells;
wherein said AAV vector is formulated for administration to the subject.

59. The pharmaceutical composition of claim 58, wherein the transgene encoding the dominant-negative tumor necrosis factor alpha (DN-TNFα) polypeptide comprises the sequence set forth in SEQ ID NO: 3 or SEQ ID NO: 4.

60. The pharmaceutical composition of claim 58 or claim 59, wherein the regulatory sequence is one or more nucleic acid sequences selected from the group consisting of Chimeric Intron (SEQ ID NO: 107), VH4 Intron (SEQ ID NO: 108), Rabbit β-globin polyA (SEQ ID NO: 109), and β-globin PolyA signal (SEQ ID NO: 110), and any of the promoters in Table 7 and Table 10.

61. The pharmaceutical composition of any one of claims 58 to 60, wherein the ITRs are (a) a 5′ ITR selected from 5′ITR-A (SEQ ID NO: 111), and 5′-ITR-B (SEQ ID NO: 112); and (b) a 3′ ITR selected from 3′-ITR AAV (SEQ ID NO: 113), and Alternative 3′-ITR (SEQ ID NO: 183).

62. The pharmaceutical composition of any one of claims 58 to 61, wherein the transgene encoding the DN-TNFα polypeptide is preceded by a nucleic acid sequence encoding signal peptide selected from the group consisting of Mutant interleukin 2 (mIL2) signal peptide (SEQ ID NO: 115), VEGF-A signal peptide (SEQ ID NO: 118), Fibulin-1 signal peptide (SEQ ID NO: 119), Vitronectin signal peptide (SEQ ID NO: 120), Complement Factor H signal peptide (SEQ ID NO: 121), Opticin signal peptide (SEQ ID NO: 122), Albumin signal peptide (SEQ ID NO: 123), Chymotrypsinogen signal peptide (SEQ ID NO: 124), Interleukin-2 signal peptide (SEQ ID NO: 125), and Trypsinogen-2 signal peptide (SEQ ID NO: 126).

63. The pharmaceutical composition of any one of claims 58 to 62, wherein the DN-TNFα polypeptide is a variant sequence relative to the sequence encoding wild-type TNF-α polypeptide.

64. The pharmaceutical composition of any one of claims 58 to 63, wherein the DN-TNFα polypeptide has the amino acid substitution A145R and Y87H.

65. A pharmaceutical composition for delivering dominant-negative tumor necrosis factor alpha (DN-TNFα) to human ocular cells, retinal pigment epithelial cells, and/or retinal cells to treat uveitis, prevent or inhibit the onset of uveitis, or reduce inflammation in uveitis, in a human subject in need or at risk thereof, the composition comprising a recombinant AAV comprising a transgene encoding DN-TNFα operably linked to a heterologous signal sequence, and one or more regulatory sequences that control expression of the transgene in human ocular cells, retinal pigment epithelial cells, and/or retinal cells, wherein the recombinant AAV is administered to the human subject at a dose of about 2×10¹⁰ GC to about 6×10¹⁰ GC per eye.

66. A method of treating uveitis, preventing or inhibiting the onset of uveitis, or reducing inflammation in uveitis, in a human subject in need or at risk thereof, the method comprising administering a recombinant adeno-associated virus (AAV) vector comprising a transgene encoding dominant-negative tumor necrosis factor alpha (DN-TNFα) to the human ocular cells, retinal pigment epithelial cells, and/or retinal cells of the subject, wherein the transgene encoding the DN-TNFα is operably linked to a heterologous signal sequence, and one or more regulatory sequences that control expression of the transgene in human ocular cells, retinal pigment epithelial cells, and/or retinal cells, wherein the recombinant AAV is administered to the human subject at a dose of about 2×10¹⁰ GC to about 6×10¹⁰ GC per eye.

67. The composition of claim 65, or the method of claim 66, wherein the regulatory sequence in the AAV vector is one or more nucleic acid sequences selected from the group consisting of Chimeric Intron (SEQ ID NO: 107), VH4 Intron (SEQ ID NO: 108), Rabbit β-globin polyA (SEQ ID NO: 109), and β-globin PolyA signal (SEQ ID NO: 110), and any of the promoters in Table 7 and Table 10.

68. The composition of claim 65, or the method of claim 66, wherein the transgene encoding the DN-TNFα polypeptide in the AAV vector is preceded by a nucleic acid sequence encoding a signal peptide selected from the group consisting of Mutant interleukin 2 (mIL2) signal peptide (SEQ ID NO: 115), VEGF-A signal peptide (SEQ ID NO: 118), Fibulin-1 signal peptide (SEQ ID NO: 119), Vitronectin signal peptide (SEQ ID NO: 120), Complement Factor H signal peptide (SEQ ID NO: 121), Opticin signal peptide (SEQ ID NO: 122), Albumin signal peptide (SEQ ID NO: 123), Chymotrypsinogen signal peptide (SEQ ID NO: 124), Interleukin-2 signal peptide (SEQ ID NO: 125), and Trypsinogen-2 signal peptide (SEQ ID NO: 126).

69. The method of any one of claims 66-68, wherein administration of the recombinant AAV results in one or more of the following parameters: reduction of visual haze, decrease of inflammatory lesions, decrease in tissue destruction, decrease in biomarkers of autoimmunity and/or inflammation, decrease in vasculitis, decrease in cellular infiltration, or decrease in edema.

70. The method of any one of claims 66-68, wherein administration of the recombinant AAV results in improvement in clinical symptoms of uveitis and/or improvement of vision.

71. The method of claim 69, wherein the level(s) of one or more parameters is/are lower by at least 20%, as compared to corresponding reference level(s) in the subject or in a control.

72. The method of claim 69, wherein the vision is enhanced by at least about 20%, as measured on one or more tests selected from the group consisting of Applanation Tonometry, Corneal Topography, Fluorescein Angiogram, Dilated Pupillary Exam, Refraction, Slit-Lamp Exam, Non-Contact Tonometry, Retinal Tomography, Ultrasound, Visual Acuity Testing, and Visual Field Test.

73. The method of any one of claims 66-72, wherein the subject is concurrently treated with one or more agents selected from the group consisting of an anti-inflammatory agent, an anti-fungal agent, and an immunosuppressive agent, a corticosteroid, an A3 adenosine receptor selective agonist), corticotropin zinc hydroxide, cyclopentolate, cyclosporine, cyclosporine A, dexchlorpheniramine, LFG-316 (anti-C5), homatropine, hyoscyamine sulfate, phenylephrine, an anti-IL-6R monoclonal antibody), an anti-IL-17A monoclonal antibody, an mTOR inhibitor, an IL-1 beta antagonist, an anti-TNF monoclonal antibody, a muscarinic receptor antagonist, methotrexate, azathioprine, acyclovir, gentamycin, neomycin, polymyxin B, rolitetracycline, sulfacetamide, valacyclovir, chloramphenicol, mycophenolate, fluocinolone, neomycin, polymyxin B, prednisolone and sulfacetamide.

74. The method of any one of claims 66-72, wherein the uveitis is anterior uveitis, intermediate uveitis and/or posterior uveitis.

75. The method of any one of claims 66-74, wherein the treatment is prophylactic for completely or partially preventing uveitis or symptoms thereof in the subject.

76. The method of any one of claims 66-75, wherein the treatment is therapeutic for partially or completely curing uveitis or symptoms associated with uveitis in the subject.

77. The method of any one of claims 66-76, wherein the recombinant AAV is administered by subretinal, intravitreal, suprachoroidal, or intracameral delivery.

78. A pharmaceutical composition for delivery of a dominant-negative tumor necrosis factor alpha (DN-TNFα) polypeptide in the human ocular cells, retinal pigment epithelial cells, and/or retinal cells of a human subject in need thereof, comprising an adeno-associated virus (AAV) vector comprising:
(a) an AAV viral capsid that infects human ocular cells, retinal pigment epithelial cells, and/or retinal cells; and
(b) an artificial genome comprising an expression cassette flanked by AAV Inverted Terminal Repeats (ITRs), wherein the expression cassette comprises a transgene encoding a DN-TNFα, operably linked to a promoter that directs expression in human ocular cells, retinal pigment epithelial cells, and/or retinal cells.

79. A method of treating uveitis, preventing or inhibiting the onset of uveitis, or reducing inflammation in uveitis, in a human subject in need or at risk thereof, the method comprising delivering a dominant-negative tumor necrosis factor alpha (DN-TNFα) polypeptide in the eye of the human subject in need thereof, comprising administering an adeno-associated virus (AAV) vector comprising:
(a) an AAV viral capsid that infects human ocular cells, retinal pigment epithelial cells, and/or retinal cells; and
(b) an artificial genome comprising an expression cassette flanked by AAV Inverted Terminal Repeats (ITRs), wherein the expression cassette comprises a transgene encoding a DN-TNFα, operably linked to a promoter that directs expression in human ocular cells, retinal pigment epithelial cells, and/or retinal cells.

80. The method of claim 79, wherein the administration of the AAV vector is subretinal, intravitreal, suprachoroidal, or intracameral.

81. The composition of claim 78, or the method of claim 79, wherein the promoter in the AAV vector is CAG (SEQ ID NO: 48); CB/CBA promoter (SEQ ID NO: 49); UbC promoter (SEQ ID NO: 50); mU1a promoter (SEQ ID NO: 51); EF-1α promoter (SEQ ID NO: 52); RPE65 promoter (SEQ ID NO: 104); red cone opsin promoter (SEQ ID NO: 105); or BST1 promoter (SEQ ID NO: 106).

82. The pharmaceutical composition of any one of claim 1, 9, 14, 30, 35, 42, 58, or 66, or the method of any one of claim 15, 31, 43, or 66, wherein the heterologous signal sequence controls the secretion of the DN-TNFα polypeptide, e.g., to the extracellular space, such as through the Endoplasmic Reticulum.

83. A method of inducing pro-inflammatory and/or pro-survival TNF receptor signaling in a human tissue, the method comprising delivering a dominant-negative tumor necrosis factor alpha (DN-TNFα) polypeptide, operably linked to a heterologous signal sequence, and one or more regulatory sequences that control expression of the DN-TNFα in human cells by administration of a recombinant adeno-associated virus (rAAV) vector comprising:
(a) an rAAV capsid and
(b) an artificial genome comprising an expression cassette flanked by AAV Inverted Terminal Repeats (ITRs), wherein the expression cassette comprises a transgene encoding the dominant-negative tumor necrosis factor alpha (DN-TNFα) polypeptide operably linked to the heterologous signal sequence and the one or more regulatory sequences.

84. A method of treating Duchenne muscular dystrophy (DMD), or reducing inflammation associated with DMD, in a human subject in need or at risk thereof, the method comprising delivering a dominant-negative tumor necrosis factor alpha (DN-TNFα) polypeptide in a muscle of the human subject in need thereof, comprising administering an adeno-associated virus (AAV) vector comprising:
(a) an AAV viral capsid that infects human muscle cells; and
(b) an artificial genome comprising an expression cassette flanked by AAV Inverted Terminal Repeats (ITRs), wherein the expression cassette comprises a transgene encoding a DN-TNFα, operably linked to a heterologous signal sequence and a promoter that directs expression in human muscle.

85. The method of claim 84, further comprising administering to the human subject an agent capable of restoring a functional fragment of dystrophin.

86. The method of claim 85, wherein the agent comprises an AAV-microdystrophin or an exon-skipping therapy.


SEQUENCE LISTING (189 sequences)

SEQ ID NO: 1 - protein, 157 aa, Homo sapiens [sequence omitted]

SEQ ID NO: 2 - protein, 157 aa, Homo sapiens
VRSSSRTPSDKPVAHVVANPQAEGQLQWLNRRANALLANGVELRDNQLVVPSEGLYLIYSQVLFKGQGCPSTHVLLTHTISRIAVSHQTKVNLLSAIKSPCQRETPEGAEAKPWYEPIYLGGVFQLEKGDRLSAEINRPDYLDFRESGQVYFGIIAL

SEQ ID NO: 3 - DNA, 474 bp, Homo sapiens
  source 1..474: mol_type = genomic DNA
gtgagatcctcctcaagaaccccttctgataaacctgtggctcatgtggtggctaaccctcaggcagaaggacagctgcagtggctgaataggagggccaatgcactgctggctaatggagtggaactgagggacaaccagctggtggtcccttcagaaggactgtacctgatctatagccaggtgctgttcaagggccagggatgtcccagtactcatgtgctgctgactcacaccatctccagaattgctgtctcccatcagacaaaggtgaacctgctgtcagccatcaaaagtccctgccagagagagacaccagaaggagctgaggccaagccatggtatgagccaatctacctggggggagtgtttcagctggagaaaggagacaggctgagtgcagaaatcaacagaccagactatctggactttagagaatcagggcaggtgtattttggcattattgcactgtga

SEQ ID NO: 4 - DNA, 474 bp, Homo sapiens
  source 1..474: mol_type = genomic DNA
gtacgctcctcctcccgcactccgtccgacaaaccggtagctcacgtagtagctaacccgcaggctgaaggtcagctgcagtggctgaaccgccgcgctaacgctctgctggctaacggtgtagaactgcgcgacaaccagctggtagtaccgtccgaaggtctgtacctgatctactcccaggtactgttcaaaggtcagggttgtccgtccactcacgtactgctgactcacactatctcccgcatcgctgtatcctaccagactaaagtaaacctgctgtccgctatcaaatccccgtgtcagcgcgaaactccggaaggtgctgaagctaaaccgtggtacgaaccgatctacctgggtggtgtattccagctggaaaaaggtgaccgcctgtccgctgaaatcaaccgcccggactacctggacttcgctgaatccggtcaggtatacttcggtatcatcgctctgtga

SEQ ID NO: 5 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAAPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQAVRTSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQAVRTSHAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 12 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 13 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQAAASNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 14 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNGTSGGSTNDNTYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNGTSGGSTNDNTYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 17 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQAAASNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 18 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 20 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 21 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSESTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSGGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 23 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLATPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 24 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSRTIGPSVGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 25 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 26 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 27 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 28 - protein, 736 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 29 - protein, 737 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 30 - protein, 735 aa, synthetic construct [sequence omitted]

SEQ ID NO: 31 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 32 - protein, 743 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTR

SEQ ID NO: 33 - protein, 733 aa, synthetic construct
MAADGYLPDWLEDNLSEGIREWWDLKPGAPKPKANQQKQDDGRGLVLPGYEYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFETAYNFEKVPFHSMYAHSQSLDGLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPAYPATTFTAARVDSFITQYSTGQVAVQIEWEIEKERSKRWNPEVQFTSNCGNQSSMLWAPDTTGKYTEPRVIGSRYLTNHL

SEQ ID NO: 34 - protein, 643 aa, synthetic construct
KAYDQQLKAGDNPYLRYNHADAEFQERLQEDTSFGGNLGRAVFQAKKRVLEPLGLVETPAKTAPGKKRPVDSPDSTSGIGKKGQQPAKKRLNFGQTGDSESVPDPQPIGEPPAGPSGLGSGTMAAGGGAPMADNNEGADGVGNASGNWHCDSTWLGDRVITTSTRTWALPTYNNHLYKQISSQSAGSTNDNVYFGYSTPWGYFDFNRFHCHFSPRDWQRLINNNWGFRPKKLNFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLARTQSNAGGTAGNRELQFYQGGPTTMAEQAKNWLPGPCFRQQRVSKTLDQNNNSNFAWTGATKYHLNRNSLVNPGVAMATHKDDEERFFPSSGVLIFGKTGAANKTTLENVLMTNEEEIRPTNPVATEEYGIVSSNLQAASTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFDKQTGVDFAVDSQGVYSEP

SEQ ID NO: 35 - protein, 735 aa, synthetic construct
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGHQPARKRLNFGQTGDADSVPDPQPLGQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRPSFYCLEYFPSQMLRTGNNFTFSYTFEDVPFHSSYAHSQSLDRLMNPLIDQYLYYLNRTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLEHVMITDEEEIRTTNPVATEQYGNVSNNLQNSNTGPTTENVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 36 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 37 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPRPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 38 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 39 - protein, 734 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLRQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYYLNGRDSLVNPGPAMASHKDDEEKFFPMHGTLIFGKEGTNATNAELENVMITDEEEIRTTNPVATEQYGYVSNNLQNSNTAASTETVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 40 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGNQPARKRLNFGQTGDADSVPDPQPLGQPPASPSGLGTNTMATGSGAPMADNNEGADGVGNSSGNWHCDSTWMGDRVVTTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDLEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTAADNNNSEYSWTGATKYHLNGRDSLVNPGPAMASHKDDEEKFFPQSGVLIFGKQGSEKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 41 - protein, 733 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPIESPDSSTGIGKKGQQPAKKKLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTVSQNNNSNFAWTGATKYHLNGRDSLVNPGVAMATHKGDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRSL

SEQ ID NO: 42 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNCHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 43 - protein, 738 aa, synthetic construct; identical to SEQ ID NO: 25

SEQ ID NO: 44 - protein, 728 aa, synthetic construct
MAADGYLPDWLEGNLSEGIREWWDLKPGAPKPKANQQKQDDGRGLVLPGYRYLGPFNGLDKGEPVNEADAAALEHDKAYDKQLEQGDNPYLKYNHADAEFQERLQEDTSFGGNLGRAVFQAKKRVLEPLGLVEEGAKTAPGKKRPIESPDSSTGIGKNGQPPAKKKLNFGQTGDSESVPDPQPLGEPPAAPSGLGSGTMAAGGGAPMADNNEGADGVGNASGNWHCDSTWLGDRVITTSTRTWALPTYNNHLYKQISSQSGATNDNHFFGYSTPWGYFDFNRFHCHFSPRDWQRLINNNWGFRPRKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGDNFEFSYTFEEVPFHSSYAHSQSLDRLMNPLIDQYLYYLARTQSTTGSTRELQFHQAGPNTMAEQSKNWLPGPCYRQQRLSKNIDSNNNSNFAWTGATKYHLNGRNSLTNPGVAMATNKDDEDQFFPINGVLVFGKTGAANKTTLENVLMTSEEEIKTTNPVATEEYGVVSSNLQSSTAGPQTQTVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYAKSNNVEFAVNNEGVYTEPRPIGTRYLTRNL

SEQ ID NO: 45 - protein, 728 aa, synthetic construct
MAADGYLPDWLEDNLSEGIREWWDLKPGAPKPKANQQKQDDGRGLVLPGYKYLGPFNGLDKGEPVNEADAAALEHDKAYDKQLEQGDNPYLKYNHADAEFQERLQEDTSFGGNLGRAVFQAKKRVLEPLGLVEEGAKTAPGKKRPIESPDSSTGIGKKGQQPAKKKLNFGQTGDSESVPDPQPLGEPPAAPSGLGSGTMAAGGGAPMADNNEGADGVGNASGNWHCDSTWLGDRVITTSTRTWALPTYNNHLYKQISSQSGATNDNHFFGYSTPWGYFDFNRFHCHFSPRDWQRLINNNWGFRPRKLRFKLFNIQVKEVTTNDGVTTIANNLTSTIQVFSDSEYQLPYVLGSAHQGCLPPFPADVFMIPQYGYLTLNNGSQSMGRSSFYCLEYFPSQMLRTGNNFEFSYTFEEVPFHSSYAHSQSLDRLMNPLIDQYLYYLARTQSTTGSTRELQFHQAGPNTMAEQSKNWLPGPCYRQQRLSKNIDSNNNSNFAWTGATKYHLNGRNSLTNPGVAMATNKDDEGQFFPINGVLVFGKTGAANKTTLENVLMTSEEEIKTTNPVATEEYGVVSSNLQSSTAGPQTQTVNSQGALPGMVWQNRDVYLQGPIWAKIPHTDGNFHPSPLMGGFGLKHPPPQILIKNTPVPANPPGVFTPALFASFITQYSTGQVSVEIEWELQKENSKRWNPEIQYTSNYAKSNNVEFAVNNEGVYTEPRPIGTRYLTRNL

SEQ ID NO: 46 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKATNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 47 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGHQPARKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVPGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQTNGAPIVGTVNSQGALPGMVWQNRDVYLQGPIWAKIPHTDGNFHPSPLMGGFGLKHPPPQILVKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 48 - DNA, 1725 bp, synthetic construct
  source 1..1725: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 49 - DNA, 594 bp, synthetic construct
  source 1..594: mol_type = other DNA
cgcgtggtacctctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcgg

SEQ ID NO: 50 - DNA, 132 bp, synthetic construct
  source 1..132: mol_type = other DNA
tatataaggacgcgccgggtgtggcacagctagttccgtcgcagccgggatttgggtcgcagttcttgtttgtggatcgctgtgatcgtcacttggtgagtagcgggctgctgggctggccggggctttcgt

SEQ ID NO: 51 - DNA, 251 bp, synthetic construct
  source 1..251: mol_type = other DNA
atggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatact

SEQ ID NO: 52 - DNA, 212 bp, synthetic construct
  source 1..212: mol_type = other DNA
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 53 - DNA, 448 bp, Homo sapiens
  source 1..448: mol_type = genomic DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 54 - DNA, 429 bp, Homo sapiens
  source 1..429: mol_type = genomic DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgc

SEQ ID NO: 55 - DNA, 477 bp, Homo sapiens
  source 1..477: mol_type = genomic DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcc

SEQ ID NO: 56 - DNA, 469 bp, Homo sapiens
  source 1..469: mol_type = genomic DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgccaattgcagcggaggagtcgtgtcgtgcctgagagcgcag

SEQ ID NO: 57 - DNA, 571 bp, Homo sapiens
  source 1..571: mol_type = genomic DNA
cctgcagggcccactagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagcc

SEQ ID NO: 58 - DNA, 229 bp, Mus musculus
  source 1..229: mol_type = genomic DNA
agctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgc

SEQ ID NO: 59 - DNA, 1675 bp, Homo sapiens
  source 1..1675: mol_type = genomic DNA
gtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagtagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgcgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttcccttttttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgattcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcat

SEQ ID NO: 60 - DNA, 2043 bp, Rattus sp.
  source 1..2043: mol_type = genomic DNA
agctctgagctcctcctctgctcgcccaatccttccaaccccctatggtggtatggctgacacagaaaatgtctgctcctgtatgggacatttgcccctcttctccaaatataagacaggatgaggcctagcttttgctgctccaaagttttaaaagaacacattgcacggcatttagggactctaaagggtggaggaggaatgagggaattgcatcatgccaaggctggtcctcatccatcactgcttccagggcccagagtggcttccaggaagtattcttacaaaggaagcccgatctgtagctaacactcagagcccattttcctgcgttaacccctcccgacctcatatacaggagtaacatgatcagtgacctgggggagctggccaaactgcgggacctgcccaagctgagggccttggtgctgctggacaacccctgtgccgatgagactgactaccgccaggaggccctggtgcagatggcacacctagagcgcctagacaaagagtactatgaggacgaggaccgggcagaagctgaggagatccgacagaggctgaaggaggaacaggagcaagaactcgacccggaccaagacatggaaccgtacctcccgccaacttagtggctcctctagcctgcagggacagtaaaggtgatggcaggaaggcagcccccggaggtcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctgggccagggaaaagatcctgcactaaaaatctgaagctaaaaataacaggacacggggtggagaggcgaaaggagggcagagtgaggcagagagactgagaggcctggggatgtgggcattccggtagggcacacagttcacttgtcttctctttttccaggaggccaaagatgctgacgtcaagaactcataataccccagtggggaccaccgcattcatagccctgttacaagaagtgggagatgttcctttttgtcccagactggaaatccgttacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggggacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatc

SEQ ID NO: 61 - DNA, 60 bp, Homo sapiens
  source 1..60: mol_type = genomic DNA
gaggaggaggagagagaccgggagggcgcccgggaggcagggcgcgcgcacactccgagg

SEQ ID NO: 62 - DNA, 1289 bp, Mus musculus
  source 1..1289: mol_type = genomic DNA
cattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagc

SEQ ID NO: 63 - DNA, 60 bp, Homo sapiens
  source 1..60: mol_type = genomic DNA
cctccgccgctcagccccggactccttacgtcagggtagcggggtcccccctccgcgcgg

SEQ ID NO: 64 - DNA, 1357 bp, Mus musculus
  source 1..1357: mol_type = genomic DNA
cgatcctatcacgagactagcctcgagaagcttgatatcagcacccacatagcagctcacaaatgtctgaaactccaattcttgggaatctgacacgatcacacatgcaggcaaaataccaatgtacatgaattaaaaaaaaaaaaaacaacctttaaaagaaacaagggttcagtaccactactgacatcttgtttccccagaggccttactttaattatttattgtttccacttagttgctcaattaattaatttagaggtttttttcttcctttctttttcttttttctttctctcttttttttcttcttaagacagggtttctctgtgtagctcaggctatcctggaactcactctgtagaccaggctggccttgtactcaaagatctgcctgcctctgcctccccagtgctgggattaaagacatgcaccatcactgccctgctttcctctttttattttgaaaattgttcatcaacagttactaaacgtgttcgaattccaagagctgactagacatataagaccattcagccttctgaataagatgtaggtgtgcccctcctcttactcctctatttggaagttggttactttctgtatgtagtatgcgaatccccctctgccaccccgctttctgttttaaaacagaaaaggctgcaacatacagtgtgtgcttctgttcttgaactggaagcttaggctgtcctggacttgggttgagacctgggctcatccagataggaaatggatttggtgaccccgccaggacttcgcaggcaccacatcgtggtcgtgtgtgggtgctgtatgcacccactgattgcgcgcgtgggttccagagcttggtggtctgcgagaggagagtgggcaagagtgggtgtgtctgtggagccccagctaggggctgctgcccgctgctcccacttgtggctcctgggcgccgccagcaggcacatctccggaggacgccgcgggatgggagctgatgacaggagagcgccgtctcccgagtgatggcagcgcacgctgctgcctcgccgcctccgccgctcagtcctgatcttacgttagggtagctgggtaccccctccgcccgggaaccagctagtagagggagaacagagcagagcgtgcggcagagccgatcccgcgtcccgccgaaccctgccaagccccgccaatcccagcagagcaggaaccagcgcagctgagccaacaccggacgccgcactgagacccagcattccccagccgccactacccggtccccgccggggtgccgggctcgtcctgtgagcccctcgtcatgcgtgtcgggctcttcgactctccagatcagttccagagcgct

SEQ ID NO: 65 - DNA, 61 bp, Homo sapiens
  source 1..61: mol_type = genomic DNA
gggacgcgcgggcggggtgggctgtgccccgcgggaaccccgccggcctgtgcgcttgctg

SEQ ID NO: 66 - DNA, 60 bp, Homo sapiens
  source 1..60: mol_type = genomic DNA
ctccctcccgcgctccccgcgctcgggcgccgcagagctgtccagcttcagtgccgaacc

SEQ ID NO: 67 - DNA, 60 bp, Homo sapiens
  source 1..60: mol_type = genomic DNA
gtactggtgtacaaggacaaggtgactttttttcttttcccagattgaaagggccaaaga

SEQ ID NO: 68 - DNA, 60 bp, Homo sapiens
  source 1..60: mol_type = genomic DNA
ccaggagagctcggcaagtatataaggacagaggagcgcgggaccaagcggcggcgaagg

SEQ ID NO: 69 - DNA, 60 bp, Homo sapiens
  source 1..60: mol_type = genomic DNA
ttccttcagctgtgtcttaaagtaaatcttgttgtggagcggagccctcagctgagggag

SEQ ID NO: 70 - DNA, 60 bp, Homo sapiens
  source 1..60: mol_type = genomic DNA
gtaagtatcttcttcttcccctcgtgagtccctccccttttccagaatcacttgcactgt

SEQ ID NO: 71 - DNA, 60 bp, Homo sapiens
  source 1..60: mol_type = genomic DNA
ggggcggagcggagacagtaccttcggagataatcctttctcctgccgcagtggagagga

SEQ ID NO: 72 - DNA, 60 bp, Homo sapiens
  source 1..60: mol_type = genomic DNA
ccctgcctagtctccatataaaagcggcgccgcctccccgccctctctcactccccgctc

SEQ ID NO: 73 - DNA, 60 bp, Homo sapiens
  source 1..60: mol_type = genomic DNA
gggcggcccagccccaggttacgtcgtccccagaaagaatctggccaacagtctggccgt

SEQ ID NO: 74 - DNA, 99 bp, Homo sapiens
  source 1..99: mol_type = genomic DNA
ggcaggttctcttacatcgaccgcttaagagtcgcgctgtaagaagcaacacctcctcctcgcctccgccatccacccggcagccgcgaagcagcaacc

SEQ ID NO: 75 - DNA, 969 bp, synthetic construct
  source 1..969: mol_type = other DNA
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 76 - DNA, 1050 bp, synthetic construct
  source 1..1050: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 77 - DNA, 1089 bp, synthetic construct
  source 1..1089: mol_type = other DNA
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 78 - DNA, 1430 bp, synthetic construct
  source 1..1430: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagagcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 79 - DNA, 1318 bp, synthetic construct
  source 1..1318: mol_type = other DNA
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtaaaacaggtaagtccgctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctggcctctactaaccatgttcatgttttctttttttttctacaggtcctgggtgacgaacag

SEQ ID NO: 80 - DNA, 1579 bp, synthetic construct
  source 1..1579: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 81 - DNA, 1080 bp, synthetic construct
  source 1..1080: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtctagaggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgtgagtatcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 82 - DNA, 568 bp, synthetic construct
  source 1..568: mol_type = other DNA
gaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 83 - DNA, 895 bp, synthetic construct
  source 1..895: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagagaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 84 - DNA, 1291 bp, synthetic construct
  source 1..1291: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 85 - DNA, 1147 bp, synthetic construct
  source 1..1147: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 86 - DNA, 1490 bp, synthetic construct
  source 1..1490: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagctttggcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 87 - DNA, 969 bp, synthetic construct; identical to SEQ ID NO: 75
  source 1..969: mol_type = other DNA

SEQ ID NO: 88 - DNA, 2074 bp, synthetic construct
  source 1..2074: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcacacatacacgaacacacatacatatacatacattcacatatatgcacacacatacacatatacacgcatacacgtacacacaaatgcacactcacacatgcacacacatacataatatacacactctcacacatgcacatacacacatacatacacatacatgtgcatgcacacacacaaatacacatgcatacatccacattcacacagatgcagacacaaatgcacacacacacacacacacacacacacacacacacacacacacgcacactgccaccctgaactagtggtggctaaatgaacaataagtctccatcaccagcttggggggaggtaggtggtagtgtaggtgcccccattgtgtgatcatgttcattgtatgagtttgtctgtgttcattcatcatagtgacagtccccatgtgggtagcagagagtacgtgtgcatgcatcatctccgtgtttgctcatgagtgtgtatgtcagtgtgttccagtctttctgtgtgagtgtcgtccccaatcccccatccccccccccagatctctaattagtggtttggggtttgttccttttccctcctgttcctttcctcagcagcgcggcagcagcggcggcagcctcggtggtagcagcagcagcagcagcagcagcagcagcagcagcagcagcagcagcaacagaagctgccgcgccgctgagtagcagcaaggactccgagtcaagagtaggattgtaggattggatctgagtgggaacaagagtgagctggcctgagagaggagcagatgcctcccagcgccctcaggccacccattgccagtaatcttcaagccagacctcttgagaggagacgggacagccaaccctagcctacccaggtacagacactgggcagttctgggggactgcccacagatgcctattggattcctggggtatgtaggactcccgggtctaccagcccttttcacctttccccatagcacccccaaggaagctctgacaacttgcccatattcctgtttcccacccgtcccctgggcacccccttttcttctctccctcccagatcccttctttggggagctcagcaaatggagcaggaaatttggaccctctgcctccctctctcgccttcctcattggatccggagtcttctccgctgggaaagctgtaattagagggtggatccctacagacagagagcagcccccccacccccaccccccagtcccttctaactttagatctcttctctcccattctcccattctccctccctctcccttctccctctcccactggctcctcggttctctccatctgcctgactccttgggacccggtccccagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 89 - DNA, 417 bp, synthetic construct
  source 1..417: mol_type = other DNA
ggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccaccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 90 - DNA, 723 bp, synthetic construct
  source 1..723: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 91 - DNA, 460 bp, synthetic construct
  source 1..460: mol_type = other DNA
gggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagat

SEQ ID NO: 92 - DNA, 865 bp, synthetic construct
  source 1..865: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcga

SEQ ID NO: 93 - DNA, 328 bp, synthetic construct
  source 1..328: mol_type = other DNA
ggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagc

SEQ ID NO: 94 - DNA, 770 bp, synthetic construct
  source 1..770: mol_type = other DNA
ctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcac

SEQ ID NO: 95 - DNA, 714 bp, synthetic construct
  source 1..714: mol_type = other DNA
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctatgctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggaccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtcctccctggggacagcccctcctggctagtcacaccctgtaggctcctctatataacccaggggcacaggggctgcccccgggtcac

SEQ ID NO: 96 - DNA, 601 bp, Mus sp.
  source 1..601: mol_type = genomic DNA
ggggtgatgtgtgtcagatctctggattgggggagcttcaaagtgggaaagaaaatggagttcaaatgtggggcttattttccatccctacctggagcccatgactcctcccggctcacctgaccacagggctacctcccctgagcttaagcatcaaggcttagtagtctgagttaagdaacccataaatggggtgcattgtggcaggtcagcaatcgtgtgtccaggtgggcagaactggggagacctttcaaacaggtaaatcttgggaagtacagaccagcagtctgcaaagcagtgacctttggcccagcacagcccttccgtgagccttggagccagttgggaggggcagacagctggggatactctccatatacggcctggtccggtcctagctacctgggccagggccagtcctctccttctttggtcagtgcaggagacccgggcggggacccaggctgagaaccagccgaaggaagggactctagtgcccgacacccaaatatggcttgggaagggcagcaacattccttcggggcggtgtggggagagctcccgggactatataaaaacctgtgcaaggggacaggcggtc

SEQ ID NO: 97 - DNA, 600 bp, Homo sapiens
  source 1..600: mol_type = genomic DNA
agccagcctcagtttcccctccactcagtccctaggaggaaggggcgcccaagcgcgggtttctggggttagactgccctccattgcaattggtccttctcccggcctctgcttcctccagctcacagggtatctgctcctcctggagccacaccttggttccccgaggtgccgctgggactcgggtaggggtgagggcccagggggcacagggggagccgagggccacaggaagggctggtggctgaaggagactcaggggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttccttt

SEQ ID NO: 98 - DNA, 600 bp, Homo sapiens
  source 1..600: mol_type = genomic DNA
ctgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccct

SEQ ID NO: 99 - DNA, 240 bp, Homo sapiens
  source 1..240: mol_type = genomic DNA
ctgcagacatgcttgctgcctgccctggcgtgccctggcgaggcttgccgtcacaggacccccgctggctgactcaggggcgcaggctcttgcgggggagctggcctcccgcccccacggccacgggccctttcctggcaggacagcgggatcttgcagctgtcaggggaggggatgacgggggactgatgtcaggaggggatacaaatagtgccgaacaaggaccggattagatctacc

SEQ ID NO: 100 - DNA, 600 bp, Homo sapiens
  source 1..600: mol_type = genomic DNA
ggagttccaggggcgtaaaggagagggagttcgccttccttcccttcctgagactcaggagtgactgcttctccaatcctcccaagcccaccactccacacgactccctcttcccggtagtcgcaagtgggagtttggggatctgagcaaagaacccgaagaggagttgaaatattggaagtcagcagtcaggcaccttcccgagcgcccagggcgctcagagtggacatggttggggaggcctttgggacaggtgcggttcccggagcgcaggcgcacacatgcacccaccggcgaacgcggtgaccctcgccccaccccatcccctccggcgggcaactgggtcgggtcaggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagc

SEQ ID NO: 101 - DNA, 600 bp, Mus sp.
  source 1..600: mol_type = genomic DNA
agaaacctgtggtctagaggcggggcggggccgatggaggcaacgcacgcccccgcaggcgcccaggccacgccctctgccgcagcattcggtgaaacctgcgttccgagaacttctgaaaactttatctgggggccttcgagaaggctcagacagtaagggtgcatgctgccaatcctgaggagctgagttcgatccctgagaccttcagggtggacagagacggactcccacatgttgttttctgacttctacatgtgtccagtcatacatacacaaatatggaataaacagatggctcatcaggtaagagtgctggctgcttttgcagaggacccaggttcgatttccagaacccacatgtcggctcaaaatcatctgtaattccagttccagggagatccagcactttcttccagggcctccacagacacacataaaataaagataaaaatctccaaaaaatattgttttaataattacaacctgaagaccttgcacaactattcctggctgagaagatggtaagggcgctagctgccaagcttgacagcctgagtttcatctccaagaaccatgaaaactgactcctgggaatta

SEQ ID NO: 102 - DNA, 600 bp, Mus sp.
  source 1..600: mol_type = genomic DNA
ggaagcagaaggccaacattcctcccaagggaaactgaggctcagagttaaaacccaggtatcagtgatatgcatgtgccccggccagggtcactctctgactaaccggtacctaccctacaggcctacctagagactcttttgaaaggatggtagagacctgtccgggctttgcccacagtcgttggaaacctcagcattttctaggcaacttgtgcgaataaaacacttcgggggtccttcttgttcattccaataacctaaaacctctcctcggagaaaatagggggcctcaaacaaacgaaattctctagcccgctttccccaggataaggcaggcatccaaatggaaaaaaaggggccggccgggggtctcctgtcagctccttgccctgtgaaacccagcaggcctgcctgtcttctgtcctcttggggctgtccaggggcgcaggcctcttgcgggggagctggcctccccgccccctcgcctgtggccgcccttttcctggcaggacagagggatcctgcagctgtcaggggaggggcgccggggggtgatgtcaggagggctacaaatagtgcagacagctaaggggctcc

SEQ ID NO: 103 - DNA, 535 bp, synthetic construct
  source 1..535: mol_type = other DNA
tcaaggaggactgtccaggtaaatcactgttctaataatcaggcacaacttgctgttaccaaaaaattaggtttacctataaaggatgaaaaatttctattactggggatattttgaaaaataacctctgaaagtaactgtaaaagtttaagtgttgagaaaaagcattatagttagaatggattgcaacctttgtttttttctatatgcaatgaagttctttttttcaagaaacagttcatgttttggaaagtgaaacctaattcactattaccaaaaaaagaggagcagagggaaattccgtaacttggaggctacaataaataataccttcgagtctgcaacatgggacttccccaggaacagccagcaggttttgctaagtcaactgtaatgcccttatccaatcagaattagggagggaaaatggctttgcagataaatatggcacactagccccacgttttctgagacattcctcaattgcttagacatattctgagcctacagcagaggaacctccagtctcagcacc

SEQ ID NO: 104 - DNA, 626 bp, synthetic construct
  source 1..626: mol_type = other DNA
aaatcaaaagtcctcaacctggttggaagaatattggcactgaatggtatcaataaggttgctagagagggttagaggtgcacaatgtgcttccataacattttatacttctccaatcttagcactaatcaaacatggttgaatactttgtttactataactcttacagagttataagatctgtgaagacagggacagggacaatacccatctctgtctggttcataggtggtatgtaatagatatttttaaaaataagtgagttaatgaatgagggtgagaatggaggcacagaggtattagggggaggtgggccccagagaatggtgccaaggtccagtggggtgactgggatcagctcaggcctgacgctggccactcccacctagctcctttctttctaatctgttctcattctccttgggaaggattgaggtctctggaaaacagccaaacaactgttatgggaacagcaagcccaaataaagccaagcatcagggggatctgagagctgaaagcaacttctgttccccctccctcagctgaaggggtggggaagggctcccaaagccataactccttttaagggatttagaaggcataaaaaggcccctggctgagaact

SEQ ID NO: 105 - DNA, 1513 bp, synthetic construct
  source 1..1513: mol_type = other DNA
agccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagccatg

SEQ ID NO: 106 - DNA, 635 bp, synthetic construct
  source 1..635: mol_type = other DNA
gtcgacagaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagagaattc

SEQ ID NO: 107 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 108 - DNA, 82 bp, synthetic construct
  source 1..82: mol_type = other DNA
gtgagtatctcagggatccagacatggggatatgggaggtgcctctgatcccagggctcactgtgggtctctctgttcacag

SEQ ID NO: 109 - DNA, 127 bp, synthetic construct
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 110 - DNA, 55 bp, synthetic construct
  source 1..55: mol_type = other DNA
ataaaggaaatttattttcattgcaatagtgtgttggaattttttgtgtctctca

SEQ ID NO: 111 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 112 - DNA, 106 bp, synthetic construct
  source 1..106: mol_type = other DNA
ctgcgcgctcgctcgctcactgaggccgcccgggcaaagcccgggcgtcgggcgacctttggtcgcccggcctcagtgagcgagcgagcgcgcagagagggagtgg

SEQ ID NO: 113 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 114 - DNA, 19 bp, synthetic construct
  source 1..19: mol_type = other DNA
tagcaggcatgctggggag

SEQ ID NO: 115 - protein, 20 aa, synthetic construct
MYRMQLLLLIALSLALVTNS

SEQ ID NO: 116 - DNA, 60 bp, synthetic construct
  source 1..60: mol_type = other DNA
atgtataggatgcaactgctcctcctgattgctctgagcctggctcttgtgaccaactct

SEQ ID NO: 117 - DNA, 60 bp, synthetic construct
  source 1..60: mol_type = other DNA
atgtatagaatgcagctgctgctgctgattgctctgtccctggctctggtgaccaactct

SEQ ID NO: 118 - protein, 26 aa, synthetic construct
MNFLLSWVHWSLALLLYLHHAKWSQA

SEQ ID NO: 119 - protein, 29 aa, synthetic construct
MERAAPSRRVPLPLLLLGGLALLAAGVDA

SEQ ID NO: 120 - protein, 19 aa, synthetic construct
MAPLRPLLILALLAWVALA

SEQ ID NO: 121 - protein, 18 aa, synthetic construct
MRLLAKIICLMLWAICVA

SEQ ID NO: 122 - protein, 19 aa, synthetic construct
MRLLAFLSLLALVLQETGT

SEQ ID NO: 123 - protein, 18 aa, synthetic construct
MKWVTFISLLFLFSSAYS

SEQ ID NO: 124 - protein, 18 aa, synthetic construct
MAFLWLLSCWALLGTTFG

SEQ ID NO: 125 - protein, 20 aa, synthetic construct
MYRMQLLSCIALILALVTNS

SEQ ID NO: 126 - protein, 15 aa, synthetic construct
MNLLLILTFVAAAVA

SEQ ID NO: 127 - protein, 17 aa, Homo sapiens
MRAWIFFLLCLAGRALA

SEQ ID NO: 128 - protein, 22 aa, Homo sapiens
MFSFVDLRLLLLLAATALLTHG

SEQ ID NO: 129 - protein, 19 aa, Homo sapiens
MKLVFLVLLFLGALGLCLA

SEQ ID NO: 130 - protein, 22 aa, Homo sapiens
MGPTSGPSLLLLLLTHLPLALG

SEQ ID NO: 131 - protein, 18 aa, Homo sapiens
MSLSAFTLFLALIGGTSG

SEQ ID NO: 132 - protein, 27 aa, Homo sapiens
MAPHRPAPALLCALSLALCALSLPVRA

SEQ ID NO: 133 - protein, 22 aa, Homo sapiens
MWATLPLLCAGAWLLGVPVCGA

SEQ ID NO: 134 - protein, 19 aa, Homo sapiens
MQALVLLLCIGALLGHSSC

SEQ ID NO: 135 - protein, 23 aa, Homo sapiens
MQMSPALTCLVLGLALVFGEGSA

SEQ ID NO: 136 - protein, 20 aa, Homo sapiens
MQPSSLLPLALCLLAAPASA

SEQ ID NO: 137 - protein, 23 aa, Homo sapiens
MAPFEPLASGILLLLWLIAPSRA

SEQ ID NO: 138 - protein, 31 aa, Homo sapiens
MLRGPGPGLLLLAVQCLGTAVPSTGASKSKR

SEQ ID NO: 139 - protein, 15 aa, Homo sapiens
MWCIVLFSLLAWVYA

SEQ ID NO: 140 - protein, 17 aa, Homo sapiens
MNPTLILAAFCLGIASA

SEQ ID NO: 141 - protein, 17 aa, Homo sapiens
MWQLWASLCCLLVLANA

SEQ ID NO: 142 - protein, 16 aa, Homo sapiens
MLLILLSVALLAFSSA

SEQ ID NO: 143 - protein, 20 aa, Homo sapiens
MWKRWLALALALVAVAWVRA

SEQ ID NO: 144 - protein, 18 aa, Homo sapiens
MKWVTFISLLFLFSSAYS

SEQ ID NO: 145 - protein, 24 aa, Homo sapiens
MPSSVSWGILLLAGLCCLVPVSLA

SEQ ID NO: 146 - protein, 18 aa, Homo sapiens
MKAAVLTLAVLFLTGSQA

SEQ ID NO: 147 - protein, 18 aa, Homo sapiens
MKLLAATVLLLTICSLEG

SEQ ID NO: 148 - protein, 27 aa, Homo sapiens
MDPPRPALLALLALPALLLLLLAGARA

SEQ ID NO: 149 - protein, 28 aa, Homo sapiens
MQRVNMIMAESPGLITICLLGYLLSAEC

SEQ ID NO: 150 - protein, 20 aa, Homo sapiens
MGPLMVLFCLLFLYPGLADS

SEQ ID NO: 151 - protein, 18 aa, Homo sapiens
MWLLVSVILISRISSVGG

SEQ ID NO: 152 - protein, 18 aa, Homo sapiens
MLLLFSVILISWVSTVGG

SEQ ID NO: 153 - protein, 19 aa, Homo sapiens
MFSMRIVCLVLSVVGTAWT

SEQ ID NO: 154 - protein, 30 aa, Homo sapiens
MKRMVSWSFHKLKTMKHLLLLLLCVFLVKS

SEQ ID NO: 155 - protein, 26 aa, Homo sapiens
MSWSLHPRNLILYFYALLFLSSTCVA

SEQ ID NO: 156 - protein, 18 aa, Homo sapiens
MKSLVLLLCLAQLWGCHS

SEQ ID NO: 157 - protein, 23 aa, Homo sapiens
MARVLGAPVALGLWSLCWSLAIA

SEQ ID NO: 158 - protein, 18 aa, Homo sapiens
MKLITILFLCSRLLLSLT

SEQ ID NO: 159 - protein, 20 aa, Homo sapiens
MSLFPSLPLLLLSMVAASYS

SEQ ID NO: 160 - protein, 19 aa, Homo sapiens
MEHKEVVLLLLLFLKSGQG

SEQ ID NO: 161 - protein, 24 aa, Homo sapiens
MAHVRGLQLPGCLALAALCSLVHS

SEQ ID NO: 162 - protein, 29 aa, Homo sapiens
MISRMEKMTMMMKILIMFALGMNYWSCSG

SEQ ID NO: 163 - protein, 32 aa, Homo sapiens
MYSNVIGTVTSGKRKVYLLSLLLIGFWDCVTC

SEQ ID NO: 164 - protein, 19 aa, Homo sapiens
MRLAVGALLVCAVLGLCLA

SEQ ID NO: 165 - DNA, 2854 bp, synthetic construct
  source 1..2854: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtatagaatgcagctgctgctgctgattgctctgtccctggctctggtgaccaactctgtgagatcctcctcaagaaccccttctgataaacctgtggctcatgtggtggctaaccctcaggcagaaggacagctgcagtggctgaataggagggccaatgcactgctggctaatggagtggaactgagggacaaccagctggtggtcccttcagaaggactgtacctgatctatagccaggtgctgttcaagggccagggatgtcccagtactcatgtgctgctgactcacaccatctccagaattgctgtctcccatcagacaaaggtgaacctgctgtcagccatcaaaagtccctgccagagagagacaccagaaggagctgaggccaagccatggtatgagccaatctacctggggggagtgtttcagctggagaaaggagacaggctgagtgcagaaatcaacagaccagactatctggactttagagaatcagggcaggtgtattttggcattattgcactgtgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 166 - DNA, 2270 bp, synthetic construct
  source 1..2270: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtatagaatgcagctgctgctgctgattgctctgtccctggctctggtgaccaactctgtgagatcctcctcaagaaccccttctgataaacctgtggctcatgtggtggctaaccctcaggcagaaggacagctgcagtggctgaataggagggccaatgcactgctggctaatggagtggaactgagggacaaccagctggtggtcccttcagaaggactgtacctgatctatagccaggtgctgttcaagggccagggatgtcccagtactcatgtgctgctgactcacaccatctccagaattgctgtctcccatcagacaaaggtgaacctgctgtcagccatcaaaagtccctgccagagagagacaccagaaggagctgaggccaagccatggtatgagccaatctacctggggggagtgtttcagctggagaaaggagacaggctgagtgcagaaatcaacagaccagactatctggactttagagaatcagggcaggtgtattttggcattattgcactgtga

SEQ ID NO: 167 - DNA, 2433 bp, synthetic construct
  source 1..2433: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtatagaatgcagctgctgctgctgattgctctgtccctggctctggtgaccaactctgtgagatcctcctcaagaaccccttctgataaacctgtggctcatgtggtggctaaccctcaggcagaaggacagctgcagtggctgaataggagggccaatgcactgctggctaatggagtggaactgagggacaaccagctggtggtcccttcagaaggactgtacctgatctatagccaggtgctgttcaagggccagggatgtcccagtactcatgtgctgctgactcacaccatctccagaattgctgtctcccatcagacaaaggtgaacctgctgtcagccatcaaaagtccctgccagagagagacaccagaaggagctgaggccaagccatggtatgagccaatctacctggggggagtgtttcagctggagaaaggagacaggctgagtgcagaaatcaacagaccagactatctggactttagagaatcagggcaggtgtattttggcattattgcactgtgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 168 - DNA, 1568 bp, synthetic construct
  source 1..1568: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcacgcgtggtaccagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtctagacaggtgagtatctcagggatccagacatggggatatgggaggtgcctctgatcccagggctcactgtgggtctctctgttcacaggtttctagaactagtagtaattcgccaccatgtatagaatgcagctgctgctgctgattgctctgtccctggctctggtgaccaactctgtgagatcctcctcaagaaccccttctgataaacctgtggctcatgtggtggctaaccctcaggcagaaggacagctgcagtggctgaataggagggccaatgcactgctggctaatggagtggaactgagggacaaccagctggtggtcccttcagaaggactgtacctgatctatagccaggtgctgttcaagggccagggatgtcccagtactcatgtgctgctgactcacaccatctccagaattgctgtctcccatcagacaaaggtgaacctgctgtcagccatcaaaagtccctgccagagagagacaccagaaggagctgaggccaagccatggtatgagccaatctacctggggggagtgtttcagctggagaaaggagacaggctgagtgcagaaatcaacagaccagactatctggactttagagaatcagggcaggtgtattttggcattattgcactgtgataactcgagagcggccatcaagcttgatctttttccctctgccaaaaattatggggacatcatgaagccccttgagcatctgacttctggctaataaaggaaatttattttcattgcaatagtgtgttggaattttttgtgtctctcactcggctagcgaagcaattctagcaggcatgctggggagagatcgatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 169 - DNA, 1102 bp, synthetic construct
  source 1..1102: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtctagacaggtgagtatctcagggatccagacatggggatatgggaggtgcctctgatcccagggctcactgtgggtctctctgttcacaggtttctagaactagtagtaattcgccaccatgtatagaatgcagctgctgctgctgattgctctgtccctggctctggtgaccaactctgtgagatcctcctcaagaaccccttctgataaacctgtggctcatgtggtggctaaccctcaggcagaaggacagctgcagtggctgaataggagggccaatgcactgctggctaatggagtggaactgagggacaaccagctggtggtcccttcagaaggactgtacctgatctatagccaggtgctgttcaagggccagggatgtcccagtactcatgtgctgctgactcacaccatctccagaattgctgtctcccatcagacaaaggtgaacctgctgtcagccatcaaaagtccctgccagagagagacaccagaaggagctgaggccaagccatggtatgagccaatctacctggggggagtgtttcagctggagaaaggagacaggctgagtgcagaaatcaacagaccagactatctggactttagagaatcagggcaggtgtattttggcattattgcactgtga

SEQ ID NO: 170 - DNA, 1254 bp, synthetic construct
  source 1..1254: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtctagacaggtgagtatctcagggatccagacatggggatatgggaggtgcctctgatcccagggctcactgtgggtctctctgttcacaggtttctagaactagtagtaattcgccaccatgtatagaatgcagctgctgctgctgattgctctgtccctggctctggtgaccaactctgtgagatcctcctcaagaaccccttctgataaacctgtggctcatgtggtggctaaccctcaggcagaaggacagctgcagtggctgaataggagggccaatgcactgctggctaatggagtggaactgagggacaaccagctggtggtcccttcagaaggactgtacctgatctatagccaggtgctgttcaagggccagggatgtcccagtactcatgtgctgctgactcacaccatctccagaattgctgtctcccatcagacaaaggtgaacctgctgtcagccatcaaaagtccctgccagagagagacaccagaaggagctgaggccaagccatggtatgagccaatctacctggggggagtgtttcagctggagaaaggagacaggctgagtgcagaaatcaacagaccagactatctggactttagagaatcagggcaggtgtattttggcattattgcactgtgataactcgagagcggccatcaagcttgatctttttccctctgccaaaaattatggggacatcatgaagccccttgagcatctgacttctggctaataaaggaaatttattttcattgcaatagtgtgttggaattttttgtgtctctcactcg

SEQ ID NO: 171 - protein, 20 aa, unidentified
MYRMQLLLLIALSLALVTNS

SEQ ID NO: 172 - protein, 7 aa, synthetic construct
LGETTRP

SEQ ID NO: 173 - protein, 9 aa, synthetic construct
LALGETTRP

SEQ ID NO: 174 - protein, 7 aa, synthetic construct
TLAAPFK

SEQ ID NO: 175 - protein, 7 aa, synthetic construct
TLAVPFK

SEQ ID NO: 176 - skipped

SEQ ID NO: 177 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 178 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 179 - protein, 734 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 180 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 181 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 182 - protein, 737 aa, synthetic construct
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 183 - DNA, 143 bp, synthetic construct
  source 1..143: mol_type = other DNA
gaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 184 - DNA, 143 bp, synthetic construct
  source 1..143: mol_type = other DNA
ttgcccactccctctctgcgcgctcgctcgctcggtggggcctgcggaccaaaggtccgcagacggcagagctctgctctgccggccccaccgagcgagcgagcgcgcagagagggagtgggcaactccatcactaggggtaa

SEQ ID NO: 185 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 186 - DNA, 143 bp, synthetic construct
  source 1..143: mol_type = other DNA
ttggccactccctctatgcgcactcgctcgctcggtggggcctggcgaccaaaggtcgccagacggacgtgctttgcacgtccggccccaccgagcgagcgagtgcgcatagagggagtggccaactccatcactagaggtat

SEQ ID NO: 187 - DNA, 146 bp, synthetic construct
  source 1..146: mol_type = other DNA
ttggccactccctctatgcgcgctcgctcactcactcggccctggagaccaaaggtctccagactgccggcctctggccggcagggccgagtgagtgagcgagcgcgcatagagggagtggccaactccatcatctaggtttgccc

SEQ ID NO: 188 - DNA, 145 bp, synthetic construct
  source 1..145: mol_type = other DNA
ttggccactccctctatgcgcgctcgctcgctcggtggggcctgcggaccaaaggtccgcagacggcagagctctgctctgccggccccaccgagcgagcgagcgcgcatagagggagtggccaactccatcactaggggtaccg

SEQ ID NO: 189 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa